SEQUENCE LISTING (293 sequences)

SEQ ID NO: 1 - protein, 4 aa, synthetic construct
GDIR

SEQ ID NO: 2 - protein, 4 aa, synthetic construct
GDIK

SEQ ID NO: 3 - protein, 4 aa, synthetic construct
VDTA

SEQ ID NO: 4 - protein, 4 aa, synthetic construct
REKR

SEQ ID NO: 5 - protein, 9 aa, synthetic construct
RRVVEREKR

SEQ ID NO: 6 - protein, 9 aa, synthetic construct
ERVVEREKE

SEQ ID NO: 7 - protein, 4 aa, synthetic construct
SEKS

SEQ ID NO: 8 - protein, 79 aa, synthetic construct
MRVMGIQRNYPQWWIWSMLGFWMLMICNGMWVTVYYGVPVWKEAKTTLFCASDAKAYEKEVHNVWATHACVPTDPNPQE

SEQ ID NO: 9 - protein, 24 aa, synthetic construct
MPMGSLQPLATLYLLGMLVASVLA

SEQ ID NO: 10 - protein, 5 aa, synthetic construct
  VARIANT 3: note = X signifies any amino acid but most commonly Ala, Ser, Glu, or a N-terminal pentaglycine repeat tag is added to the envelope trimer gene
LPXTG

SEQ ID NO: 11 - DNA, 1947 bp, synthetic construct
  source 1..1947: mol_type = other DNA
atgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgaacacctctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggactgataa

SEQ ID NO: 12 - DNA, 1932 bp, synthetic construct
  source 1..1932: mol_type = other DNA
atgggatctctgcagcctctggccacactgtacctgctgggaatgctggtggcctcttgtctgggcgttgagaagctgtgggtcaccgtgtactatggcgtgcccgtgtggaaagaagcctgcaccacactgttctgtgccagcgacgccaaggcctacgataccaaggtgcgcaatgtgtgggccactcactgctgcgtgcccaccgatcctaatcctcaagaggtggtgctggaaaacgtgaccgagcacttcaacatgtggaagaacaacatggtcgagcagatgcaagaggacatcatcagcctgtgggaccagagcctgaagccttgcgtgaagctgacccctctgtgcgtgaccctgaactgtagcaccgccaccgtgaacaacagagccgtggacgagatgaagaactgcagcttcaacatcaccacctccatcagagacaaggtgcagaaagagtacgccctgttctacaagctggacgtggtgcccatcgacaacaacaacaccagctacagactgatcagctgcgacaccagcgtgatcacccaggcctgtcctaagatcagcttcgagcccattcctatccactactgtgcccctgccggcttcgccatcctgaagtgcaacgacaagaccttcaacggcaagggcccctgcaagaacgtgtccaccgtgcagtgtacccacggcatcagaccagtggtgtctacccagctgctgctgaatggctctctggccgaggaagaagtggtcatcagaagcgacaacttcaccaacaacgccaagaccatcatcgtgcagctgaaagagagcgtcgagatcaactgcacccggcctaacaacaatacccggaagtccatccacatcggccctggcagatggttttacaccaccggcgagattatcggcgacatcagacaggcccactgcaacatcagccgggccaagtggaacgacaccctgaagcagatcgtgatcaagctgagagagcagttcgagaacaagacgatcgtgttcaaccacagctctggcggcgaccccgagattgtgatgcactcctttaactgtggcggcgagttcttctactgcaacagcacccagctgttcaactccacctggaacaacaacacagagggcagcaacaataccgagggcaacaccatcacactgccctgccggatcaagcagatcatcaatatgtggcaagagatcggcaaggctatgtacgcccctcctatccggggccagatcagatgcagcagcaatatcacaggcctgctgctcaccagagatggcggcatcaacgagaacggcaccgagatcttcagacccggcggaggcgacatgcgggacaattggagaagcgagctgtacaagtacaaggtggtcaagatcgagcccctgggcgtcgcccctaccaagtgcaagagaagagtcgtgggccgtagaaggcggcggagagctgttggaattggcgccgtgttcctgggctttctgggagccgctggatctacaatgggcgctgccagcatgaccctgacagtgcaggctagacagctgctgtccggcattgtccagcagcagaacaactgcctgagagcccctgagtgtcaacaaagaatgctgcagctgaccgtgtggggcatcaaacagctgcaggccagagttctggccgtggaaagatacctgggcgaccagcagctcctcggcatctggggatgttctggcaagctgatttgctgcaccgccgtgccttggaatgccagctggtctaacaagagcctggaccggatctggaacaatatgacctggatggaatgggagcgcgagatcgacaactacacctccgagatctacaccctgatcgaggaaagccagaaccagcaagagaaaaacgagcaagagctgctcgagctggattaatga

SEQ ID NO: 13 - DNA, 1944 bp, synthetic construct
  source 1..1944: mol_type = other DNA
atgggctcgctccagccgctcgcgacgctgtacctcctgggcatgctcgtggcgtccgtgctggcggccgagaacctgtgggtgacggtgtactacggcgtgcccgtgtggaaggaggccaacaccacgctgttctgcgccagcgacgccaaggcctacgacaccgaggtgcacaacgtgtgggcgacccacgcctgcgtgccgacggaccccaacccccaggagatcgtgctggagaacgtgaccgagaacttcaacatgtggaagaacaacatggtggagcagatgcacgaggacatcatctcgctgtgggaccagtccctgaagccgtgcgtgaagctgacgcccctgtgcgtgaccctgaactgtagcaccgccaccgtgaacaacagagccgtggacgagatgaagaactgctccttcaacatcaccaccgagatccgcgacaagaagcagaaggtgtacgcgctgttctaccggctggacgtggtgccgatcgacgacaacaacaacaactccagcaactaccgcctgatcaactgcaacaccagcgcctgcacccaggcctgcccgaaggtgtccttcgagcccatccccatccactactgcgcgccggccggcttcgccatcctgaagtgcaacgacaagaagttcaacggcaccggcccctgcaagaacgtgtccaccgtgcagtgcacccacgggatcaagcccgtggtgtccacgcagctgctgctgaacggctccctggccgaggaggagatcatcatccgctccgagaacatcacgaacaacgccaagaccatcatcgtgcagctgaacgagtccgtggagatcaactgcaccaggcccaacaacaacacccgcaagtccatccggatcggccctggccaggcgttctacgccaccggcgacatcatcggcgacatccgccaggcgcactgcaacatctcgggcacgaagtggaacaagaccctgcagcaggtggcgaagaagctgcgcgagcacttcaacaacaagaccatcatcttcaagcccagctccggcggcgacctggagatcacgacccactccttcaactgccgcggcgagttcttctactgcaacacctccggcctgttcaactcgacgtggatcgggaacggcacgaagaacaacaacaacaccaacgacaccatcaccctgccctgccgcatcaagcagatcatcaacatgtggcagggcgtgggccagtgcatgtacgcgccgcccatcgagggcaagatcacctgcaagtccaacatcaccggcctgctcctgacgcgcgacggcggcaacaacaacaccaacgagaccgagatcttcaggccgggcggcggcgacatgcgcgacaactggcgctcggagctgtacaagtacaaggtggtgaagatcgagcccctgggcgtggcgccgacgcgctgcaagagacgcgtggtgggccgcagacgaaggagacgggccgtgggcatcggcgcggtgttcctgggcttcctgggagcagctggttcgacgatgggcgcagcttccatgaccctgacagtgcaggcacgcaacctgctctccggcatcgtccagcagcagtcgaacctgcttcgagcccccgaggcgcagcagcacctcctcaagctgaccgtgtggggcatcaagcagctgcaggcacgcgtgctagccgtggagcgctacctccgcgaccagcagctgctcggaatctggggctgctcgggcaagctgatctgctgcaccaacgtgccgtggaacagctcctggtccaaccgcaacctctcggagatctgggacaacatgacctggctccagtgggacaaggagatctcgaactacacccagatcatctacggcctgctggaggagtcccagaaccagcaggagaagaacgagcaggacctgctggccctggactgataa

SEQ ID NO: 14 - DNA, 1947 bp, synthetic construct
  source 1..1947: mol_type = other DNA
atgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggactgataa

SEQ ID NO: 15 - DNA, 2559 bp, synthetic construct
  source 1..2559: mol_type = other DNA
atgagagtgacggggatactgaggaattatccacaatggtggatatggggcatcttaggcttttggatgctaatgacttgtaatggggaaggaaacttgtgggtcacagtctactatggggtaccagtatggaaagaagcaaaaactactctgttttgtgcatcagatgccaaagcgtataagaaagaagtgcataatgtctgggcgacacatgcctgtgtacccacagaccccagcccacaagaactgtttttggaaaatgtaacagaaaattttaacatgtggaaaaatgatatggtagatcagatgcatgaggatataatcagtctatgggatcaaagcctaaagccatgtgtaaagttgaccccactctgtgtcactttaatttgtagtactgctactgttaacaatagagcagttgatgaaatgaaaaattgctccttcaatacaaccacagaaataagagataagaaaaagaaggaatatgcacttttttatagatctgatgtagtaccacttgatgaaacaaacaataccagtgagtatagattaataaattgtaatacctcagccgtaacacaagcctgtccaaaggtcacttttgaaccaattcctatacattattgtgctccagctggttatgcgattctaaagtgcaatgatgagacattcaatggaacaggaccatgcagtaatgtcagcacagtacaatgtacacatggaattaggccagtggtatcaacccaactactgttaaatggtagtctggcagaaaaagagatagtaattagatctgaaaacctgacaaacaatgccaaaataataatagtccatcttcacacccctgtagaaattgtgtgtacaaggcccggccataatacaaggaaaagtgtgagaataggcccaggacaaacattctatgcaacaggagacataataggagatataagacaagcacattgtaacattaatgaaagtaaatggaatgaaactttacaaaaggtaggtatagaattgcaaaaacacttccctaataagacaataaagtataaccaatccgcaggaggagacatggaaattacaacacatagctttaattgtggaggagaatttttctattgcaatacatcaaagctgtttaatagtacatacaatggtacatacataagtacaaacagcacaaacagtacttcatacatcacgcttcaatgcagaataaaacaaattataaacatgtggcagggggtaggaagagcaatgtatgctcctcccattgcaggaaacataacatgtagatcaaatatcacagggctactattgacacgtgatggagggatcaataatgttagcaacgagacagagacattcaggcctgcaggaggagatatgagggataattggagaagtgaattatataaatataaagtagtagaagttcagccattaggaatagcaccaactggtgcaaaaaggagagtggtggagagagaaaaaagagcagcaggactaggagctttgttccttgggttcttgggagcagcaggaagcactatgggcgcagcatcaataacgctgacggtacaggccagacaattgttgtctggtatagtgcaacagcaaagcaatttgctgagggctatagaggcgcaacagcatatgttgcaactcacggtctggggcatcaagcagctccaggcaagagtcctggctctggaaagatacctaaaggatcaacagctcctagggatgtggggctgctctggaaaactcatctgcaccactaatgtgccttggaacactagttggagtaataaatctgaaatggatatttggaataacatgacatggatgcagtgggaaagagaaattagcaattacacagagacaatatacatgttgcttgaagactcgcaacgccagcaggagagaaatgaaaaagatttactagcattggacagttggaacagtctgtggaattggtttaacataacaaactggctgtggtatataaaaatattcataatgatagtagggggcttgataggtttaagaatagtttttgctgtgctatctatagtgaatagagtcaggcagggatactcacctttgtcgttgcagacccttaccccaaacccgagggaacccgacaggctcagaggaatcgaagaagaaggtggagagcaagacagagacagatccattcgattagtgagcggattcttgccaattgtctgggacgacctgcggagcctgtgcctcttcagttaccaccgattgagagactttctattgctggcagcgagagtggtggaacttctgggacacagcagtctcaggggactgcagagggggtgggaagtccttaagtatctgggaagtcttgtgcagtattggggtctggaactaaaaaggagtgctattagtctttttgataccctagcaatagcagtagctgaaggaacagataggattatagaattaatacaaggattttgtagagctatccgcaacatacctacaagaataagacaaggctttgaagcatctttgctataataa

SEQ ID NO: 16 - DNA, 1947 bp, synthetic construct
  source 1..1947: mol_type = other DNA
atgggatctctgcagcctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcacacaggcttgccccaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcaccagacctggccacaacacccggaagtctgtgcggattggacccggccagaccttttatgccaccggcgatatcatcggcgacatcagacaggcccactgtaacatcaacgagagcaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagatgcatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggactgataa

SEQ ID NO: 17 - DNA, 1947 bp, synthetic construct
  source 1..1947: mol_type = other DNA
atgggatctctgcagcctctggccacactgtacctgctgggaatgctggtggcttctgtgctggccgccgagaatctgtgggtcacagtgtactatggcgtgcccgtgtggaaagaggcctgtaccacactgttctgtgccagcgacgccaaggcctacaagaaaaaggtgcgcaacgtctgggccacacactgctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgacacaggcttgccccaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcaccagacctggccacaacacccggaagtctgtgcggattggacccggccagtggttttatgccaccggcgatatcatcggcgacatcagacaggcccactgtaacatcaacgagagcaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagagctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaattgcctcagagcccctgagtgtcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggactgataa

SEQ ID NO: 18 - protein, 647 aa, synthetic construct
MGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 19 - protein, 642 aa, synthetic construct
MGSLQPLATLYLLGMLVASCLGVEKLWVTVYYGVPVWKEACTTLFCASDAKAYDTKVRNVWATHCCVPTDPNPQEVVLENVTEHFNMWKNNMVEQMQEDIISLWDQSLKPCVKLTPLCVTLNCSTATVNNRAVDEMKNCSFNITTSIRDKVQKEYALFYKLDVVPIDNNNTSYRLISCDTSVITQACPKISFEPIPIHYCAPAGFAILKCNDKTFNGKGPCKNVSTVQCTHGIRPVVSTQLLLNGSLAEEEVVIRSDNFTNNAKTIIVQLKESVEINCTRPNNNTRKSIHIGPGRWFYTTGEIIGDIRQAHCNISRAKWNDTLKQIVIKLREQFENKTIVFNHSSGGDPEIVMHSFNCGGEFFYCNSTQLFNSTWNNNTEGSNNTEGNTITLPCRIKQIINMWQEIGKAMYAPPIRGQIRCSSNITGLLLTRDGGINENGTEIFRPGGGDMRDNWRSELYKYKVVKIEPLGVAPTKCKRRVVGRRRRRRAVGIGAVFLGFLGAAGSTMGAASMTLTVQARQLLSGIVQQQNNCLRAPECQQRMLQLTVWGIKQLQARVLAVERYLGDQQLLGIWGCSGKLICCTAVPWNASWSNKSLDRIWNNMTWMEWEREIDNYTSEIYTLIEESQNQQEKNEQELLELD

SEQ ID NO: 20 - protein, 646 aa, synthetic construct
MGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCSTATVNNRAVDEMKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 21 - protein, 647 aa, synthetic construct
MGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 22 - protein, 851 aa, synthetic construct
MRVTGILRNYPQWWIWGILGFWMLMTCNGEGNLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPGHNTRKSVRIGPGQTFYATGDIIGDIRQAHCNINESKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVEVQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSEMDIWNNMTWMQWEREISNYTETIYMLLEDSQRQQERNEKDLLALDSWNSLWNWFNITNWLWYIKIFIMIVGGLIGLRIVFAVLSIVNRVRQGYSPLSLQTLTPNPREPDRLRGIEEEGGEQDRDRSIRLVSGFLPIVWDDLRSLCLFSYHRLRDFLLLAARVVELLGHSSLRGLQRGWEVLKYLGSLVQYWGLELKRSAISLFDTLAIAVAEGTDRIIELIQGFCRAIRNIPTRIRQGFEASLL

SEQ ID NO: 23 - protein, 647 aa, synthetic construct
MGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPGHNTRKSVRIGPGQTFYATGDIIGDIRQAHCNINESKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 24 - protein, 647 aa, synthetic construct
MGSLQPLATLYLLGMLVASVLAAENLWVTVYYGVPVWKEACTTLFCASDAKAYKKKVRNVWATHCCVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPGHNTRKSVRIGPGQWFYATGDIIGDIRQAHCNINESKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNCLRAPECQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 25 - protein, 6 aa, synthetic construct
LPSTGG

SEQ ID NO: 26 - protein, 5 aa, synthetic construct
LPSTG

SEQ ID NO: 27 - protein, 655 aa, synthetic construct
MPMGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDLPSTGG

SEQ ID NO: 28 - DNA, 1989 bp, synthetic construct
  source 1..1989: mol_type = other DNA
gtcgacgccaccatgcccatgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacctgcctagcaccggaggatgataaggatcc

SEQ ID NO: 29 - protein, 30 aa, synthetic construct
CVKLTPLCVTLICSDATVKTGTVEEMKNCS

SEQ ID NO: 30 - protein, 30 aa, synthetic construct
CVKLTPLCVTLICSNATVKNGTVEEMKNCS

SEQ ID NO: 31 - protein, 30 aa, synthetic construct
CVKLTPLCVTLICSTATVNNRAVDEMKNCS

SEQ ID NO: 32 - protein, 44 aa, synthetic construct
VEINCTRPNNNTRKSIHIGPGRAFYTTGEIIGDIRQAHCNISRA

SEQ ID NO: 33 - protein, 44 aa, synthetic construct
VEIVCTRPNNNTRKSVRIGPGQTFYATGDIIGDIKQAHCNISEE

SEQ ID NO: 34 - protein, 44 aa, synthetic construct
VEIVCTRPGHNTRKSVRIGPGQTFYATGDIIGDIRQAHCNINES

SEQ ID NO: 35 - protein, 43 aa, synthetic construct
NCSFNTTTEIRDKEKKEYALFYKPDIVPLSETNNTSEYRLINC

SEQ ID NO: 36 - protein, 43 aa, synthetic construct
NCSFNTTTEIRDKKKKEYALFYRSDVVPLDETNNTSEYRLINC

SEQ ID NO: 37 - DNA, 2445 bp, synthetic construct
  source 1..2445: mol_type = other DNA
gtcgacgccaccatgcctatgggatctctgcagcctctggccacactgtacctgctgggaatgctggtggctagcgtgctggccaagggaaagctgtgggtcacagtgtactacggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcagacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtggaaatccagccactgggaatcgccccaacaggctgcaagagaagagtggttgaaggcggcggaggatctggcggaggcggatctgcagttggaatcggagctgtgttcctgggctttctgggagccgccggatctacaatgggagctgccagcatgaccctgaccgtgcaggctagaaatctgctgagcggcaaccccgactggctgcctgatatgacagtgtggggcatcaagcagctgcaggccagagtgctggccgtggaaagatacctgagagatcagcagctcctcggaatctggggctgtagcggcaagctgatctgctgcaccaacgtgccctggaacagctcctggtccaacagaaacctgtccgagatctgggataacatgacctggctgcagtgggacaaagagatctccaactacacccagatcatctacggcctgctggaagagagccagaaccagcaagagaaaaacgagcaggacctgctggccctggatggcggaggaagcggagatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagatcgagctgatcggcaacgagaaccacggcctgtatctggccgaccagtacgtgaagggcattgccaagagccggaagtcctgatgaggatcc

SEQ ID NO: 38 - DNA, 2472 bp, synthetic construct
  source 1..2472: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggcctgcaccacactgttttgcgcctccgatgccagagcctacgagaagaaagtgcacaacgtgtgggccacacactgctgcgtgccaaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcacacaggcttgccccaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcacctgaataccagcgtcgagatcgtgtgcacccggcctaacaacaacacccggaagtctgtgcggatcggccctggccagtggttttatgccaccggcgatatcatcggcgacatcagacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaattgtctgagagcccctgagtgccagcagcatctgctgaaactgaccgtgtggggcatcaagcagctgcaggcaagagtgctggcagtggaaagatacctgcgggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcgacattatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaggtgctgttcaaggacatcctggacaagattgagctgatcggcaacgagaaccacggcctgtatctggccgaccagtacgtgaagggaatcgccaagagcagaaagagctgatgaggatcc

SEQ ID NO: 39 - DNA, 2472 bp, synthetic construct
  source 1..2472: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtggccaacgtttgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgccgtgacacaggcttgccccaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcacctgaataccagcgtcgagatcgtgtgcacccggcctaacaacaacacccggaagtctgtgcggatcggccctggccagacattctatgccaccggcgatatcatcggcgacatcagacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtgggcagagctatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggccagagtgctgacagtggaaagatacgccagggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggctctggatggcggaggaagcggagatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 40 - DNA, 2472 bp, synthetic construct
  source 1..2472: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacatctgggccacacacgcctgcgtgccaaccgatccatctcctcaagaactggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgccgtgacacaagtgtgccccaagctgaccttcgagcccattcctatccactactgtgcccctgccggctacgccatcctgaagtgcaacaacgagacattcaacggcacaggcccctgcagcaatgtgtccaccgtgcagtgtacccacggcatcagacctgtgctgagcacacagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcagacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtgggcagagctatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagcagctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcgacattatcaagctgctgaacgagcaagtgaacaaagaaatgaacagctccaacctgtacatgagcatgtccagctggtgctacacccacagtcttgatggcgccggactgttcctgtttgaccacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 41 - DNA, 2472 bp, synthetic construct
  source 1..2472: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacatctgggccacacacgcctgcgtgccaaccgatccatctcctcaagaactggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacaagcgccgtgacaatggtctgccccaagctgaccttcgagcccattcctatccactactgtgcccctgccggctacgccatcctgaagtgcaacaacgagacattcaacggcacaggcccctgcagcaatgtgtccaccgtgcagtgtacccacggcatcagacctgtgctgagcacacagctgctgctgaatggaagcctggccgagaaagaaatcgtgatcagaagcgagaacctgaccaacaacgccaagatcatcatcgtccacctgaacacctccgtggaaatcgtgtgcacccggcctctgaatctgaccagaaagagcgtgcggatcggccctggccagaccttttatgccatgggcgacatcatcggcgacatccggcaggcccactgcaacatctctgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagatgatcattaacatgtggcaaggcgtcggcagggctatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagcagctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcgatatcatcaagctgctgaacgagcaagtgaacaaagaaatgaacagctccaacctgtacatgagcatgtccagctggtgctacacccacagtcttgatggcgccggactgttcctgtttgaccacgccgccgaggaatacgagcacgccaagaagctgatcattttcctgaacgagaacaacgtgccagtgcagctgaccagcattagcgccccagagcacaagttcgagggcctgacacagatctttcagaaggcctacgaacacgagcagcacatcagcgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 42 - DNA, 2478 bp, synthetic construct
  source 1..2478: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtggtgccagtctctgaagccctgcgtgaagctgacccctctgtgtgtgaccctgatctgctctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgccgtgacacaggcttgccccaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcagacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggctgcggcgtgggcagagctatgtatgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgttgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcgacattatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 43 - DNA, 2472 bp, synthetic construct
  source 1..2472: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcagacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcgacattatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 44 - protein, 807 aa, synthetic construct
MPMGSLQPLATLYLLGMLVASVLAKGKLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVEIQPLGIAPTGCKRRVVEGGGGSGGGGSAVGIGAVFLGFLGAAGSTMGAASMTLTVQARNLLSGNPDWLPDMTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 45 - protein, 816 aa, synthetic construct
MPMGSLQPLATLYLLGMLVASVLAAENLWVTVYYGVPVWKEACTTLFCASDARAYEKKVHNVWATHCCVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQWFYATGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNCLRAPECQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 46 - protein, 816 aa, synthetic construct
MPMGSLQPLATLYLLGMLVASVLAAENLWVTVYYGVPVWKEAKTTLFCASDARAYEKEVANVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLTVERYARDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 47 - protein, 816 aa, synthetic construct
MPMGSLQPLATLYLLGMLVASVLAAENLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 48 - protein, 816 aa, synthetic construct
MPMGSLQPLATLYLLGMLVASVLAAENLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTMVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPLNLTRKSVRIGPGQTFYAMGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKMIINMWQGVGRAMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 49 - protein, 818 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWCQSLKPCVKLTPLCVTLICSDATVKTGTVEEMKNCSFNTTTEIRDKKKKEYALFYKPDIVPLSETNNTSEYRLINCNTSAVTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGCGVGRAMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 50 - protein, 816 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 51 - DNA, 2532 bp, synthetic construct
  source 1..2532: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcggcggaggaagcggaggcggaggctcaggtggtggcggatctggagatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 52 - DNA, 2517 bp, synthetic construct
  source 1..2517: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcggcggaggaagcggaggcggaggctcaggtgatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 53 - DNA, 2502 bp, synthetic construct
  source 1..2502: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcggcggaggaagcggagatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 54 - DNA, 2487 bp, synthetic construct
  source 1..2487: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcgatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 55 - DNA, 2541 bp, synthetic construct
  source 1..2541: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcggcggaggaagcggaggcggaggctcaggtggtggcggatctggactgagcaaggatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 56 - DNA, 2526 bp, synthetic construct
  source 1..2526: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcggcggaggaagcggaggcggaggctcaggtctgagcaaggatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 57 - DNA, 2511 bp, synthetic construct
  source 1..2511: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcggcggaggaagcggactgagcaaggatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 58 - DNA, 2496 bp, synthetic construct
  source 1..2496: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcctgagcaaggatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 59 - DNA, 2481 bp, synthetic construct
  source 1..2481: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcctgagcaaggatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 60 - protein, 836 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGGGGSGGGGSGGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 61 - protein, 831 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGGGGSGGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 62 - protein, 826 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 63 - protein, 821 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 64 - protein, 839 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGGGGSGGGGSGGGGSGLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 65 - protein, 834 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGGGGSGGGGSGLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 66 - protein, 829 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGGGGSGLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 67 - protein, 824 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 68 - protein, 819 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 69 - DNA, 2475 bp, synthetic construct
  source 1..2475: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacatctgggccacacacgcctgcgtgccaaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccatcaccatggtctgccccaagctgaccttcgagcccattcctatccactactgtgcccctgccggctacgccatcctgaagtgcaacgacgagacattcaacggcacaggcccctgcagcaatgtgtccaccgtgcagtgtacccacggcatcagacctgtgctgagcacacagctgctgctgaatggaagcctggccgagaaagaaatcgtgatcagaagcgagaacctgaccaacaacgccaagatcatcattgtgcatctgcacacccctgtggaaatcgtgtgcacccggcctctgaatctgaccagaaagagcgtgcggatcggccctggccagaccttttatgccatgggcgacatcatcggcgatatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagatgatcattaacatgtggcaaggcgtcggcagggctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagcagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggcccttgatggtggtggaagcggagatatcatcaagctgctgaacgagcaagtgaacaaagaaatgaacagctccaacctgtacatgagcatgtccagctggtgttacacccacagcctggatggcgccggactgttcctgtttgatcacgccgccgaggaatacgagcacgccaagaagctgatcattttcctgaacgagaacaacgtgccagtgcagctgaccagcatctctgcccctgagcacaagttcgagggcctgacacagatctttcagaaggcctacgaacacgagcagcacatctccgagtccatcaacaatatcgtggaccacgccatcaagagcaaggatcacgccaccttcaactttctccagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 70 - DNA, 2475 bp, synthetic construct
  source 1..2475: mol_type = other DNA
gtcgacgccaccatgcccatgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacggcggaggatctggcgacattatcaagctgctgaacgagcaagtgaacaaagagatgaacagctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgataaggatcc

SEQ ID NO: 71 - DNA, 2562 bp, synthetic construct
  source 1..2562: mol_type = other DNA
gtcgacgccaccatgcctatgggatctctgcagcctctggccacactgtacctgctgggaatgctggtggcttctgtgctggccgagggcaatctgtgggtcacagtgtactatggcgtgcccgtgtggaaagaggccaagaccacactgttctgtgccagcgacgccaaggcctacaagaaagaggtggccaacgtctgggccacacacgcctgtgttcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgacacaggcttgccccaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagagctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtggaagtgcagcccctgggaatcgctccaacaggcgctaagagaagagtggtggaacgcgagaaaagagccgctggactgggagccctgtttctgggttttctgggagccgccggaagcacaatgggagctgcctctatcacactgaccgtgcaggctagacagctgctgagcggaattgtgcagcagcagagcaacctgctgagagccattgaagcccagcagcacatgctgcagctgaccgtgtggggaatcaaacagctgcaggccagagtgctgaccctggaaagatacgccaaggaccagcagctcctcggcatgtggggatgttctggcaagctgatctgcaccaccaacgtgccctggaacacctcctggtccaacaagagcgagatggacatctggaacaacatgacctggatgcagtgggagagagagatcagcaactacaccgagacaatctacatgctgctcgaggacagccagcggcagcaagagagaaacgagaaggatctgctggccctggactcctggaatagcctgtggaactggttcaatatcaccaactggctgtggtacatcaagatcttcatcatgatcgtcggcggcctgatcggcctgagaatcgtgtttgccgtgctgagcatcgtgaacagagtgcggcagggaatcagcccactgagcctgcaaaccctgacacctaatcctagagagcccgaccggctgagaggcatcgaagaagaaggcggcgagcaggacagagacagatccatcagactggtgtccggcttcctgcctatcgtgtgggacgatctgagaagcctgtgcctgttcagctaccaccggctgcgggattttctgctgcttgccgccagagtggttgaactgctgggacacagctctctgcggggactgcaaagaggctgggaagtcctgaagtacctgggcagcctggtgcagtattggggcctcgagctgaagagaagcgccattagcctgttcgacaccctggccattgctgtggccgaaggcaccgacagaatcattgagctgatccagggcttctgccgggccatcagaaacatccctaccagaatccggcagggcttcgaggcttccctgctgtgataaggatcc

SEQ ID NO: 72 - protein, 655 aa, synthetic construct; identical to SEQ ID NO: 27

SEQ ID NO: 73 - protein, 817 aa, synthetic construct
MPMGSLQPLATLYLLGMLVASVLAAENLWVTVYYGVPVWKEAKTTLFCASDAKAYKKEVHNIWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAITMVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPLNLTRKSVRIGPGQTFYAMGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKMIINMWQGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 74 - protein, 817 aa, synthetic construct
MPMGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 75 - protein, 846 aa, synthetic construct
MPMGSLQPLATLYLLGMLVASVLAEGNLWVTVYYGVPVWKEAKTTLFCASDAKAYKKEVANVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVEVQPLGIAPTGAKRRVVEREKRAAGLGALFLGFLGAAGSTMGAASITLTVQARQLLSGIVQQQSNLLRAIEAQQHMLQLTVWGIKQLQARVLTLERYAKDQQLLGMWGCSGKLICTTNVPWNTSWSNKSEMDIWNNMTWMQWEREISNYTETIYMLLEDSQRQQERNEKDLLALDSWNSLWNWFNITNWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGHSSLRGLQRGWEVLKYLGSLVQYWGLELKRSAISLFDTLAIAVAEGTDRIIELIQGFCRAIRNIPTRIRQGFEASLL

SEQ ID NO: 76 - DNA, 2223 bp, synthetic construct
  source 1..2223: mol_type = other DNA
gtcgacgccaccatgcctatgggatctctgcagcctctggccacactgtacctgctgggaatgctggtggcttctgtgctggccgagggcaatctgtgggtcacagtgtactatggcgtgcccgtgtggaaagaggccaagaccacactgttctgtgccagcgacgccaaggcctacaagaaagaggtggccaacgtctgggccacacacgcctgtgttcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgacacaggcttgccccaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagagctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtggaagtgcagcccctgggaatcgctccaacaggcgctaagagaagagtggtggaacgcgagaaaagagccgctggactgggagccctgtttctgggttttctgggagccgccggaagcacaatgggagctgcctctatcacactgaccgtgcaggctagacagctgctgagcggaattgtgcagcagcagagcaacctgctgagagccattgaagcccagcagcacatgctgcagctgaccgtgtggggaatcaaacagctgcaggccagagtgctgaccctggaaagatacgccaaggaccagcagctcctcggcatgtggggatgttctggcaagctgatctgcaccaccaacgtgccctggaacacctcctggtccaacaagagcgagatggacatctggaacaacatgacctggatgcagtgggagagagagatcagcaactacaccgagacaatctacatgctgctcgaggacagccagcggcagcaagagagaaacgagaaggatctgctggccctggactcctggaatagcctgtggaactggttcaatatcaccaactggctgtggtacatcaagatcttcatcatgatcgtcggcggcctgatcggcctgagaatcgtgtttgccgtgctgagcatcgtgaacagagtgcggcagggaatcagcccactgagcctgcaaaccctgacacctaatcctagagagcccgaccggctgagaggcatcgaagaagaaggcggcgagcaggacagagacagatccatcagactggtgtcctgataaggatcc

SEQ ID NO: 77 - DNA, 2109 bp, synthetic construct
  source 1..2109: mol_type = other DNA
gtcgacgccaccatgcctatgggatctctgcagcctctggccacactgtacctgctgggaatgctggtggcttctgtgctggccgagggcaatctgtgggtcacagtgtactatggcgtgcccgtgtggaaagaggccaagaccacactgttctgtgccagcgacgccaaggcctacaagaaagaggtggccaacgtctgggccacacacgcctgtgttcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgacacaggcttgccccaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagagctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtggaagtgcagcccctgggaatcgctccaacaggcgctaagagaagagtggtggaacgcgagaaaagagccgctggactgggagccctgtttctgggttttctgggagccgccggaagcacaatgggagctgcctctatcacactgaccgtgcaggctagacagctgctgagcggaattgtgcagcagcagagcaacctgctgagagccattgaagcccagcagcacatgctgcagctgaccgtgtggggaatcaaacagctgcaggccagagtgctgaccctggaaagatacgccaaggaccagcagctcctcggcatgtggggatgttctggcaagctgatctgcaccaccaacgtgccctggaacacctcctggtccaacaagagcgagatggacatctggaacaacatgacctggatgcagtgggagagagagatcagcaactacaccgagacaatctacatgctgctcgaggacagccagcggcagcaagagagaaacgagaaggatctgctggccctggactcctggaatagcctgtggaactggttcaatatcaccaactggctgtggtacatcaagatcttcatcatgatcgtcggcggcctgatcggcctgagaatcgtgtttgccgtgctgagcatcgtgaacagagtgcggcagggatactgataaggatcc

SEQ ID NO: 78 - DNA, 2475 bp, synthetic construct
  source 1..2475: mol_type = other DNA
gtcgacgccaccatgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtggtgccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgaacacctctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggctgcggcgtcggccgggctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacggcggaggatctggcgacattatcaagctgctgaacgagcaagtgaacaaagagatgaacagctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgataaggatcc

SEQ ID NO: 79 - DNA, 2469 bp, synthetic construct
  source 1..2469: mol_type = other DNA
gtcgacgccaccatgggatctctgcaacctctggccacactgtacctgctgggaatgctggtggcttctgtgctggccgccgagaatctgtgggtcacagtgtactatggcgtgcccgtgtggaaagaggcctgcaccacactgttctgtgccagcgacgccaaggcctacaagaaaaaggtgcggaacgtctgggccacacactgctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgaacacctctgtggaaatcgtgtgcacccggcctaacaacaacacccggaagtctgtgcggatcggccctggccagtggttctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaattgcctcagagcccctgagtgccagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacggcggaggatctggcgacattatcaagctgctgaacgagcaagtgaacaaagagatgaacagctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgataaggatcc

SEQ ID NO: 80 - DNA, 2469 bp, synthetic construct
  source 1..2469: mol_type = other DNA
gtcgacgccaccatgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacatctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgactatggtgtgtcctaaactgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtgctgtctacccagctgctgctgaatggaagcctggccgagaaagaaatcgtgatcagaagcgagaacctgaccaacaacgccaagatcatcattgtgcatctgaacacctctgtggaaatcgtgtgcacccggcctctgaacctgacccggaagtctgtgcggatcggccctggccagacattctatgccatgggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagatgatcatcaatatgtggcaaggcgtcggccgggctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacggcggaggatctggcgacattatcaagctgctgaacgagcaagtgaacaaagagatgaacagctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgataaggatcc

SEQ ID NO: 81 - DNA, 2469 bp, synthetic construct
  source 1..2469: mol_type = other DNA
gtcgacgccaccatgggatctctgcaacctctggccacactgtacctgctgggaatgctggtggcttctgtgctggccgccgagaatctgtgggtcacagtgtactatggcgtgcccgtgtggaaagaggcctgcaccacactgttctgtgccagcgacgccaaggcctacaagaaaaaggtgcggaacgtctgggccacacactgctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgaacacctctgtggaaatcgtgtgcacccggcctaacaacaacacccggaagtctgtgcggatcggccctggccagtggttctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccgggctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaattgcctcagagcccctgagtgccagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacggcggaggatctggcgacattatcaagctgctgaacgagcaagtgaacaaagagatgaacagctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgataaggatcc

SEQ ID NO: 82 - DNA, 2469 bp, synthetic construct
  source 1..2469: mol_type = other DNA
gtcgacgccaccatgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgaacacctctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacggcggaggatctggcgacattatcaagctgctgaacgagcaagtgaacaaagagatgaacagctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgataaggatcc

SEQ ID NO: 83 - DNA, 2475 bp, synthetic construct
  source 1..2475: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacatctgggccacacacgcctgcgtgccaaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgacacaagtgtgccccaagctgaccttcgagcccattcctatccactactgtgcccctgccggctacgccatcctgaagtgcaacaacgagacattcaacggcacaggcccctgcagcaatgtgtccaccgtgcagtgtacccacggcatcagacctgtgctgagcacacagctgctgctgaatggaagcctggccgagaaagaaatcgtgatcagaagcgagaacctgaccaacaacgccaagatcatcatcgtgcacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagagctatgtacgcccctcctatcgccggcaacatcacctgtcggagcaatatcacaggcctgctgctcaccagagatggcggcatcaacaacgtgtccaacgaaaccgaaaccttccggcctgccggcggagacatgagagacaattggagaagcgagctgtacaagtacaaggtggtcaagatcgagcccctgggcgtcgcaccaacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaacagcagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggatggcggaggatctggcgacattatcaagctgctgaacgagcaagtgaacaaagaaatgaacagctccaacctgtacatgagcatgtccagctggtgttacacccacagccttgatggcgccggactgttcctgtttgatcacgccgccgaggaatacgagcacgccaagaagctgatcatcttcctgaacgagaacaatgtgcccgtgcagctgaccagcatctctgcccctgagcacaagttcgagggcctgacacagatctttcagaaggcctacgaacacgagcagcacatctccgagtccatcaacaatatcgtggaccacgccattaagagcaaggatcacgccaccttcaactttctccagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 84 - DNA, 2448 bp, synthetic construct
  source 1..2448: mol_type = other DNA
gtcgacgccaccatgcctatgggatctctgcagcctctggccacactgtacctgctgggaatgctggtggcttctgtgctggccgagggcaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtgcacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgaaaccgaaaccttccggcctgccggcggagacatgagagacaattggagaagcgagctgtacaagtacaaggtggtggaagtgcagcccctgggaatcgctcctaccggctgcaagagaagagtggttgaaggcggcggaggatctggcggaggcggatctgctgttggaatcggagctgtgttcctgggctttctgggagccgccggatctacaatgggagctgccagcatgaccctgaccgtgcaggctagaaatctgctgagcggcaaccccgactggctgcctgatatgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggatggcggaggaagcggagatatcatcaagctgctgaacgagcaagtgaacaaagaaatgaacagcagcaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcatctctgcccctgagcacaagttcgagggcctgacacagatctttcagaaggcctacgaacacgagcagcacatctccgagtccatcaacaatatcgtggaccacgccattaagagcaaggatcacgccaccttcaactttctccagtggtacgtggccgaacagcacgaggaagaagtgctgttcaaggacatcctggacaagatcgagctgatcggcaacgagaaccacggcctgtatctggccgaccagtacgtgaagggaatcgccaagagccggaagtcctgatgaggatcc

SEQ ID NO: 85 - DNA, 2475 bp, synthetic construct
  source 1..2475: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtggccaacgtctgggccacacacgcctgtgttcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgacacaggcttgccccaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtgcacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagagctatgtacgcccctcctatcgccggcaacatcacctgtcggagcaatatcacaggcctgctgctcaccagagatggcggcatcaacaacgtgtccaacgaaaccgaaaccttccggcctgccggcggagacatgagagacaattggagaagcgagctgtacaagtacaaggtggtcaagatcgagcccctgggcgtcgcaccaacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctgaccgtggaaagatacgccagagatcagcagctcctcggaatctggggctgtagcggcaagctgatctgctgcaccaacgtgccctggaactccagctggtccaaccggaatctgagcgagatctgggataacatgacctggctgcagtgggacaaagagatctccaactacacccagatcatctacggcctgctggaagagagccagaaccagcaagagaaaaacgagcaggacctgctggccctggatggcggaggatctggcgacattatcaagctgctgaacgagcaagtgaacaaagaaatgaacagcagcaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcatctctgcccctgagcacaagttcgagggcctgacacagatctttcagaaggcctacgaacacgagcagcacatctccgagtccatcaacaatatcgtggaccacgccattaagagcaaggatcacgccaccttcaactttctccagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 86 - protein, 733 aa, synthetic construct
MPMGSLQPLATLYLLGMLVASVLAEGNLWVTVYYGVPVWKEAKTTLFCASDAKAYKKEVANVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVEVQPLGIAPTGAKRRVVEREKRAAGLGALFLGFLGAAGSTMGAASITLTVQARQLLSGIVQQQSNLLRAIEAQQHMLQLTVWGIKQLQARVLTLERYAKDQQLLGMWGCSGKLICTTNVPWNTSWSNKSEMDIWNNMTWMQWEREISNYTETIYMLLEDSQRQQERNEKDLLALDSWNSLWNWFNITNWLWYIKIFIMIVGGLIGLRIVFAVLSIVNRVRQGISPLSLQTLTPNPREPDRLRGIEEEGGEQDRDRSIRLVS

SEQ ID NO: 87 - protein, 695 aa, synthetic construct
MPMGSLQPLATLYLLGMLVASVLAEGNLWVTVYYGVPVWKEAKTTLFCASDAKAYKKEVANVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVEVQPLGIAPTGAKRRVVEREKRAAGLGALFLGFLGAAGSTMGAASITLTVQARQLLSGIVQQQSNLLRAIEAQQHMLQLTVWGIKQLQARVLTLERYAKDQQLLGMWGCSGKLICTTNVPWNTSWSNKSEMDIWNNMTWMQWEREISNYTETIYMLLEDSQRQQERNEKDLLALDSWNSLWNWFNITNWLWYIKIFIMIVGGLIGLRIVFAVLSIVNRVRQGY

SEQ ID NO: 88 - protein, 817 aa, synthetic construct
MGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWCQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGCGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 89 - protein, 815 aa, synthetic construct
MGSLQPLATLYLLGMLVASVLAAENLWVTVYYGVPVWKEACTTLFCASDAKAYKKKVRNVWATHCCVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQWFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNCLRAPECQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 90 - protein, 815 aa, synthetic construct
MGSLQPLATLYLLGMLVASVLAAENLWVTVYYGVPVWKEAKTTLFCASDAKAYKKEVHNIWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTMVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPLNLTRKSVRIGPGQTFYAMGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKMIINMWQGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 91 - protein, 815 aa, synthetic construct
MGSLQPLATLYLLGMLVASVLAAENLWVTVYYGVPVWKEACTTLFCASDAKAYKKKVRNVWATHCCVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQWFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNCLRAPECQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 92 - protein, 815 aa, synthetic construct
MGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 93 - protein, 817 aa, synthetic construct
MPMGSLQPLATLYLLGMLVASVLAAENLWVTVYYGVPVWKEAKTTLFCASDAKAYKKEVHNIWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 94 - protein, 808 aa, synthetic construct
MPMGSLQPLATLYLLGMLVASVLAEGNLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVEVQPLGIAPTGCKRRVVEGGGGSGGGGSAVGIGAVFLGFLGAAGSTMGAASMTLTVQARNLLSGNPDWLPDMTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 95 - protein, 817 aa, synthetic construct
MPMGSLQPLATLYLLGMLVASVLAAENLWVTVYYGVPVWKEAKTTLFCASDAKAYKKEVANVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLTVERYARDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 96 - DNA, 2454 bp, synthetic construct
  source 1..2454: mol_type = other DNA
atgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaaacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattctggcggagacattatcaagctgctgaacgagcaagtgaacaaagaaatgcagtcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtccggctcctgatga

SEQ ID NO: 97 - DNA, 1968 bp, synthetic construct
  source 1..1968: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattgatgaggatcc

SEQ ID NO: 98 - DNA, 1968 bp, synthetic construct
  source 1..1968: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctaacgccaccgtgaaaaacggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattgatgaggatcc

SEQ ID NO: 99 - DNA, 2472 bp, synthetic construct
  source 1..2472: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctaacgccaccgtgaaaaacggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaaacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattctggcggagacattatcaagctgctgaacgagcaagtgaacaaagaaatgcagtcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtccggctcctgatgaggatcc

SEQ ID NO: 100 - protein, 792 aa, synthetic construct
AENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 101 - protein, 624 aa, synthetic construct
AENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 102 - protein, 624 aa, synthetic construct
AENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSNATVKNGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 103 - protein, 792 aa, synthetic construct
AENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSNATVKNGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 104 - DNA, 1989 bp, synthetic construct
  source 1..1989: mol_type = other DNA
gtcgacgccaccatgcctatgggatctctgcaacctctggccacactgtacctgctgggaatgctggtggcttctgtgctggccgccgagaatctgtgggtcacagtgtactatggcgtgcccgtgtgggaagatgccgacacacctctgttttgtgccagcgacgccaaggcctacagcaccgagtctcacaatgtgtgggccactcacgcctgcgtgccaaccgatccatctcctcaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccacatgcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaggccatctatgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgtgggccagtgtatgtatgcccctcctatcgccggcaacatcacatgcagaagcaatatcaccggcctgctgctcaccagagatggcggcggaaacaacaccaacgagacagaaaccttccggcctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggctgtggaaagatacctgagggaccagcagctcctcggaatctggggctgttctggcaagctgatctgctgcaccaatgtgccctggaacagctcctggtccaaccggaatctgagcgagatctgggacaatatgacctggctgcagtgggacaaagagattagcaactacacccagatcatctacggactgctggaagagagccagaaccagcaagagaaaaacgagcaggacctgctggccctggactgataaggatcc

SEQ ID NO: 105 - DNA, 1989 bp, synthetic construct
  source 1..1989: mol_type = other DNA
gtcgacgccaccatgcctatgggatctctgcagcctctggccacactgtacctgctgggaatgctggtggctagcgtgctggcctccaacaatctgtgggtcaccgtgtactacggcgtgcccgtttgggaagatgccgacacacctctgttctgtgcctccgatgccaaggcctacagcaccgagtctcacaacgtgtgggccactcatgcctgcgtgccaaccgatccatctcctcaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccaccatcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaggccatctatgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgttggccaggctatgtatgcccctcctatcgccggcaacatcacatgcagaagcaatatcaccggcctgctgctcaccagagatggcggcggaaacaacaccaacgagacagaaaccttccggcctgccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgcctctatcaccctgaccgtgcaagttcgacagctgctgtctggcatcgtgcagcagcagagcaacctgctgagagcccctgaagctcagcagcatctcctgcagctgacagtgtggggaatcaagcagctccaggccagaatcctggccgtcgagagatacctgaaggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 106 - DNA, 1989 bp, synthetic construct
  source 1..1989: mol_type = other DNA
gtcgacgccaccatgcctatgggatctctgcagcctctggccacactgtacctgctgggaatgctggtggctagcgtgctggcctccaacaatctgtgggtcaccgtgtactacggcgtgcccgtttgggaagatgccgacacacctctgttctgtgcctccgatgccaaggcctacagcaccgagtctcacaacgtgtgggccactcatgcctgcgtgccaaccgatccatctcctcaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccacctgcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaggccatctatgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgttggccagtgcatgtatgcccctcctatcgccggcaacatcacatgcagaagcaatatcaccggcctgctgctcaccagagatggcggcggaaacaacaccaacgagacagaaaccttccggcctgccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgcctctatcaccctgaccgtgcaagttcgacagctgctgtctggcatcgtgcagcagcagagcaacctgctgagagcccctgaagctcagcagcatctcctgcagctgacagtgtggggaatcaagcagctccaggccagaatcctggccgtcgagagatacctgaaggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 107 - DNA, 1950 bp, synthetic construct
  source 1..1950: mol_type = other DNA
gtcgacgccaccatgcctatgggatctctgcagcctctggccacactgtacctgctgggaatgctggtggctagcgtgctggcctccaacaatctgtgggtcaccgtgtactacggcgtgcccgtttgggaagatgccgacacacctctgttctgtgcctccgatgccaaggcctacagcaccgagtctcacaacgtgtgggccactcatgcctgcgtgccaaccgatccatctcctcaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccacatgcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaggccatctatgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgtgggccagtgtatgtatgcccctcctatcgccggcaacatcacatgcagaagcaatatcaccggcctgctgctcaccagagatggcggcggaaacaacaccaacgagacagaaaccttccggcctgccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgcctctatcaccctgaccgtgcaagttcgacagctgctgagcggcaaccctgactggctgcctgatatgacagtgtggggaatcaagcagctgcaggccagaatcctggccgtggaaagatacctgaaggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 108 - DNA, 1989 bp, synthetic construct
  source 1..1989: mol_type = other DNA
gtcgacgccaccatgcctatgggatctctgcagcctctggccacactgtacctgctgggaatgctggtggctagcgtgctggcctccaacaatctgtgggtcaccgtgtactacggcgtgcccgtttgggaagatgcctgcacacctctgttctgtgcctccgatgccaaggcctacagcaccaagagcagaaacgtgtgggccacctgttgctgcgtgcccacagatccatctccacaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccaccatcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaagcttggtacgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgttggccaggctatgtatgcccctcctatcgccggcaacatcacatgcagaagcaatatcaccggcctgctgctcaccagagatggcggcggaaacaacaccaacgagacagagacatttcggcctgccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgccagcatgacactgaccgtgcaagttcgacagctgctgagcggaatcgtgcagcagcagagcaactgtctgagagcccctgagtgccagcagtgtctgctgcaactgacagtgtggggaatcaagcagctgcaggccagaatcctggccgtggaaagatacctgaaggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 109 - DNA, 1989 bp, synthetic construct
  source 1..1989: mol_type = other DNA
gtcgacgccaccatgcctatgggatctctgcagcctctggccacactgtacctgctgggaatgctggtggctagcgtgctggcctccaacaatctgtgggtcaccgtgtactacggcgtgcccgtttgggaagatgccgagacacctctgttctgtgcctccgatgccaaggcctacagcaccgagaagcacaacgtgtgggccactcatgcctgcgtgccaaccgatccatctcctcaagagatcagcctggacaacgtgaccgagaacttcaacatgtggaagaacaacatggtcgagcagatgcacaccgacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaaaaaacaggtgcacgccctgttctaccggctggacatcgtgcccatcaacggcaacagcaactccaacagctccgtgggcgactacagactgatcaactgcaacgtgtccaccatcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacaccagaaagagcatcagactcggcccaggccaggccatctatgccacaggcgatatcatcggcaacatccggcaggcccactgcaatatctctggcgccgattggggcaacatgatccggaacgtgtccgaaaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgttggccaggctatgtatgctcctcctatcgccggcaatatcacctgtcggagcaacattaccggcctgctgctcaccagagatggcggcggaaacaataccaacgagacagaaaccttccggccagccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgcctctatcaccctgaccgtgcaagtgcgcaatctgctgtctggcatcgtgcagcagcagagcaacctgctgagagcccctgaagctcagcagcatctcctgcagctgacagtgtggggaatcaagcagctccaggccagaatcctggccgtcgagagatacctgagggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 110 - DNA, 2022 bp, synthetic construct
  source 1..2022: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagagccgagaatctgtgggtcacagtgtactatggcgtgcccgtgtgggaagatgccgacacacctctgttttgtgccagcgacgccaaggcctacagcaccgagtctcacaatgtgtgggccactcacgcctgcgtgccaaccgatccatctcctcaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccacatgcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaggccatctatgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgtgggccagtgtatgtatgcccctcctatcgccggcaacatcacatgcagaagcaatatcaccggcctgctgctcaccagagatggcggcggaaacaacaccaacgagacagaaaccttccggcctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggctgtggaaagatacctgagggaccagcagctcctcggaatctggggctgttctggcaagctgatctgctgcaccaatgtgccctggaacagctcctggtccaaccggaatctgagcgagatctgggacaatatgacctggctgcagtgggacaaagagattagcaactacacccagatcatctacggactgctggaagagagccagaaccagcaagagaaaaacgagcaggacctgctggccctggactgataaggatcc

SEQ ID NO: 111 - DNA, 2022 bp, synthetic construct
  source 1..2022: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagatccaacaatctgtgggtcaccgtgtactacggcgtgcccgtttgggaagatgccgacacacctctgttctgtgcctccgatgccaaggcctacagcaccgagtctcacaacgtgtgggccactcatgcctgcgtgccaaccgatccatctcctcaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccaccatcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaggccatctatgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgttggccaggctatgtatgcccctcctatcgccggcaacatcacatgcagaagcaatatcaccggcctgctgctcaccagagatggcggcggaaacaacaccaacgagacagaaaccttccggcctgccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgcctctatcaccctgaccgtgcaagttcgacagctgctgtctggcatcgtgcagcagcagagcaacctgctgagagcccctgaagctcagcagcatctcctgcagctgacagtgtggggaatcaagcagctccaggccagaatcctggccgtcgagagatacctgaaggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 112 - DNA, 2022 bp, synthetic construct
  source 1..2022: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagatccaacaatctgtgggtcaccgtgtactacggcgtgcccgtttgggaagatgccgacacacctctgttctgtgcctccgatgccaaggcctacagcaccgagtctcacaacgtgtgggccactcatgcctgcgtgccaaccgatccatctcctcaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccacctgcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaggccatctatgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgttggccagtgcatgtatgcccctcctatcgccggcaacatcacatgcagaagcaatatcaccggcctgctgctcaccagagatggcggcggaaacaacaccaacgagacagaaaccttccggcctgccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgcctctatcaccctgaccgtgcaagttcgacagctgctgtctggcatcgtgcagcagcagagcaacctgctgagagcccctgaagctcagcagcatctcctgcagctgacagtgtggggaatcaagcagctccaggccagaatcctggccgtcgagagatacctgaaggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 113 - DNA, 1983 bp, synthetic construct
  source 1..1983: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagatccaacaatctgtgggtcaccgtgtactacggcgtgcccgtttgggaagatgccgacacacctctgttctgtgcctccgatgccaaggcctacagcaccgagtctcacaacgtgtgggccactcatgcctgcgtgccaaccgatccatctcctcaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccacatgcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaggccatctatgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgtgggccagtgtatgtatgcccctcctatcgccggcaacatcacatgcagaagcaatatcaccggcctgctgctcaccagagatggcggcggaaacaacaccaacgagacagaaaccttccggcctgccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgcctctatcaccctgaccgtgcaagttcgacagctgctgagcggcaaccctgactggctgcctgatatgacagtgtggggaatcaagcagctgcaggccagaatcctggccgtggaaagatacctgaaggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 114 - DNA, 2022 bp, synthetic construct
  source 1..2022: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagatccaacaatctgtgggtcaccgtgtactacggcgtgcccgtttgggaagatgcctgcacacctctgttctgtgcctccgatgccaaggcctacagcaccaagagcagaaacgtgtgggccacctgttgctgcgtgcccacagatccatctccacaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccaccatcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaagcttggtacgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgttggccaggctatgtatgcccctcctatcgccggcaacatcacatgcagaagcaatatcaccggcctgctgctcaccagagatggcggcggaaacaacaccaacgagacagagacatttcggcctgccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgccagcatgacactgaccgtgcaagttcgacagctgctgagcggaatcgtgcagcagcagagcaactgtctgagagcccctgagtgccagcagtgtctgctgcaactgacagtgtggggaatcaagcagctgcaggccagaatcctggccgtggaaagatacctgaaggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 115 - DNA, 2022 bp, synthetic construct
  source 1..2022: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagatccaacaatctgtgggtcaccgtgtactacggcgtgcccgtttgggaagatgccgagacacctctgttctgtgcctccgatgccaaggcctacagcaccgagaagcacaacgtgtgggccactcatgcctgcgtgccaaccgatccatctcctcaagagatcagcctggacaacgtgaccgagaacttcaacatgtggaagaacaacatggtcgagcagatgcacaccgacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaaaaaacaggtgcacgccctgttctaccggctggacatcgtgcccatcaacggcaacagcaactccaacagctccgtgggcgactacagactgatcaactgcaacgtgtccaccatcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacaccagaaagagcatcagactcggcccaggccaggccatctatgccacaggcgatatcatcggcaacatccggcaggcccactgcaatatctctggcgccgattggggcaacatgatccggaacgtgtccgaaaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgttggccaggctatgtatgctcctcctatcgccggcaatatcacctgtcggagcaacattaccggcctgctgctcaccagagatggcggcggaaacaataccaacgagacagaaaccttccggccagccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgcctctatcaccctgaccgtgcaagtgcgcaatctgctgtctggcatcgtgcagcagcagagcaacctgctgagagcccctgaagctcagcagcatctcctgcagctgacagtgtggggaatcaagcagctccaggccagaatcctggccgtcgagagatacctgagggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 116 - DNA, 1956 bp, synthetic construct
  source 1..1956: mol_type = other DNA
gtcgacgccaccatgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacgatcccgaagtgcacaatgtgtgggccactcacgcctgcgtgcccaccgatcctaatcctcaagagatgttcctggaaaacgtgaccgagaacttcaacatgtggaagaacgacatggtcgagcagatgcaccaggacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaagaagcacgccctgttctaccggctggacatcgtgcccctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacctttttcgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcaaggcgtgggccagtgtatgtacgcccctcctatcgccggcaacatcatctgcagaagcaagatcaccggcctgctgctcacaagagatggcggcatcaacaagagcgaggaaatcttcagacccggcggaggcaacatgaaggacaactggcgggacaattggcggagcgagctgtacaagtacaaggtggtcaagatcgagcccctgggcgtcgcacctacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtccagcagcagagcaatctgctcagagcccctgaggctcagcagcatctcctgaaactgaccgtgtggggaatcaagcagctgcaggccagagttctggccgtggaaagatacctgagggaccagcagctcctcggcatctggggctgttctggcaagctgatctgctgcaccaacgtgccctggaacagctcctggtccaacagaaacctgagcgagatctgggacaacatgacctggctgcagtgggacaaagagatcagcaactacacccagatcatctacggactgctggaagagagccagaaccagcaagagaaaaacgagcaggacctgctggccctggactgataaggatcc

SEQ ID NO: 117 - DNA, 1941 bp, synthetic construct
  source 1..1941: mol_type = other DNA
gtcgacgccaccatgggatctctgcaacctctggccacactgtacctgctgggaatgctggtggctagcgtgctggccaatctgtgggtcacagtgtactacggcgtgcccgtgtggaaagaggccaagaccacactgttctgtgccagcgacgccaaggcctacgatcccgaagtgcacaatgtgtgggccactcacgcctgcgtgcccaccgatcctaatcctcaagagatgttcctggaaaacgtgaccgagaacttcaacatgtggaagaacgacatggtcgagcagatgcaccaggacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaagaagcacgccctgttctaccggctggacatcgtgcccctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacctttttcgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcaaggcgtgggccagtgtatgtacgcccctcctatcgccggcaacatcatctgcagaagcaagatcaccggcctgctgctcacaagagatggcggcatcaacaagagcgaggaaatcttcagacccggcggaggcaacatgaaggacaattggagaagcgagctgtacaagtacaaggtggtggaaatcaagcccctgggcgtcgcccctacaaagtgcaagagaagagtggtggaacggcggagaaggcggagagctgttgttggactgggagccgtgttcctgggctttcttggagccgccggatctacaatgggagccgcctctatcaccctgaccgtgcaggctagacagctgctgagcggaattgtccagcagcagaacaacctgctgagagcccctgaagcacagcaacacatgctgcagctgaccgtgtggggaatcaaacagctgcaggccagagtcctggccatcgagagatacctgaaggaccagcagctcctcggcatctggggctgttctggcaagctgatctgctgtactgccgtgccttggaacagctcctggtccaacaagtcccaagaggacatctgggacaacatgacctggatgcagtgggacagagagatcagcaactacaccaacaccatctaccgcctgctggaagatagccagacacagcaagagaaaaacgagaaggacctgctggccctggactgatgaggatcc

SEQ ID NO: 118 - DNA, 1941 bp, synthetic construct
  source 1..1941: mol_type = other DNA
gtcgacgccaccatgggatctctgcaacctctggccacactgtacctgctgggaatgctggtggctagcgtgctggccaatctgtgggtcacagtgtactacggcgtgcccgtgtggaaagaggcctgcaccacactgttttgtgccagcgacgccaaggcctacgatcccaaagtgcggaatgtgtgggccactcactgctgcgtgcccaccgatcctaatcctcaagagatgttcctggaaaacgtgaccgagaacttcaacatgtggaagaacgacatggtcgagcagatgcaccaggacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaagaagcacgccctgttctaccggctggacatcgtgcccctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacttggtttgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcaaggcgtgggccagtgtatgtacgcccctcctatcgccggcaacatcatctgcagaagcaagatcaccggcctgctgctcacaagagatggcggcatcaacaagagcgaggaaatcttcagacccggcggaggcaacatgaaggacaattggagaagcgagctgtacaagtacaaggtggtggaaatcaagcccctgggcgtcgcccctacaaagtgcaagagaagagtggtggaacggcggagaaggcggagagctgttgttggactgggagccgtgttcctgggctttcttggagccgccggatctacaatgggagccgcctctatgaccctgaccgtgcaggctagacagctgctgagcggaattgtccagcagcagaacaactgcctgagagcccctgagtgccagcagtgtatgctgcagctgacagtgtggggaatcaagcagctgcaggccagagttctggccatcgagagatacctgaaggaccagcagctcctcggcatctggggctgttctggcaagctgatctgctgtactgccgtgccttggaacagctcctggtccaacaagtcccaagaggacatctgggacaacatgacctggatgcagtgggacagagagatcagcaactacaccaacaccatctaccgcctgctggaagatagccagacacagcaagagaaaaacgagaaggacctgctggccctggactgatgaggatcc

SEQ ID NO: 119 - DNA, 1902 bp, synthetic construct
  source 1..1902: mol_type = other DNA
gtcgacgccaccatgggatctctgcaacctctggccacactgtacctgctgggaatgctggtggctagcgtgctggccaatctgtgggtcacagtgtactacggcgtgcccgtgtggaaagaggccaagaccacactgttctgtgccagcgacgccaaggcctacgatcccgaagtgcacaatgtgtgggccactcacgcctgcgtgcccaccgatcctaatcctcaagagatgttcctggaaaacgtgaccgagaacttcaacatgtggaagaacgacatggtcgagcagatgcaccaggacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaagaagcacgccctgttctaccggctggacatcgtgcccctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacctttttcgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcaaggcgtgggccagtgtatgtacgcccctcctatcgccggcaacatcatctgcagaagcaagatcaccggcctgctgctcacaagagatggcggcatcaacaagagcgaggaaatcttcagacccggcggaggcaacatgaaggacaattggagaagcgagctgtacaagtacaaggtggtggaaatcaagcccctgggcgtcgcccctacaaagtgcaagagaagagtggtggaacggcggagaaggcggagagctgttgttggactgggagccgtgttcctgggctttcttggagccgccggatctacaatgggagccgcctctatcaccctgaccgtgcaggctagacagctgctgagcggcaatcctgactggctgcctgatatgaccgtgtggggaatcaagcagctgcaggccagagttctggccatcgagagatacctgaaggaccagcagctcctcggcatctggggctgttctggcaagctgatctgctgtactgccgtgccttggaacagctcctggtccaacaagtcccaagaggacatctgggacaacatgacctggatgcagtgggacagagagatcagcaactacaccaacaccatctaccgcctgctggaagatagccagacacagcaagagaaaaacgagaaggacctgctggccctggactgatgaggatcc

SEQ ID NO: 120 - DNA, 1941 bp, synthetic construct
  source 1..1941: mol_type = other DNA
gtcgacgccaccatgggatctctgcaacctctggccacactgtacctgctgggaatgctggtggctagcgtgctggccaatctgtgggtcacagtgtactacggcgtgcccgtgtggaaggatgccgagacaacactgttctgtgccagcgacgccaaggcctacgatcccgagaaacacaacgtgtgggccactcacgcctgcgtgccaaccgatcctaatcctcaagagatgttcctggaaaacgtgaccgagaacttcaacatgtggaagaacgacatggtcgagcagatgcacaccgacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaaagtgcacgccctgttctaccggctggacatcgttcctctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacctttttcgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcagcgcgtgggccagtgtatgtacgcccctccaatcgccggcaacatcatctgcagaagcaagatcaccggcctgctgctcacaagagatggcggcatcaacaagagcgaggaaatcttcagacccggcggaggcaacatgaaggacaattggagaagcgagctgtacaagtacaaggtggtggaaatcaagcccctgggcgtcgcccctacacggtgcaaaagaagagtggtcgagcggcggagaaggcggagagctgttgttggactgggagccgtgttcctgggctttcttggagccgccggatctacaatgggagccgcctctatcaccctgaccgtgcaggctagaaatctgctgagcggcatcgtccagcagcagagcaatctgctcagagcccctgaggcacagcaacacatgctgcagctgacagtgtggggaatcaagcagctgcaggccagagttctggccatcgagagatacctgagggaccagcagctcctcggcatctggggctgttctggcaagctgatctgctgtactgccgtgccttggaacagctcctggtccaacaagtcccaagaggacatctgggacaacatgacctggatgcagtgggacagagagatcagcaactacaccaacaccatctaccgcctgctggaagatagccagacacagcaagagaagaacgagaaggacctgctggccctggactgatgaggatcc

SEQ ID NO: 121 - DNA, 1995 bp, synthetic construct
  source 1..1995: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagagccgagaatctgtgggtcacagtgtactatggcgtgcccgtgtggaaagaggccaagaccacactgttctgtgccagcgacgccaaggcctacgatcccgaagtgcacaatgtgtgggccactcacgcctgcgtgcccaccgatcctaatcctcaagagatgttcctggaaaacgtgaccgagaacttcaacatgtggaagaacgacatggtcgagcagatgcaccaggacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaagaagcacgccctgttctaccggctggacatcgtgcccctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacctttttcgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcaaggcgtgggccagtgtatgtacgcccctcctatcgccggcaacatcatctgcagaagcaagatcaccggcctgctgctcacaagagatggcggcatcaacaagagcgaggaaatcttcagacccggcggaggcaacatgaaggacaactggcgggacaattggcggagcgagctgtacaagtacaaggtggtcaagatcgagcccctgggcgtcgcacctacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtccagcagcagagcaatctgctcagagcccctgaggctcagcagcatctcctgaaactgaccgtgtggggaatcaagcagctgcaggccagagttctggccgtggaaagatacctgagggaccagcagctcctcggcatctggggctgttctggcaagctgatctgctgcaccaacgtgccctggaacagctcctggtccaacagaaacctgagcgagatctgggacaacatgacctggctgcagtgggacaaagagatcagcaactacacccagatcatctacggactgctggaagagagccagaaccagcaagagaaaaacgagcaggacctgctggccctggactgataaggatcc

SEQ ID NO: 122 - DNA, 1980 bp, synthetic construct
  source 1..1980: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagaaatctgtgggtcacagtgtactacggcgtgcccgtgtggaaagaggccaagaccacactgttctgtgccagcgacgccaaggcctacgatcccgaagtgcacaatgtgtgggccactcacgcctgcgtgcccaccgatcctaatcctcaagagatgttcctggaaaacgtgaccgagaacttcaacatgtggaagaacgacatggtcgagcagatgcaccaggacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaagaagcacgccctgttctaccggctggacatcgtgcccctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacctttttcgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcaaggcgtgggccagtgtatgtacgcccctcctatcgccggcaacatcatctgcagaagcaagatcaccggcctgctgctcacaagagatggcggcatcaacaagagcgaggaaatcttcagacccggcggaggcaacatgaaggacaattggagaagcgagctgtacaagtacaaggtggtggaaatcaagcccctgggcgtcgcccctacaaagtgcaagagaagagtggtggaacggcggagaaggcggagagctgttgttggactgggagccgtgttcctgggctttcttggagccgccggatctacaatgggagccgcctctatcaccctgaccgtgcaggctagacagctgctgagcggaattgtccagcagcagaacaacctgctgagagcccctgaagcacagcaacacatgctgcagctgaccgtgtggggaatcaaacagctgcaggccagagtcctggccatcgagagatacctgaaggaccagcagctcctcggcatctggggctgttctggcaagctgatctgctgtactgccgtgccttggaacagctcctggtccaacaagtcccaagaggacatctgggacaacatgacctggatgcagtgggacagagagatcagcaactacaccaacaccatctaccgcctgctggaagatagccagacacagcaagagaaaaacgagaaggacctgctggccctggactgatgaggatcc

SEQ ID NO: 123 - DNA, 1980 bp, synthetic construct
  source 1..1980: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagaaatctgtgggtcacagtgtactacggcgtgcccgtgtggaaagaggcctgcaccacactgttttgtgccagcgacgccaaggcctacgatcccaaagtgcggaatgtgtgggccactcactgctgcgtgcccaccgatcctaatcctcaagagatgttcctggaaaacgtgaccgagaacttcaacatgtggaagaacgacatggtcgagcagatgcaccaggacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaagaagcacgccctgttctaccggctggacatcgtgcccctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacttggtttgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcaaggcgtgggccagtgtatgtacgcccctcctatcgccggcaacatcatctgcagaagcaagatcaccggcctgctgctcacaagagatggcggcatcaacaagagcgaggaaatcttcagacccggcggaggcaacatgaaggacaattggagaagcgagctgtacaagtacaaggtggtggaaatcaagcccctgggcgtcgcccctacaaagtgcaagagaagagtggtggaacggcggagaaggcggagagctgttgttggactgggagccgtgttcctgggctttcttggagccgccggatctacaatgggagccgcctctatgaccctgaccgtgcaggctagacagctgctgagcggaattgtccagcagcagaacaactgcctgagagcccctgagtgccagcagtgtatgctgcagctgacagtgtggggaatcaagcagctgcaggccagagttctggccatcgagagatacctgaaggaccagcagctcctcggcatctggggctgttctggcaagctgatctgctgtactgccgtgccttggaacagctcctggtccaacaagtcccaagaggacatctgggacaacatgacctggatgcagtgggacagagagatcagcaactacaccaacaccatctaccgcctgctggaagatagccagacacagcaagagaaaaacgagaaggacctgctggccctggactgatgaggatcc

SEQ ID NO: 124 - DNA, 1941 bp, synthetic construct
  source 1..1941: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagaaatctgtgggtcacagtgtactacggcgtgcccgtgtggaaagaggccaagaccacactgttctgtgccagcgacgccaaggcctacgatcccgaagtgcacaatgtgtgggccactcacgcctgcgtgcccaccgatcctaatcctcaagagatgttcctggaaaacgtgaccgagaacttcaacatgtggaagaacgacatggtcgagcagatgcaccaggacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaagaagcacgccctgttctaccggctggacatcgtgcccctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacctttttcgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcaaggcgtgggccagtgtatgtacgcccctcctatcgccggcaacatcatctgcagaagcaagatcaccggcctgctgctcacaagagatggcggcatcaacaagagcgaggaaatcttcagacccggcggaggcaacatgaaggacaattggagaagcgagctgtacaagtacaaggtggtggaaatcaagcccctgggcgtcgcccctacaaagtgcaagagaagagtggtggaacggcggagaaggcggagagctgttgttggactgggagccgtgttcctgggctttcttggagccgccggatctacaatgggagccgcctctatcaccctgaccgtgcaggctagacagctgctgagcggcaatcctgactggctgcctgatatgaccgtgtggggaatcaagcagctgcaggccagagttctggccatcgagagatacctgaaggaccagcagctcctcggcatctggggctgttctggcaagctgatctgctgtactgccgtgccttggaacagctcctggtccaacaagtcccaagaggacatctgggacaacatgacctggatgcagtgggacagagagatcagcaactacaccaacaccatctaccgcctgctggaagatagccagacacagcaagagaaaaacgagaaggacctgctggccctggactgatgaggatcc

SEQ ID NO: 125 - DNA, 1980 bp, synthetic construct
  source 1..1980: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagaaatctgtgggtcacagtgtactacggcgtgcccgtgtggaaggatgccgagacaacactgttctgtgccagcgacgccaaggcctacgatcccgagaaacacaacgtgtgggccactcacgcctgcgtgccaaccgatcctaatcctcaagagatgttcctggaaaacgtgaccgagaacttcaacatgtggaagaacgacatggtcgagcagatgcacaccgacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaaagtgcacgccctgttctaccggctggacatcgttcctctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacctttttcgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcagcgcgtgggccagtgtatgtacgcccctccaatcgccggcaacatcatctgcagaagcaagatcaccggcctgctgctcacaagagatggcggcatcaacaagagcgaggaaatcttcagacccggcggaggcaacatgaaggacaattggagaagcgagctgtacaagtacaaggtggtggaaatcaagcccctgggcgtcgcccctacacggtgcaaaagaagagtggtcgagcggcggagaaggcggagagctgttgttggactgggagccgtgttcctgggctttcttggagccgccggatctacaatgggagccgcctctatcaccctgaccgtgcaggctagaaatctgctgagcggcatcgtccagcagcagagcaatctgctcagagcccctgaggcacagcaacacatgctgcagctgacagtgtggggaatcaagcagctgcaggccagagttctggccatcgagagatacctgagggaccagcagctcctcggcatctggggctgttctggcaagctgatctgctgtactgccgtgccttggaacagctcctggtccaacaagtcccaagaggacatctgggacaacatgacctggatgcagtgggacagagagatcagcaactacaccaacaccatctaccgcctgctggaagatagccagacacagcaagagaagaacgagaaggacctgctggccctggactgatgaggatcc

SEQ ID NO: 126 - DNA, 1971 bp, synthetic construct
  source 1..1971: mol_type = other DNA
gtcgacgccaccatgcctatgggatctctgcagcctctggccacactgtacctgctgggaatgctggtggctagcgtgctggccaagggaaagctgtgggtcacagtgtactacggcgtgcccgtgtggaaagaggccaagaccacactgttctgcgcctccgatgccagagcctacgagaaagaggtgcacaacgtctgggccacacacgcctgtgtgcctaccgatccatctcctcaagagctgttcctgaagaacgtgaccgagaacttcaacatgtggaagaacgacatggtggaccagatgcacgaggacatcatcagcctgtgggaccagagcctgaagccttgcgtgaagctgacccctctgtgcgtgaccctgatctgcagcaatgccaccgtgaagaaccggaccgtggaagagatgaagaactgcagcttcaacaccaccaccgagatccgggacaaagagaagaaagagtacgccctgttctacaagcccgacgtggtgcccctggacgagacaaacaataccagcgagtaccggctgatcaactgcaacaccagcgcctgcactcaggcctgtcctaaagtgaccttcgagcccattcctatccactactgtgcccctgccggctacgccatcctgaagtgcaacaacgagacattcaacggcacaggcccctgcagcaacgtgtccacagtgcagtgtacccacggcatcagacccgtggtgtctacacagctgctgctgaatggaagcctggccgagaaagaaatcgtgatcagaagcgagaacctgaccaacaacgccaagatcatcatcgtgcacctgaacacccctgtggaaatcgtgtgcacccggcctaacaacaacaccagaaagagcgtgcggatcggccctggccagaccttttatgccaccggcgatatcatcggcgacatcagacaggcccactgtaacatcagcgagaagcagtggaacgacaccctgcagaaagtgggcaaagagctgaagaagcacttccccaacaagaccattaagtacgcccagagcgctggcggcgacatggaaatcaccacacacagcttcaattgtggcggcgagttcttctactgcaataccgccaagctgttcaacggaacctacaacggcaccgacatcagcaccaacagcagcgccgacagcaacagcaccatcactctgcagtgccggatcaagcagatcatcaatatgtggcaaggcgtcggccggtgtatgtacgcccctcctatcgccggcaacatcacatgcaagagccacatcacaggcctgctgctcacaagagatggcggcacaaacagctccggcaaagaggaaatcttccggcctgccggcggagacatgcgggataattggagaagcgagctgtacaagtacaaggtggtggaaatccagccactgggaatcgccccaacaggctgcaagagaagagtggtcgagcggagaagaaggcggagagctgctggactgggtgccctgtttctgggctttcttggagccgccggaagcacaatgggagccgcctctattaccctgaccgtgcaggctagacagctgctctctggaatcgtgcagcagcagagcaacctgctgagagcccctgaagctcagcagcacatgctgcagctgacagtgtggggcatcaagcagctgcaggcaagagtgctggccctggaaagatacctgaaggatcagcagctcctcggcatgtggggctgttctggcaagctgatctgctgcaccaacgtgccctggaacacctcttggagcaacaagagcgagacagacatctgggacaacatgacctggatgcagtgggagagagagatcagcaactacaccgagacaatctacaaactgctcgaggacagccagaaccagcaagagagaaacgagcaggacctgctggctctggactgatgaggatcc

SEQ ID NO: 127 - DNA, 2004 bp, synthetic construct
  source 1..2004: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagaaagggaaagctgtgggtcacagtgtactacggcgtgcccgtgtggaaagaggccaagaccacactgttctgcgcctccgatgccagagcctacgagaaagaggtgcacaacgtctgggccacacacgcctgtgtgcctaccgatccatctcctcaagagctgttcctgaagaacgtgaccgagaacttcaacatgtggaagaacgacatggtggaccagatgcacgaggacatcatcagcctgtgggaccagagcctgaagccttgcgtgaagctgacccctctgtgcgtgaccctgatctgcagcaatgccaccgtgaagaaccggaccgtggaagagatgaagaactgcagcttcaacaccaccaccgagatccgggacaaagagaagaaagagtacgccctgttctacaagcccgacgtggtgcccctggacgagacaaacaataccagcgagtaccggctgatcaactgcaacaccagcgcctgcactcaggcctgtcctaaagtgaccttcgagcccattcctatccactactgtgcccctgccggctacgccatcctgaagtgcaacaacgagacattcaacggcacaggcccctgcagcaacgtgtccacagtgcagtgtacccacggcatcagacccgtggtgtctacacagctgctgctgaatggaagcctggccgagaaagaaatcgtgatcagaagcgagaacctgaccaacaacgccaagatcatcatcgtgcacctgaacacccctgtggaaatcgtgtgcacccggcctaacaacaacaccagaaagagcgtgcggatcggccctggccagaccttttatgccaccggcgatatcatcggcgacatcagacaggcccactgtaacatcagcgagaagcagtggaacgacaccctgcagaaagtgggcaaagagctgaagaagcacttccccaacaagaccattaagtacgcccagagcgctggcggcgacatggaaatcaccacacacagcttcaattgtggcggcgagttcttctactgcaataccgccaagctgttcaacggaacctacaacggcaccgacatcagcaccaacagcagcgccgacagcaacagcaccatcactctgcagtgccggatcaagcagatcatcaatatgtggcaaggcgtcggccggtgtatgtacgcccctcctatcgccggcaacatcacatgcaagagccacatcacaggcctgctgctcacaagagatggcggcacaaacagctccggcaaagaggaaatcttccggcctgccggcggagacatgcgggataattggagaagcgagctgtacaagtacaaggtggtggaaatccagccactgggaatcgccccaacaggctgcaagagaagagtggtcgagcggagaagaaggcggagagctgctggactgggtgccctgtttctgggctttcttggagccgccggaagcacaatgggagccgcctctattaccctgaccgtgcaggctagacagctgctctctggaatcgtgcagcagcagagcaacctgctgagagcccctgaagctcagcagcacatgctgcagctgacagtgtggggcatcaagcagctgcaggcaagagtgctggccctggaaagatacctgaaggatcagcagctcctcggcatgtggggctgttctggcaagctgatctgctgcaccaacgtgccctggaacacctcttggagcaacaagagcgagacagacatctgggacaacatgacctggatgcagtgggagagagagatcagcaactacaccgagacaatctacaaactgctcgaggacagccagaaccagcaagagagaaacgagcaggacctgctggctctggactgatgaggatcc

SEQ ID NO: 128 - DNA, 1971 bp, synthetic construct
  source 1..1971: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctgttcctgaagaacgtgaccgagaacttcaacatgtggaagaacgacatggtggaccagatgcacgaggacatcatcagcctgtgggaccagagcctgaagccttgcgtgaagctgacccctctgtgcgtgaccctgatctgcagcaatgccaccgtgaagaaccggaccgtggaagagatgaagaactgcagcttcaacaccaccaccgagatccgggacaaagagaagaaagagtacgccctgttctacaagcccgacgtggtgcccctggacgagacaaacaataccagcgagtaccggctgatcaactgcaacaccagcgcctgcactcaggcctgtcctaaagtgaccttcgagcccattcctatccactactgtgcccctgccggctacgccatcctgaagtgcaacaacgagacattcaacggcacaggcccctgcagcaacgtgtccacagtgcagtgtacccacggcatcagacccgtggtgtctacacagctgctgctgaatggaagcctggccgagaaagaaatcgtgatcagaagcgagaacctgaccaacaacgccaagatcatcatcgtgcacctgaacacccctgtggaaatcgtgtgcacccggcctaacaacaacaccagaaagagcgtgcggatcggccctggccagaccttttatgccaccggcgatatcatcggcgacatcagacaggcccactgtaacatcagcgagaagcagtggaacgacaccctgcagaaagtgggcaaagagctgaagaagcacttccccaacaagaccattaagtacgcccagagcgctggcggcgacatggaaatcaccacacacagcttcaattgtggcggcgagttcttctactgcaataccgccaagctgttcaacggaacctacaacggcaccgacatcagcaccaacagcagcgccgacagcaacagcaccatcactctgcagtgccggatcaagcagatcatcaatatgtggcaaggcgtcggccggtgtatgtacgcccctcctatcgccggcaacatcacatgcaagagccacatcacaggcctgctgctcacaagagatggcggcacaaacagctccggcaaagaggaaatcttccggcctgccggcggagacatgcgggataattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagcagctggtccaacagaaacctgagcgagatctgggataacatgacctggctgcagtgggacaaagaaatcagcaactacacccagatcatctacggcctgctggaagagagccagaaccagcaagagaaaaacgagcaggacctgctggccctggactgataaggatcc

SEQ ID NO: 129 - DNA, 2013 bp, synthetic construct
  source 1..2013: mol_type = other DNA
gtcgacgccaccatgcctatgggatctctgcagcctctggccacactgtacctgctgggaatgctggtggcttctgtgctggccgagggcaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctgaagaacgtgaccgagaacttcaacatgtggaagaacgacatggtggaccagatgcacgaggacatcatcagcctgtgggaccagagcctgaagccttgcgtgaagctgacccctctgtgcgtgaccctgatctgcagcaacgccatcgtgaagaacagcaccaccgagaagatgagcaatgtgaccgtgaacaacatcaccattgaggaaatgaagaactgcagcttcaacaccaccacagagatccgggacaagattaagaaagagcgggccctgttctacagacccgacatcgtgcccctgaacgacgagacaaacaacaccagcaagtaccggctgatcaactgcaacacctccgcctgcactcaggcctgtcctaaagtgaccttcgagcccattcctatccactactgtgcccctgccggctatgccatcctgaagtgcaacgacgaaaccttcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacgcccctgtggaaatcgtgtgcacccggccttacaacaacacccggaagtctgtgcggatcggccctggccagacattctatgccaccggcgatatcatcggcgaccctagacaggcccactgtaacatcagcaaagagatctggaacaagaccctgcaagaagtgggcaaagagctgcagaagcacttccccaacaagaccatccggtacaaccagtctgccggcggagacatggaaatcaccacacacagcttcaattgtggcggcgagttcttctactgcaataccagcaacctgttcaacagcacctacaacgacacctacatcagccccaactccaccaatagcaccagcatcatcaccctgcagtgcaagatcaagcagatcatcaatatgtggcaaggcgtcggccggtgtatgtacgcccctcctatcgccggcaacatcacatgcaagagcaatatcaccggcctgctgctcaccagagatggcggcattaacaacgtgtccaacgagacagagacattcagacccgctggcggcgacatgcgggacaattggagaagcgagctgtacaagtacaaggtggtggaagtgcagcccctgggaatcgctcctaccggctgcaagagaagagtggtggaaagacgcagacggcggagagctgttggactgggtgctctgttcctgggctttctgggagccgccggatctacaatgggagctgccagcatcacactgaccgtgcaggctagacagctgctgagcggaattgtgcagcagcagagcaacctgctgagagcccctgaagcacagcagcacatgctgcagctgacagtgtggggcatcaaacagctgcaggccagagtgctggccctggaaagatacctgaaggaccagcagctcctcggaatctggggctgttctggcaagctgatctgctgcaccaacgtgccctggaacacctcctggtccaacaagagcgagacagacatttggaacaacatgacctggatgcagtgggagagagagatcagcaactacaccggcaccatctacaagctgctggaagatagccagaaccagcaagagcggaacgagaaggacctgctggctctggactgatgaggatcc

SEQ ID NO: 130 - DNA, 2046 bp, synthetic construct
  source 1..2046: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagagagggcaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctgaagaacgtgaccgagaacttcaacatgtggaagaacgacatggtggaccagatgcacgaggacatcatcagcctgtgggaccagagcctgaagccttgcgtgaagctgacccctctgtgcgtgaccctgatctgcagcaacgccatcgtgaagaacagcaccaccgagaagatgagcaatgtgaccgtgaacaacatcaccattgaggaaatgaagaactgcagcttcaacaccaccacagagatccgggacaagattaagaaagagcgggccctgttctacagacccgacatcgtgcccctgaacgacgagacaaacaacaccagcaagtaccggctgatcaactgcaacacctccgcctgcactcaggcctgtcctaaagtgaccttcgagcccattcctatccactactgtgcccctgccggctatgccatcctgaagtgcaacgacgaaaccttcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacgcccctgtggaaatcgtgtgcacccggccttacaacaacacccggaagtctgtgcggatcggccctggccagacattctatgccaccggcgatatcatcggcgaccctagacaggcccactgtaacatcagcaaagagatctggaacaagaccctgcaagaagtgggcaaagagctgcagaagcacttccccaacaagaccatccggtacaaccagtctgccggcggagacatggaaatcaccacacacagcttcaattgtggcggcgagttcttctactgcaataccagcaacctgttcaacagcacctacaacgacacctacatcagccccaactccaccaatagcaccagcatcatcaccctgcagtgcaagatcaagcagatcatcaatatgtggcaaggcgtcggccggtgtatgtacgcccctcctatcgccggcaacatcacatgcaagagcaatatcaccggcctgctgctcaccagagatggcggcattaacaacgtgtccaacgagacagagacattcagacccgctggcggcgacatgcgggacaattggagaagcgagctgtacaagtacaaggtggtggaagtgcagcccctgggaatcgctcctaccggctgcaagagaagagtggtggaaagacgcagacggcggagagctgttggactgggtgctctgttcctgggctttctgggagccgccggatctacaatgggagctgccagcatcacactgaccgtgcaggctagacagctgctgagcggaattgtgcagcagcagagcaacctgctgagagcccctgaagcacagcagcacatgctgcagctgacagtgtggggcatcaaacagctgcaggccagagtgctggccctggaaagatacctgaaggaccagcagctcctcggaatctggggctgttctggcaagctgatctgctgcaccaacgtgccctggaacacctcctggtccaacaagagcgagacagacatttggaacaacatgacctggatgcagtgggagagagagatcagcaactacaccggcaccatctacaagctgctggaagatagccagaaccagcaagagcggaacgagaaggacctgctggctctggactgatgaggatcc

SEQ ID NO: 131 - DNA, 2013 bp, synthetic construct
  source 1..2013: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctgaagaacgtgaccgagaacttcaacatgtggaagaacgacatggtggaccagatgcacgaggacatcatcagcctgtgggaccagagcctgaagccttgcgtgaagctgacccctctgtgcgtgaccctgatctgcagcaacgccatcgtgaagaacagcaccaccgagaagatgagcaatgtgaccgtgaacaacatcaccattgaggaaatgaagaactgcagcttcaacaccaccacagagatccgggacaagattaagaaagagcgggccctgttctacagacccgacatcgtgcccctgaacgacgagacaaacaacaccagcaagtaccggctgatcaactgcaacacctccgcctgcactcaggcctgtcctaaagtgaccttcgagcccattcctatccactactgtgcccctgccggctatgccatcctgaagtgcaacgacgaaaccttcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacgcccctgtggaaatcgtgtgcacccggccttacaacaacacccggaagtctgtgcggatcggccctggccagacattctatgccaccggcgatatcatcggcgaccctagacaggcccactgtaacatcagcaaagagatctggaacaagaccctgcaagaagtgggcaaagagctgcagaagcacttccccaacaagaccatccggtacaaccagtctgccggcggagacatggaaatcaccacacacagcttcaattgtggcggcgagttcttctactgcaataccagcaacctgttcaacagcacctacaacgacacctacatcagccccaactccaccaatagcaccagcatcatcaccctgcagtgcaagatcaagcagatcatcaatatgtggcaaggcgtcggccggtgtatgtacgcccctcctatcgccggcaacatcacatgcaagagcaatatcaccggcctgctgctcaccagagatggcggcattaacaacgtgtccaacgagacagagacattcagacccgctggcggcgacatgcgggacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgacactgaccgtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaactccagctggtccaacagaaacctgagcgagatctgggataacatgacctggctgcagtgggacaaagaaatcagcaactacacccagatcatctacggactgctggaagagagccagaaccagcaagagaaaaacgagcaggacctgctggccctggactgataaggatcc

SEQ ID NO: 132 - DNA, 1974 bp, synthetic construct
  source 1..1974: mol_type = other DNA
gtcgacgccaccatgcctatgggatctctgcagcctctggccacactgtacctgctgggaatgctggtggcttctgtgctggccgagggcaatctgtgggtcacagtgtactatggcgtgcccgtgtggaaagaggccaagaccacactgttctgtgccagcgacgccaaggcctacgtgaaagaggtgcacaacgtctgggccacacacgcctgtgtgcctaccgatccatctcctcaagagctgttcctgaagaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagcaagttcgaggaaatgaagaactgcagcttcaacaccaccaccgagatccgggacaaagagaagaaagagtacgccctgttctacaggcccgacatcgtgcccctggacaacgagacaagcaacatcagcgagtaccggctgatcaactgcaacaccagcgcctgcactcaggcctgtcctaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacaccagaaagagcgtgcggatcggccctggccagaccttttatgccaccggcgacatcatcggcaaccccagacaggcccactgtaacatcagcaaagagcggtggaacgacaccctgcagaaagtgggcaaagagctgcagaagcacttccccaacaagaccatcaagtacaaccagagcgctggcggcgacatggaaatcaccacacacagcttcaattgtggcggcgagttcttctactgcaatacctccaacctgttcaacagcacctacaacgacacctacatcagcaccaactccaccaatagcaccagctacattaccctgcagtgccggatcaagcagatcatcaatatgtggcaaggcgtcggccggtgtatgtacgcccctcctatcgccggcaacatcacatgcaagtccaacatcaccggcctgctgctcacaagagatggcggcatcaacaaggacagcaacgaaaccgaaaccttccggcctgccggcggagacatgagagacaattggagaagcgagctgtacaagtacaaggtggtggaagtgcagcccctgggaatcgctcctaccggctgcaagagaagagtggtggaaagacgcagacggcggagagctgctggactgggtgctctgtttctgggctttctgggagccgccggatctacaatgggagctgccagcatcaccctgaccgtgcaggctagacaactgctgtctggcatcgtgcagcagcagagcaacctgctgagagcccctgaagctcagcagcacatgctgcagctgacagtgtggggcatcaaacagctgcaggccagagttctggccctggaaagatacctgaaggatcagcagctcctcggcatgtggggctgttctggcaagctgatctgctgcaccaacgtgccctggaacacctcctggtccaacaagagcgagacagacatctgggacaacatgacctggatgcagtgggagagagagatcagcaactacaccggcaccatctacaagctgctcgaggacagccagaaccagcaagagagaaacgagcaggacctgctggctctggactgatgaggatcc

SEQ ID NO: 133 - DNA, 2007 bp, synthetic construct
  source 1..2007: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagagagggcaatctgtgggtcacagtgtactatggcgtgcccgtgtggaaagaggccaagaccacactgttctgtgccagcgacgccaaggcctacgtgaaagaggtgcacaacgtctgggccacacacgcctgtgtgcctaccgatccatctcctcaagagctgttcctgaagaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagcaagttcgaggaaatgaagaactgcagcttcaacaccaccaccgagatccgggacaaagagaagaaagagtacgccctgttctacaggcccgacatcgtgcccctggacaacgagacaagcaacatcagcgagtaccggctgatcaactgcaacaccagcgcctgcactcaggcctgtcctaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacaccagaaagagcgtgcggatcggccctggccagaccttttatgccaccggcgacatcatcggcaaccccagacaggcccactgtaacatcagcaaagagcggtggaacgacaccctgcagaaagtgggcaaagagctgcagaagcacttccccaacaagaccatcaagtacaaccagagcgctggcggcgacatggaaatcaccacacacagcttcaattgtggcggcgagttcttctactgcaatacctccaacctgttcaacagcacctacaacgacacctacatcagcaccaactccaccaatagcaccagctacattaccctgcagtgccggatcaagcagatcatcaatatgtggcaaggcgtcggccggtgtatgtacgcccctcctatcgccggcaacatcacatgcaagtccaacatcaccggcctgctgctcacaagagatggcggcatcaacaaggacagcaacgaaaccgaaaccttccggcctgccggcggagacatgagagacaattggagaagcgagctgtacaagtacaaggtggtggaagtgcagcccctgggaatcgctcctaccggctgcaagagaagagtggtggaaagacgcagacggcggagagctgctggactgggtgctctgtttctgggctttctgggagccgccggatctacaatgggagctgccagcatcaccctgaccgtgcaggctagacaactgctgtctggcatcgtgcagcagcagagcaacctgctgagagcccctgaagctcagcagcacatgctgcagctgacagtgtggggcatcaaacagctgcaggccagagttctggccctggaaagatacctgaaggatcagcagctcctcggcatgtggggctgttctggcaagctgatctgctgcaccaacgtgccctggaacacctcctggtccaacaagagcgagacagacatctgggacaacatgacctggatgcagtgggagagagagatcagcaactacaccggcaccatctacaagctgctcgaggacagccagaaccagcaagagagaaacgagcaggacctgctggctctggactgatgaggatcc

SEQ ID NO: 134 - DNA, 1974 bp, synthetic construct
  source 1..1974: mol_type = other DNA
gtcgacgccaccatgcctatgggatctctgcagcctctggccacactgtacctgctgggaatgctggtggcttctgtgctggccgccgagaatctggtcacagtgtactatggcgtgcccgtgtggaaagaggccaagaccacactgttctgtgccagcgacgccaaggcctacgtgaaagaggtgcacaacgtctgggccacacacgcctgtgtgcctaccgatccatctcctcaagagctgttcctgaagaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagcaagttcgaggaaatgaagaactgcagcttcaacaccaccaccgagatccgggacaaagagaagaaagagtacgccctgttctacaggcccgacatcgtgcccctggacaacgagacaagcaacatcagcgagtaccggctgatcaactgcaacaccagcgcctgcactcaggcctgtcctaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacaccagaaagagcgtgcggatcggccctggccagaccttttatgccaccggcgacatcatcggcaaccccagacaggcccactgtaacatcagcaaagagcggtggaacgacaccctgcagaaagtgggcaaagagctgcagaagcacttccccaacaagaccatcaagtacaaccagagcgctggcggcgacatggaaatcaccacacacagcttcaattgtggcggcgagttcttctactgcaatacctccaacctgttcaacagcacctacaacgacacctacatcagcaccaactccaccaatagcaccagctacattaccctgcagtgccggatcaagcagatcatcaatatgtggcaaggcgtcggccggtgtatgtacgcccctcctatcgccggcaacatcacatgcaagtccaacatcaccggcctgctgctcacaagagatggcggcatcaacaaggacagcaacgaaaccgaaaccttccggcctgccggcggagacatgagagacaattggagaagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaactccagctggtccaacagaaacctgagcgagatctgggataacatgacctggctgcagtgggacaaagaaatcagcaactacacccagatcatctacggactgctggaagagagccagaaccagcaagagaaaaacgagcaggacctgctggccctggactgataaggatcc

SEQ ID NO: 135 - protein, 655 aa, synthetic construct
MPMGSLQPLATLYLLGMLVASVLAAENLWVTVYYGVPVWEDADTPLFCASDAKAYSTESHNVWATHACVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTCKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQCMYAPPIAGNITCRSNITGLLLTRDGGGNNTNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 136 - protein, 655 aa, synthetic construct
MPMGSLQPLATLYLLGMLVASVLASNNLWVTVYYGVPVWEDADTPLFCASDAKAYSTESHNVWATHACVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTIKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQAMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASITLTVQVRQLLSGIVQQQSNLLRAPEAQQHLLQLTVWGIKQLQARILAVERYLKDQQLLGIWGCSGKLICCTNVPWNASWSNRSYNDIWENLTWVEWEREISNYTQQIYSLLEESQNQQEKNEQDLLALD

SEQ ID NO: 137 - protein, 655 aa, synthetic construct
MPMGSLQPLATLYLLGMLVASVLASNNLWVTVYYGVPVWEDADTPLFCASDAKAYSTESHNVWATHACVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTCKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQCMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASITLTVQVRQLLSGIVQQQSNLLRAPEAQQHLLQLTVWGIKQLQARILAVERYLKDQQLLGIWGCSGKLICCTNVPWNASWSNRSYNDIWENLTWVEWEREISNYTQQIYSLLEESQNQQEKNEQDLLALD

SEQ ID NO: 138 - protein, 642 aa, synthetic construct
MPMGSLQPLATLYLLGMLVASVLASNNLWVTVYYGVPVWEDADTPLFCASDAKAYSTESHNVWATHACVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTCKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQCMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASITLTVQVRQLLSGNPDWLPDMTVWGIKQLQARILAVERYLKDQQLLGIWGCSGKLICCTNVPWNASWSNRSYNDIWENLTWVEWEREISNYTQQIYSLLEESQNQQEKNEQDLLALD

SEQ ID NO: 139 - protein, 655 aa, synthetic construct
MPMGSLQPLATLYLLGMLVASVLASNNLWVTVYYGVPVWEDACTPLFCASDAKAYSTKSRNVWATCCCVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTIKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAWYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQAMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASMTLTVQVRQLLSGIVQQQSNCLRAPECQQCLLQLTVWGIKQLQARILAVERYLKDQQLLGIWGCSGKLICCTNVPWNASWSNRSYNDIWENLTWVEWEREISNYTQQIYSLLEESQNQQEKNEQDLLALD

SEQ ID NO: 140 - protein, 655 aa, synthetic construct
MPMGSLQPLATLYLLGMLVASVLASNNLWVTVYYGVPVWEDAETPLFCASDAKAYSTEKHNVWATHACVPTDPSPQEISLDNVTENFNMWKNNMVEQMHTDIISLWDESLKPCVKLTPLCVTLNCTNVNNSSATNNSMVDDREGLKNCSFNITTELRDKKKQVHALFYRLDIVPINGNSNSNSSVGDYRLINCNVSTIKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSIRLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQAMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASITLTVQVRNLLSGIVQQQSNLLRAPEAQQHLLQLTVWGIKQLQARILAVERYLRDQQLLGIWGCSGKLICCTNVPWNASWSNRSYNDIWENLTWVEWEREISNYTQQIYSLLEESQNQQEKNEQDLLALD

SEQ ID NO: 141 - protein, 666 aa, synthetic construct
MDAMKRGLCCVLLLCGAVFVSPSQEIHARFRRGARAENLWVTVYYGVPVWEDADTPLFCASDAKAYSTESHNVWATHACVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTCKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQCMYAPPIAGNITCRSNITGLLLTRDGGGNNTNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 142 - protein, 666 aa, synthetic construct
MDAMKRGLCCVLLLCGAVFVSPSQEIHARFRRGARSNNLWVTVYYGVPVWEDADTPLFCASDAKAYSTESHNVWATHACVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTIKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQAMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASITLTVQVRQLLSGIVQQQSNLLRAPEAQQHLLQLTVWGIKQLQARILAVERYLKDQQLLGIWGCSGKLICCTNVPWNASWSNRSYNDIWENLTWVEWEREISNYTQQIYSLLEESQNQQEKNEQDLLALD

SEQ ID NO: 143 - protein, 666 aa, synthetic construct
MDAMKRGLCCVLLLCGAVFVSPSQEIHARFRRGARSNNLWVTVYYGVPVWEDADTPLFCASDAKAYSTESHNVWATHACVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTCKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQCMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASITLTVQVRQLLSGIVQQQSNLLRAPEAQQHLLQLTVWGIKQLQARILAVERYLKDQQLLGIWGCSGKLICCTNVPWNASWSNRSYNDIWENLTWVEWEREISNYTQQIYSLLEESQNQQEKNEQDLLALD

SEQ ID NO: 144 - protein, 653 aa, synthetic construct
MDAMKRGLCCVLLLCGAVFVSPSQEIHARFRRGARSNNLWVTVYYGVPVWEDADTPLFCASDAKAYSTESHNVWATHACVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTCKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQCMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASITLTVQVRQLLSGNPDWLPDMTVWGIKQLQARILAVERYLKDQQLLGIWGCSGKLICCTNVPWNASWSNRSYNDIWENLTWVEWEREISNYTQQIYSLLEESQNQQEKNEQDLLALD

SEQ ID NO: 145 - protein, 666 aa, synthetic construct
MDAMKRGLCCVLLLCGAVFVSPSQEIHARFRRGARSNNLWVTVYYGVPVWEDACTPLFCASDAKAYSTKSRNVWATCCCVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTIKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAWYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQAMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASMTLTVQVRQLLSGIVQQQSNCLRAPECQQCLLQLTVWGIKQLQARILAVERYLKDQQLLGIWGCSGKLICCTNVPWNASWSNRSYNDIWENLTWVEWEREISNYTQQIYSLLEESQNQQEKNEQDLLALD

SEQ ID NO: 146 - protein, 666 aa, synthetic construct
MDAMKRGLCCVLLLCGAVFVSPSQEIHARFRRGARSNNLWVTVYYGVPVWEDAETPLFCASDAKAYSTEKHNVWATHACVPTDPSPQEISLDNVTENFNMWKNNMVEQMHTDIISLWDESLKPCVKLTPLCVTLNCTNVNNSSATNNSMVDDREGLKNCSFNITTELRDKKKQVHALFYRLDIVPINGNSNSNSSVGDYRLINCNVSTIKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSIRLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQAMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASITLTVQVRNLLSGIVQQQSNLLRAPEAQQHLLQLTVWGIKQLQARILAVERYLRDQQLLGIWGCSGKLICCTNVPWNASWSNRSYNDIWENLTWVEWEREISNYTQQIYSLLEESQNQQEKNEQDLLALD

SEQ ID NO: 147 - protein, 644 aa, synthetic construct
MGSLQPLATLYLLGMLVASVLAAENLWVTVYYGVPVWKEAKTTLFCASDAKAYDPEVHNVWATHACVPTDPNPQEMFLENVTENFNMWKNDMVEQMHQDIISLWSQSLKPCVKLTPLCVTLNCSDVINSTGTMKNCSFSVTTELRDRKQKKHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTFFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQGVGQCMYAPPIAGNIICRSKITGLLLTRDGGINKSEEIFRPGGGNMKDNWRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 148 - protein, 639 aa, synthetic construct
MGSLQPLATLYLLGMLVASVLANLWVTVYYGVPVWKEAKTTLFCASDAKAYDPEVHNVWATHACVPTDPNPQEMFLENVTENFNMWKNDMVEQMHQDIISLWSQSLKPCVKLTPLCVTLNCSDVINSTGTMKNCSFSVTTELRDRKQKKHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTFFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQGVGQCMYAPPIAGNIICRSKITGLLLTRDGGINKSEEIFRPGGGNMKDNWRSELYKYKVVEIKPLGVAPTKCKRRVVERRRRRRAVVGLGAVFLGFLGAAGSTMGAASITLTVQARQLLSGIVQQQNNLLRAPEAQQHMLQLTVWGIKQLQARVLAIERYLKDQQLLGIWGCSGKLICCTAVPWNSSWSNKSQEDIWDNMTWMQWDREISNYTNTIYRLLEDSQTQQEKNEKDLLALD

SEQ ID NO: 149 - protein, 639 aa, synthetic construct
MGSLQPLATLYLLGMLVASVLANLWVTVYYGVPVWKEACTTLFCASDAKAYDPKVRNVWATHCCVPTDPNPQEMFLENVTENFNMWKNDMVEQMHQDIISLWSQSLKPCVKLTPLCVTLNCSDVINSTGTMKNCSFSVTTELRDRKQKKHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTWFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQGVGQCMYAPPIAGNIICRSKITGLLLTRDGGINKSEEIFRPGGGNMKDNWRSELYKYKVVEIKPLGVAPTKCKRRVVERRRRRRAVVGLGAVFLGFLGAAGSTMGAASMTLTVQARQLLSGIVQQQNNCLRAPECQQCMLQLTVWGIKQLQARVLAIERYLKDQQLLGIWGCSGKLICCTAVPWNSSWSNKSQEDIWDNMTWMQWDREISNYTNTIYRLLEDSQTQQEKNEKDLLALD

SEQ ID NO: 150 - protein, 626 aa, synthetic construct
MGSLQPLATLYLLGMLVASVLANLWVTVYYGVPVWKEAKTTLFCASDAKAYDPEVHNVWATHACVPTDPNPQEMFLENVTENFNMWKNDMVEQMHQDIISLWSQSLKPCVKLTPLCVTLNCSDVINSTGTMKNCSFSVTTELRDRKQKKHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTFFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQGVGQCMYAPPIAGNIICRSKITGLLLTRDGGINKSEEIFRPGGGNMKDNWRSELYKYKVVEIKPLGVAPTKCKRRVVERRRRRRAVVGLGAVFLGFLGAAGSTMGAASITLTVQARQLLSGNPDWLPDMTVWGIKQLQARVLAIERYLKDQQLLGIWGCSGKLICCTAVPWNSSWSNKSQEDIWDNMTWMQWDREISNYTNTIYRLLEDSQTQQEKNEKDLLALD

SEQ ID NO: 151 - protein, 639 aa, synthetic construct
MGSLQPLATLYLLGMLVASVLANLWVTVYYGVPVWKDAETTLFCASDAKAYDPEKHNVWATHACVPTDPNPQEMFLENVTENFNMWKNDMVEQMHTDIISLWSQSLKPCVKLTPLCVTLNCSDVINSTGTMKNCSFSVTTELRDRKQKVHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTFFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQRVGQCMYAPPIAGNIICRSKITGLLLTRDGGINKSEEIFRPGGGNMKDNWRSELYKYKVVEIKPLGVAPTRCKRRVVERRRRRRAVVGLGAVFLGFLGAAGSTMGAASITLTVQARNLLSGIVQQQSNLLRAPEAQQHMLQLTVWGIKQLQARVLAIERYLRDQQLLGIWGCSGKLICCTAVPWNSSWSNKSQEDIWDNMTWMQWDREISNYTNTIYRLLEDSQTQQEKNEKDLLALD

SEQ ID NO: 152 - protein, 657 aa, synthetic construct
MDAMKRGLCCVLLLCGAVFVSPSQEIHARFRRGARAENLWVTVYYGVPVWKEAKTTLFCASDAKAYDPEVHNVWATHACVPTDPNPQEMFLENVTENFNMWKNDMVEQMHQDIISLWSQSLKPCVKLTPLCVTLNCSDVINSTGTMKNCSFSVTTELRDRKQKKHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTFFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQGVGQCMYAPPIAGNIICRSKITGLLLTRDGGINKSEEIFRPGGGNMKDNWRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 153 - protein, 652 aa, synthetic construct
MDAMKRGLCCVLLLCGAVFVSPSQEIHARFRRGARNLWVTVYYGVPVWKEAKTTLFCASDAKAYDPEVHNVWATHACVPTDPNPQEMFLENVTENFNMWKNDMVEQMHQDIISLWSQSLKPCVKLTPLCVTLNCSDVINSTGTMKNCSFSVTTELRDRKQKKHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTFFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQGVGQCMYAPPIAGNIICRSKITGLLLTRDGGINKSEEIFRPGGGNMKDNWRSELYKYKVVEIKPLGVAPTKCKRRVVERRRRRRAVVGLGAVFLGFLGAAGSTMGAASITLTVQARQLLSGIVQQQNNLLRAPEAQQHMLQLTVWGIKQLQARVLAIERYLKDQQLLGIWGCSGKLICCTAVPWNSSWSNKSQEDIWDNMTWMQWDREISNYTNTIYRLLEDSQTQQEKNEKDLLALD

SEQ ID NO: 154 - protein, 652 aa, synthetic construct
MDAMKRGLCCVLLLCGAVFVSPSQEIHARFRRGARNLWVTVYYGVPVWKEACTTLFCASDAKAYDPKVRNVWATHCCVPTDPNPQEMFLENVTENFNMWKNDMVEQMHQDIISLWSQSLKPCVKLTPLCVTLNCSDVINSTGTMKNCSFSVTTELRDRKQKKHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTWFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQGVGQCMYAPPIAGNIICRSKITGLLLTRDGGINKSEEIFRPGGGNMKDNWRSELYKYKVVEIKPLGVAPTKCKRRVVERRRRRRAVVGLGAVFLGFLGAAGSTMGAASMTLTVQARQLLSGIVQQQNNCLRAPECQQCMLQLTVWGIKQLQARVLAIERYLKDQQLLGIWGCSGKLICCTAVPWNSSWSNKSQEDIWDNMTWMQWDREISNYTNTIYRLLEDSQTQQEKNEKDLLALD

SEQ ID NO: 155 - protein, 639 aa, synthetic construct
MDAMKRGLCCVLLLCGAVFVSPSQEIHARFRRGARNLWVTVYYGVPVWKEAKTTLFCASDAKAYDPEVHNVWATHACVPTDPNPQEMFLENVTENFNMWKNDMVEQMHQDIISLWSQSLKPCVKLTPLCVTLNCSDVINSTGTMKNCSFSVTTELRDRKQKKHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTFFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQGVGQCMYAPPIAGNIICRSKITGLLLTRDGGINKSEEIFRPGGGNMKDNWRSELYKYKVVEIKPLGVAPTKCKRRVVERRRRRRAVVGLGAVFLGFLGAAGSTMGAASITLTVQARQLLSGNPDWLPDMTVWGIKQLQARVLAIERYLKDQQLLGIWGCSGKLICCTAVPWNSSWSNKSQEDIWDNMTWMQWDREISNYTNTIYRLLEDSQTQQEKNEKDLLALD

SEQ ID NO: 156 - protein, 652 aa, synthetic construct
MDAMKRGLCCVLLLCGAVFVSPSQEIHARFRRGARNLWVTVYYGVPVWKDAETTLFCASDAKAYDPEKHNVWATHACVPTDPNPQEMFLENVTENFNMWKNDMVEQMHTDIISLWSQSLKPCVKLTPLCVTLNCSDVINSTGTMKNCSFSVTTELRDRKQKVHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTFFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQRVGQCMYAPPIAGNIICRSKITGLLLTRDGGINKSEEIFRPGGGNMKDNWRSELYKYKVVEIKPLGVAPTRCKRRVVERRRRRRAVVGLGAVFLGFLGAAGSTMGAASITLTVQARNLLSGIVQQQSNLLRAPEAQQHMLQLTVWGIKQLQARVLAIERYLRDQQLLGIWGCSGKLICCTAVPWNSSWSNKSQEDIWDNMTWMQWDREISNYTNTIYRLLEDSQTQQEKNEKDLLALD

SEQ ID NO: 157 - protein, 649 aa, synthetic construct
MPMGSLQPLATLYLLGMLVASVLAKGKLWVTVYYGVPVWKEAKTTLFCASDARAYEKEVHNVWATHACVPTDPSPQELFLKNVTENFNMWKNDMVDQMHEDIISLWDQSLKPCVKLTPLCVTLICSNATVKNRTVEEMKNCSFNTTTEIRDKEKKEYALFYKPDVVPLDETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTPVEIVCTRPNNNTRKSVRIGPGQTFYATGDIIGDIRQAHCNISEKQWNDTLQKVGKELKKHFPNKTIKYAQSAGGDMEITTHSFNCGGEFFYCNTAKLFNGTYNGTDISTNSSADSNSTITLQCRIKQIINMWQGVGRCMYAPPIAGNITCKSHITGLLLTRDGGTNSSGKEEIFRPAGGDMRDNWRSELYKYKVVEIQPLGIAPTGCKRRVVERRRRRRAAGLGALFLGFLGAAGSTMGAASITLTVQARQLLSGIVQQQSNLLRAPEAQQHMLQLTVWGIKQLQARVLALERYLKDQQLLGMWGCSGKLICCTNVPWNTSWSNKSETDIWDNMTWMQWEREISNYTETIYKLLEDSQNQQERNEQDLLALD

SEQ ID NO: 158 - protein, 660 aa, synthetic construct
MDAMKRGLCCVLLLCGAVFVSPSQEIHARFRRGARKGKLWVTVYYGVPVWKEAKTTLFCASDARAYEKEVHNVWATHACVPTDPSPQELFLKNVTENFNMWKNDMVDQMHEDIISLWDQSLKPCVKLTPLCVTLICSNATVKNRTVEEMKNCSFNTTTEIRDKEKKEYALFYKPDVVPLDETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTPVEIVCTRPNNNTRKSVRIGPGQTFYATGDIIGDIRQAHCNISEKQWNDTLQKVGKELKKHFPNKTIKYAQSAGGDMEITTHSFNCGGEFFYCNTAKLFNGTYNGTDISTNSSADSNSTITLQCRIKQIINMWQGVGRCMYAPPIAGNITCKSHITGLLLTRDGGTNSSGKEEIFRPAGGDMRDNWRSELYKYKVVEIQPLGIAPTGCKRRVVERRRRRRAAGLGALFLGFLGAAGSTMGAASITLTVQARQLLSGIVQQQSNLLRAPEAQQHMLQLTVWGIKQLQARVLALERYLKDQQLLGMWGCSGKLICCTNVPWNTSWSNKSETDIWDNMTWMQWEREISNYTETIYKLLEDSQNQQERNEQDLLALD

SEQ ID NO: 159 - protein, 649 aa, synthetic construct
MPMGSLQPLATLYLLGMLVASVLAAENLWVTVYYGVPVWKEAKTTLFCASDARAYEKEVHNVWATHACVPTDPSPQELFLKNVTENFNMWKNDMVDQMHEDIISLWDQSLKPCVKLTPLCVTLICSNATVKNRTVEEMKNCSFNTTTEIRDKEKKEYALFYKPDVVPLDETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTPVEIVCTRPNNNTRKSVRIGPGQTFYATGDIIGDIRQAHCNISEKQWNDTLQKVGKELKKHFPNKTIKYAQSAGGDMEITTHSFNCGGEFFYCNTAKLFNGTYNGTDISTNSSADSNSTITLQCRIKQIINMWQGVGRCMYAPPIAGNITCKSHITGLLLTRDGGTNSSGKEEIFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 160 - protein, 663 aa, synthetic construct
MPMGSLQPLATLYLLGMLVASVLAEGNLWVTVYYGVPVWKEAKTTLFCASDAKAYKKEVHNVWATHACVPTDPSPQELFLKNVTENFNMWKNDMVDQMHEDIISLWDQSLKPCVKLTPLCVTLICSNAIVKNSTTEKMSNVTVNNITIEEMKNCSFNTTTEIRDKIKKERALFYRPDIVPLNDETNNTSKYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHAPVEIVCTRPYNNTRKSVRIGPGQTFYATGDIIGDPRQAHCNISKEIWNKTLQEVGKELQKHFPNKTIRYNQSAGGDMEITTHSFNCGGEFFYCNTSNLFNSTYNDTYISPNSTNSTSIITLQCKIKQIINMWQGVGRCMYAPPIAGNITCKSNITGLLLTRDGGINNVSNETETFRPAGGDMRDNWRSELYKYKVVEVQPLGIAPTGCKRRVVERRRRRRAVGLGALFLGFLGAAGSTMGAASITLTVQARQLLSGIVQQQSNLLRAPEAQQHMLQLTVWGIKQLQARVLALERYLKDQQLLGIWGCSGKLICCTNVPWNTSWSNKSETDIWNNMTWMQWEREISNYTGTIYKLLEDSQNQQERNEKDLLALD

SEQ ID NO: 161 - protein, 674 aa, synthetic construct
MDAMKRGLCCVLLLCGAVFVSPSQEIHARFRRGAREGNLWVTVYYGVPVWKEAKTTLFCASDAKAYKKEVHNVWATHACVPTDPSPQELFLKNVTENFNMWKNDMVDQMHEDIISLWDQSLKPCVKLTPLCVTLICSNAIVKNSTTEKMSNVTVNNITIEEMKNCSFNTTTEIRDKIKKERALFYRPDIVPLNDETNNTSKYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHAPVEIVCTRPYNNTRKSVRIGPGQTFYATGDIIGDPRQAHCNISKEIWNKTLQEVGKELQKHFPNKTIRYNQSAGGDMEITTHSFNCGGEFFYCNTSNLFNSTYNDTYISPNSTNSTSIITLQCKIKQIINMWQGVGRCMYAPPIAGNITCKSNITGLLLTRDGGINNVSNETETFRPAGGDMRDNWRSELYKYKVVEVQPLGIAPTGCKRRVVERRRRRRAVGLGALFLGFLGAAGSTMGAASITLTVQARQLLSGIVQQQSNLLRAPEAQQHMLQLTVWGIKQLQARVLALERYLKDQQLLGIWGCSGKLICCTNVPWNTSWSNKSETDIWNNMTWMQWEREISNYTGTIYKLLEDSQNQQERNEKDLLALD

SEQ ID NO: 162 - protein, 663 aa, synthetic construct
MPMGSLQPLATLYLLGMLVASVLAAENLWVTVYYGVPVWKEAKTTLFCASDAKAYKKEVHNVWATHACVPTDPSPQELFLKNVTENFNMWKNDMVDQMHEDIISLWDQSLKPCVKLTPLCVTLICSNAIVKNSTTEKMSNVTVNNITIEEMKNCSFNTTTEIRDKIKKERALFYRPDIVPLNDETNNTSKYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHAPVEIVCTRPYNNTRKSVRIGPGQTFYATGDIIGDPRQAHCNISKEIWNKTLQEVGKELQKHFPNKTIRYNQSAGGDMEITTHSFNCGGEFFYCNTSNLFNSTYNDTYISPNSTNSTSIITLQCKIKQIINMWQGVGRCMYAPPIAGNITCKSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 163 - protein, 650 aa, synthetic construct
MPMGSLQPLATLYLLGMLVASVLAEGNLWVTVYYGVPVWKEAKTTLFCASDAKAYVKEVHNVWATHACVPTDPSPQELFLKNVTENFNMWKNDMVDQMHEDIISLWDQSLKPCVKLTPLCVTLICSTATVNNSKFEEMKNCSFNTTTEIRDKEKKEYALFYRPDIVPLDNETSNISEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGNPRQAHCNISKERWNDTLQKVGKELQKHFPNKTIKYNQSAGGDMEITTHSFNCGGEFFYCNTSNLFNSTYNDTYISTNSTNSTSYITLQCRIKQIINMWQGVGRCMYAPPIAGNITCKSNITGLLLTRDGGINKDSNETETFRPAGGDMRDNWRSELYKYKVVEVQPLGIAPTGCKRRVVERRRRRRAAGLGALFLGFLGAAGSTMGAASITLTVQARQLLSGIVQQQSNLLRAPEAQQHMLQLTVWGIKQLQARVLALERYLKDQQLLGMWGCSGKLICCTNVPWNTSWSNKSETDIWDNMTWMQWEREISNYTGTIYKLLEDSQNQQERNEQDLLALD

SEQ ID NO: 164 - protein, 661 aa, synthetic construct
MDAMKRGLCCVLLLCGAVFVSPSQEIHARFRRGAREGNLWVTVYYGVPVWKEAKTTLFCASDAKAYVKEVHNVWATHACVPTDPSPQELFLKNVTENFNMWKNDMVDQMHEDIISLWDQSLKPCVKLTPLCVTLICSTATVNNSKFEEMKNCSFNTTTEIRDKEKKEYALFYRPDIVPLDNETSNISEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGNPRQAHCNISKERWNDTLQKVGKELQKHFPNKTIKYNQSAGGDMEITTHSFNCGGEFFYCNTSNLFNSTYNDTYISTNSTNSTSYITLQCRIKQIINMWQGVGRCMYAPPIAGNITCKSNITGLLLTRDGGINKDSNETETFRPAGGDMRDNWRSELYKYKVVEVQPLGIAPTGCKRRVVERRRRRRAAGLGALFLGFLGAAGSTMGAASITLTVQARQLLSGIVQQQSNLLRAPEAQQHMLQLTVWGIKQLQARVLALERYLKDQQLLGMWGCSGKLICCTNVPWNTSWSNKSETDIWDNMTWMQWEREISNYTGTIYKLLEDSQNQQERNEQDLLALD

SEQ ID NO: 165 - protein, 650 aa, synthetic construct
MPMGSLQPLATLYLLGMLVASVLAAENLVTVYYGVPVWKEAKTTLFCASDAKAYVKEVHNVWATHACVPTDPSPQELFLKNVTENFNMWKNDMVDQMHEDIISLWDQSLKPCVKLTPLCVTLICSTATVNNSKFEEMKNCSFNTTTEIRDKEKKEYALFYRPDIVPLDNETSNISEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGNPRQAHCNISKERWNDTLQKVGKELQKHFPNKTIKYNQSAGGDMEITTHSFNCGGEFFYCNTSNLFNSTYNDTYISTNSTNSTSYITLQCRIKQIINMWQGVGRCMYAPPIAGNITCKSNITGLLLTRDGGINKDSNETETFRPAGGDMRDNWRRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 166 - DNA, 1999 bp, synthetic construct
  source 1..1999: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacgagaaggaggtgcacaacgtgtgggccacccacgcctgcgtgcccaccgacccctccccccaggagctggtgctgaagaacgtgaccgagaacttcaacatgtggaagaacgacatggtggaccagatgcacgaggacatcatctccctgtgggaccagtccctgaagccctgcgtgaagctgacccccctgtgcgtgaccctgaactgctccaacgcccgctccaacgtgaacgtgacctccatcaacaacaccatcatgggcgagatgaagaactgctccttcaacaccaccaccgagatccgcgacaaggagaagaaggagtacgccctgttctacaagcccgacgtggtgcccctgaacgagacctccaacacctccgagtaccgcctgatcaactgcaacacctccgcctgcacccaggcctgccccaaggtgaccttcgagcccatccccatccactactgcgcccccgccggctacgccatcctgaagtgcaacaacaagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgaacacctccgtggagatcgtgtgcacccgccccggcaacaacacccgcaagtccgtgcgcatcggccccggccagaccttctacgccaccggcgacatcatcggcgacatccgccaggcccactgcaacatctccgagggccagtggaacaagaccctgcacgaggtgtccaaggagctgcagaagcacttccccaacaagaccatcaagtacgagcgctccgccggcggcgacatggagatcgccacccactccttcaactgcggcggcgagttcttctactgcaacacctccaacctgttcaacggcacctacaacggcacctacatcaacacctcctccacctcctacatcaccctgcagtgccgcatcaagcagatcatcaacatgtggcagggcgtgggccgctgcatgtacgccccccccatcgccggcaacatcacctgcaagtccaacatcaccggcctgctgctgacccgcgacggcggcaccaagaacaactccaacgaggagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactagtaagcggccgcggatcc

SEQ ID NO: 167 - DNA, 1999 bp, synthetic construct
  source 1..1999: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacgagaagaaggtgcacaacgtgtgggccacccacgcctgcgtgcccaccgacccctccccccaggagctggtgctgaagaacgtgaccgagaacttcaacatgtggaagaacgacatggtggaccagatgcacgaggacatcatctccctgtgggaccagtccctgaagccctgcgtgaagctgacccccctgtgcgtgaccctgaactgctccaacgcccgctccaacgtgaacgtgacctccatcaacaacaccatcatgggcgagatgaagaactgctccttcaacaccaccaccgagatccgcgacaaggagaagaaggagtacgccctgttctacaagcccgacgtggtgcccctgaacgagacctccaacacctccgagtaccgcctgatcaactgcaacacctccgccgtgacccaggcctgccccaaggtgaccttcgagcccatccccatccactactgcgcccccgccggctacgccatcctgaagtgcaacaacaagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgaacacctccgtggagatcgtgtgcacccgccccggcaacaacacccgcaagtccgtgcgcatcggccccggccagtggttctacgccaccggcgacatcatcggcgacatccgccaggcccactgcaacatctccgagggccagtggaacaagaccctgcacgaggtgtccaaggagctgcagaagcacttccccaacaagaccatcaagtacgagcgctccgccggcggcgacatggagatcgccacccactccttcaactgcggcggcgagttcttctactgcaacacctccaacctgttcaacggcacctacaacggcacctacatcaacacctcctccacctcctacatcaccctgcagtgccgcatcaagcagatcatcaacatgtggcagggcgtgggccgcgccatgtacgccccccccatcgccggcaacatcacctgcaagtccaacatcaccggcctgctgctgacccgcgacggcggcaccaagaacaactccaacgaggagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactagtaagcggccgcggatcc

SEQ ID NO: 168 - DNA, 1999 bp, synthetic construct
  source 1..1999: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacgagaaggaggtgcacaacgtgtgggccacccacgcctgcgtgcccaccgacccctccccccaggagctggtgctgaagaacgtgaccgagaacttcaacatgtggaagaacgacatggtggaccagatgcacgaggacatcatctccctgtgggaccagtccctgaagccctgcgtgaagctgacccccctgtgcgtgaccctgaactgctccaacgcccgctccaacgtgaacgtgacctccatcaacaacaccatcatgggcgagatgaagaactgctccttcaacaccaccaccgagatccgcgacaaggagaagaaggagtacgccctgttctacaagcccgacgtggtgcccctgaacgagacctccaacacctccgagtaccgcctgatcaactgcaacacctccgcctgcacccaggcctgccccaaggtgaccttcgagcccatccccatccactactgcgcccccgccggctacgccatcctgaagtgcaacaacaagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgaacacctccgtggagatcgtgtgcacccgccccggcaacaacacccgcaagtccgtgcgcatcggccccggccagaccttctacgccaccggcgacatcatcggcgacatccgccaggcccactgcaacatctccgagggccagtggaacaagaccctgcacgaggtgtccaaggagctgcagaagcacttccccaacaagaccatcaagtacgagcgctccgccggcggccgcatggagatcgccacccactccttcaactgcggcggcgagttcttctactgcaacacctccaacctgttcaacggcacctacaacggcacctacatcaacacctcctccacctcctacatcaccctgcagtgccgcatcaagcagatcatcaacatgtggcagggcgtgggccgctgcatgtacgccccccccatcgccggcaacatcacctgcaagtccaacatcaccggcctgctgctgacccgcgacggcggcaccaagaacaactccaacgaggagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactagtaagcggccgcggatcc

SEQ ID NO: 169 - DNA, 2017 bp, synthetic construct
  source 1..2017: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacgagaaggaggtgcacaacgtgtgggccacccacgcctgcgtgcccaccgacccctccccccaggagctggtgctgaagaacgtgaccgagaacttcaacatgtggaagaacgacatggtggaccagatgcacgaggacatcatctccctgtgggaccagtccctgaagccctgcgtgaagctgacccccctgtgcgtgaccctgaactgctccaacgcccgctccaacgtgaacgtgacctccatcaacaacaccatcatgggcgagatgaagaactgctccttcaacaccaccaccgagatccgcgacaaggagaagaaggagtacgccctgttctacaagcccgacgtggtgcccctgaacgagacctccaacacctccgagtaccgcctgatcaactgcaacacctccgcctgcacccaggcctgccccaaggtgaccttcgagcccatccccatccactactgcgcccccgccggctacgccatcctgaagtgcaacaacaagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgaacacctccgtggagatcgtgtgcacccgccccggcaacaacacccgcaagtccgtgcgcatcggccccggccagaccttctacgccaccggcgacatcatcggcgacatccgccaggcccactgcaacatctccgagggccagtggaacaagaccctgcacgaggtgtccaaggagctgcagaagcacttccccaacaagaccatcaagtacgagcgctccgccggcggcgacatggagatcgccacccactccttcaactgcggcggcgagttcttctactgcaacacctccaacctgttcaacggcacctacaacggcacctacatcaacacctcctccacctcctacatcaccctgcagtgccgcatcaagcagatcatcaacatgtggcagggcgtgggccgctgcatgtacgccccccccatcgccggcaacatcacctgcaagtccaacatcaccggcctgctgctgacccgcgacggcggcaccaagaacaactccaacgaggagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacctgcctgagaccggaggatagtaagcggccgcggatcc

SEQ ID NO: 170 - DNA, 2503 bp, synthetic construct
  source 1..2503: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacgagaaggaggtgcacaacgtgtgggccacccacgcctgcgtgcccaccgacccctccccccaggagctggtgctgaagaacgtgaccgagaacttcaacatgtggaagaacgacatggtggaccagatgcacgaggacatcatctccctgtgggaccagtccctgaagccctgcgtgaagctgacccccctgtgcgtgaccctgaactgctccaacgcccgctccaacgtgaacgtgacctccatcaacaacaccatcatgggcgagatgaagaactgctccttcaacaccaccaccgagatccgcgacaaggagaagaaggagtacgccctgttctacaagcccgacgtggtgcccctgaacgagacctccaacacctccgagtaccgcctgatcaactgcaacacctccgcctgcacccaggcctgccccaaggtgaccttcgagcccatccccatccactactgcgcccccgccggctacgccatcctgaagtgcaacaacaagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgaacacctccgtggagatcgtgtgcacccgccccggcaacaacacccgcaagtccgtgcgcatcggccccggccagaccttctacgccaccggcgacatcatcggcgacatccgccaggcccactgcaacatctccgagggccagtggaacaagaccctgcacgaggtgtccaaggagctgcagaagcacttccccaacaagaccatcaagtacgagcgctccgccggcggcgacatggagatcgccacccactccttcaactgcggcggcgagttcttctactgcaacacctccaacctgttcaacggcacctacaacggcacctacatcaacacctcctccacctcctacatcaccctgcagtgccgcatcaagcagatcatcaacatgtggcagggcgtgggccgctgcatgtacgccccccccatcgccggcaacatcacctgcaagtccaacatcaccggcctgctgctgacccgcgacggcggcaccaagaacaactccaacgaggagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggtggtggaagcggagatatcatcaagctgctgaacgagcaagtgaacaaagagatgaacagctccaacctgtacatgagcatgtccagctggtgctacacccacagccttgatggcgccggactgttcctgtttgatcacgccgccgaggaatacgagcacgccaagaagctgatcattttcctgaacgagaacaacgtgccagtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctagtaagcggccgcggatcc

SEQ ID NO: 171 - DNA, 2029 bp, synthetic construct
  source 1..2029: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacaagaaggaggtgcacaacgtgtgggccacccacgcctgcgtgcccaccgacccctccccccaggagctgttcctgaagaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccaacgccatcgtgaagaactccaccaccgaggagatgtccaccgccctggtgaagaactccaccaccgaggccatgaagaactgctccttcaacaccaccaccgagatccgcgacaaggagaagaaggagtacgccctgttctaccgccccgacatcgtgcccctgaacaacgagaccggcaacatctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccaaggaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacccccgcaaggcccactgcaacatctccgagaaggactggaacaagaccctgcaggaggtgggcaaggagctgcagaagcacttccccaacaagaccatccgctacaaccagtccgccggcggcgacatggagatcaccacccactccttcaactgcggcggcgagttcttctactgcaacacctccaagctgttcaactccacctacaacgacacctacatctccaccaactcctccgccaacaactcctccaccatcaccctgcagtgccgcatcaagcagatcatcaacatgtggcagggcgtgggccgctgcatgtacgccccccccatcgccggcaacatcacctgcaagtccaacatcaccggcctgctgctgacccgcgacggcggccccgactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactagtaagcggccgcggatcc

SEQ ID NO: 172 - DNA, 2029 bp, synthetic construct
  source 1..2029: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacaagaagaaggtgcacaacgtgtgggccacccacgcctgcgtgcccaccgacccctccccccaggagctgttcctgaagaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccaacgccatcgtgaagaactccaccaccgaggagatgtccaccgccctggtgaagaactccaccaccgaggccatgaagaactgctccttcaacaccaccaccgagatccgcgacaaggagaagaaggagtacgccctgttctaccgccccgacatcgtgcccctgaacaacgagaccggcaacatctccgagtaccgcctgatcaactgcaacacctccgccgtgacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccaaggaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagtggttctacgccaccggcgacatcatcggcgacccccgcaaggcccactgcaacatctccgagaaggactggaacaagaccctgcaggaggtgggcaaggagctgcagaagcacttccccaacaagaccatccgctacaaccagtccgccggcggcgacatggagatcaccacccactccttcaactgcggcggcgagttcttctactgcaacacctccaagctgttcaactccacctacaacgacacctacatctccaccaactcctccgccaacaactcctccaccatcaccctgcagtgccgcatcaagcagatcatcaacatgtggcagggcgtgggccgcgccatgtacgccccccccatcgccggcaacatcacctgcaagtccaacatcaccggcctgctgctgacccgcgacggcggccccgactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactagtaagcggccgcggatcc

SEQ ID NO: 173 - DNA, 2029 bp, synthetic construct
  source 1..2029: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacaagaaggaggtgcacaacgtgtgggccacccacgcctgcgtgcccaccgacccctccccccaggagctgttcctgaagaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccaacgccatcgtgaagaactccaccaccgaggagatgtccaccgccctggtgaagaactccaccaccgaggccatgaagaactgctccttcaacaccaccaccgagatccgcgacaaggagaagaaggagtacgccctgttctaccgccccgacatcgtgcccctgaacaacgagaccggcaacatctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccaaggaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacccccgcaaggcccactgcaacatctccgagaaggactggaacaagaccctgcaggaggtgggcaaggagctgcagaagcacttccccaacaagaccatccgctacaaccagtccgccggcggccgcatggagatcaccacccactccttcaactgcggcggcgagttcttctactgcaacacctccaagctgttcaactccacctacaacgacacctacatctccaccaactcctccgccaacaactcctccaccatcaccctgcagtgccgcatcaagcagatcatcaacatgtggcagggcgtgggccgctgcatgtacgccccccccatcgccggcaacatcacctgcaagtccaacatcaccggcctgctgctgacccgcgacggcggccccgactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactagtaagcggccgcggatcc

SEQ ID NO: 174 - DNA, 2047 bp, synthetic construct
  source 1..2047: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacaagaaggaggtgcacaacgtgtgggccacccacgcctgcgtgcccaccgacccctccccccaggagctgttcctgaagaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccaacgccatcgtgaagaactccaccaccgaggagatgtccaccgccctggtgaagaactccaccaccgaggccatgaagaactgctccttcaacaccaccaccgagatccgcgacaaggagaagaaggagtacgccctgttctaccgccccgacatcgtgcccctgaacaacgagaccggcaacatctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccaaggaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacccccgcaaggcccactgcaacatctccgagaaggactggaacaagaccctgcaggaggtgggcaaggagctgcagaagcacttccccaacaagaccatccgctacaaccagtccgccggcggcgacatggagatcaccacccactccttcaactgcggcggcgagttcttctactgcaacacctccaagctgttcaactccacctacaacgacacctacatctccaccaactcctccgccaacaactcctccaccatcaccctgcagtgccgcatcaagcagatcatcaacatgtggcagggcgtgggccgctgcatgtacgccccccccatcgccggcaacatcacctgcaagtccaacatcaccggcctgctgctgacccgcgacggcggccccgactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacctgcctgagaccggaggatagtaagcggccgcggatcc

SEQ ID NO: 175 - DNA, 2533 bp, synthetic construct
  source 1..2533: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacaagaaggaggtgcacaacgtgtgggccacccacgcctgcgtgcccaccgacccctccccccaggagctgttcctgaagaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccaacgccatcgtgaagaactccaccaccgaggagatgtccaccgccctggtgaagaactccaccaccgaggccatgaagaactgctccttcaacaccaccaccgagatccgcgacaaggagaagaaggagtacgccctgttctaccgccccgacatcgtgcccctgaacaacgagaccggcaacatctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccaaggaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacccccgcaaggcccactgcaacatctccgagaaggactggaacaagaccctgcaggaggtgggcaaggagctgcagaagcacttccccaacaagaccatccgctacaaccagtccgccggcggcgacatggagatcaccacccactccttcaactgcggcggcgagttcttctactgcaacacctccaagctgttcaactccacctacaacgacacctacatctccaccaactcctccgccaacaactcctccaccatcaccctgcagtgccgcatcaagcagatcatcaacatgtggcagggcgtgggccgctgcatgtacgccccccccatcgccggcaacatcacctgcaagtccaacatcaccggcctgctgctgacccgcgacggcggccccgactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggtggtggaagcggagatatcatcaagctgctgaacgagcaagtgaacaaagagatgaacagctccaacctgtacatgagcatgtccagctggtgctacacccacagccttgatggcgccggactgttcctgtttgatcacgccgccgaggaatacgagcacgccaagaagctgatcattttcctgaacgagaacaacgtgccagtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctagtaagcggccgcggatcc

SEQ ID NO: 176 - protein, 652 aa, synthetic construct
MPMGSLQPLATLYLLGMLVASVLAAENLWVTVYYGVPVWKEAKTTLFCASDAKAYEKEVHNVWATHACVPTDPSPQELVLKNVTENFNMWKNDMVDQMHEDIISLWDQSLKPCVKLTPLCVTLNCSNARSNVNVTSINNTIMGEMKNCSFNTTTEIRDKEKKEYALFYKPDVVPLNETSNTSEYRLINCNTSACTQACPKVTFEPIPIHYCAPAGYAILKCNNKTFNGTGPCSNVSTVQCTHGIRPVVSTQLLLNGSLAEKEIVIRSENLTNNAKIIIVHLNTSVEIVCTRPGNNTRKSVRIGPGQTFYATGDIIGDIRQAHCNISEGQWNKTLHEVSKELQKHFPNKTIKYERSAGGDMEIATHSFNCGGEFFYCNTSNLFNGTYNGTYINTSSTSYITLQCRIKQIINMWQGVGRCMYAPPIAGNITCKSNITGLLLTRDGGTKNNSNEETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 177 - protein, 652 aa, synthetic construct
MPMGSLQPLATLYLLGMLVASVLAAENLWVTVYYGVPVWKEAKTTLFCASDAKAYEKKVHNVWATHACVPTDPSPQELVLKNVTENFNMWKNDMVDQMHEDIISLWDQSLKPCVKLTPLCVTLNCSNARSNVNVTSINNTIMGEMKNCSFNTTTEIRDKEKKEYALFYKPDVVPLNETSNTSEYRLINCNTSAVTQACPKVTFEPIPIHYCAPAGYAILKCNNKTFNGTGPCSNVSTVQCTHGIRPVVSTQLLLNGSLAEKEIVIRSENLTNNAKIIIVHLNTSVEIVCTRPGNNTRKSVRIGPGQWFYATGDIIGDIRQAHCNISEGQWNKTLHEVSKELQKHFPNKTIKYERSAGGDMEIATHSFNCGGEFFYCNTSNLFNGTYNGTYINTSSTSYITLQCRIKQIINMWQGVGRAMYAPPIAGNITCKSNITGLLLTRDGGTKNNSNEETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 178 - protein, 652 aa, synthetic construct
MPMGSLQPLATLYLLGMLVASVLAAENLWVTVYYGVPVWKEAKTTLFCASDAKAYEKEVHNVWATHACVPTDPSPQELVLKNVTENFNMWKNDMVDQMHEDIISLWDQSLKPCVKLTPLCVTLNCSNARSNVNVTSINNTIMGEMKNCSFNTTTEIRDKEKKEYALFYKPDVVPLNETSNTSEYRLINCNTSACTQACPKVTFEPIPIHYCAPAGYAILKCNNKTFNGTGPCSNVSTVQCTHGIRPVVSTQLLLNGSLAEKEIVIRSENLTNNAKIIIVHLNTSVEIVCTRPGNNTRKSVRIGPGQTFYATGDIIGDIRQAHCNISEGQWNKTLHEVSKELQKHFPNKTIKYERSAGGRMEIATHSFNCGGEFFYCNTSNLFNGTYNGTYINTSSTSYITLQCRIKQIINMWQGVGRCMYAPPIAGNITCKSNITGLLLTRDGGTKNNSNEETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 179 - protein, 658 aa, synthetic construct
MPMGSLQPLATLYLLGMLVASVLAAENLWVTVYYGVPVWKEAKTTLFCASDAKAYEKEVHNVWATHACVPTDPSPQELVLKNVTENFNMWKNDMVDQMHEDIISLWDQSLKPCVKLTPLCVTLNCSNARSNVNVTSINNTIMGEMKNCSFNTTTEIRDKEKKEYALFYKPDVVPLNETSNTSEYRLINCNTSACTQACPKVTFEPIPIHYCAPAGYAILKCNNKTFNGTGPCSNVSTVQCTHGIRPVVSTQLLLNGSLAEKEIVIRSENLTNNAKIIIVHLNTSVEIVCTRPGNNTRKSVRIGPGQTFYATGDIIGDIRQAHCNISEGQWNKTLHEVSKELQKHFPNKTIKYERSAGGDMEIATHSFNCGGEFFYCNTSNLFNGTYNGTYINTSSTSYITLQCRIKQIINMWQGVGRCMYAPPIAGNITCKSNITGLLLTRDGGTKNNSNEETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDLPETGG

SEQ ID NO: 180 - protein, 820 aa, synthetic construct
MPMGSLQPLATLYLLGMLVASVLAAENLWVTVYYGVPVWKEAKTTLFCASDAKAYEKEVHNVWATHACVPTDPSPQELVLKNVTENFNMWKNDMVDQMHEDIISLWDQSLKPCVKLTPLCVTLNCSNARSNVNVTSINNTIMGEMKNCSFNTTTEIRDKEKKEYALFYKPDVVPLNETSNTSEYRLINCNTSACTQACPKVTFEPIPIHYCAPAGYAILKCNNKTFNGTGPCSNVSTVQCTHGIRPVVSTQLLLNGSLAEKEIVIRSENLTNNAKIIIVHLNTSVEIVCTRPGNNTRKSVRIGPGQTFYATGDIIGDIRQAHCNISEGQWNKTLHEVSKELQKHFPNKTIKYERSAGGDMEIATHSFNCGGEFFYCNTSNLFNGTYNGTYINTSSTSYITLQCRIKQIINMWQGVGRCMYAPPIAGNITCKSNITGLLLTRDGGTKNNSNEETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 181 - protein, 662 aa, synthetic construct
MPMGSLQPLATLYLLGMLVASVLAAENLWVTVYYGVPVWKEAKTTLFCASDAKAYKKEVHNVWATHACVPTDPSPQELFLKNVTENFNMWKNDMVDQMHEDIISLWDQSLKPCVKLTPLCVTLICSNAIVKNSTTEEMSTALVKNSTTEAMKNCSFNTTTEIRDKEKKEYALFYRPDIVPLNNETGNISEYRLINCNTSACTQACPKVTFEPIPIHYCAPAGYAILKCNDETFNGTGPCSNVSTVQCTHGIRPVVSTQLLLNGSLAKEEIVIRSENLTNNAKIIIVHLHTPVEIVCTRPNNNTRKSVRIGPGQTFYATGDIIGDPRKAHCNISEKDWNKTLQEVGKELQKHFPNKTIRYNQSAGGDMEITTHSFNCGGEFFYCNTSKLFNSTYNDTYISTNSSANNSSTITLQCRIKQIINMWQGVGRCMYAPPIAGNITCKSNITGLLLTRDGGPDSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 182 - protein, 662 aa, synthetic construct
MPMGSLQPLATLYLLGMLVASVLAAENLWVTVYYGVPVWKEAKTTLFCASDAKAYKKKVHNVWATHACVPTDPSPQELFLKNVTENFNMWKNDMVDQMHEDIISLWDQSLKPCVKLTPLCVTLICSNAIVKNSTTEEMSTALVKNSTTEAMKNCSFNTTTEIRDKEKKEYALFYRPDIVPLNNETGNISEYRLINCNTSAVTQACPKVTFEPIPIHYCAPAGYAILKCNDETFNGTGPCSNVSTVQCTHGIRPVVSTQLLLNGSLAKEEIVIRSENLTNNAKIIIVHLHTPVEIVCTRPNNNTRKSVRIGPGQWFYATGDIIGDPRKAHCNISEKDWNKTLQEVGKELQKHFPNKTIRYNQSAGGDMEITTHSFNCGGEFFYCNTSKLFNSTYNDTYISTNSSANNSSTITLQCRIKQIINMWQGVGRAMYAPPIAGNITCKSNITGLLLTRDGGPDSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 183 - protein, 662 aa, synthetic construct
MPMGSLQPLATLYLLGMLVASVLAAENLWVTVYYGVPVWKEAKTTLFCASDAKAYKKEVHNVWATHACVPTDPSPQELFLKNVTENFNMWKNDMVDQMHEDIISLWDQSLKPCVKLTPLCVTLICSNAIVKNSTTEEMSTALVKNSTTEAMKNCSFNTTTEIRDKEKKEYALFYRPDIVPLNNETGNISEYRLINCNTSACTQACPKVTFEPIPIHYCAPAGYAILKCNDETFNGTGPCSNVSTVQCTHGIRPVVSTQLLLNGSLAKEEIVIRSENLTNNAKIIIVHLHTPVEIVCTRPNNNTRKSVRIGPGQTFYATGDIIGDPRKAHCNISEKDWNKTLQEVGKELQKHFPNKTIRYNQSAGGRMEITTHSFNCGGEFFYCNTSKLFNSTYNDTYISTNSSANNSSTITLQCRIKQIINMWQGVGRCMYAPPIAGNITCKSNITGLLLTRDGGPDSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 184 - protein, 668 aa, synthetic construct
MPMGSLQPLATLYLLGMLVASVLAAENLWVTVYYGVPVWKEAKTTLFCASDAKAYKKEVHNVWATHACVPTDPSPQELFLKNVTENFNMWKNDMVDQMHEDIISLWDQSLKPCVKLTPLCVTLICSNAIVKNSTTEEMSTALVKNSTTEAMKNCSFNTTTEIRDKEKKEYALFYRPDIVPLNNETGNISEYRLINCNTSACTQACPKVTFEPIPIHYCAPAGYAILKCNDETFNGTGPCSNVSTVQCTHGIRPVVSTQLLLNGSLAKEEIVIRSENLTNNAKIIIVHLHTPVEIVCTRPNNNTRKSVRIGPGQTFYATGDIIGDPRKAHCNISEKDWNKTLQEVGKELQKHFPNKTIRYNQSAGGDMEITTHSFNCGGEFFYCNTSKLFNSTYNDTYISTNSSANNSSTITLQCRIKQIINMWQGVGRCMYAPPIAGNITCKSNITGLLLTRDGGPDSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDLPETGG

SEQ ID NO: 185 - protein, 830 aa, synthetic construct
MPMGSLQPLATLYLLGMLVASVLAAENLWVTVYYGVPVWKEAKTTLFCASDAKAYKKEVHNVWATHACVPTDPSPQELFLKNVTENFNMWKNDMVDQMHEDIISLWDQSLKPCVKLTPLCVTLICSNAIVKNSTTEEMSTALVKNSTTEAMKNCSFNTTTEIRDKEKKEYALFYRPDIVPLNNETGNISEYRLINCNTSACTQACPKVTFEPIPIHYCAPAGYAILKCNDETFNGTGPCSNVSTVQCTHGIRPVVSTQLLLNGSLAKEEIVIRSENLTNNAKIIIVHLHTPVEIVCTRPNNNTRKSVRIGPGQTFYATGDIIGDPRKAHCNISEKDWNKTLQEVGKELQKHFPNKTIRYNQSAGGDMEITTHSFNCGGEFFYCNTSKLFNSTYNDTYISTNSSANNSSTITLQCRIKQIINMWQGVGRCMYAPPIAGNITCKSNITGLLLTRDGGPDSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 186 - DNA, 1949 bp, synthetic construct
  source 1..1949: mol_type = other DNA
atgggctccctgcagcccctggccaccctgtacctgctgggcatgctggtggcctccgtgctggccgccgagaacctgtgggtgaccgtgtactactacggcgtgccgtggaaggaggccaagaccaccctgttctgcgcctccgacgcccgcgcctacgagaaggaggtgcacaacgtgtgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccgacgccggctccggcggcgtggaggagatgaagaactgctccttcaacaccaccaccgagatccgcgacaaggagaagaaggagtacgccctgttctacaagcccgacatcgtgcccctgtccgagaccaacaacacctccgagtaccgcctgatcaactgcaacacctccgcctgcacccaggcctgccccaaggtgaccttcgagcccatccccatccactactgcgccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgcgcctccatgacctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactgataagcggccgcggatcc

SEQ ID NO: 187 - protein, 644 aa, synthetic construct
MGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGSGGVEEMKNCSFNTTTEIRDKEKKEYALFYKPDIVPLSETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 188 - protein, 622 aa, synthetic construct
AENLWVTVYYGVPVWKEAKTTLFCASDARAYEKEVHNVWATHACVPTDPSPQELVLGNVTENFNNWKNDNVDQNHEDIISLWDQSLKPCVKLTPLCVTLICSDAGSGGVEEMKNCSFNTTTEIRDKEKKEYALFYKPDIVPLSETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRLGPGQTFYATGDIIGDIKQAHCNISEEKWNDTLQKVGIELQKHFPNKTIKYNQSAGGDMEITTHSFNCGGEFFYCMTSNLFNGTYNGTYISTNSSANSTSTITLQCRIKQIINMWQGVGRCMYATTIAGNITCRSNITGLLLTRDGGTNSNETETFRPAGGDMRDMWRSELYKYKVVKIEPLGVAPTRCKRRVVGRRRRRRAVGIGAVFLGFLGAAGSTMGAASMPLTVQARNLLSGIVQQQSNLLRAPPAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 189 - skipped

SEQ ID NO: 190 - protein, 20 aa, synthetic construct
ARGGWISLYYDSSYYPNFDY

SEQ ID NO: 191 - protein, 23 aa, synthetic construct
ARNAIRIYGVVALGEWFHYGMDV

SEQ ID NO: 192 - protein, 26 aa, synthetic construct
ARTLHGRRIYGIVAFNEWFTYFYMDV

SEQ ID NO: 193 - protein, 19 aa, synthetic construct
ARGGWISLYYDSGYPNFDY

SEQ ID NO: 194 - protein, 19 aa, synthetic construct
ARGGWISLAYDSGYPNFDY

SEQ ID NO: 195 - protein, 19 aa, synthetic construct
ARGGWISLCYDSGYPNFDY

SEQ ID NO: 196 - protein, 19 aa, synthetic construct
ARGGWISLFYDSGYPNFDY

SEQ ID NO: 197 - protein, 19 aa, synthetic construct
ARGGWISLSYDSGYPNFDY

SEQ ID NO: 198 - protein, 19 aa, synthetic construct
ARGGWISLDYDSGYPNFDY

SEQ ID NO: 199 - protein, 19 aa, synthetic construct
ARGGWISLTYDSGYPNFDY

SEQ ID NO: 200 - protein, 19 aa, synthetic construct
ARGGWISLYDDSGYPNFDY

SEQ ID NO: 201 - protein, 19 aa, synthetic construct
ARGGWISLYEDSGYPNFDY

SEQ ID NO: 202 - protein, 19 aa, synthetic construct
ARGGWISLYLDSGYPNFDY

SEQ ID NO: 203 - protein, 19 aa, synthetic construct
ARGGWISLYYDRGYPNFDY

SEQ ID NO: 204 - protein, 19 aa, synthetic construct
ARGGWISLYYDWGYPNFDY

SEQ ID NO: 205 - protein, 19 aa, synthetic construct
ARGGWISLYYDKGYPNFDY

SEQ ID NO: 206 - protein, 19 aa, synthetic construct
ARGGWISLYYDMGYPNFDY

SEQ ID NO: 207 - protein, 20 aa, synthetic construct
ARGGWISLYYDSSGYPNFDY

SEQ ID NO: 208 - protein, 13 aa, synthetic construct
DVGTEGSLLHFDY

SEQ ID NO: 209 - protein, 20 aa, synthetic construct
ARAGFITYYYDSSGYCGFDY

SEQ ID NO: 210 - protein, 20 aa, synthetic construct
ARASRVTYYYDSSGYADFDY

SEQ ID NO: 211 - protein, 20 aa, synthetic construct
ARGSRVTYYYDSSGYADFDY

SEQ ID NO: 212 - protein, 20 aa, synthetic construct
ARSGWSTYYYDSSGYHGFDY

SEQ ID NO: 213 - protein, 20 aa, synthetic construct
ATASRLTYYYDSSGYPAFDY

SEQ ID NO: 214 - protein, 20 aa, synthetic construct
ARAPRITYYYDSSGYPNLDY

SEQ ID NO: 215 - protein, 20 aa, synthetic construct
AAGSRTTYYYDSSGYPDFDY

SEQ ID NO: 216 - protein, 20 aa, synthetic construct
AGSTFRTYYYDSSGYAKFDY

SEQ ID NO: 217 - protein, 20 aa, synthetic construct
ARGGYETYYYDSSGYPIFDY

SEQ ID NO: 218 - protein, 20 aa, synthetic construct
ARGGGVTYYYDSSGYTSFDY

SEQ ID NO: 219 - protein, 20 aa, synthetic construct
AKGAFTTYYYDSSGYSDFDY

SEQ ID NO: 220 - protein, 20 aa, synthetic construct
AKSAYITYYYDSSGYSYLDY

SEQ ID NO: 221 - protein, 20 aa, synthetic construct
ARATYMTYYYDSSGYPALDY

SEQ ID NO: 222 - protein, 20 aa, synthetic construct
ARGSYLTYYYDSSGYGGLDY

SEQ ID NO: 223 - protein, 20 aa, synthetic construct
ARAGGETYYYDSSGYANFDY

SEQ ID NO: 224 - protein, 20 aa, synthetic construct
ARGGRSTYYYDSSGYSDFDY

SEQ ID NO: 225 - protein, 20 aa, synthetic construct
ARADYLKYYYDSSGWNYFDY

SEQ ID NO: 226 - protein, 20 aa, synthetic construct
ARGGYNTYYYDSSGYLDLDY

SEQ ID NO: 227 - protein, 19 aa, synthetic construct
ARGGWISLYYDSSGYPNFD

SEQ ID NO: 228 - protein, 19 aa, synthetic construct
ARGSFVMYYYDSSGYYSFD

SEQ ID NO: 229 - protein, 19 aa, synthetic construct
ARAGFITYYYDSSGYCGFD

SEQ ID NO: 230 - protein, 19 aa, synthetic construct
ARGGGVTYYYDSSGYTSFD

SEQ ID NO: 231 - protein, 19 aa, synthetic construct
AKSAYITYYYDSSGYSYLD

SEQ ID NO: 232 - protein, 19 aa, synthetic construct
ARASRVTYYYDSSGYADFD

SEQ ID NO: 233 - protein, 19 aa, synthetic construct
ARGGFSTYYYDSSGYRNFD

SEQ ID NO: 234 - protein, 19 aa, synthetic construct
ARSGWSTYYYDSSGYHGFD

SEQ ID NO: 235 - protein, 19 aa, synthetic construct
ARGSRVTYYYDSSGYRNFD

SEQ ID NO: 236 - protein, 20 aa, synthetic construct
ARGSFVMYYYDSSGYYSFDY

SEQ ID NO: 237 - protein, 17 aa, synthetic construct
CSDATVKTGTVEEMKNC

SEQ ID NO: 238 - protein, 15 aa, synthetic construct
CSDAGSGGVEEMKNC

SEQ ID NO: 239 - protein, 6 aa, synthetic construct
  MOD_RES 2: note = Asparagine in a potential N-linked glycosylation site motif
  MOD_RES 4: note = Asparagine in a potential N-linked glycosylation site motif
DNNNDQ

SEQ ID NO: 240 - protein, 6 aa, synthetic construct
  MOD_RES 2: note = Asparagine in a potential N-linked glycosylation site motif
  MOD_RES 4: note = Asparagine in a potential N-linked glycosylation site motif
DNNNDK

SEQ ID NO: 241 - protein, 6 aa, synthetic construct
  MOD_RES 2: note = Asparagine in a potential N-linked glycosylation site motif
  MOD_RES 4: note = Asparagine in a potential N-linked glycosylation site motif
DNYNDQ

SEQ ID NO: 242 - protein, 6 aa, synthetic construct
  MOD_RES 2: note = Asparagine in a potential N-linked glycosylation site motif
  MOD_RES 4: note = Asparagine in a potential N-linked glycosylation site motif
DNNNNQ

SEQ ID NO: 243 - protein, 6 aa, synthetic construct
  MOD_RES 3: note = Asparagine in a potential N-linked glycosylation site motif
IVNTSD

SEQ ID NO: 244 - protein, 6 aa, synthetic construct
  MOD_RES 3: note = Asparagine in a potential N-linked glycosylation site motif
IFNTSD

SEQ ID NO: 245 - protein, 6 aa, synthetic construct
  MOD_RES 3: note = Asparagine in a potential N-linked glycosylation site motif
NFNTSD

SEQ ID NO: 246 - protein, 6 aa, synthetic construct
  MOD_RES 3: note = Asparagine in a potential N-linked glycosylation site motif
NFNTSN

SEQ ID NO: 247 - protein, 6 aa, synthetic construct
  MOD_RES 3: note = Asparagine in a potential N-linked glycosylation site motif
MSNTSN

SEQ ID NO: 248 - protein, 6 aa, synthetic construct
IFNISD

SEQ ID NO: 249 - protein, 6 aa, synthetic construct
  MOD_RES 2: note = Asparagine in a potential N-linked glycosylation site motif
DNYYDQ

SEQ ID NO: 250 - protein, 7 aa, synthetic construct
NCSFTEV

SEQ ID NO: 251 - protein, 10 aa, synthetic construct
NCSFSVTTEL

SEQ ID NO: 252 - protein, 8 aa, synthetic construct
TRPYDKVS

SEQ ID NO: 253 - protein, 8 aa, synthetic construct
TRPNNNIR

SEQ ID NO: 254 - protein, 6 aa, synthetic construct
  MOD_RES 1: note = Asparagine in a potential N-linked glycosylation site motif
  MOD_RES 2: note = Asparagine in a potential N-linked glycosylation site motif
NNNNDE

SEQ ID NO: 255 - protein, 6 aa, synthetic construct
IVDVRD

SEQ ID NO: 256 - protein, 6 aa, synthetic construct
  MOD_RES 1: note = Asparagine in a potential N-linked glycosylation site motif
  MOD_RES 4: note = Asparagine in a potential N-linked glycosylation site motif
NKGNDQ

SEQ ID NO: 257 - protein, 6 aa, synthetic construct
  MOD_RES 4: note = Asparagine in a potential N-linked glycosylation site motif
DKYNDQ

SEQ ID NO: 258 - protein, 6 aa, synthetic construct
  MOD_RES 2: note = Asparagine in a potential N-linked glycosylation site motif
  MOD_RES 4: note = Asparagine in a potential N-linked glycosylation site motif
DNYNDK

SEQ ID NO: 259 - DNA, 1946 bp, synthetic construct
  source 1..1946: mol_type = other DNA
atgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgcccgcgcctacgagaaggaggtgcacaacgtgtgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccgacgccggctccggcggcgtggaggagatgaagaactgctccttcaacaccaccaccgagatccgcgacaaggagaagaaggagtacgccctgttctacaagcccgacatcgtgcccctgtccgagaccaacaacacctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactgataagcggccgc

SEQ ID NO: 260 - DNA, 1944 bp, synthetic construct
  source 1..1944: mol_type = other DNA
atgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccggctccggcggcgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattgatga

SEQ ID NO: 261 - DNA, 2448 bp, synthetic construct
  source 1..2448: mol_type = other DNA
atgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccggctccggcggcgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaaacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattctggcggagacattatcaagctgctgaacgagcaagtgaacaaagaaatgcagtcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtccggctcctgatga

SEQ ID NO: 262 - DNA, 2448 bp, synthetic construct
  source 1..2448: mol_type = other DNA
atgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccggcaacggctccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaaacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattctggcggagacattatcaagctgctgaacgagcaagtgaacaaagaaatgcagtcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtccggctcctgatga

SEQ ID NO: 263 - DNA, 2448 bp, synthetic construct
  source 1..2448: mol_type = other DNA
atgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccggctccggcggcgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaaacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccgcctccaccagcaccatcactctgcagtgccggatcaagcagatcatcaatatgtggcaaggcgtcggccggtgtatgtacgcccctcctatcgccggcgccatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattctggcggagacattatcaagctgctgaacgagcaagtgaacaaagaaatgcagtcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtccggctcctgatga

SEQ ID NO: 264 - DNA, 2448 bp, synthetic construct
  source 1..2448: mol_type = other DNA
atgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccggcaacggctccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaaacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccgcctccaccagcaccatcactctgcagtgccggatcaagcagatcatcaatatgtggcaaggcgtcggccggtgtatgtacgcccctcctatcgccggcgccatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattctggcggagacattatcaagctgctgaacgagcaagtgaacaaagaaatgcagtcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtccggctcctgatga

SEQ ID NO: 265 - DNA, 2022 bp, synthetic construct
  source 1..2022: mol_type = other DNA
atgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccggctccggcggcgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggcctggtgcctcagcagagcggcggcctgaacgacatcttcgaggcccagaagatcgagtggcacgaaggctgatga

SEQ ID NO: 266 - DNA, 1944 bp, synthetic construct
  source 1..1944: mol_type = other DNA
atgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccggcaacggctccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattgatga

SEQ ID NO: 267 - DNA, 2022 bp, synthetic construct
  source 1..2022: mol_type = other DNA
atgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccggcaacggctccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggcctggtgcctcagcagagcggcggcctgaacgacatcttcgaggcccagaagatcgagtggcacgaaggctgatga

SEQ ID NO: 268 - DNA, 2082 bp, synthetic construct
  source 1..2082: mol_type = other DNA
atgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgcccgcgcctacgagaaggaggtgcacaacgtgtgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccgacgccggctccggcggcgtggaggagatgaagaactgctccttcaacaccaccaccgagatccgcgacaaggagaagaaggagtacgccctgttctacaagcccgacatcgtgcccctgtccgagaccaacaacacctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtgataa

SEQ ID NO: 269 - DNA, 2016 bp, synthetic construct
  source 1..2016: mol_type = other DNA
atgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgcccgcgcctacgagaaggaggtgcacaacgtgtgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccgacgccggctccggcggcgtggaggagatgaagaactgctccttcaacaccaccaccgagatccgcgacaaggagaagaaggagtacgccctgttctacaagcccgacatcgtgcccctgtccgagaccaacaacacctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggaggaggcctggtgccacagcagagcggaggcctgaacgacatcttcgaggcccagaagatcgagtggcacgagggctgataa

SEQ ID NO: 270 - DNA, 2016 bp, synthetic construct
  source 1..2016: mol_type = other DNA
atgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgcccgcgcctacgagaaggaggtgcacaacgtgtgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccgacgccggcaacggcagcgtggaggagatgaagaactgctccttcaacaccaccaccgagatccgcgacaaggagaagaaggagtacgccctgttctacaagcccgacatcgtgcccctgtccgagaccaacaacacctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggaggaggcctggtgccacagcagagcggaggcctgaacgacatcttcgaggcccagaagatcgagtggcacgagggctgataa

SEQ ID NO: 271 - DNA, 2082 bp, synthetic construct
  source 1..2082: mol_type = other DNA
atgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgcccgcgcctacgagaaggaggtgcacaacgtgtgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccgacgccggcaacggcagcgtggaggagatgaagaactgctccttcaacaccaccaccgagatccgcgacaaggagaagaaggagtacgccctgttctacaagcccgacatcgtgcccctgtccgagaccaacaacacctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtgataa

SEQ ID NO: 272 - protein, 646 aa, synthetic construct
MGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGSGGVEEMKNCSFNTTTEIRDKEKKEYALFYKPDIVPLSETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDAA

SEQ ID NO: 273 - protein, 646 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGSGGVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 274 - protein, 814 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGSGGVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 275 - protein, 1638 aa, synthetic construct
  VARIANT 815..824: note = X can be any amino acid
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGNGSVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGSXXXXXXXXXXMPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGSGGVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSAASTSTITLQCRIKQIINMWQGVGRCMYAPPIAGAITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 276 - protein, 814 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGNGSVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSAASTSTITLQCRIKQIINMWQGVGRCMYAPPIAGAITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 277 - protein, 672 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGSGGVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGLVPQQSGGLNDIFEAQKIEWHEG

SEQ ID NO: 278 - protein, 646 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGNGSVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 279 - protein, 672 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGNGSVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGLVPQQSGGLNDIFEAQKIEWHEG

SEQ ID NO: 280 - protein, 692 aa, synthetic construct
MGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGSGGVEEMKNCSFNTTTEIRDKEKKEYALFYKPDIVPLSETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDLEVLFQGPGHHHHHHHHSAWSHPQFEKGGGSGGGGSGGSAWSHPQFEK

SEQ ID NO: 281 - protein, 670 aa, synthetic construct
MGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGSGGVEEMKNCSFNTTTEIRDKEKKEYALFYKPDIVPLSETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGLVPQQSGGLNDIFEAQKIEWHEG

SEQ ID NO: 282 - protein, 670 aa, synthetic construct
MGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGNGSVEEMKNCSFNTTTEIRDKEKKEYALFYKPDIVPLSETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGLVPQQSGGLNDIFEAQKIEWHEG

SEQ ID NO: 283 - protein, 692 aa, synthetic construct
MGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGNGSVEEMKNCSFNTTTEIRDKEKKEYALFYKPDIVPLSETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDLEVLFQGPGHHHHHHHHSAWSHPQFEKGGGSGGGGSGGSAWSHPQFEK

SEQ ID NO: 284 - protein, 4 aa, synthetic construct
GSGG

SEQ ID NO: 285 - protein, 6 aa, synthetic construct
  VARIANT 3: note = X signifies any amino acid but most commonly Ala, Ser, Glu
LPXTGG

SEQ ID NO: 286 - protein, 6 aa, synthetic construct
  MOD_RES 1: note = Asparagine in a potential N-linked glycosylation site motif
  MOD_RES 2: note = Asparagine in a potential N-linked glycosylation site motif
  MOD_RES 4: note = Asparagine in a potential N-linked glycosylation site motif
NNGNDQ

SEQ ID NO: 287 - protein, 6 aa, synthetic construct
  MOD_RES 1: note = Asparagine in a potential N-linked glycosylation site motif
  MOD_RES 2: note = Asparagine in a potential N-linked glycosylation site motif
  MOD_RES 4: note = Asparagine in a potential N-linked glycosylation site motif
NNNNDQ

SEQ ID NO: 288 - protein, 6 aa, synthetic construct
  MOD_RES 1: note = Asparagine in a potential N-linked glycosylation site motif
  MOD_RES 2: note = Asparagine in a potential N-linked glycosylation site motif
  MOD_RES 4: note = Asparagine in a potential N-linked glycosylation site motif
NNNNNQ

SEQ ID NO: 289 - protein, 5 aa, synthetic construct
RKTSP

SEQ ID NO: 290 - protein, 6 aa, synthetic construct
TAVHVA

SEQ ID NO: 291 - protein, 6 aa, synthetic construct
FNLIDF

SEQ ID NO: 292 - protein, 7 aa, synthetic construct
RASNSPM

SEQ ID NO: 293 - protein, 20 aa, synthetic construct
ARGGWISLYYDSSGYPNFDY

(19) United States
(12) Patent Application Publication
(10) Pub. No.: US 2025/0340597 A1
(43) Pub. Date: Nov. 6, 2025

(54) COMPOSITIONS COMPRISING HIV ENVELOPES TO INDUCE HIV-1 ANTIBODIES

(71) Applicant: Duke University, Durham, NC (US)

(72) Inventors: Mihai AZOITEI, Durham, NC (US); Kevin O. SAUNDERS, Durham, NC (US); Barton F. HAYNES, Durham, NC (US); Kevin J. WIEHE, Durham, NC (US)

(21) Appl. No.: 18/700,677

(22) PCT Filed: Oct. 11, 2022
(86) PCT No.: PCT/US2022/046289

§ 371 Date: Apr. 11, 2024

Related U.S. Application Data
(60) Provisional application No. 63/254,506, filed on Oct. 11, 2021.

(51) Int. Cl. C07K 14/005; A61K 39/00; A61K 39/21

(52) U.S. Cl. CPC C07K 14/005; A61K 39/21; A61K 2039/53; A61K 2039/55555; C12N 2740/16122; C12N 2740/16134

(57) ABSTRACT

The invention is directed to modified HIV-1 envelopes, compositions comprising these modified envelopes, nucleic acids encoding these modified envelopes, compositions comprising these nucleic acids, and methods of using these modified HIV-1 envelopes and/or these nucleic acids to induce immune responses.

This International Patent application claims the benefit of and priority to U.S. Application No. 63/254,506, filed Oct. 11, 2021, entitled “Compositions comprising HIV envelopes to induce HIV-1 antibodies,” the contents of which are hereby incorporated by reference in their entireties.

This invention was made with government support from the NIH, NIAID, Division of AIDS for UMI grant AI144371 for the Consortium for HIV/AIDS Vaccine Development (CHAVD). The government has certain rights in the invention.

### TECHNICAL FIELD

The present invention relates in general, to a composition suitable for use in inducing anti-HIV-1 antibodies, and, in particular, to immunogenic compositions comprising envelope proteins and nucleic acids to induce cross-reactive neutralizing antibodies and increase their breadth of coverage. The invention also relates to methods of inducing such broadly neutralizing anti-HIV-1 antibodies using such compositions.

### BACKGROUND

The development of a safe and effective HIV-1 vaccine is one of the highest priorities of the scientific community working on the HIV-1 epidemic. While anti-retroviral treatment (ART) has dramatically prolonged the lives of HIV-1 infected patients, ART is not routinely available in developing countries.

### SUMMARY OF THE INVENTION

In certain embodiments, the invention provides compositions and methods for induction of an immune response, for example cross-reactive (broadly) neutralizing (bn) Ab induction.

In certain aspects the invention provides a recombinant protein or nucleic acid encoding a recombinant protein as described in Table 5. In certain aspects the invention provides a selection of HIV-1 envelopes for use as prime and boost immunogens in methods to induce HIV-1 neutralizing antibodies. In certain aspects, the invention provides a selection of HIV-1 envelopes for use as a boost immunogen in methods to induce HIV-1 neutralizing antibodies.

In certain aspects the invention provides a selection of a series of immunogens and immunogen designs for induction of neutralizing HIV-1 antibodies, e.g. but not limited to V3 glycan epitope targeting antibodies, the selection comprising envelopes as follows: 1) CH848.d0949.10.17 DT (also referred to as CH848.d0949.10.17.N133D.N138T), 2) CH848.d0949.10.17 (also referred to as CH848.d0949.10.17WT), 3) CH848.d0808.15.15, 4) CH848.d0358.80.06, 5) CH848.d1432.5.41, 6) CH848.d1621.4.44 and 7) CH848.d1305.10.35 (see Tables 3 and 4). In some embodiments, the selection further comprises any HIV-1 envelope sequence as described in Table 5. In some embodiments, the selection further comprises any HIV-1 envelope sequence with the modification to the V1 loop described herein. In some embodiments the selection comprises additional HIV-1 Envs, P0402.c2.11 and ZM246F.

In certain aspects the invention provides a selection of a series of immunogens and immunogen designs for induction of neutralizing HIV-1 antibodies, e.g. but not limited to V3 glycan epitope targeting antibodies, the selection comprising envelopes as follows: 1) CH848.d0949.10.17 DT (also referred to as CH848.d0949.10.17.N133D.N138T), 2) CH848.d0949.10.17 (also referred to as CH848.d0949.10.17WT), 3) CH848.d0808.15.15, 4) CH848.d0358.80.06, 5) CH848.d1432.5.41, 6) CH848.d1621.4.44, 7) CH848.d1305.10.35, (see Tables 3 and 4) and 8) any HIV-1 envelope sequence as described in Table 5 or any HIV-1 envelope sequence with the modification to the V1 loop described herein. In some embodiments the selection comprises additional HIV-1 Envs, P0402.c2.11 and ZM246F.

In certain embodiments, the methods use compositions comprising HIV-1 envelope immunogens designed to bind to precursors, and/or unmutated common ancestors (UCAs) of different HIV-1 bnAbs. In certain embodiments, these are UCAs of VIV2 glycan and V3 glycan binding antibodies. Thus, in certain embodiments the invention provides HIV-1 envelope immunogen designs with multimerization and variable region sequence optimization for enhanced UCA-targeting. In certain embodiments the invention provides HIV-1 envelope immunogen designs with multimerization and variable region sequence optimization for enhanced targeting and inductions of multiple antibody lineages, e.g. but not limited to V3 lineage, VIV2 lineages of antibodies.

In certain aspects the invention provides compositions comprising a selection of HIV-1 envelopes and/or nucleic acids encoding these envelopes as described herein for example but not limited to designs as described herein. Without limitations, these selected combinations comprise envelopes which provide representation of the sequence (genetic) and antigenic diversity of the HIV-1 envelope variants which lead to the induction of VIV2 glycan and V3 glycan antibody lineages.

In certain aspects the invention provides compositions comprising recombinant HIV-1 envelopes and/or nucleic acids encoding these envelopes with a modifications to the V1 loop to connect V1 residues 104 and 109 (HBX2 numbering) to with linker “GSGG”. Such a modification can be incorporated into any HIV-1 envelope sequences from the CH848 infected individual and variants thereof. See e.g., US2020/0113997 incorporated herein by reference in its entirety including FIGS. 40A-C, 41A-41C, 44A-D, 45, 46, 47A, 49A-B, 50A-D, 51, 52A-B, 53A, 53D, 54A-F, 77A-L, and 78A-B and SEQ ID NOs disclosed therein. In some embodiments, such a modification can be incorporated into envelope CH848.3.D0949.10.17 (also referred to as CH848.d0949.10.17WT) and variants thereof, including, but not limited to, CH848.d0949.10.17 DT (also referred to as CH848.d0949.10.17.N133D.N138T). In some embodiments, such a modification can be incorporated into envelope CH848.d0808.15.15 and variants thereof. In some embodiments, such a modification can be incorporated into envelope CH848.d0358.80.06 and variants thereof. In some embodiments, such a modification can be incorporated into envelope CH848.d1432.5.41 and variants thereof. In some embodiments, such a modification can be incorporated into envelope CH848.d1621.4.44 and variants thereof. In some embodiments, such a modification can be incorporated into envelope CH848.d1305.10.35 and variants thereof. In some embodiments, such a modification can be incorporated into envelope CH848.0358.80.06. In some embodiments, such a modification can be incorporated into envelope CH848.1432.5.41. In certain embodiments, the invention provides compositions comprising recombinant HIV-1 envelope CH848.3.D0949.10.17chim.6R.DS.SOSIP.664_N133D_GS135-40 and/or nucleic acids encoding this envelopes.

In certain aspects the recombinant HIV-1 envelope optionally comprises any combinations of additional modifications, such as the modifications described in Table 2. In certain aspects the invention provides a recombinant HIV-1 envelope comprising a shortened V1 region (e.g., 17 amino acid (17aa) or shorter V1 region), lacking glycosylation at position N133 and N138 (HXB2 numbering), comprising glycosylation at N301 (HXB2 numbering) and N332 (HXB2 numbering), comprising modifications wherein glycan holes are filled (D230N_H289N_P291S (HXB2 numbering)), comprising the “GDIR” or “GDIK” motif at the position corresponding to the amino acid changes #3 in the sequences depicted in FIG. 8B, or any trimer stabilization modifications, UCA targeting modification, immunogenicity modification, or combinations thereof, for example but not limited to these described in Table 2, FIG. 8B (amino acid changes numbered 1-5), and/or FIGS. 21A-25B. In certain embodiments the recombinant envelope optionally comprises any combinations of these modifications.

In certain embodiments, the recombinant HIV-1 envelope binds to precursors, and/or UCAs of different HIV-1 bnAbs. In certain embodiments, these are UCAs of VIV2 glycan and V3 glycan antibodies. In certain embodiments the envelope is 19CV3. In certain embodiments the envelope is any one of the envelopes listed in Table 1, Table 2 or FIGS. 21A-25B. In certain embodiments, the envelope is not CH848 10.17 DT variant described previously in US2020/0113997.

In certain embodiments the envelope is a protomer which could be comprised in a stable trimer.

In certain embodiments the envelope comprises additional mutations stabilizing the envelope trimer. In certain embodiments these include, but are not limited to, SOSIP mutations. In certain embodiments mutations are selected from sets F1-F14, VT1-VT8 mutations described herein, or any combination or subcombination within a set. In certain embodiments, the selected mutations are F14. In other embodiments, the selected mutations are VT8. In certain embodiments, the selected mutations are F14 and VT8 combined.

In certain embodiments, the invention provides a recombinant HIV-1 envelope of Table 5. In certain embodiments, the invention provides a recombinant HIV-1 envelope of FIG. 1, FIG. 2, FIG. 3, or FIGS. 21A-25B, 27-29. In certain embodiments, the invention provides a nucleic acid encoding any of the recombinant envelopes. In certain embodiments, the nucleic acids comprise an mRNA formulated for use as a pharmaceutical composition.

In certain embodiments the inventive designs comprise specific changes (D230N_H289N_P291S (HXB2 numbering)), as shown in FIGS. 21A-21B, which fill glycan holes with the introduction of new glycosylation sites to prevent the binding of strain-specific antibodies that could hinder broad neutralizing antibody development (Wagh, Kshitij et al. “Completeness of HIV-1 Envelope Glycan Shield at Transmission Determines Neutralization Breadth.” Cell reports vol. 25,4 (2018): 893-908.e7. doi: 10.1016/j.celrep.2018.09 087; Crooks, Ema T et al. “Vaccine-Elicited Tier 2 HIV-1 Neutralizing Antibodies Bind to Quaternary Epitopes Involving Glycan-Deficient Patches Proximal to the CD4 Binding Site.” PLOS pathogens vol. 11,5 e1004932. 29 May. 2015, doi: 10.1371/journal.ppat. 1004932).

In certain embodiments the inventive designs comprise specific changes E169K (HXB2 numbering), as shown in FIGS. 21A-21B. In certain embodiments, CH848.d0949.10.17DT envelope comprises additional modifications D230N.H289N.P291S.E169K and is referred to as CH848.d0949.10.17 Dte. In certain embodiments, CH848.d0949.10.17 envelope comprises additional modifications D230N.H289N.P291S.E169K and is referred to as CH848.d0949.10.17WTe.

In non-limiting embodiments, the envelope in the selections for immunization are included as trimers, protein and/or mRNA. In non-limiting embodiments, the envelope in the selections for immunization are included as nanoparticles, protein and/or mRNA. The designation scNP refers to a non-limiting embodiment of a protein nanoparticle formed by sortase conjugation reaction. In non-limiting embodiments, nanoparticles comprise fusion proteins, for example ferritin-envelope fusion proteins.

In certain embodiments, the inventive designs comprise modifications, including without limitation fusion of the HIV-1 envelope with ferritin using linkers between the HIV-1 envelope and ferritin designed to optimize ferritin nanoparticle assembly.

In certain embodiments, the invention provides HIV-1 envelopes comprising Lys327 (HXB2 numbering) optimized for administration as a prime to initiate V3 glycan antibody lineage, e.g. DH270 antibody lineage.

In certain embodiments, the invention provides HIV-1 envelopes comprising Lys169 (HXB2 numbering).

In certain embodiments, the invention provides a composition comprising any one of the inventive envelopes, e.g., as disclosed in Table 5, or nucleic acid sequences encoding the same. In certain embodiments, the nucleic acid is mRNA. In certain embodiments, the mRNA is comprised in a lipid nano-particle (LNP).

In certain embodiments, the invention provides compositions comprising a nanoparticle which comprises any one of the envelopes of the invention, e.g., as disclosed in Table 5.

In certain embodiments, the invention provides compositions comprising a nanoparticle which comprises any one of the envelopes of the invention, e.g., as disclosed in Table 5, wherein the nanoparticle is a ferritin self-assembling nanoparticle.

In certain aspects, the invention provides a composition comprising a nanoparticle and a carrier, wherein the nanoparticle comprises trimers of any of the recombinant HIV-1 envelopes, e.g. as disclosed in Table 5. In certain embodiments, the nanoparticle is a ferritin self-assembling nanoparticle. In certain embodiments, the nanoparticle comprises multimers of trimers. Provided also are method for using these compositions comprising nanoparticles.

In certain embodiments, the invention provides a method of inducing an immune response in a subject comprising administering an immunogenic composition comprising any one of the recombinant HIV-1 envelopes of the invention e.g., as disclosed in Table 5, or compositions comprising these recombinant HIV-1 envelopes, in an amount sufficient to induce an immune response. In certain embodiments, the composition is administered as a prime and/or a boost. In certain embodiments, the composition is administered as a prime. In certain embodiments, the composition is administered as a boost. In certain embodiments, the composition comprises nanoparticles. In certain embodiments, methods of the invention further comprise administering an adjuvant.

In certain embodiments, the invention provides a composition comprising a plurality of nanoparticles comprising a plurality of the recombinant HIV-1 envelopes or trimers of the invention, e.g., as disclosed in Table 5. In non-limiting embodiments, the envelopes/trimers of the invention are multimeric when comprised in a nanoparticle. The nanoparticle size is suitable for delivery. In non-liming embodiments the nanoparticles are ferritin based nanoparticles.

In certain aspects, the invention provides nucleic acids comprising sequences encoding proteins of the invention, e.g., as disclosed in Table 5. In certain embodiments, the nucleic acids are DNAs. In certain embodiments, the nucleic acids are mRNAs, modified or unmodified, suitable for use any use, e.g. but not limited to use as pharmaceutical compositions. In certain aspects, the invention provides expression vectors comprising the nucleic acids of the invention.

In certain aspects, the invention provides a pharmaceutical composition comprising mRNAs encoding the inventive HIV-1 envelopes, e.g., as disclosed in Table 5. In certain embodiments, these are optionally formulated in lipid nanoparticles (LNPs). In certain embodiments, the mRNAs are modified. Modifications include without limitations modified ribonucleotides, poly-A tail, 5′cap.

In certain embodiments, the nucleic acids are formulated in lipid, such as but not limited to LNPs. Non-limiting embodiments include LNPs without polyethylene glycol.

In certain aspects the invention provides nucleic acids encoding the inventive protein designs. In non-limiting embodiments, the nucleic acids are mRNA, modified or unmodified, suitable for any use, e g but not limited to use as pharmaceutical compositions. In certain embodiments, the nucleic acids are formulated in lipid, such as but not limited to LNPs.

In certain aspects the invention provides a method of inducing an immune response comprising administering an immunogenic composition comprising a prime immunogen followed by at least one boost immunogen from Table 5, wherein the boost immunogens are administered in an amount sufficient to induce an immune response. In certain embodiments, the prime is one of the CH848.0949.10.17DT, CH848.0949.10.17Dte, CH848.d0949.10.17DT GS, or CH848.d0949.10.17DT.GS comprising additional modifications D230N.H289N.P291S.E169K designs. See Table 2 and WO2022/087031 which content is herein incorporated by reference in its entirety. In certain embodiments, the first boost is one of the CH848.0949.10.17WT, CH848.0949.10.17Wte designs. See Table 2 and WO2022/087031 which content is herein incorporated by reference in its entirety. In certain embodiments, the first boost is one of the CH848.0949.10.17DT or CH848.0949.10.17Dte designs. See Table 2. In certain embodiments, the boost is CH848.0358.80.06 or CH848.1432.5.41. In some embodiments, the modification to the V1 loop described herein can be incorporated into the envelope used as the prime and/or boost. In some embodiments, the method further comprises administering an immunogenic composition comprising any HIV-1 envelope sequence from the CH848 infected individual and variants thereof comprising the modification to the V1 loop described herein. In some embodiments, the method comprises administering an immunogenic composition comprising any HIV-1 envelope sequence from the CH848 infected individual and variants thereof comprising the modification to the V1 loop described herein as a prime.

In certain embodiments, the methods further comprise administering a boost from Table 4, wherein the boost is CH848.0808.15.15 in any suitable form.

In certain embodiments, the methods further comprise administering a boost from Table 4, wherein the boost is CH848.0358.80.06 in any suitable form.

In certain embodiments, the methods further comprise administering a boost from Table 4, wherein the boost is CH848.1432.5.41 in any suitable form.

In certain embodiments, the methods further comprise administering a boost from Table 4, wherein the boost is CH848.1621.4.44 in any suitable form.

In certain embodiments, the methods further comprise administering a boost from Table 4, wherein the boost is CH848.1305.10.35 in any suitable form.

In certain embodiments, the methods further comprise comprising administering a boost from Table 4, wherein the boost is PO402.c2.11 (G) in any suitable form.

In certain embodiments, the methods further comprise administering a boost from Table 4, wherein the boost is ZM246F (C) in any suitable form.

In certain embodiments, the methods further comprise administering a boost CH848.0358.80.06 in any suitable form.

In certain embodiments, the methods further comprise administering a boost CH848. 1432 5.41 in any suitable form.

In certain embodiments, the methods further comprise administering a boost from Table 5, wherein the boost is an envelope from Table 5 in any suitable form. In certain embodiments, the boost comprises envelope CH848.0949.10.17WT, CH848.0949.10.17WTe, or CH848.0808.15.15. In certain embodiments, the boost comprises envelope CH848.0949.10.17WT, CH848.0949.10.17WTe, or CH848.0808.15.15 comprising a modifications to the V1 loop to connect V1 residues 104 and 109 (HBX2 numbering) to with linker “GSGG”. In certain embodiments, the boost envelope comprises CH848.3.D0949.10.17chim.6R.DS.SOSIP.664_N133D_GS135-40.

In certain embodiments, the prime and/or boost immunogen are administered as a nanoparticle. In certain embodiments, the nanoparticle is a ferritin nanoparticle. In certain embodiments, the methods further comprise administering the prime and/or boost immunogen as a mRNA-LNP formulation.

In certain embodiments, the methods further comprise administering any suitable adjuvant.

### BRIEF DESCRIPTION OF DRAWINGS

The patent or application file contains at least one drawing executed in color. To conform to the requirements for PCT patent applications, many of the figures presented herein are black and white representations of images originally created in color.

FIG. 1 shows non-limiting embodiments of nucleic acid sequences of envelopes of the invention.

FIG. 2 shows non-limiting embodiments of amino acid sequences of envelopes of the invention.

FIG. 3 shows non-limiting embodiments of the sortase design of an envelope of the invention.

FIG. 4 shows that CH0848 10.17DT SOSIP engages the DH270 UCA Fab with 60 nM affinity.

FIG. 5 shows natural envelopes with 17 aa V1 loops lacking N133/N138 glycans exist in vivo.

FIG. 6 shows CH0848.D1305.10.19, and CH0848.D949.10.17 VIV2 loop alignment and that CH0848.D1305.10.19 lacks N133 and N138 glycans in the V1 region of HIV-1 Env.

FIG. 7 shows DH270 UCA does not bind natural Env CH0848.D1305. 10.19 that has a 17 aa V1 loop and lacks N133 and N138 glycans.

FIGS. 8A and 8B show that the CH0848 natural Eny with a 17 aa V1 loop and no N133 and N138 glycan has eliminated the N295, N301, and N332 glycan. The figure shows JRFL, CH0848 D1305.10.19, and CH0848 D949.10.17 V3 loop alignment.

FIGS. 9A and 9B show that the DH270-resistant CH0848 natural Env with a 17 aa V1 loop and no N133 and N138 glycan acquire V2 apex bnAb binding. Potential V3-glycan escape variant is recognized by V2 apex bnAbs.

FIG. 10 shows CH0848.D1305.10.19, and CH0848.D949.10.17 V2 loop alignment and that CH0848.D949.10.17 clone encodes E169 instead of K169. K169E mutations are known to eliminate binding of VIV2 glycan bnAbs.

FIG. 11 shows the design of V3 chimeric CH0848 Envelope antigenic for V1V2 glycan and V3 glycan.

FIG. 12 shows that 19CV3 binds to UCAs of VIV2 glycan and V3 glycan antibodies.

FIG. 13 shows non-limiting embodiments of prime boost regimens combining germline targeting and B cell mosaic Envs.

FIG. 14 shows biolayer interferometry binding by different members of the DH270 V3-glycan antibody lineage. The precursor of the lineage is DH270 UCA3. Somatically mutated lineage members (DH270UCA3 is the unmutated common ancestor, DH270 14, DH270.1 and DH270.6 have increasing somatic mutations) bind better to Arg327 than Lys327. The germline precursor requires Lys327 in order to bind and stay bound to CH848.3.D0949.10.17_N133D_N138T_D230N_H289N_P219S DS SOSIP gp140 trimer.

FIGS. 15A-B show that the addition of E169K enables binding of VIV2-glycan broadly neutralizing antibody PGT145 while retaining V3-glycan antibody binding. Antibody binding was measured by biolayer interferometry. The red vertical line demarks the change from association phase to dissociation phase. Binding curves to CH848.D949.10.17 N133D/N138T is shown in FIG. 15A and CH848.D949.10.17 N133D/N138T/E169K is shown in FIG. 15B. Antibody DH542 is the same as antibody DH270.6.

FIGS. 16A-B show 19CV3 induces serum binding antibody responses in DH270 germline precursor knockin mice. Knockin mice were immunized with CH848.D1305.10.19_D949V3 gp140 trimer plus adjuvant (red, n=6) or adjuvant alone (silver, n=2). Serum antibody binding to the CH848.D1305.10.19_D949V3 Env trimer used for immunization (FIG. 16A) or the gp 120 subunit from a related virus (FIG. 16B) Group mean values are shown.

FIGS. 17A-B show 19CV3 induces serum antibodies that neutralize HIV-1 with and without V1 glycans removed. Serum antibody neutralization of HIV-1 infection of TZM-bl cells. DH270 germline precursor knockin mice were immunized with CH848.D1305.10.19_D949V3 plus adjuvant (circles, n=6) or adjuvant alone (squares, n=2). Serum was tested for neutralization of HIV-1 isolates CH848.D949.10.17 N133D/N138T (FIG. 17A) and CH848.D949.10.17 (FIG. 17B). Neutralization titers are shown as the reciprocal dilution of serum required to inhibit 50% of virus replication. The neutralization titer for the group were averaged as the geometric mean.

FIGS. 18A-B show vaccine-induced serum HIV-1 antibody responses in CHO1 germline precursor knock-in mice. Knock-in mice were immunized with CH848.D1305.10.19_D949V3 (19CV3) plus adjuvant (circles, n=6) or adjuvant alone (squares, n=3). FIG. 18A shows serum antibody binding to the CH848.D1305.10.19_D949V3 Env trimer used for immunization. Group mean values are shown. FIG. 18B shows serum antibody neutralization of HIV-1 infection of TZM-bl cells. Serum was tested for neutralization against three genetically distinct HIV-1 isolates from CRF AG, clade A, and clade C. Neutralization titers are shown as the reciprocal dilution of serum required to inhibit 50% of virus replication. The group geometric mean neutralization titer is indicated with a horizontal bar. Serum lacked neutralization of the negative control murine leukemia virus.

FIG. 19 shows CH848.D1305.10.19_D949V3 (19CV3) DS SOSIP gp 140 elicits V3 glycan directed binding antibodies in rhesus macaques. Serum antibodies were examined for binding to CH848 Env trimers with (WT) and without the N332 glycan (N332A) over the course of vaccination. Binding titers were higher for CH848 Env trimers with the N332 glycan present. This is significant because broadly neutralizing antibodies target the N332 glycan and require it for binding to Env trimers. Arrows indicate time of immunization. Mean and standard error are shown for the group of 3 macaques.

FIGS. 20A-B show vaccination of rhesus macaques with CH848.D1305.10.19_D949V3 (19CV3) DS.SOSIP gp 140 elicits glycan-dependent serum neutralizing antibodies. FIG. 20A shows serum neutralization of kifunensine-treated JR-FL or murine leukemia virus. Kifunensine treatment of virus results in Man9GlcNAc2 glycosylation of HIV-1 envelope. Neutralization of Man9GlcNAc2-enriched virus can suggest the presence of mannose-reactive neutralizing HIV-1 antibodies. DH270 bnAbs require Man9GlcNAc2-enrichment for neutralization early in their development, thus serum neutralization of Man9GlcNAc2-enriched JR-FL may indicate elicitation of precursors of DH270-like antibodies. FIG. 20B shows serum neutralization of a panel of autologous CH848 viruses and heterologous genetically distinct HIV-1 isolates. Neutralization of JRFL was dependent on Man9GlcNAc2-enrichment. Murine leukemia virus was used as a non-HIV negative control for neutralization. Neutralization titers are shown as reciprocal plasma dilution that inhibits 50% of virus replication (ID50). Each symbol represents an individual macaque. Horizontal bars show the group geometric mean (n=3).

FIGS. 21A-B show non-limiting embodiments for sequences of the invention comprising amino acid Arg327 (K327R). In the amino acid sequences (FIG. 21B), underlined is the signal peptide and the preceding four amino acids indicate the cloning site/kozak sequence (VDTA) neither of which that would not be part of the final recombinant protein.

FIGS. 22A-B show non-limiting embodiments of sequences of the invention comprising varying linkers between the envelope and ferritin proteins. In the amino acid sequences (FIG. 22B), underlined is the signal peptide and the preceding four amino acids indicate the cloning site/kozak sequence (VDTA) neither of which that would not be part of the final recombinant protein.

FIGS. 23A-B show non-limited embodiments of designs of 19CV3 sequences. In the amino acid sequences (FIG. 23B), underlined is the signal peptide and the preceding four amino acids indicate the cloning site/kozak sequence (VDTA) neither of which that would not be part of the final recombinant protein.

FIGS. 24A-B show non-limited embodiments of designs of 19CV3 sequences. Amino acids H66A_A582T_L587A are referred to JS2 or “joe2” mutations. In the amino acid sequences (FIG. 24B), underlined is the signal peptide and the preceding four amino acids indicate the cloning site/kozak sequence (VDTA) neither of which that would not be part of the final recombinant protein.

FIGS. 25A-B show a summary of non-limiting embodiments of envelope designs of the invention.

FIG. 26 shows one embodiment of a design for the production of trimeric HIV-1 Env on ferritin nanoparticles.

FIG. 27 shows non-limiting examples of envelopes designs and sequences described in Table 3.

FIG. 28 shows non-limiting examples of envelope designs and sequences described in Table 4-envelopes CH848.0808.15.15, CH848.1621.4.44, CH848.1305.10.35, P0402.c2.11 (G), ZM246F (C).

FIGS. 29A and 29B show non-limiting examples of designs and sequences. FIG. 29A shows non-limiting examples of designs and sequences based on envelope CH848.0358.80.06 and CH848.1432.5.41. FIG. 29B shows non-limiting examples of envelopes designs and sequences described in Table 5.

FIGS. 30A to 30F show data from Example 5. FIG. 30A depicts Glycan-V3 bnAbs having long CDR H3 loops and CDR H3 contacts which are critical for function and precursor engagement. FIG. 30B depicts Glycan-V3 bnAbs having have long CDR H3 loops containing long non-templated N-nucleotide addition regions. FIG. 30C depicts single site saturation library of the DH270UCA3 CDR H3 loop. It displays a library of developed DH270UCA3 Ab variants that contain all the single amino acid mutations in the CDR H3 loop. FIG. 30D depicts DH270UCA3.CDRH3 library selection. FIG. 30E depicts DH270UCA3.CDRH3 substitutions recognized by 10.17DT. FIG. 30F depicts 10.17DT's limited recognition of DH270UCA3 CDR H3 loop variants.

FIGS. 31A to 31C depict single residue substitutions tolerated in the CDRH3 of CH235UCA by CH505.M5.G458Y/GnTI-.

FIGS. 32A to 32B depict the identification of naturally occurring CDR H3 loops that can be recognized by 10.17DT.

FIG. 33 depicts selection and characterization of DH270UCA_CDRH3 chimeras.

FIG. 34 depicts data demonstrating that 10.17 has limited recognition of naturally occurring CDR H3 loops.

FIGS. 35A to 35C show data from Example 5. FIG. 35A depicts 10.17DT. V1 loop contacts with the DH270UCA3 weaken interaction with the V3 loop. FIG. 35B depicts design of a 10.17DT variant with a shorter V1 loop. FIG. 35C demonstrates that 10.17DT.GS shows tighter binding to DH270UCA3 and DH270UCA3 G57R.

FIGS. 36A to 36B depicts that 10.17DT.GS recognizes diverse DH270UCA3 CDR H3 loops variants.

FIGS. 37A-37X show a summary of the analyses and selection of a new set of immunogens for induction of HIV-I neutralizing antibodies. FIG. 37A depicts a DH270 bNAb lineage. FIG. 37B depicts heterologous and autologous panels. FIG. 37C depicts signature phenotypes. FIG. 37D depicts reduced heterologous dataset for signature analyses. FIG. 37E depicts a reduced autologous dataset for signature analysis. Roughly day 700 is represented by a cyan box. FIG. 37F depicts IA1 breadth gain signature in an autologous panel. FIG. 37G depicts a comparison between a global panel and an autologous panel of signatures. Color-coding is according to autologous signatures. FIG. 37H depicts IA1 breadth gain heterologous signatures. FIG. 37I depicts bNAb education. FIG. 37J depicts the systematic definition of “bnab education” sites. FIG. 37K depicts positional characterization of bNAb education signature sites. FIG. 37L depicts logos for bNAb education sites. FIG. 37M depicts longitudinal evolution of variants. FIGS. 37N and 37O depict structural relevance of select mutations. FIG. 37P depicts IA1 breadth gain loop signatures. FIGS. 37Q and 37R depict previous (FIG. 37Q) and new (FIG. 37R) immunogen designs. FIG. 37S depicts an improved breadth of DH270.6. FIG. 37T depict immunogens for breadth gain signatures of 10-1074 and PGT128 over DH270.6. FIG. 37U depicts new sequential immunogens. FIG. 37V depicts potential issues with UG021.16. FIGS. 37W and 37X depict previous (FIG. 37W) and proposed (FIG. 37X) DH270 UCA knock-in mice immunization studies.

FIGS. 38A-38Q show signature analyses for a new set of immunogens for induction of HIV-1 neutralizing antibodies. FIG. 38A depicts key mutations for DH270 lineage Abs. FIG. 38B depicts IA4 heterologous signatures. FIG. 38C depicts IA4 autologous signatures. FIG. 38D depicts IA4 signatures longitudinally. FIG. 38E depicts IA4 variable loop signatures. FIG. 38F depicts longitudinal loop evolution. FIG. 38G depicts IA2 breadth gain signatures. FIG. 38H depicts IA2 breadth gain signatures longitudinally. FIG. 38I depicts IA2 signatures. FIG. 38J IA 1 breadth gain signatures. FIG. 38K depicts signature strengths. AbsMCC=absolute Matthew's correlation coefficients. +1 is perfect association, 0 is no association. Rlcl, rlc2, r2c1, r2c2 is the contingency table: R1=Ab Sensitive/Breadth gain; R2=Ab Resistant/common sensitivity; C1=Viruses with amino acid/glycan; C2=Viruses without amino acid/glycan. Table=T1 (simple) or T2/T3 (two flavors of phylogenetic tests). Tlg or T3g are glycan tests. FIG. 38L depicts breadth gain signatures of 10-1074 and PCT128 over DH270.6. FIG. 38M depicts immunogens for breadth gain beyond DH270.6. See also FIG. 37T. FIG. 38N depicts the two heterologous viruses lacking NxST332 and NxST334 that are sensitive to DH270.6. FIG. 38O depicts alternate selections. FIG. 38P depicts IA1 signatures. FIG. 38Q depicts IA1 heterologous signatures.

FIGS. 39A-39R show data for vaccine elicitation of serum neutralization in mouse study MUS98. FIG. 39A depicts the DH270 UCA4 VH+/−, VL+/− knockin mouse protein immunization regimen. CH848 was down selected using GS135-40 in DH270 UCA4 HCLC het/het (VH+/−, VL+/−) mice FIGS. 39B-39R depicts serum binding antibody responses in DH270 UCA4 VH+/−, VL+/− knockin mice. Knockin mice were immunized with envelopes as indicated, including CH848.3.D0949.10.17chim.6R.DS.SOSIP.664_N133D_GS135-40. FIG. 39J shows a comparison of serum binding antibody responses between the different envelopes indicated.

FIG. 40 depicts results from neutralization assays in TZM-bl cells.

FIGS. 41A-41N show next generation sequencing of heavy chain and light chain variable regions shows vaccine selection of critical functional improbable mutations needed for DH270 antibody affinity maturation. FIGS. 41A-41N show the frequency of the observed somatic mutation at the recited positions as inferred from nucleic acid sequencing of antibodies from the mice in Group 1 of mouse study MU598 (see FIG. 39A). Each of FIGS. 41A-41N depict one or more amino acid frequency at a recited position.

FIGS. 42A-42D depicts mutation frequency in individual mice. FIG. 42A depicts mutation frequency in the heavy chain of the human antibody. FIG. 42B depicts mutation frequency in the heavy chain of the mouse antibody. FIG. 42C depicts mutation frequency in the kappa chain of the human antibody. FIG. 42D depicts mutation frequency in the kappa chain of the mouse antibody.

FIGS. 43A and 43B depict logo plots representing the frequency of amino acids at each position of the antibody sequencesfrom the mice. FIG. 43A depicts logo plots of the heavy chain. FIG. 43B depicts logo plots of the kappa chain.

FIGS. 44A and 44B depicts mutation frequencies of the V gene in the mice. FIG. 44A depicts mutation frequencies in the heavy chain. FIG. 44B depicts mutation frequencies in the kappa chain.

### DETAILED DESCRIPTION

The development of a safe, highly efficacious prophylactic HIV-1 vaccine is of paramount importance for the control and prevention of HIV-1 infection. A major goal of HIV-1 vaccine development is the induction of broadly neutralizing antibodies (bnAbs) (Immunol. Rev. 254:225-244, 2013). BnAbs are protective in rhesus macaques against SHIV challenge, but as yet, are not induced by current vaccines.

For the past 25 years, the HIV vaccine development field has used single or prime boost heterologous Envs as immunogens, but to date has not found a regimen to induce high levels of bnAbs.

Recently, a new paradigm for design of strategies for induction of broadly neutralizing antibodies was introduced, that of B cell lineage immunogen design (Nature Biotech. 30:423, 2012) in which the induction of bnAb lineages is recreated. It was recently demonstrated the power of mapping the co-evolution of bnAbs and founder virus for elucidating the Env evolution pathways that lead to bnAb induction (Nature 496:469, 2013). The invention provides methods of using these pan bnAb envelope immunogens.

In certain aspect, the invention provides compositions for immunizations to induce lineages of broad neutralizing antibodies. In certain embodiments, there is some variance in the immunization regimen; in some embodiments, the selection of HIV-1 envelopes may be grouped in various combinations of primes and boosts, either as nucleic acids, proteins, or combinations thereof. In certain embodiments the compositions are pharmaceutical compositions which are immunogenic. In certain embodiments, the compositions comprise amounts of envelopes which are therapeutic and/or immunogenic.

In one aspect the invention provides a composition for a prime boost immunization regimen comprising any one of the envelopes described herein, or any combination thereof wherein the envelope is a prime or boost immunogen. In certain embodiments the composition for a prime boost immunization regimen comprises one or more envelopes described herein.

In certain embodiments, the compositions contemplate nucleic acid, as DNA and/or RNA, or recombinant protein immunogens either alone or in any combination. In certain embodiments, the methods contemplate genetic, as DNA and/or RNA, immunization either alone or in combination with recombinant envelope protein(s).

In some embodiments the antigens are nucleic acids, including but not limited to mRNAs which could be modified and/or unmodified. See US Pub 20180028645A1, US Pub 20170369532, US Pub 20090286852, US Pub 20130111615, US Pub 20130197068, US Pub 20130261172, US Pub 20150038558, US Pub 20160032316, US Pub 20170043037, US Pub 20170327842, each content is incorporated by reference in its entirety. mRNAs delivered in LNP formulations have advantages over non-LNPs formulations. See US Pub 20180028645A1.

In certain embodiments the nucleic acid encoding an envelope is operably linked to a promoter inserted an expression vector. In certain aspects the compositions comprise a suitable carrier. In certain aspects the compositions comprise a suitable adjuvant.

In certain embodiments the induced immune response includes induction of antibodies, including but not limited to autologous and/or cross-reactive (broadly) neutralizing antibodies against HIV-1 envelope. Various assays that analyze whether an immunogenic composition induces an immune response, and the type of antibodies induced are known in the art and are also described herein.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

The envelope used in the compositions and methods of the invention can be a gp160, gp150, gp145, gp140, gp120, gp41, N-terminal deletion variants as described herein, cleavage resistant variants as described herein, or codon optimized sequences thereof. In certain embodiments the composition comprises envelopes as trimers. In certain embodiments, envelope proteins are multimerized, for example trimers are attached to a particle such that multiple copies of the trimer are attached and the multimerized envelope is prepared and formulated for immunization in a human. In certain embodiments, the compositions comprise envelopes, including but not limited to trimers as a particulate, high-density array on liposomes or other particles, for example but not limited to nanoparticles. In some embodiments, the trimers are in a well ordered, near native like or closed conformation. In some embodiments the trimer compositions comprise a homogenous mix of native like trimers. In some embodiments the trimer compositions comprise at least 85%, 90%, 95% native like trimers.

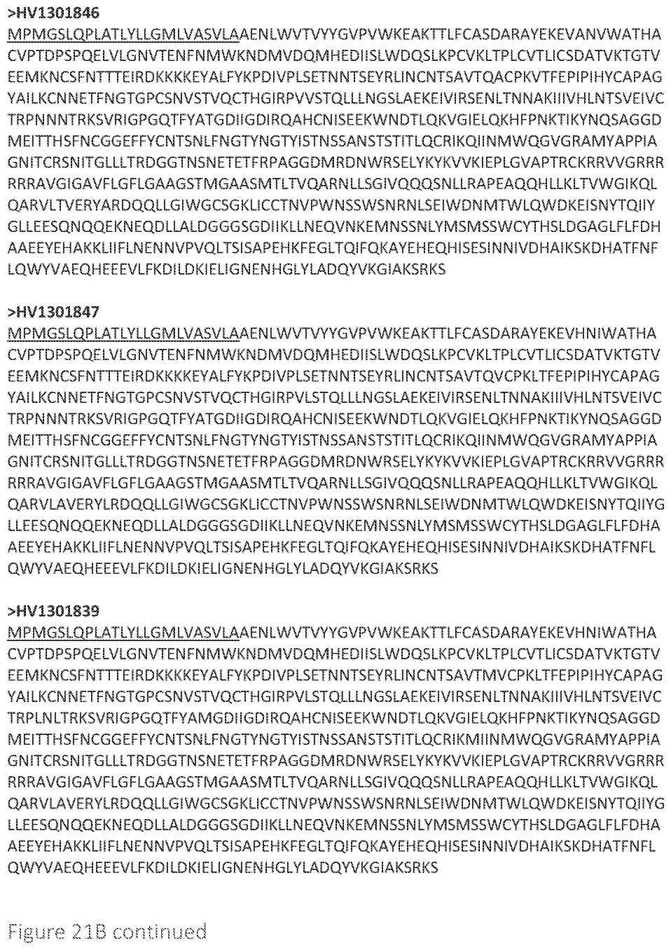

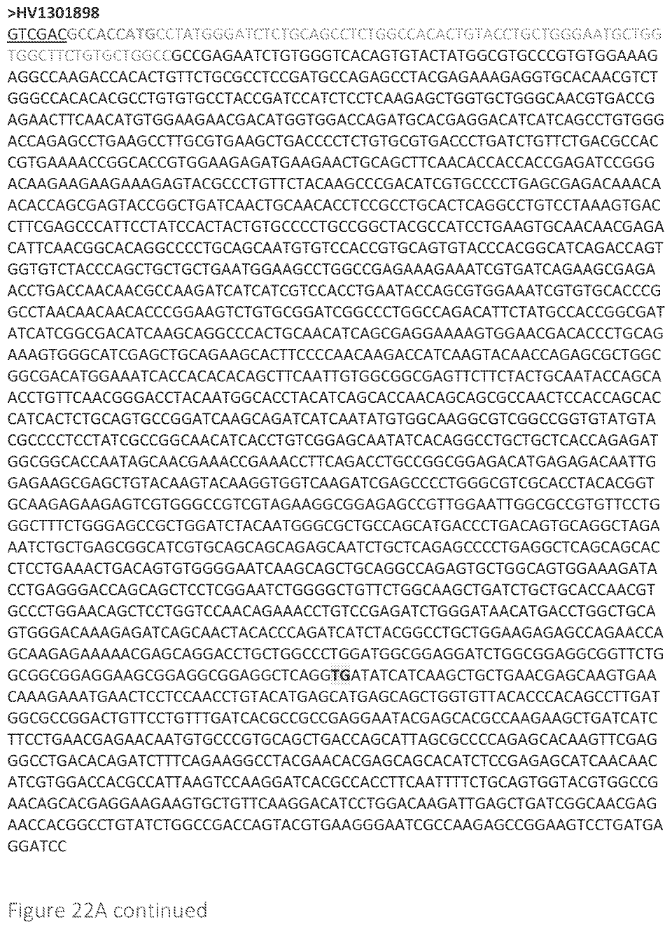

In certain embodiments the envelope is any of the forms of HIV-1 envelope. In certain embodiments the envelope is gp 120, gp140, gp145 (i.e. with a transmembrane domain), or gp150. In certain embodiments, gp 140 is designed to form a stable trimer. See Table 1, 2, FIGS. 21-25 for non-limiting examples of sequence designs. In certain embodiments envelope protomers form a trimer which is not a SOSIP timer. In certain embodiment the trimer is a SOSIP based trimer wherein each protomer comprises additional modifications. In certain embodiments, envelope trimers are recombinantly produced. In certain embodiments, envelope trimers are purified from cellular recombinant fractions by antibody binding and reconstituted in lipid comprising formulations. See for example WO2015/127108 titled “Trimeric HIV-1 envelopes and uses thereof” and US2020/0002383 which content is herein incorporated by reference in its entirety. In certain embodiments the envelopes of the invention are engineered and comprise non-naturally occurring modifications.

In certain embodiments, the envelope is in a liposome. In certain embodiments the envelope comprises a transmembrane domain with a cytoplasmic tail, wherein the transmembrane domain is embedded in a liposome. In certain embodiments, the nucleic acid comprises a nucleic acid sequence which encodes a gp 120, gp140, gp145, gp150, or gp160.

In certain embodiments, where the nucleic acids are operably linked to a promoter and inserted in a vector, the vector is any suitable vector. Non-limiting examples include, VSV, replicating rAdenovirus type 4, MVA, Chimp adenovirus vectors, pox vectors, and the like. In certain embodiments, the nucleic acids are administered in NanoTaxi block polymer nanospheres. In certain embodiments, the composition and methods comprise an adjuvant. Non-limiting examples include, 3M052, AS01 B, AS01 E, gla/SE, alum, Poly I poly C (poly IC), polyIC/long chain (LC) TLR agonists, TLR7/8 and 9 agonists, or a combination of TLR7/8 and TLR9 agonists (see Moody et al. (2014) J. Virol. March 2014 vol. 88 no. 6 3329-3339), or any other adjuvant. Non-limiting examples of TLR7/8 agonist include TLR7/8 ligands, Gardiquimod, Imiquimod and R848 (resiquimod) A non-limiting embodiment of a combination of TLR7/8 and TLR9 agonist comprises R848 and oCpG in STS (see Moody et al. (2014) J. Virol. March 2014 vol. 88 no. 6 3329-3339). In non-limiting embodiments, the adjuvant is an LNP. See e.g., without limitation Shirai et al. “Lipid Nanoparticle Acts as a Potential Adjuvant for Influenza Split Vaccine without Inducing Inflammatory Responses” Vaccines 2020, 8, 433; doi: 10.3390/vaccines8030433, published 3 Aug. 2020.

In non-limiting embodiments, LNPs used as adjuvants for proteins or mRNA compositions are composed of an ionizable lipid, cholesterol, lipid conjugated with polyethylene glycol, and a helper lipid. Non-limiting embodiments include LNPs without polyethylene glycol.

In certain aspects the invention provides a cell comprising a nucleic acid encoding any one of the envelopes of the invention suitable for recombinant expression. In certain aspects, the invention provides a clonally derived population of cells encoding any one of the envelopes of the invention suitable for recombinant expression. In certain aspects, the invention provides a stable pool of cells encoding any one of the envelopes of the invention suitable for recombinant expression.

In certain aspects, the invention provides a recombinant HIV-1 envelope polypeptide as described here, wherein the polypeptide is a non-naturally occurring protomer designed to form an envelope trimer. The invention also provides nucleic acids encoding these recombinant polypeptides. Non-limiting examples of amino acids and nucleic acid of such protomers are disclosed herein.

In certain aspects the invention provides a recombinant trimer comprising three identical protomers of an envelope. In certain aspects the invention provides an immunogenic composition comprising the recombinant trimer and a carrier, wherein the trimer comprises three identical protomers of an HIV-1 envelope as described herein. In certain aspects the invention provides an immunogenic composition comprising nucleic acid encoding these recombinant HIV-1 envelope and a carrier.

Described herein are nucleic and amino acids sequences of HIV-1 envelopes. The sequences for use as immunogens are in any suitable form. In certain embodiments, the described HIV-1 envelope sequences are gp 160s. In certain embodiments, the described HIV-1 envelope sequences are gp120s. Other sequences, for example but not limited to stable SOSIP trimer designs, gp145s, gp140s, both cleaved and uncleaved, gp 140 Envs with the deletion of the cleavage (C) site, fusion (F) and immunodominant (I) region in gp41—named as gp 140ACFI (gp140CFI), gp140 Envs with the deletion of only the cleavage (C) site and fusion (F) domain—named as gp140ACF (gp140CF), gp140 Envs with the deletion of only the cleavage (C)—named gp 140AC (gp140C) (See e.g. Liao et al. Virology 2006, 353, 268-282), gp150s, gp41s, can be readily derived from the nucleic acid and amino acid gp 160 sequences. In certain embodiments the nucleic acid sequences are codon optimized for optimal expression in a host cell, for example a mammalian cell, a rBCG cell or any other suitable expression system.

An HIV-I envelope has various structurally defined fragments/forms: gp160; gp140—including cleaved gp140 and uncleaved gp 140 (gp140C), gp140CF, or gp140CFI; gp120 and gp41. A skilled artisan appreciates that these fragments/forms are defined not necessarily by their crystal structure, but by their design and bounds within the full length of the gp 160 envelope. While the specific consecutive amino acid sequences of envelopes from different strains are different, the bounds and design of these forms are well known and characterized in the art.

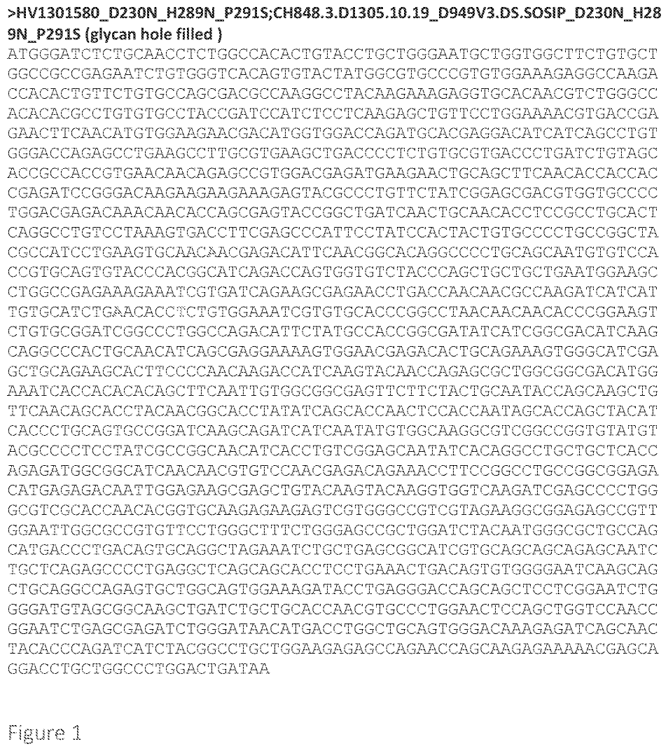

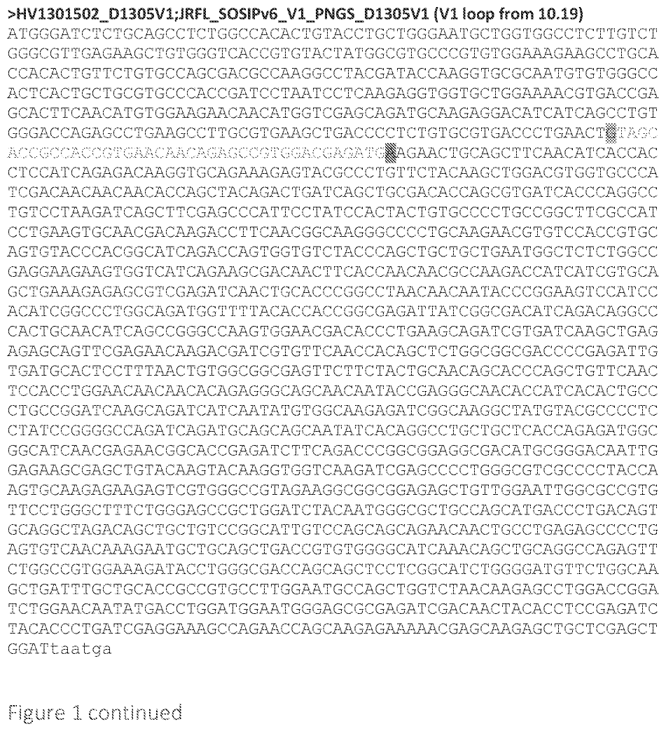

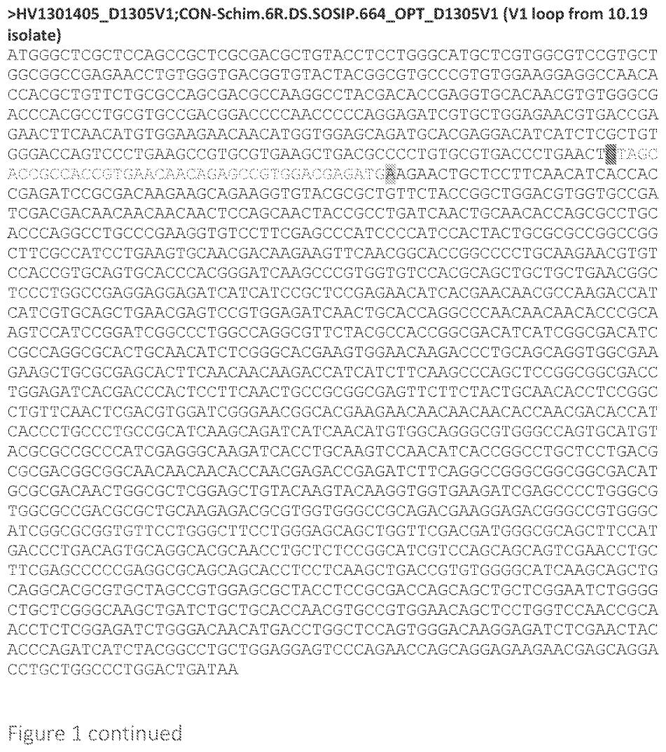

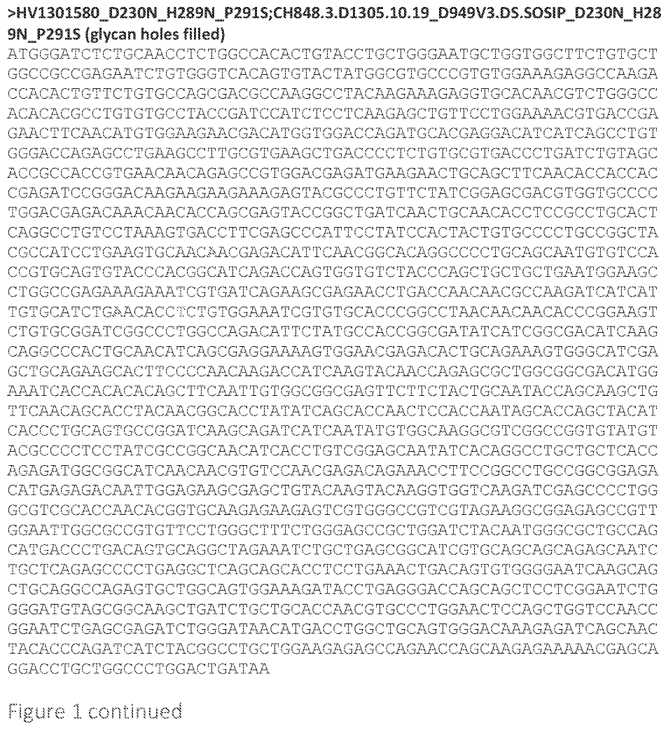

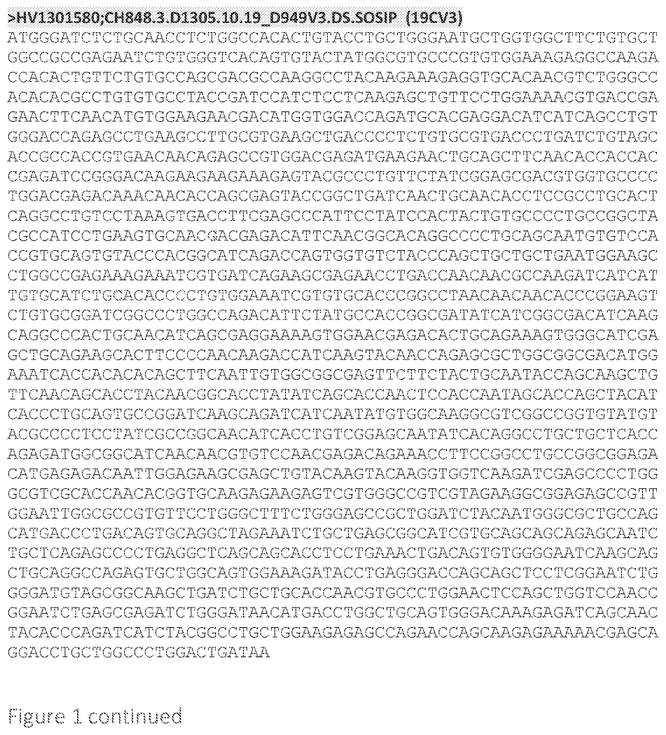

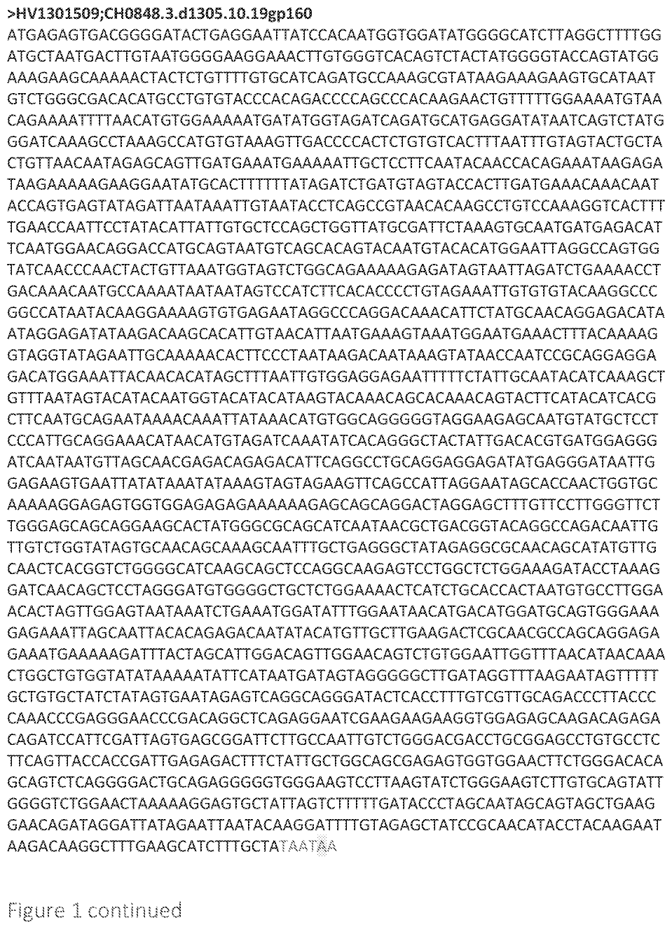

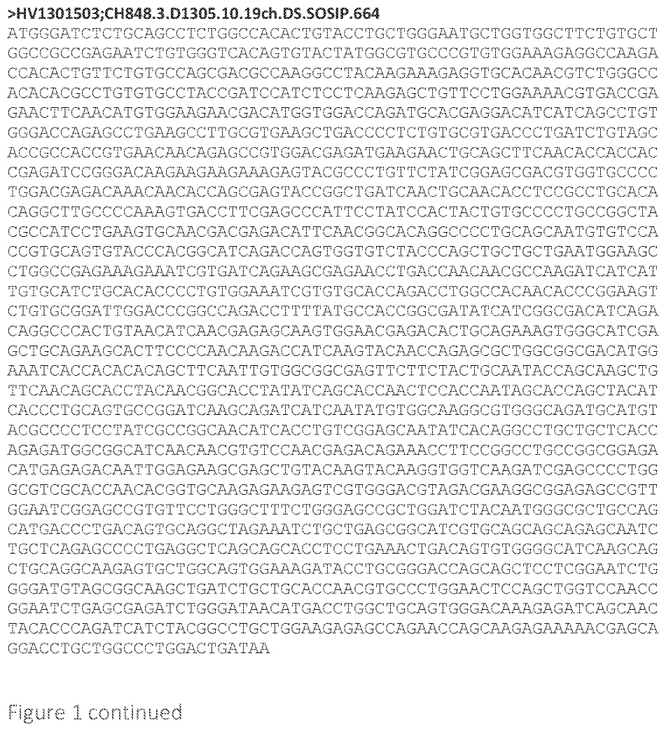

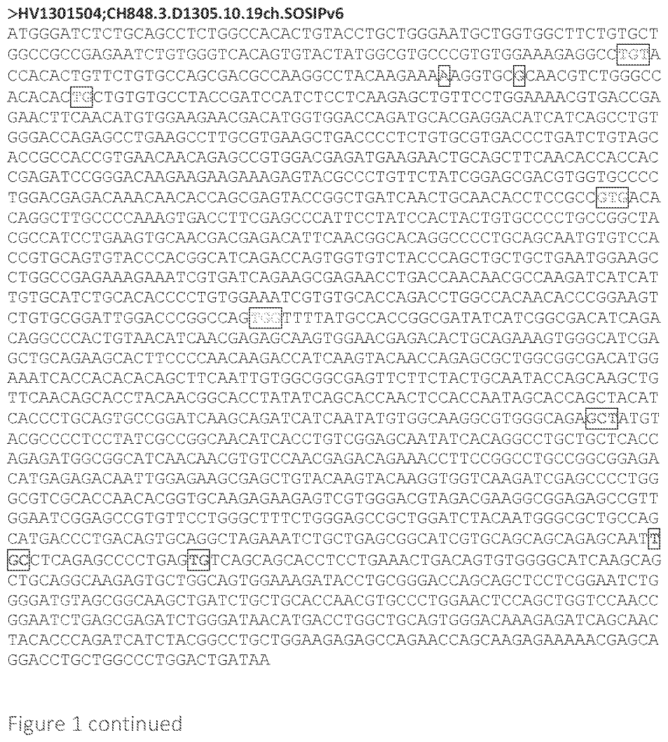

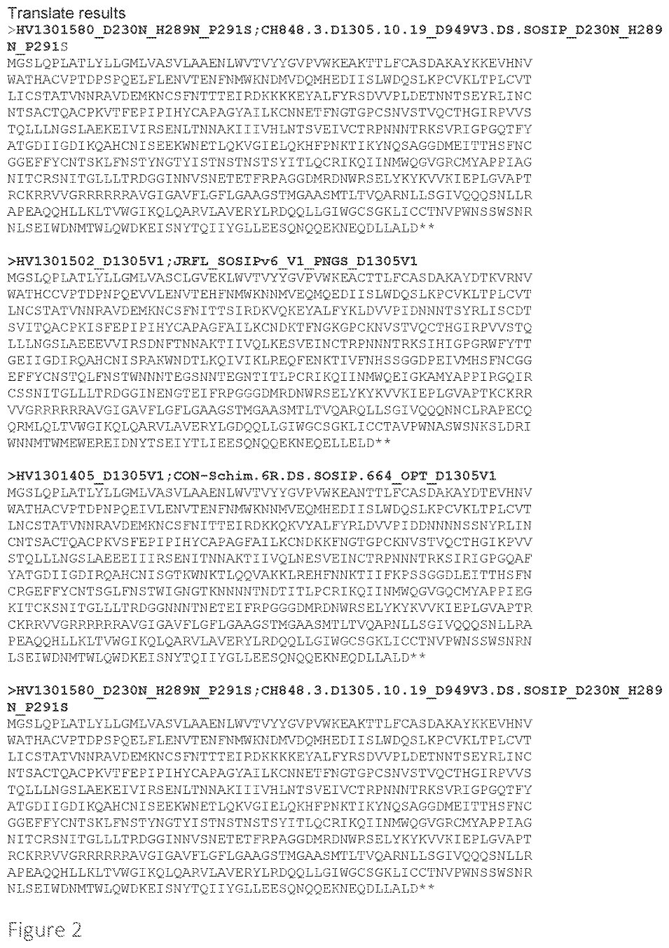

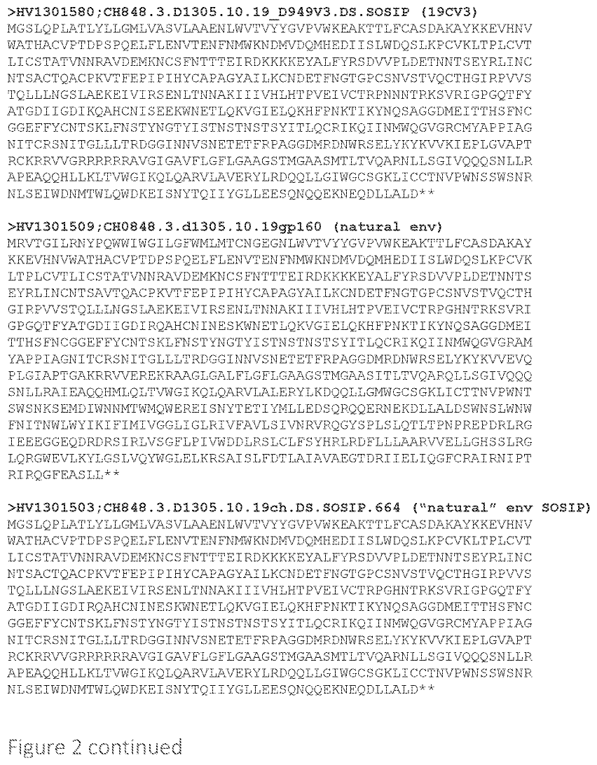

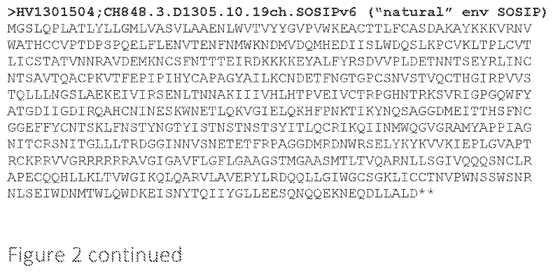

For example, it is well known in the art that during its transport to the cell surface, the gp160 polypeptide is processed and proteolytically cleaved to gp 120 and gp41 proteins. Cleavages of gp 160 to gp120 and gp41 occurs at a conserved cleavage site “REKR.” See Chakrabarti et al. Journal of Virology vol. 76, pp. 5357-5368 (2002); see, e.g., FIG. 1, and second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353 (2): 268-282 (2006).

The role of the furin cleavage site was well understood both in terms of improving cleavage efficiency, see Binley et al. supra, and eliminating cleavage, see Bosch and Pawlita, Virology 64 (5): 2337-2344 (1990); Guo et al. Virology 174:217-224 (1990); McCune et al. Cell 53:55-67 (1988); Liao et al. J Virol. Apr;87 (8): 4185-201 (2013).

Likewise, the design of gp 140 envelope forms is also well known in the art, along with the various specific changes which give rise to the gp 140C (uncleaved envelope), gp140CF and gp140CFI forms. Envelope gp140 forms are designed by introducing a stop codon within the gp41 sequence. See Chakrabarti et al. at FIG. 1.

Envelope gp 140C refers to a gp 140 HIV-1 envelope design with a functional deletion of the cleavage (C) site, so that the gp140 envelope is not cleaved at the furin cleavage site. The specification describes cleaved and uncleaved forms, and various furin cleavage site modifications that prevent envelope cleavage are known in the art. In some embodiments of the gp 140C form, two of the R residues in and near the furin cleavage site are changed to E, e.g., RRVVEREKR is changed to ERVVEREKE, and is one example of an uncleaved gp140 form. Another example is the gp140C form which has the REKR site changed to SEKS. See supra for references.

Envelope gp 140CF refers to a gp140 HIV-1 envelope design with a deletion of the cleavage (C) site and fusion (F) region. Envelope gp140CFI refers to a gp 140 HIV-1 envelope design with a deletion of the cleavage (C) site, fusion (F) and immunodominant (I) region in gp41. See Chakrabarti et al. Journal of Virology vol. 76, pp. 5357-5368 (2002) at for example FIG. 1, and Second paragraph in the Introduction on p. 5357; Binley et al. Journal of Virology vol. 76, pp. 2606-2616 (2002) for example at Abstract: Gao et al. Journal of Virology vol. 79, pp. 1154-1163 (2005); Liao et al. Virology vol. 353 (2): 268-282 (2006).

In certain embodiments, the envelope design in accordance with the present invention involves deletion of residues (e.g., 5-11, 5, 6, 7, 8, 9, 10, or 11 amino acids) at the N-terminus. For delta N-terminal design, amino acid residues ranging from 4 residues or even fewer to 14 residues or even more are deleted. These residues are between the maturation (signal peptide, usually ending with CXX, wherein X can be any amino acid) and “VPVXXXX . . . “. In case of CH505 T/F Env as an example, 8 amino acids (italicized and underlined in the below sequence) were deleted: MRVMGIQRNYPQWWIWSMLGFWMLMICNGMWVIVYYGVPVWKEAKTTLFCASDA KAYEKEVHNVWATHACVPTDPNPQE . . . (rest of envelope sequence is indicated as” . . . ”). In other embodiments, the delta N-design described for CH505 T/F envelope can be used to make delta N-designs of other envelopes. In certain embodiments, the invention relates generally to an HIV-1 envelope immunogen, gp160, gp120, or gp140, without an N-terminal Herpes Simplex gD tag substituted for amino acids of the N-terminus of gp 120, with an HIV leader sequence (or other leader sequence), and without the original about 4 to about 25, for example 4, 5, 6, 7, 8, 9, 10, 11, 12, 13, 14, 15, 16, 17, 18, 19, 20, 21, 22, 23, 24, 25 amino acids of the N-terminus of the envelope (e.g. gp120). See US2014/0248311, e.g. at paragraphs [0043]-[0050], the contents of which publication is hereby incorporated by reference in its entirety.

The general strategy of deletion of N-terminal amino acids of envelopes results in proteins, for example gp 120s, expressed in mammalian cells that are primarily monomeric, as opposed to dimeric, and, therefore, solves the production and scalability problem of commercial gp120 Env vaccine production. In other embodiments, the amino acid deletions at the N-terminus result in increased immunogenicity of the envelopes.

In certain aspects, the invention provides composition and methods which use a selection of Envs, as gp120s, gp 140s cleaved and uncleaved, gp145s, gp150s and gp160s, stabilized and/or multimerized trimers, as proteins, DNAs, RNAs, or any combination thereof, administered as primes and boosts to elicit an immune response. Envs as proteins could be co-administered with nucleic acid vectors containing Envs to amplify antibody induction. In certain embodiments, the compositions and methods include any immunogenic HIV-1 sequences to give the best coverage for T cell help and cytotoxic T cell induction. In certain embodiments, the compositions and methods include mosaic and/or consensus HIV-1 genes to give the best coverage for T cell help and cytotoxic T cell induction. In certain embodiments, the compositions and methods include mosaic group M and/or consensus genes to give the best coverage for T cell help and cytotoxic T cell induction. In some embodiments, the mosaic genes are any suitable gene from the HIV-1 genome. In some embodiments, the mosaic genes are Env genes, Gag genes, Pol genes, Nef genes, or any combination thereof. See e.g. U.S. Pat. No. 7,951,377. In some embodiments the mosaic genes are bivalent mosaics. In some embodiments the mosaic genes are trivalent. In some embodiments, the mosaic genes are administered in a suitable vector with each immunization with Env gene inserts in a suitable vector and/or as a protein. In some embodiments, the mosaic genes, for example as bivalent mosaic Gag group M consensus genes, are administered in a suitable vector, for example but not limited to HSV2, would be administered with each immunization with Env gene inserts in a suitable vector, for example but not limited to HSV-2.

In certain aspects the invention provides compositions and methods of Env genetic immunization either alone or with Env proteins to recreate the swarms of evolved viruses that have led to bnAb induction. Nucleotide-based vaccines offer a flexible vector format to immunize against virtually any protein antigen. Currently, two types of genetic vaccination are available for testing—DNAs and mRNAs.

In certain aspects the invention contemplates using immunogenic compositions wherein immunogens are delivered as DNA. See Graham BS, Enama ME, Nason MC, Gordon IJ, Peel SA, et al. (2013) DNA Vaccine Delivered by a Needle-Free Injection Device Improves Potency of Priming for Antibody and CD8+ T-Cell Responses after rAd5 Boost in a Randomized Clinical Trial. PLOS ONE 8 (4): e59340, page 9. Various technologies for delivery of nucleic acids, as DNA and/or RNA, so as to elicit immune response, both T-cell and humoral responses, are known in the art and are under developments. In certain embodiments, DNA can be delivered as naked DNA. In certain embodiments, DNA is formulated for delivery by a gene gun. In certain embodiments, DNA is administered by electroporation, or by a needle-free injection technologies, for example but not limited to Biojector® device. In certain embodiments, the DNA is inserted in vectors. The DNA is delivered using a suitable vector for expression in mammalian cells. In certain embodiments the nucleic acids encoding the envelopes are optimized for expression. In certain embodiments DNA is optimized, e.g. codon optimized, for expression. In certain embodiments the nucleic acids are optimized for expression in vectors and/or in mammalian cells. In non-limiting embodiments these are bacterially derived vectors, adenovirus based vectors, rAdenovirus (e.g. Barouch DH, et al. Nature Med. 16:319-23, 2010), recombinant mycobacteria (e.g. rBCG or M smegmatis) (Yu, JS et al. Clinical Vaccine Immunol. 14:886-093,2007; ibid 13:1204-11,2006), and recombinant vaccinia type of vectors (Santra S. Nature Med. 16:324-8, 2010), for example but not limited to ALVAC, replicating (Kibler KV et al., PLOS One 6: e25674, 2011 Nov. 9.) and non-replicating (Perreau M et al. J. virology 85:9854-62, 2011) NYVAC, modified vaccinia Ankara (MVA)), adeno-associated virus, Venezuelan equine encephalitis (VEE) replicons, Herpes Simplex Virus vectors, and other suitable vectors.

In certain aspects the invention contemplates using immunogenic compositions wherein immunogens are delivered as DNA or RNA in suitable formulations. Various technologies which contemplate using DNA or RNA, or may use complexes of nucleic acid molecules and other entities to be used in immunization. In certain embodiments, DNA or RNA is administered as nanoparticles consisting of low dose antigen-encoding DNA formulated with a block copolymer (amphiphilic block copolymer 704). See Cany et al., Journal of Hepatology 2011 vol. 54 j 115-121; Arnaoty et al., Chapter 17 in Yves Bigot (ed.), Mobile Genetic Elements: Protocols and Genomic Applications, Methods in Molecular Biology, vol. 859, pp293-305 (2012); Arnaoty et al. (2013) Mol Genet Genomics. 2013 August;288 (7-8): 347-63. Nanocarrier technologies called Nanotaxi® for immunogenic macromolecules (DNA, RNA, Protein) delivery are under development. See for example technologies developed by incellart.

In certain aspects, the invention provides nucleic acids comprising sequences encoding envelopes of the invention. In certain embodiments, the nucleic acids are DNAs. In certain embodiments, the nucleic acids are mRNAs. In certain aspects, the invention provides expression vectors comprising the nucleic acids of the invention.

In certain aspects, the invention provides a pharmaceutical composition comprising mRNAs encoding the inventive antibodies. In certain embodiments, these are optionally formulated in lipid nanoparticles (LNPs). In certain embodiments, the mRNAs are modified. Modifications include without limitations modified ribonucleotides, poly-A tail, 5′cap.

In certain aspects the invention provides nucleic acids encoding the inventive envelopes. In non-limiting embodiments, the nucleic acids are mRNA, modified or unmodified, suitable for use any use, e.g. but not limited to use as pharmaceutical compositions. In certain embodiments, the nucleic acids are formulated in lipid, such as but not limited to LNPs.

In some embodiments the immunogens are administered as nucleic acids, including but not limited to mRNAs which could be modified and/or unmodified. See US Pub 20180028645A1, US Pub 20090286852, US Pub 20130111615, US Pub 20130197068, US Pub 20130261172, US Pub 20150038558, US Pub 20160032316, US Pub 20170043037, US Pub 20170327842, U.S. Pat. Nos. 10,006,007, 9,371,511, 9,012,219, US Pub 20180265848, US Pub 20170327842, US Pub 20180344838A1 at least at paragraphs [0260]-[0281], US Pub 20190153425 for non-limiting embodiments of chemical modifications, wherein each content is incorporated by reference in its entirety.

mRNAs delivered in LNP formulations have advantages over non-LNPs formulations. See US Pub 20180028645A1, US Pub 20190274968, US Pub 20180303925, wherein each content is incorporated by reference in its entirety.

In certain embodiments the nucleic acid encoding an envelope is operably linked to a promoter inserted an expression vector. In certain aspects the compositions comprise a suitable carrier. In certain aspects the compositions comprise a suitable adjuvant.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

In one embodiment, the nucleic acid is an RNA molecule. In one embodiment, the RNA molecule is transcribed from a DNA sequence described herein. In some embodiments, the RNA molecule is encoded by one of the inventive sequences. In another embodiment, the nucleotide sequence comprises an RNA sequence transcribed by a DNA sequence encoding any one of the polypeptide sequence of the sequences of the invention, or a variant thereof or a fragment thereof. Accordingly, in one embodiment, the invention provides an RNA molecule encoding one or more of inventive envelopes. The RNA may be plus-stranded. Accordingly, in some embodiments, the RNA molecule can be translated by cells without needing any intervening replication steps such as reverse transcription.

In some embodiments, a RNA molecule of the invention may have a 5′ cap (e.g. but not limited to a 7-methylguanosine, 7 mG (5′) ppp (5′) NImpNp) This cap can enhance in vivo translation of the RNA. The S′ nucleotide of an RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. In some embodiments, a RNA molecule useful with the invention may be single-stranded. In some embodiments, a RNA molecule useful with the invention may comprise synthetic RNA.

The recombinant nucleic acid sequence can be an optimized nucleic acid sequence. Such optimization can increase or alter the immunogenicity of the envelope. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

Methods for in vitro transfection of mRNA and detection of envelope expression are known in the art.

Methods for expression and immunogenicity determination of nucleic acid encoded envelopes are known in the art.

In certain aspects the invention contemplates using immunogenic compositions wherein immunogens are delivered as recombinant proteins. Various methods for production and purification of recombinant proteins, including trimers such as but not limited to SOSIP based trimers, suitable for use in immunization are known in the art. In certain embodiments recombinant proteins are produced in CHO cells.

It is readily understood that the envelope glycoproteins referenced in various examples and figures comprise a signal/leader sequence. It is well known in the art that HIV-1 envelope glycoprotein is a secretory protein with a signal or leader peptide sequence that is removed during processing and recombinant expression (without removal of the signal peptide, the protein is not secreted). See for example Li et al. Control of expression, glycosylation, and secretion of HIV-1 gp120 by homologous and heterologous signal sequences. Virology 204 (1): 266-78 (1994) (“Li et al. 1994”), at first paragraph, and Li et al. Effects of inefficient cleavage of the signal sequence of HIV-1 gp120 on its association with calnexin, folding, and intracellular transport. PNAS 93:9606-9611 (1996) (“Li et al. 1996”), at 9609. Any suitable signal sequence could be used. In some embodiments the leader sequence is the endogenous leader sequence. Most of the gp 120 and gp 160 amino acid sequences include the endogenous leader sequence. In other non-limiting examples, the leader sequence is human Tissue Plasminogen Activator (TPA) sequence, human CD5 leader sequence (e.g. MPMGSLQPLATLYLLGMLVASVLA). Most of the chimeric designs include CD5 leader sequence. A skilled artisan appreciates that when used as immunogens, and for example when recombinantly produced, the amino acid sequences of these proteins do not comprise the leader peptide sequences.

The immunogenic envelopes can also be administered as a protein prime and/or boost alone or in combination with a variety of nucleic acid envelope primes (e.g., HIV-1 Envs delivered as DNA expressed in viral or bacterial vectors).

Dosing of proteins and nucleic acids can be readily determined by a skilled artisan. A single dose of nucleic acid can range from a few nanograms (ng) to a few micrograms (μg) or milligram of a single immunogenic nucleic acid. Recombinant protein dose can range from a few μg micrograms to a few hundred micrograms, or milligrams of a single immunogenic polypeptide.

Administration: The compositions can be formulated with appropriate carriers using known techniques to yield compositions suitable for various routes of administration. In certain embodiments the compositions are delivered via intramuscular (IM), via subcutaneous, via intravenous, via nasal, via mucosal routes, or any other suitable route of immunization.

The compositions can be formulated with appropriate carriers and adjuvants using techniques to yield compositions suitable for immunization. The compositions can include an adjuvant, such as, for example but not limited to 3M052, alum, poly IC, MF-59 or other squalene-based adjuvant, ASOIB, or other liposomal based adjuvant suitable for protein or nucleic acid immunization. In certain embodiments, the adjuvant is GSK ASO1E adjuvant containing MPL and QS21. This adjuvant has been shown by GSK to be as potent as the similar adjuvant AS01B but to be less reactogenic using HBsAg as vaccine antigen (Leroux-Roels et al., IABS Conference, April 2013). In certain embodiments, TLR agonists are used as adjuvants. In other embodiment, adjuvants which break immune tolerance are included in the immunogenic compositions.

In certain embodiments, the compositions and methods comprise any suitable agent or immune modulation which could modulate mechanisms of host immune tolerance and release of the induced antibodies. In non-limiting embodiments modulation includes PD-1 blockade; T regulatory cell depletion; CD40L hyperstimulation; soluble antigen administration, wherein the soluble antigen is designed such that the soluble agent eliminates B cells targeting dominant epitopes, or a combination thereof. In certain embodiments, an immunomodulatory agent is administered in at time and in an amount sufficient for transient modulation of the subject's immune response so as to induce an immune response which comprises broad neutralizing antibodies against HIV-1 envelope. Non-limiting examples of such agents is any one of the agents described herein: e.g. chloroquine (CQ), PTPIB Inhibitor-CAS 765317-72-4-Calbiochem or MSI 1436 clodronate or any other bisphosphonate; a Foxol inhibitor, e.g. 344355 Foxol Inhibitor, AS1842856-Calbiochem; Gleevac, anti-CD25 antibody, anti-CCR4 Ab, an agent which binds to a B cell receptor for a dominant HIV-1 envelope epitope, or any combination thereof. In non-limiting embodiments, the modulation includes administering an anti-CTLA4 antibody, OX-40 agonists, or a combination thereof. Non-limiting examples are of CTLA-1 antibody are ipilimumab and tremelimumab. In certain embodiments, the methods comprise administering a second immunomodulatory agent, wherein the second and first immunomodulatory agents are different.

### Multimeric Envelopes

Presentation of antigens as particulates reduces the B cell receptor affinity necessary for signal transduction and expansion (see Baptista et al. EMBO J. 2000 Feb. 15; 19 (4): 513-520). Displaying multiple copies of the antigen on a particle provides an avidity effect that can overcome the low affinity between the antigen and B cell receptor. The initial B cell receptor specific for pathogens can be low affinity, which precludes vaccines from being able to stimulate and expand B cells of interest. In particular, very few naïve B cells from which HIV-1 broadly neutralizing antibodies arise can bind to soluble HIV-1 Envelope. Provided are envelopes, including but not limited to trimers as particulate, high-density array on liposomes or other particles, for example but not limited to nanoparticles. See, e.g. He et al. Nature Communications 7, Article number: 12041 (2016), doi: 10.1038/ncomms12041; Bamrungsap et al. Nanomedicine, 2012, 7 (8), 1253-1271.

For development as a vaccine immunogen, we have also created multimeric nanoparticles that comprise and/or display HIV envelope protein or fragments on their surface.

The nanoparticle immunogens are composed of various forms of HIV-envelope protein, e.g. without limitation envelope trimer, and self-assembling protein, e.g. without limitation ferritin protein. Any suitable ferritin could be used in the immunogens of the invention. In non-limiting embodiments, the ferritin is derived from Helicobacter pylori. In non-limiting embodiments, the ferritin is insect ferritin. In non-limiting embodiments, each nanoparticle displays 24 copies of the envelope protein on its surface.

Presenting multiple copies of antigens to B cells has been a longstanding approach to improving B cell receptor recognition and antigen uptake (See Batista et al. EMBO J. 2000 Feb. 15; 19 (4): 513-520). The improved recognition of antigen is due to the avid interaction of multiple antigens with multiple B cell receptors on a single B cells, which results in clustering of B cells and stronger cell signaling. Furthermore, multimeric presentation improves antigen binding to mannose binding lectin which promotes antigen trafficking to B cell follicles. Self-assembling complexes comprising multiple copies of an antigen are one strategy of immunogen design approach for arraying multiple copies of an antigen for recognition by the B cell receptors on B cells (Kanekiyo, M., Wei, C.J., Yassine, H.M., McTamney, P.M., Boyington, J.C., Whittle, J.R., Rao, S.S., Kong, W.P., Wang, L., and Nabel, G.J. (2013). Self-assembling influenza nanoparticle vaccines elicit broadly neutralizing HINI antibodies. Nature 499, 102-106; Ueda, G., Antanasijevic, A., Fallas, J.A., Sheffler, W., Copps, J., Ellis, D., Hutchinson, G.B., Moyer, A., Yasmeen, A., Tsybovsky, Y., et al. (2020). Tailored design of protein nanoparticle scaffolds for multivalent presentation of viral glycoprotein antigens. Elife).

In some instances, the gene of an antigen is fused via a linker/spacer to a gene of a protein which could self-assemble. Upon translation, a fusion protein is made that can self-assemble into a multimeric complex—also referred to as a nanoparticle displaying multiple copies of the antigen. In other instances, the protein antigen could be conjugated to the self-assembling protein via an enzymatic reaction, thereby forming a nanoparticle displaying multiple copies of the antigen. Non-limiting embodiments of enzymatic conjugation include without limitation sortase mediated conjugation. In some embodiments, linkers for use in any of the designs of the invention could be 2-50 amino acids long, e.g. 2, 3, 4, 5, 6, 7, 8, 9, 10, 11, 12, 13, 14, 15, 16, 17, 18, 19, 20, 25, 30, 35, 40, 45, or 50 amino acids long. In certain embodiments, these linkers comprise glycine and serine amino acid in any suitable combination, and/or repeating units of combinations of glycine, serine and/or alanine.

Ferritin is a well-known protein that self-assembles into a hollow particle composed of repeating subunits. In some species ferritin nanoparticles are composed of 24 copies of a single subunit, whereas in other species it is composed of 12 copies each of two subunits.

Non-limiting embodiments of sortase linkers could be used so long as their position allows multimerization of the envelopes. In a non-limiting embodiment, a C-terminal tag is LPXTG, where X signifies any amino acid but most commonly Ala, Ser, Glu, or a N-terminal pentaglycine repeat tag is added to the envelope trimer gene. In a non-limiting embodiment, a C-terminal tag is LPXTGG, where X signifies any amino acid but most commonly Ala, Ser, Glu.

To improve the interaction between the naïve B cell receptor and immunogens, in some embodiments, the envelope design is created so the envelope is presented on particles, e.g. but not limited to nanoparticle. In some embodiments, the HIV-1 Envelope trimer could be fused to ferritin. Ferritin protein self assembles into a small nanoparticle with three fold axis of symmetry. At these axes the envelope protein is fused. Therefore, the assembly of the three-fold axis also clusters three HIV-1 envelope protomers together to form an envelope trimer. Each ferritin particle has 8 axes which equates to 8 trimers being displayed per particle. See e.g. Sliepen et al. Retrovirology 2015 12.82, DOI: 10.1186/s12977-015-0210-4.

Any suitable ferritin sequence could be used. In non-limiting embodiments, ferritin sequences are disclosed in US2019/0330279, the content of which is hereby incorporated by reference in its entirety.

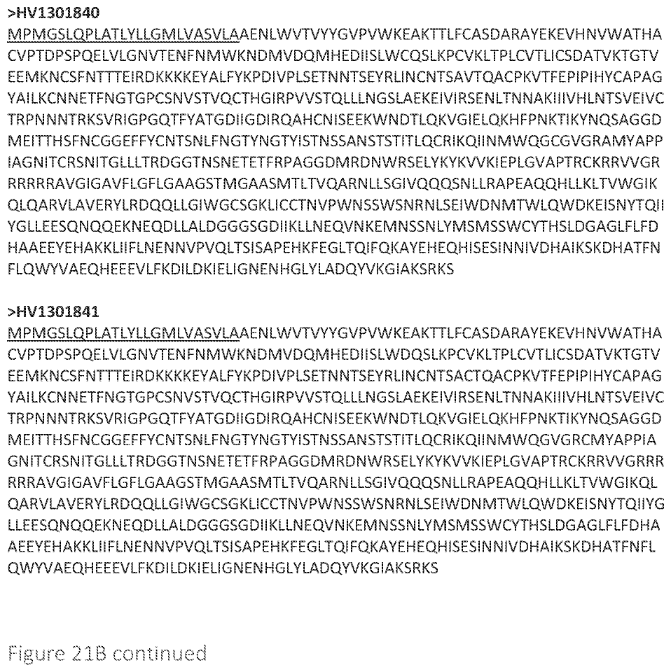

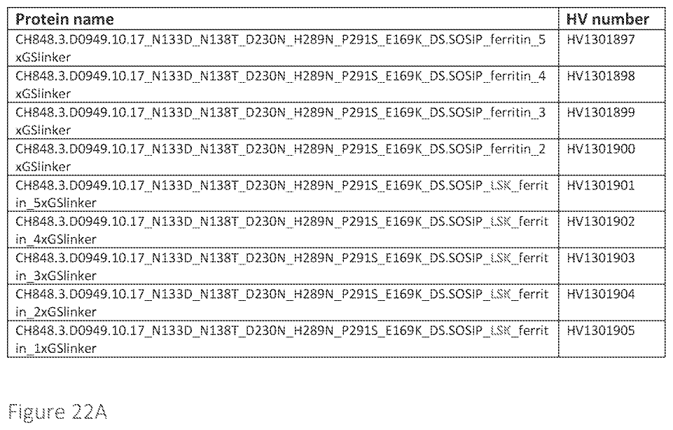

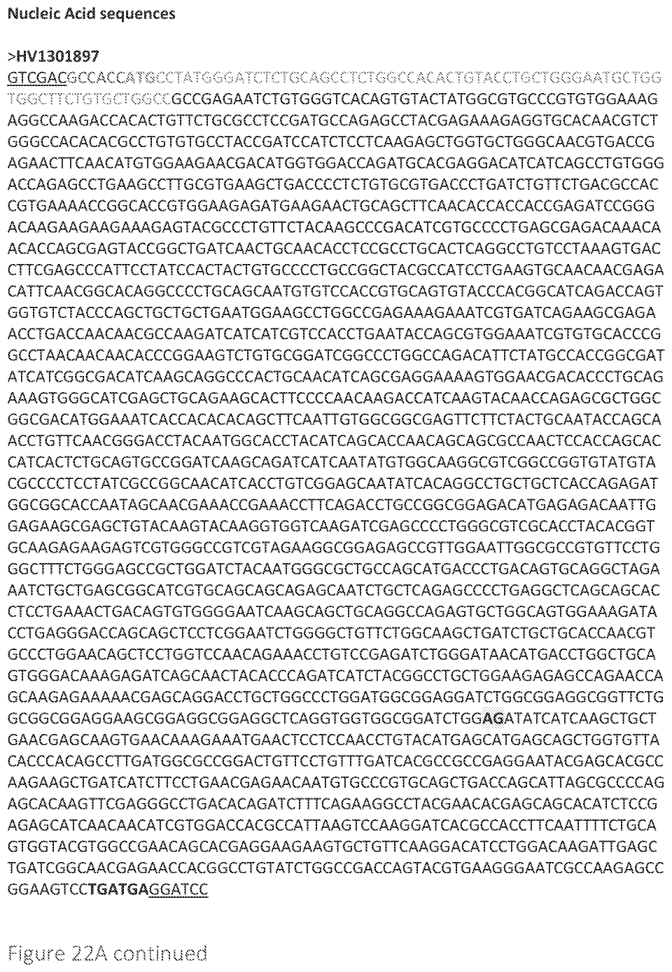

Ferritin nanoparticle linkers: The ability to form HIV-1 envelope ferritin nanoparticles relies self-assembly of 24 ferritin subunits into a single ferritin nanoparticle. The addition of a ferritin subunit to the c-terminus of HIV-I envelope may interfere with the ability of the ferritin subunit to fold properly and or associate with other ferritin subunits. When expressed alone ferritin readily forms 24-subunit nanoparticles, however appending it to envelope only yields nanoparticles for certain envelopes. Since the ferritin nanoparticle forms in the absence of envelope, the envelope could be sterically hindering the association of ferritin subunits. Thus, ferritin can be designed with elongated glycine-serine linkers to further distance the envelope from the ferritin subunit. To make sure that the glycine linker is attached to ferritin at the correct position, constructs can be created that attach at second amino acid position or the fifth amino acid position. The first four n-terminal amino acids of natural Helicobacter pylori ferritin are not needed for nanoparticle formation but may be critical for proper folding and oligomerization when appended to envelope. Thus, constructs can be designed with and without the leucine, serine, and lysine amino acids following the glycine-serine linker. The goal will be to find a linker length that is suitable for formation of envelope nanoparticles when ferritin is appended to most envelopes. For non-limiting embodiments, linker designs see FIGS. 22A-B. Any suitable linker between the envelope and ferritin could be uses, so long as the fusion protein is expressed and the trimer is formed.

Another approach to multimerize expression constructs uses staphylococcus sortase A transpeptidase ligation to conjugate inventive envelope trimers, for example but not limited to cholesterol. The trimers can then be embedded into liposomes via the conjugated cholesterol. To conjugate the trimer to cholesterol either a C-terminal LPXTG tag or a N-terminal pentaglycine repeat tag is added to the envelope trimer gene. Cholesterol is also synthesized with these two tags. Sortase A is then used to covalently bond the tagged envelope to the cholesterol. The sortase A-tagged trimer protein can also be used to conjugate the trimer to other peptides, proteins, or fluorescent labels. In non-limiting embodiments, the sortase A tagged trimers are conjugated to ferritin to form nanoparticles. See FIG. 26.

The invention provides design of envelopes and trimer designs wherein the envelope comprises a linker which permits addition of a lipid, such as but not limited to cholesterol, via a sortase A reaction. See e.g. Tsukiji, S. and Nagamune, T. (2009), Sortase-Mediated Ligation: A Gift from Gram-Positive Bacteria to Protein Engineering. ChemBioChem, 10:787-798. doi: 10.1002/cbic.200800724; Proft, T. Sortase-mediated protein ligation: an emerging biotechnology tool for protein modification and immobilisation. Biotechnol Lett (2010) 32:1. doi: 10.1007/s10529-009-0116-0; Lena Schmohl, Dirk Schwarzer, Sortase-mediated ligations for the site-specific modification of proteins, Current Opinion in Chemical Biology, Volume 22, October 2014, Pages 122-128, ISSN 1367-5931, dx.doi.org/10 1016/j.cbpa. 2014.09.020; Tabata et al. Anticancer Res. 2015 Aug,35 (8): 4411-7; Pritz et al. J. Org. Chem. 2007, 72, 3909-3912.

The lipid modified envelopes and trimers could be formulated as liposomes. Any suitable liposome composition is contemplated.

The lipid modified and multimerized envelopes and trimers could be formulated as liposomes. Any suitable liposome composition is contemplated.

Nomenclature for trimers: chim.6R.DS.SOSIP.664 is SOSIP.I CHIM.6R.SOSIP.664 is SOSIP.II; CHIM.6R.SOSIP.664V4.1 is SOSIP.III.

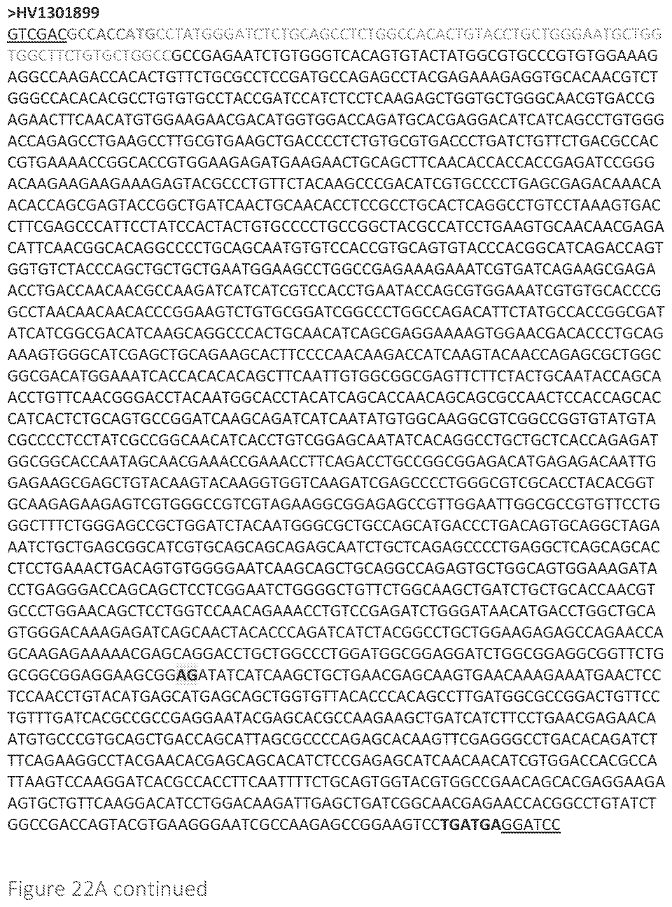

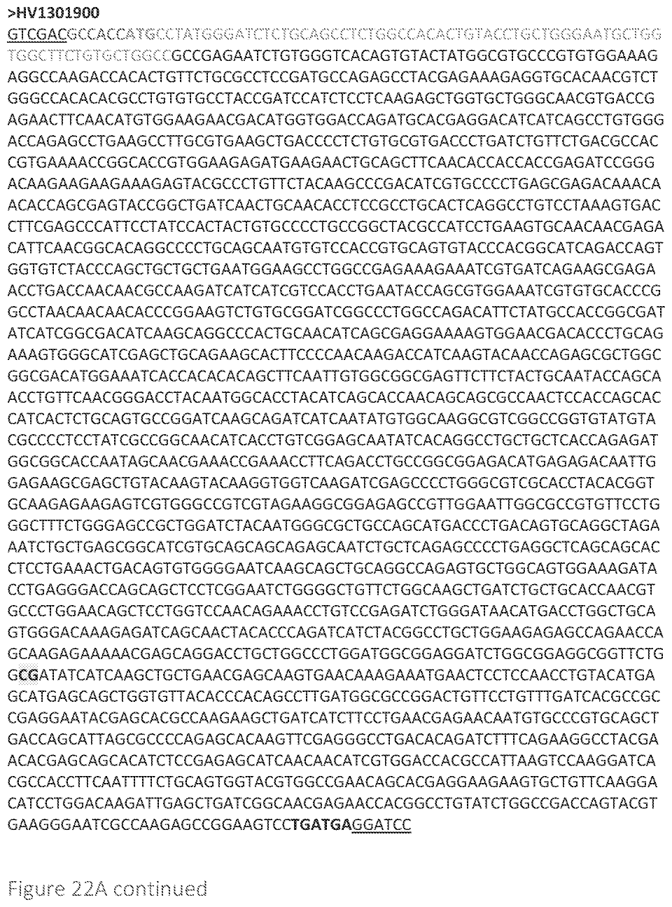

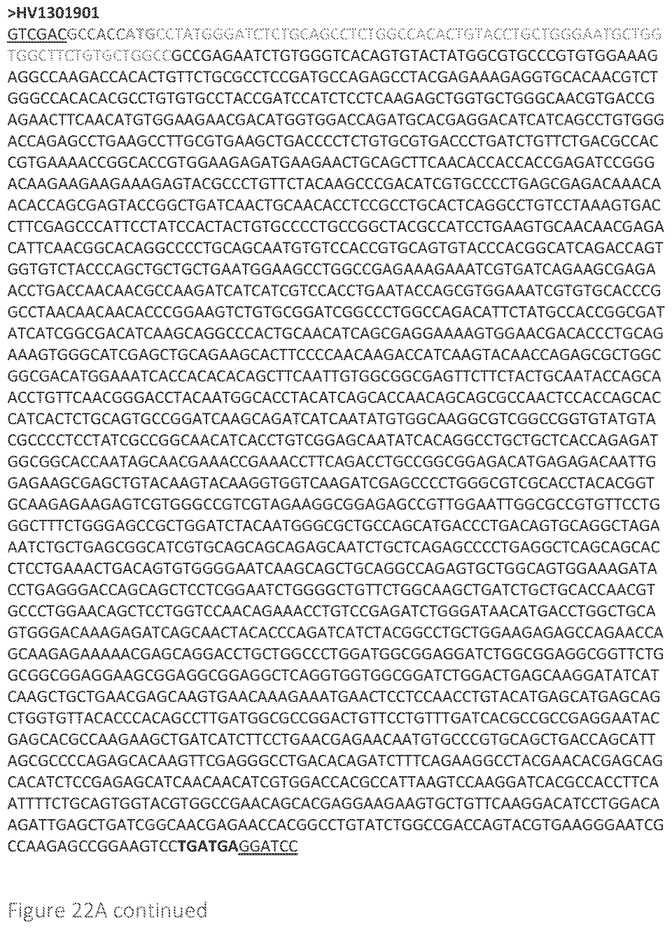

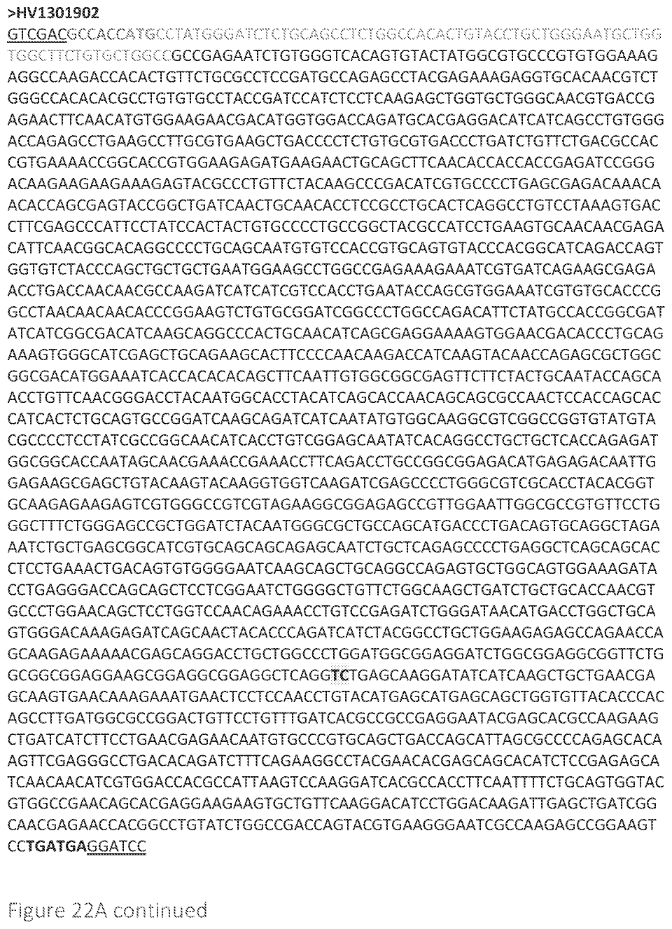

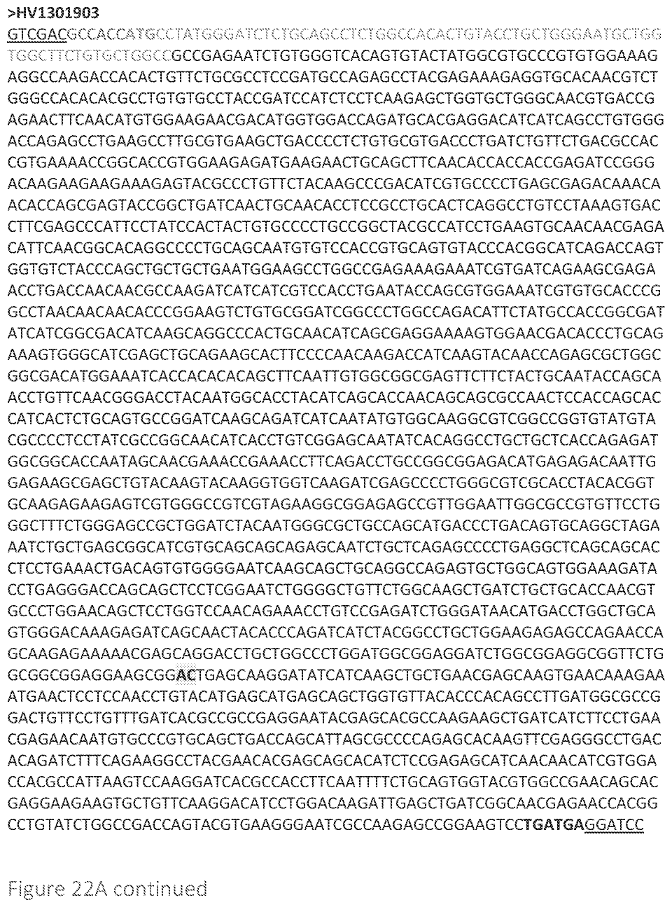

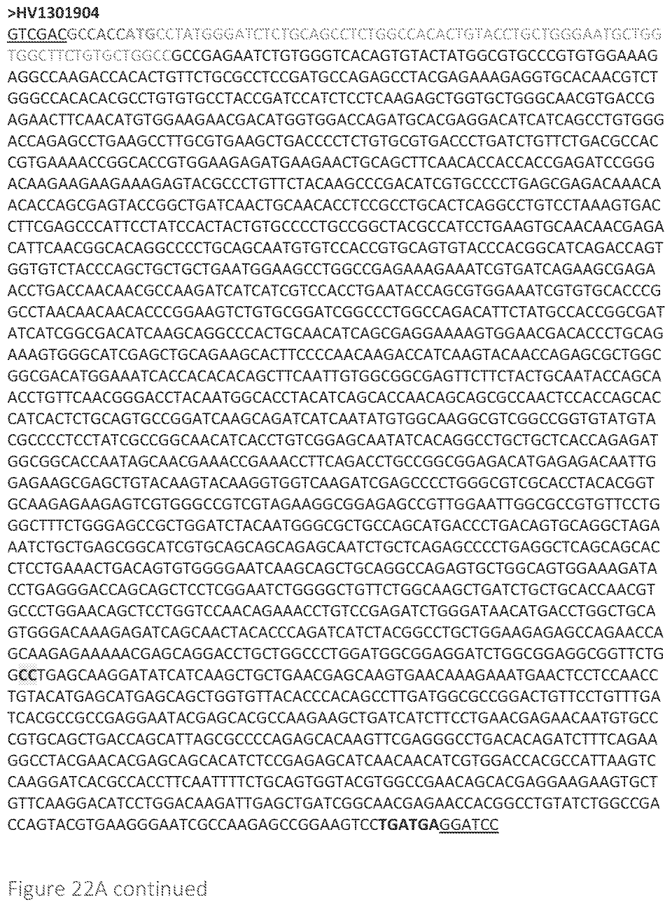

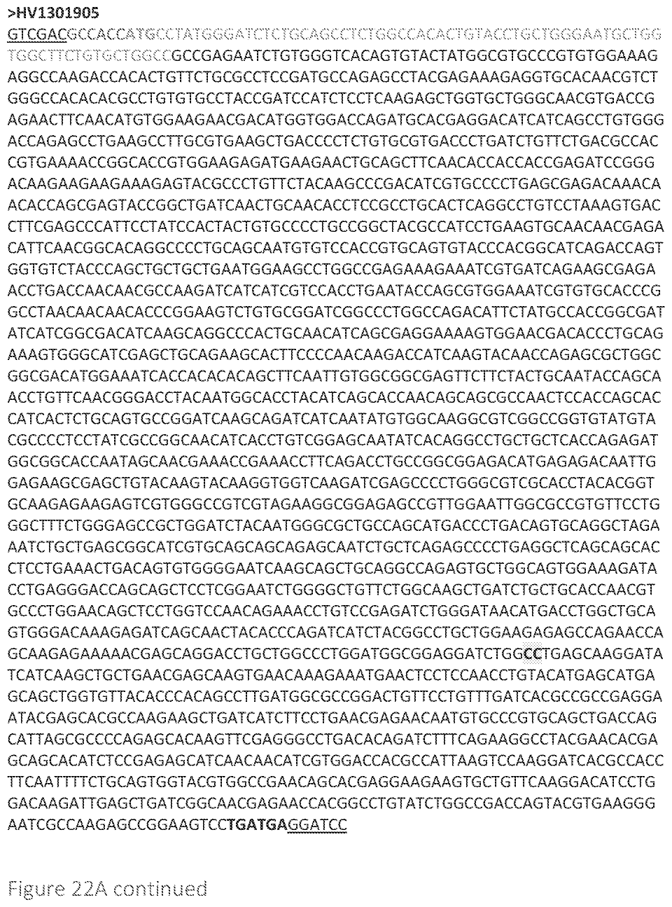

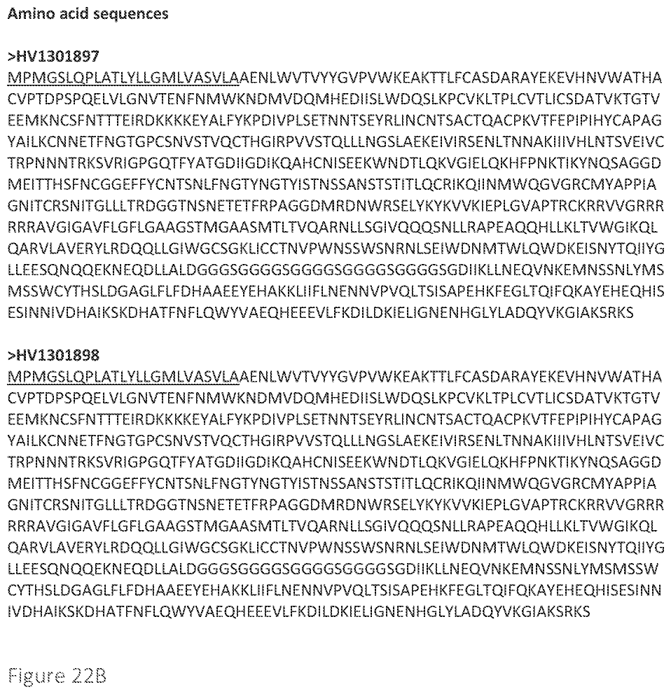

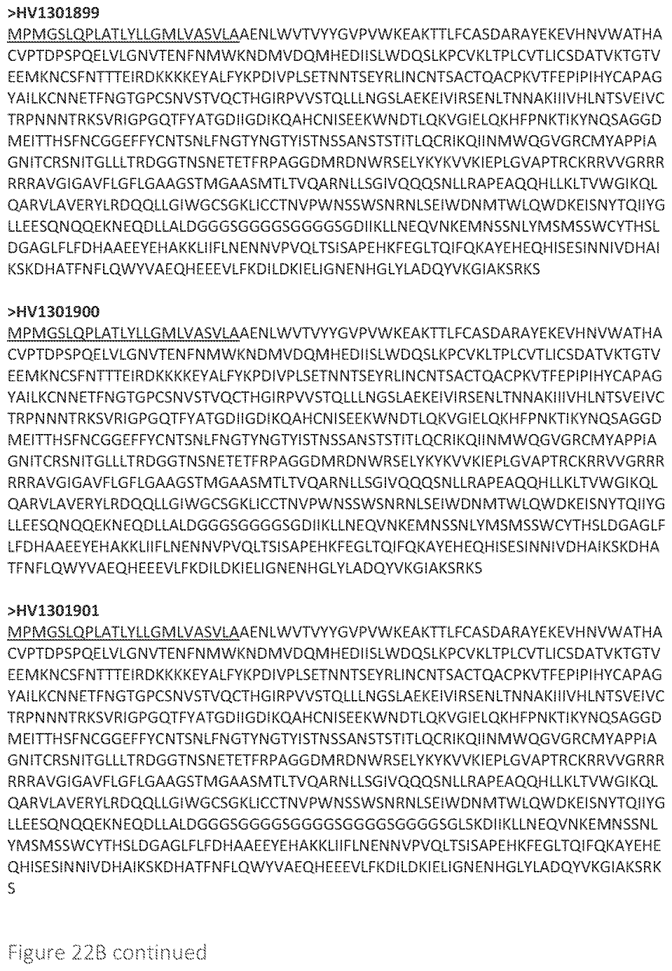

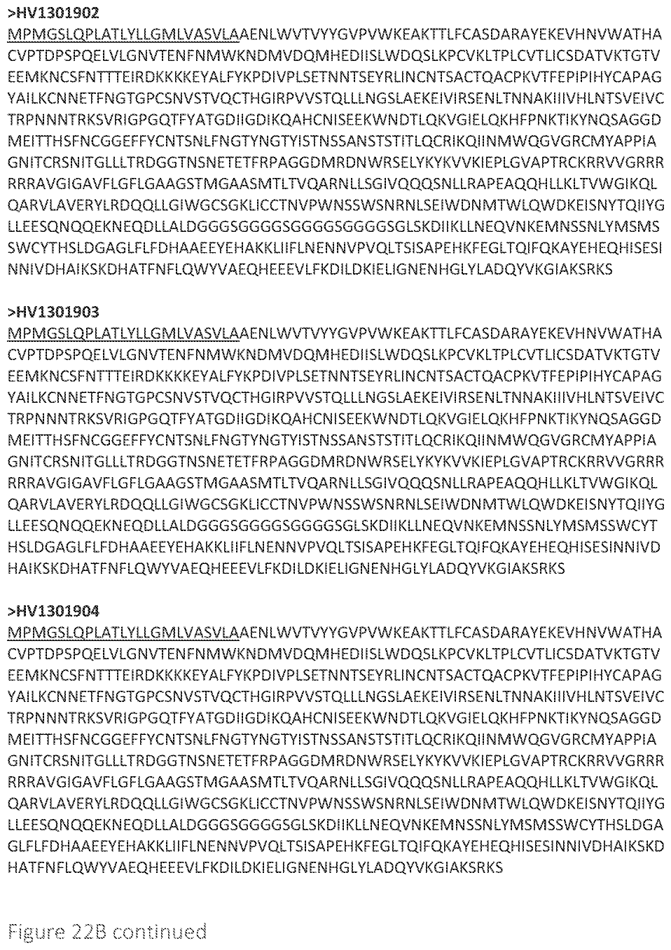

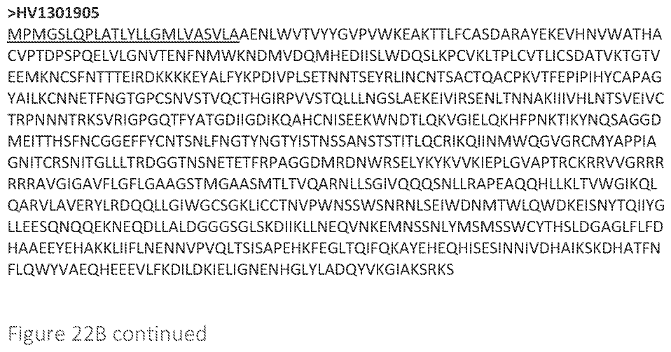

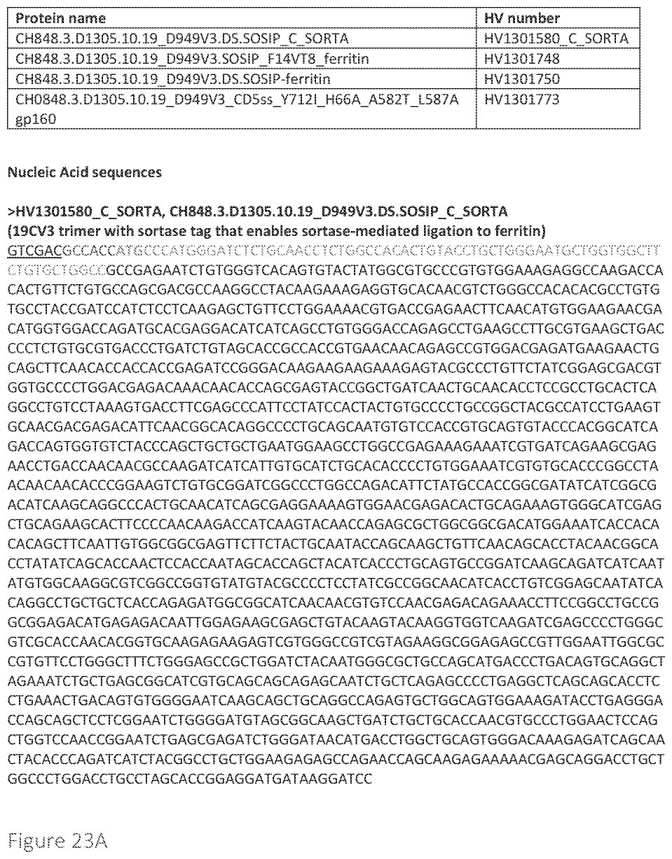

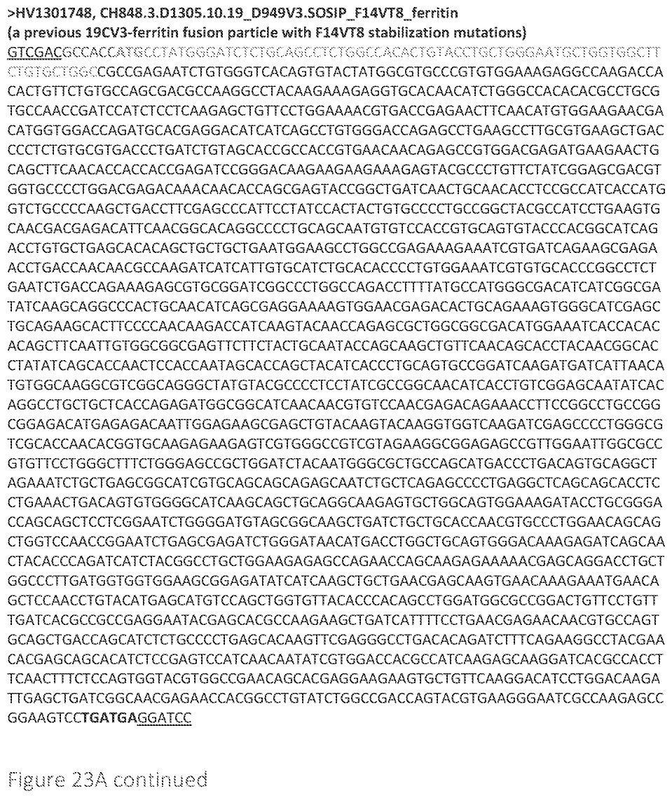

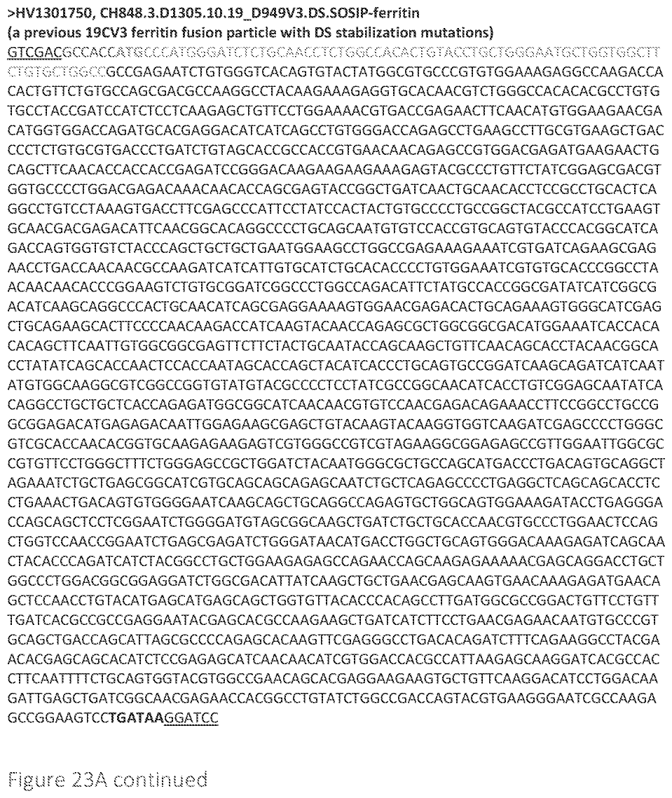

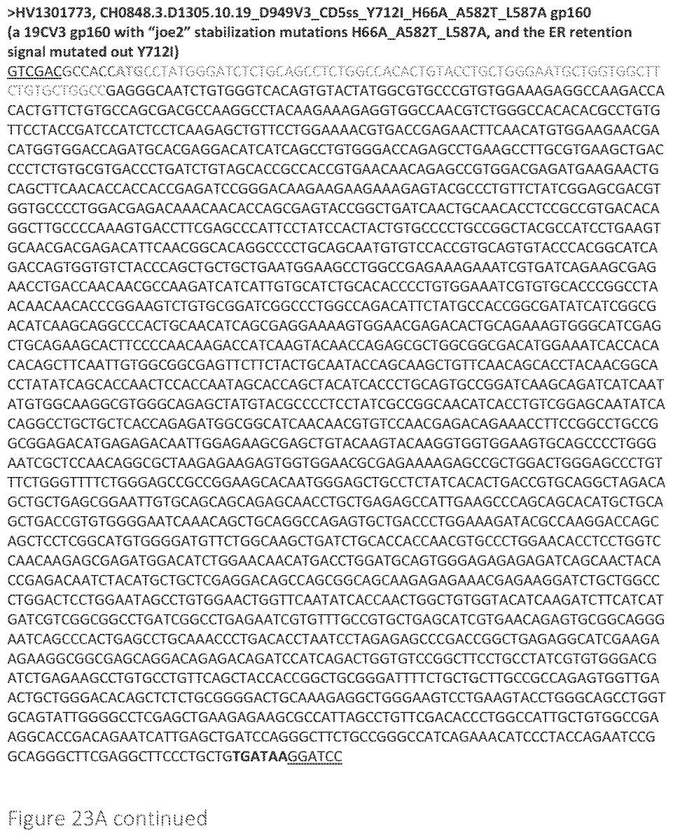

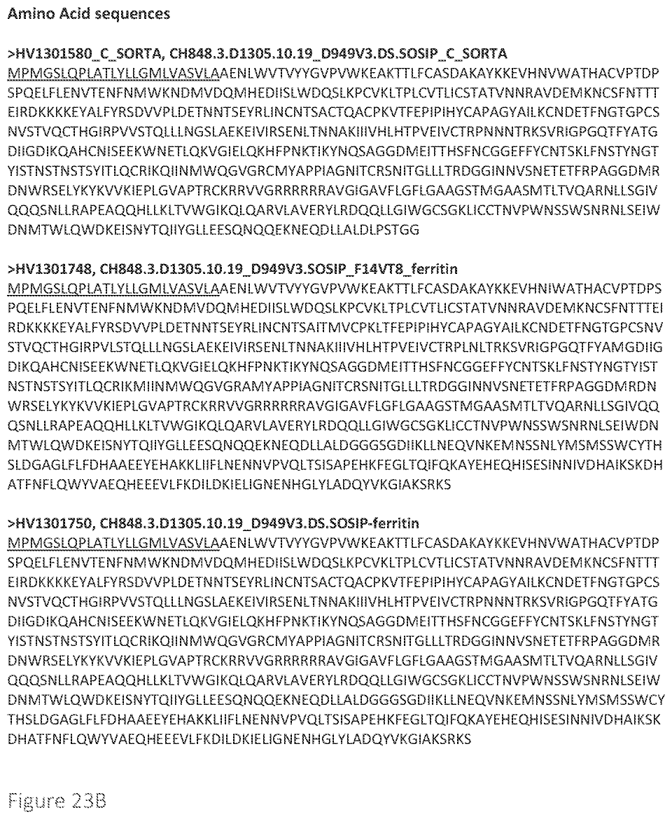

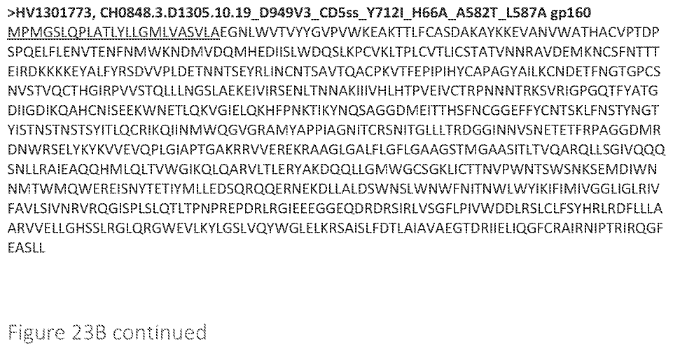

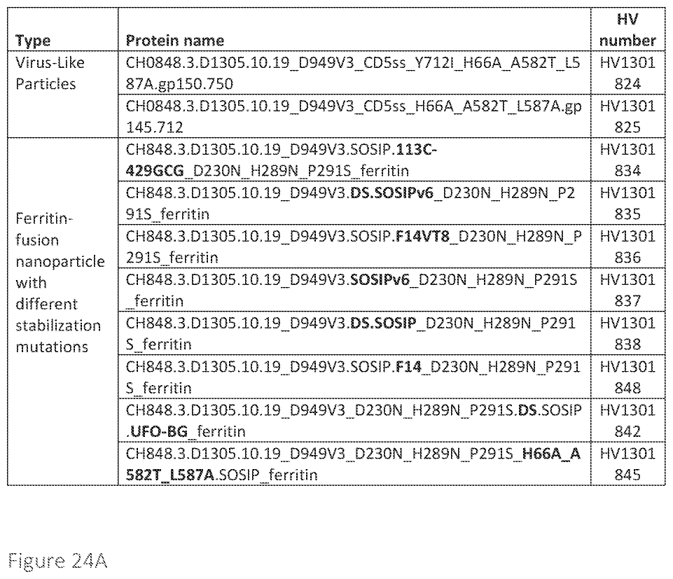

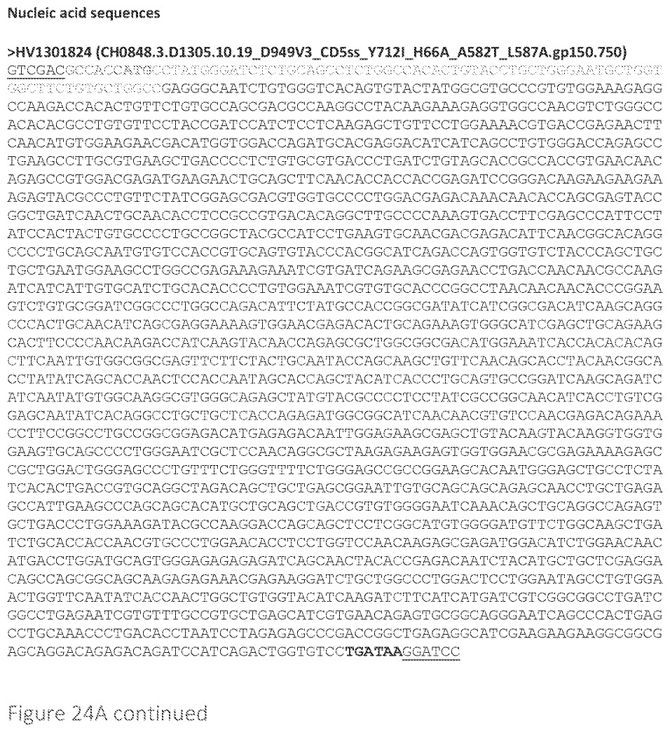

Non-limiting embodiments of envelope designs for use in sortase A reaction are shown in FIG. 24B-D of US2020/0002383, incorporated by reference in its entirety.

Additional sortase linkers could be used so long as their position allows multimerization of the envelopes. In a non-limiting embodiment, a C-terminal tag is LPXTG, where X signifies any amino acid but most commonly Ala, Ser, Glu, or a N-terminal pentaglycine repeat tag is added to the envelope trimer gene. In a non-limiting embodiment, a C-terminal tag is LPXTGG, where X signifies any amino acid but most commonly Ala, Ser, Głu.

Table 1 shows a summary of sequences described herein.

| | Amino acid, | | |
|---|---|---|---|
| Name | nucleic acid | Design | FIG. |
| HV1301580_D230N_H289N_P291S; CH848.3.D1305.10.19_{—} | Nt | Modified | 1 |
| D949V3.DS.SOSIP_D230N_H289N_P291S (glycan hole | aa | CH848.3.D1305.10.19 | 2 |
| filled) | | D949 | |
| >HV1301502_D1305V1; JRFL_SOSIPv6_V1_PNGS_D1305V1 | Nt | Modified | 1 |
| (V1 loop from 10.19) | aa | CH848.3.D1305.10.19 | 2 |
| | | D949 | |
| >HV1301405_D1305V1; CON- | Nt | Modified | 1 |
| Schim.6R.DS.SOSIP.664_OPT_D1305V1 (V1 loop from | aa | CH848.3.D1305.10.19 | 2 |
| 10.19 isolate) | | D949 | |
| >HV1301580_D230N_H289N_P291S; CH848.3.D1305.10.19_{—} | Nt | Modified | 1 |
| D949V3.DS.SOSIP_D230N_H289N_P291S (glycan holes | aa | CH848.3.D1305.10.19 | 2 |
| filled) | | D949 | |
| >HV1301580; CH848.3.D1305.10.19_D949V3.DS.SOSIP | Nt | 19CV3; DSSOSIP | 1 |
| (19CV3) | aa | soluble | 2 |
| | | trimer | |
| >HV1301509; CH0848.3.d1305.10.19gp160 | Nt | Full length | 1 |
| | aa | gp160 | 2 |
| >HV1301503; CH848.3.D1305.10.19ch.DS.SOSIP.664 | Nt | Chimeric | 1 |
| | aa | (different | 2 |
| | | gp41) | |
| | | DSSOSIP | |
| | | soluble | |
| | | trimer | |
| >HV1301504; CH848.3.D1305.10.19ch.SOSIPv6 | Nt | v6 | 1 |
| | aa | modifications | 2 |
| >HV1301580_C_SORTA; CH848.3.D1305.10.19_{—} | Aa | Sortase | 3 |
| D949V3.DS.SOSIP_C_SORTA | nt | acceptor site | 3 |
| | | for | |
| | | nanoparticle | |
| | | formation | |

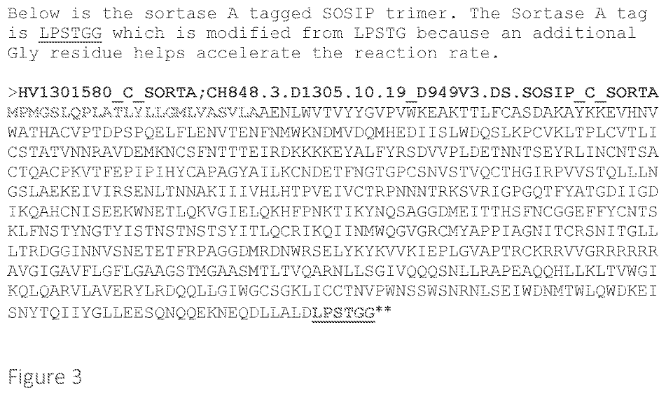

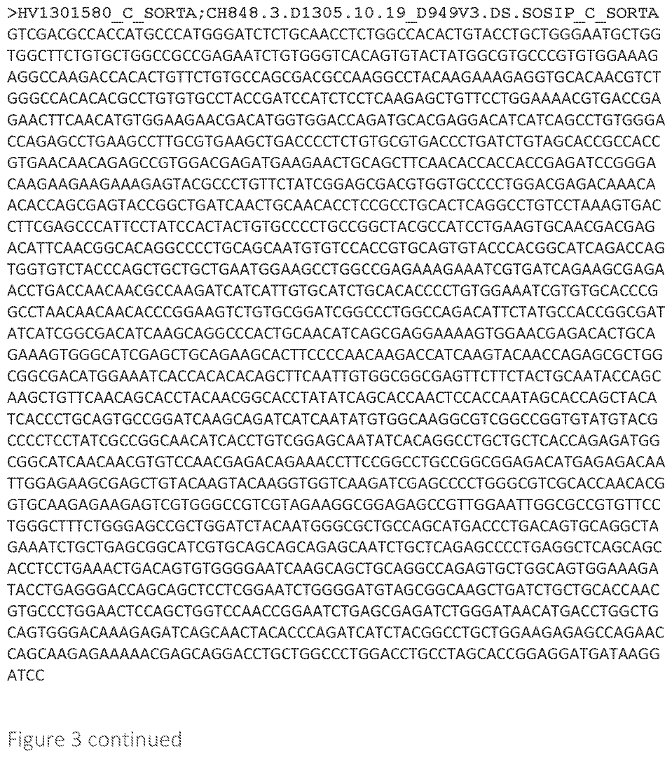

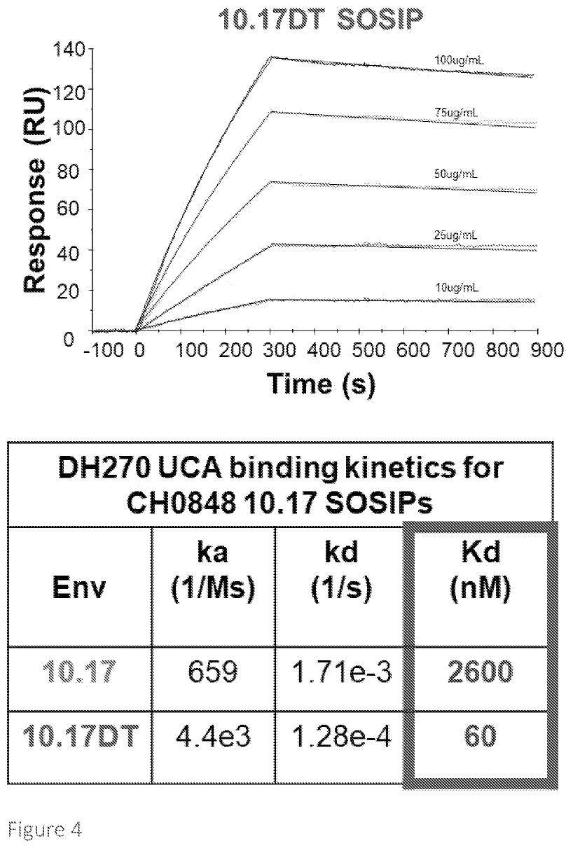

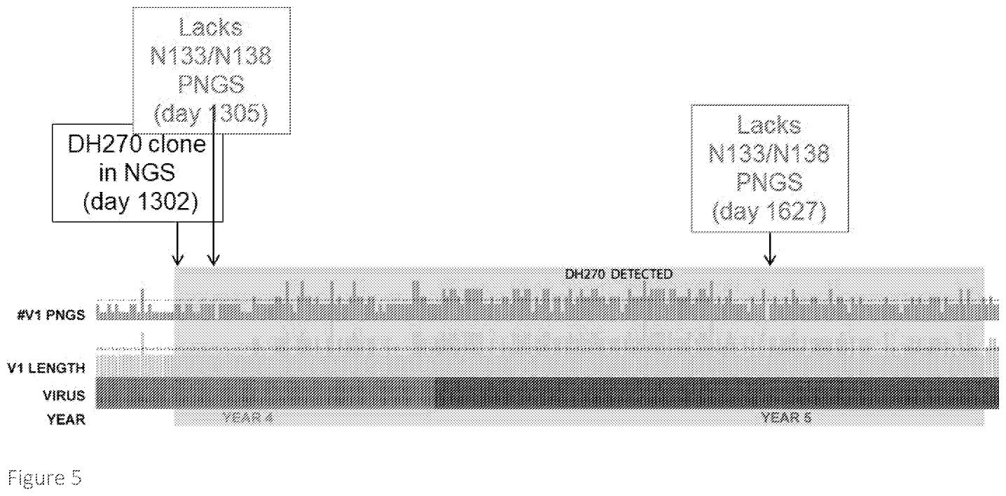

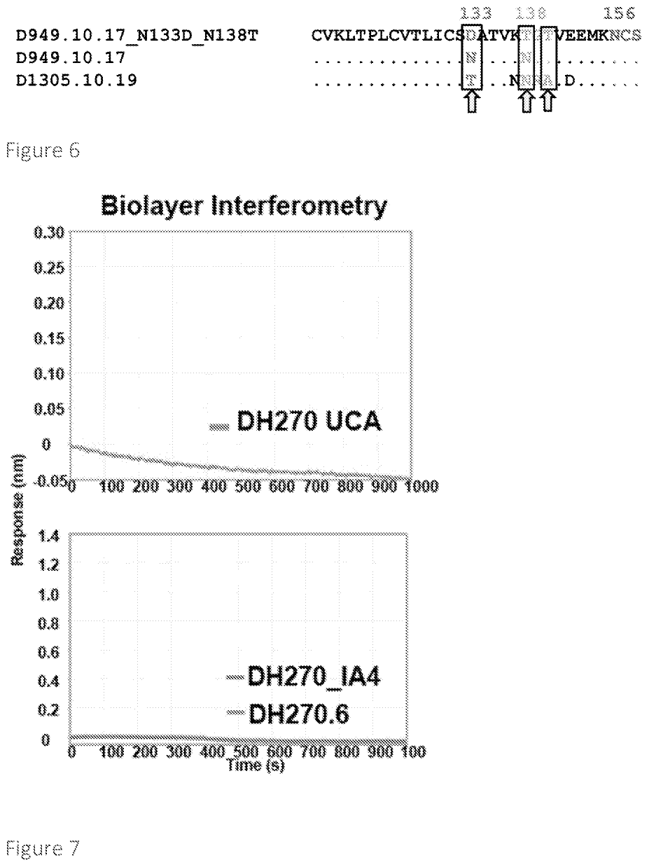

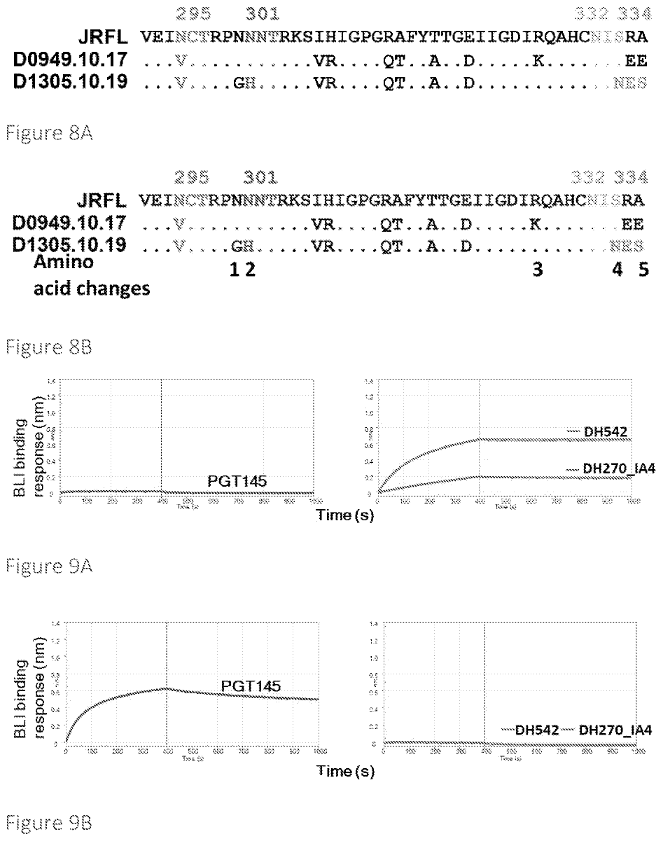

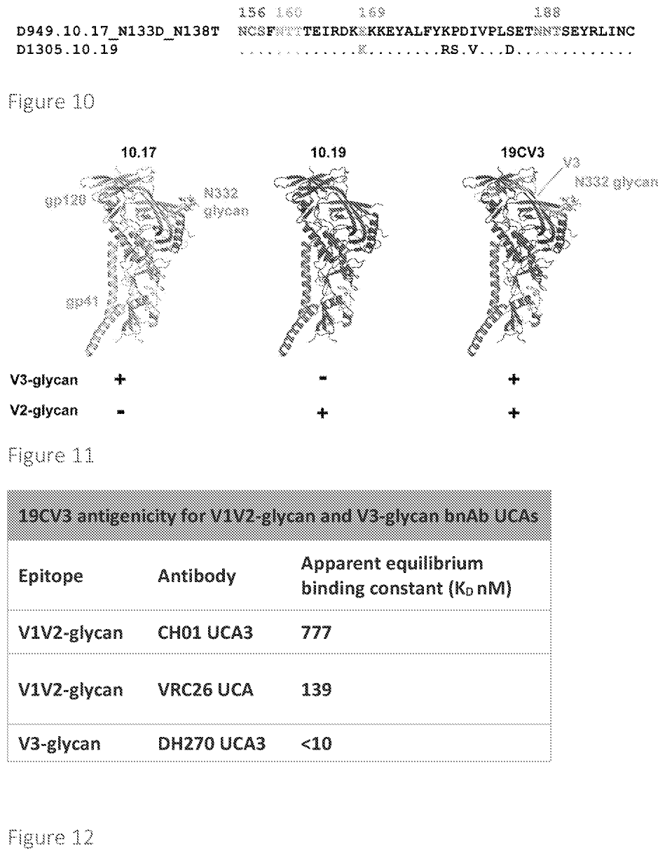

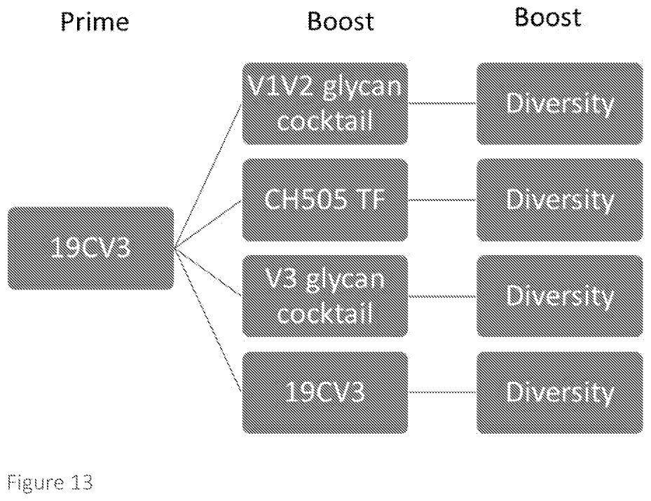

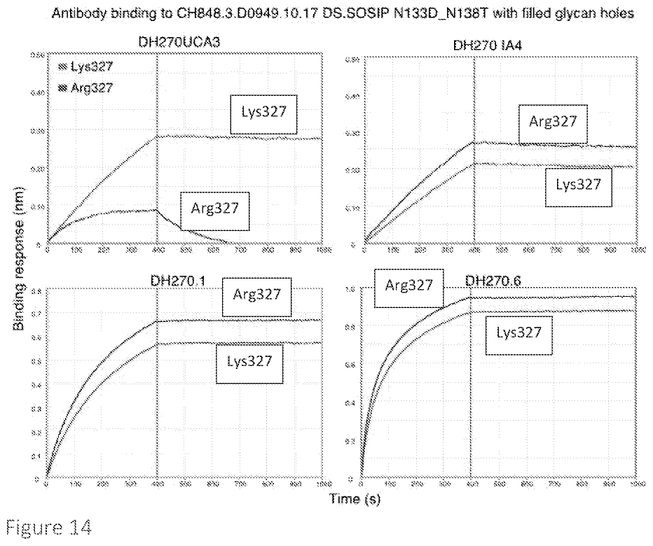

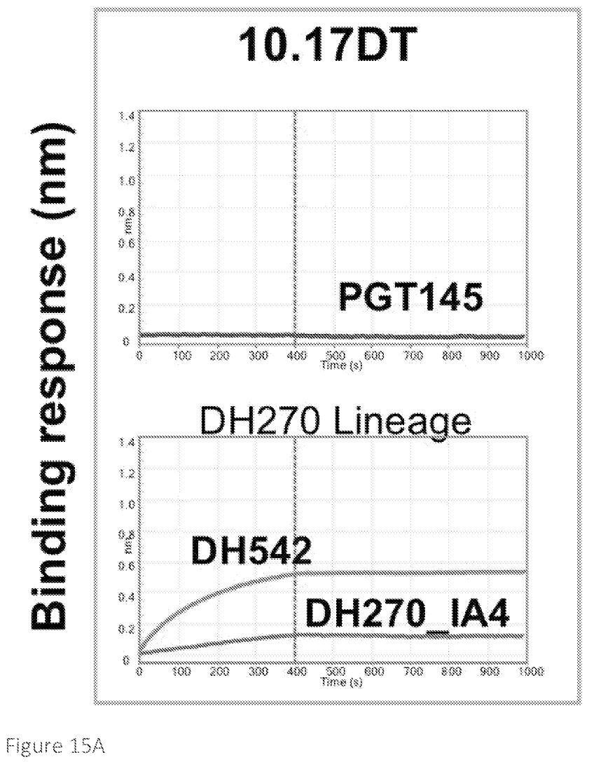

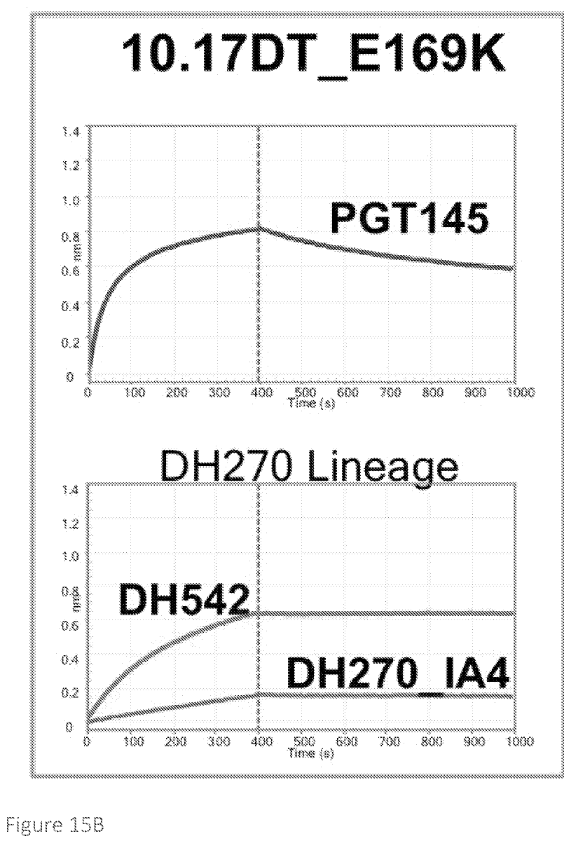

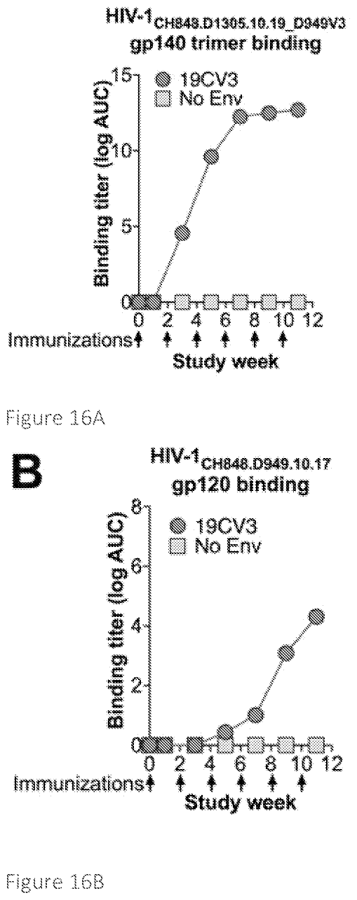

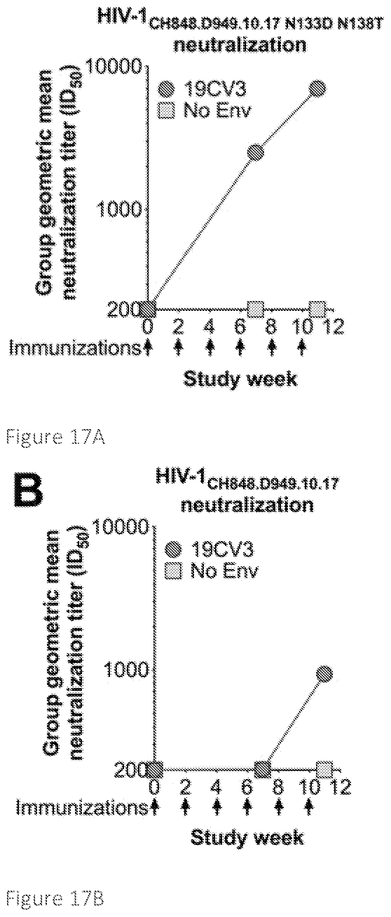

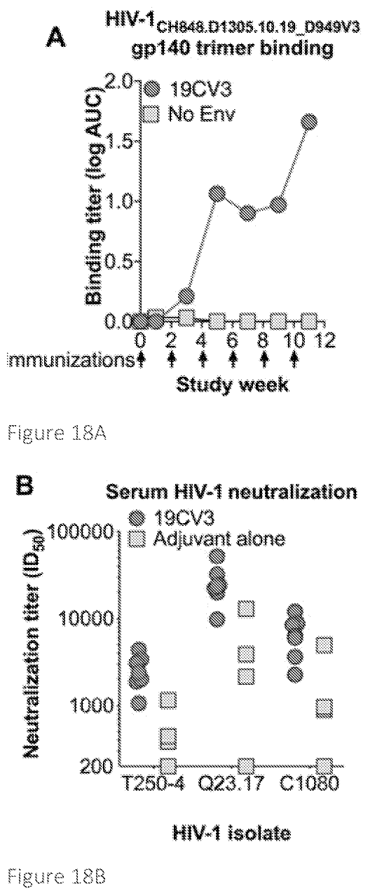

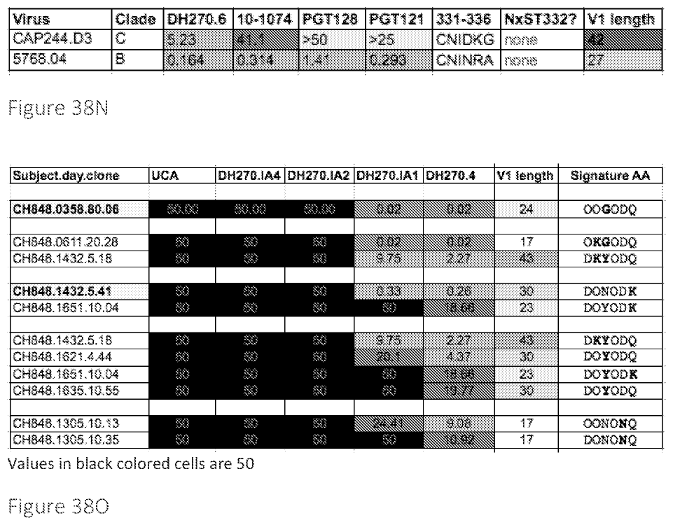

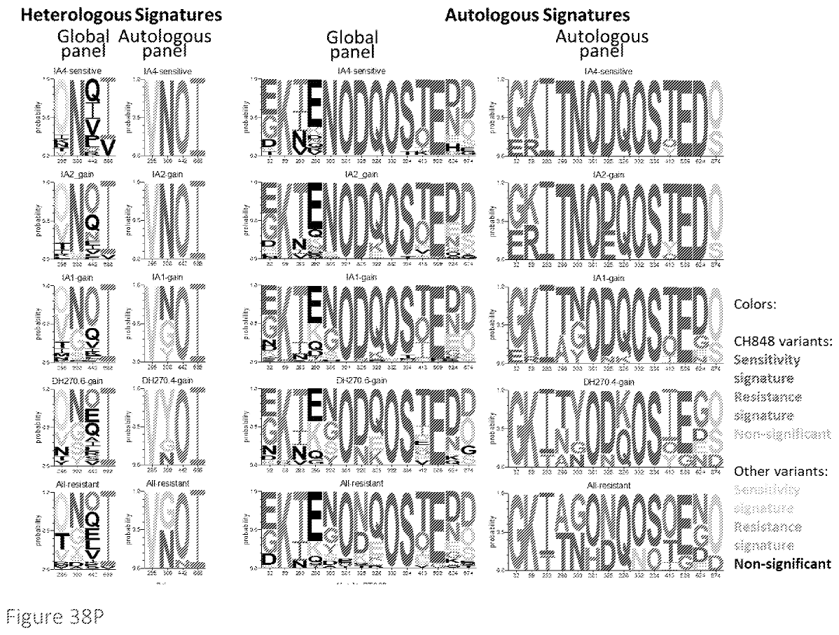

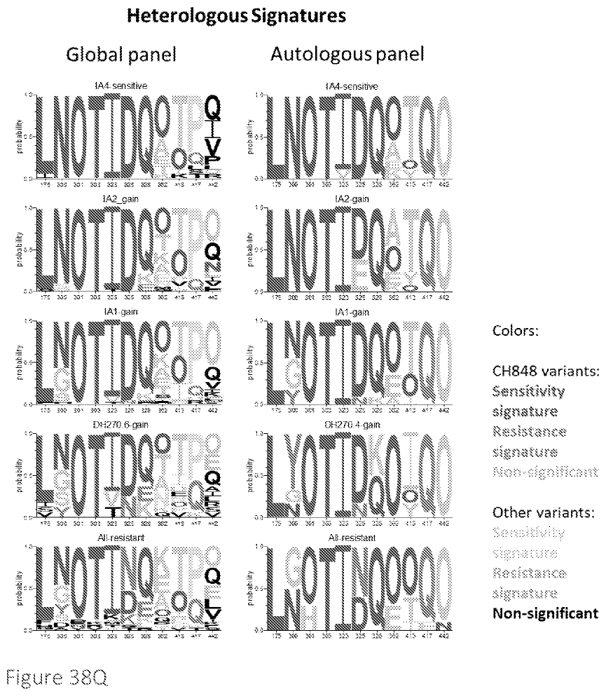

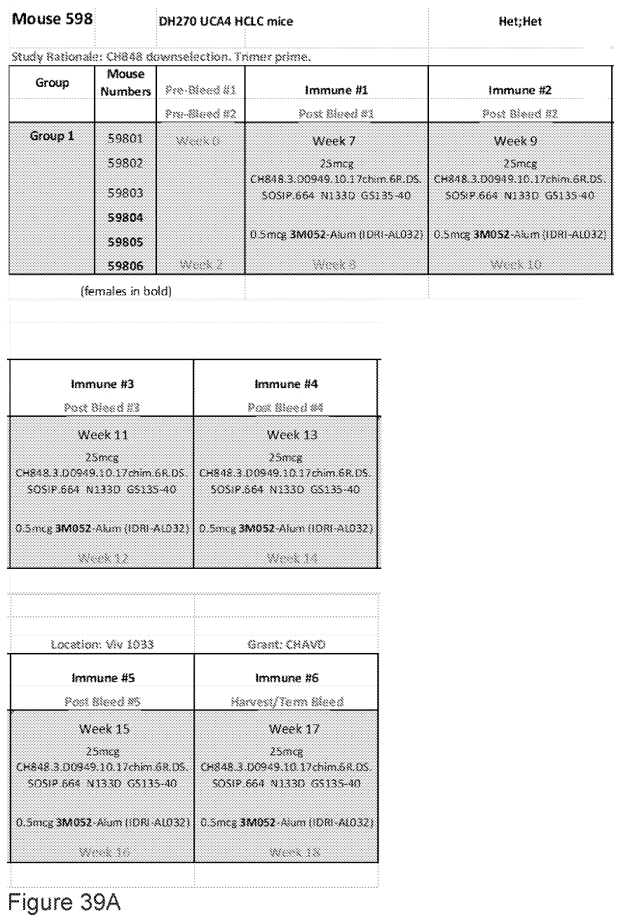

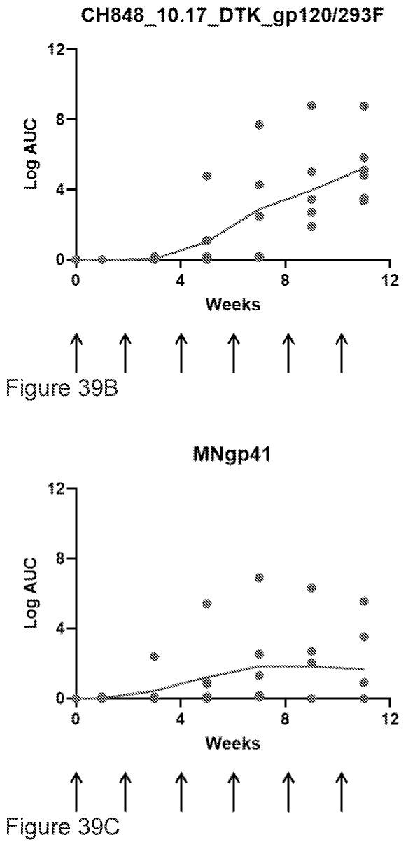

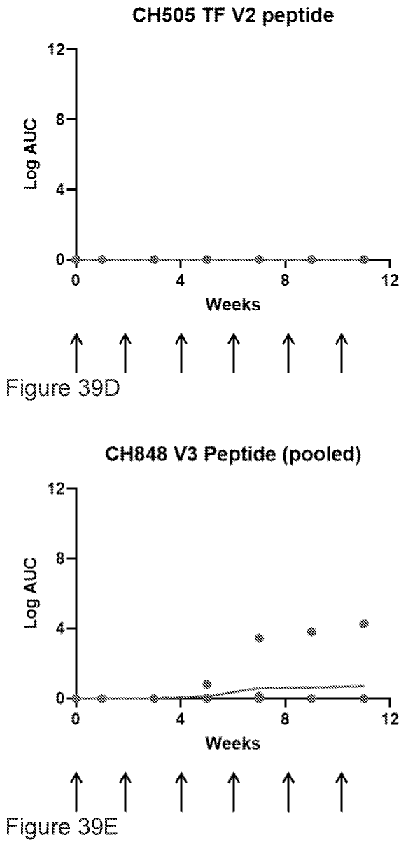

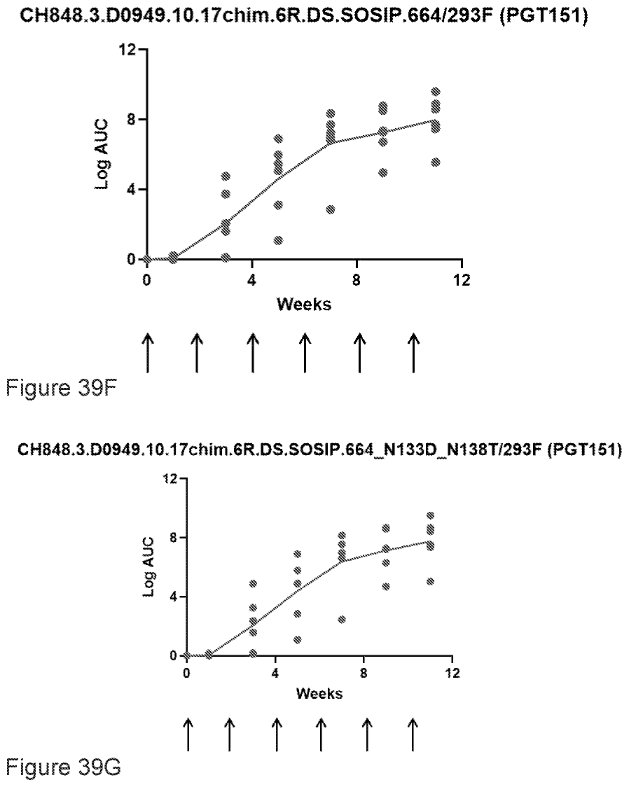

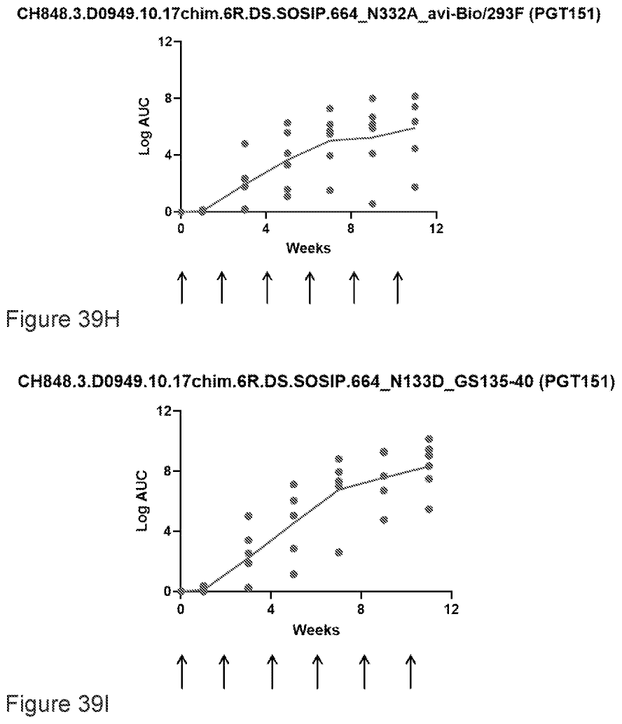

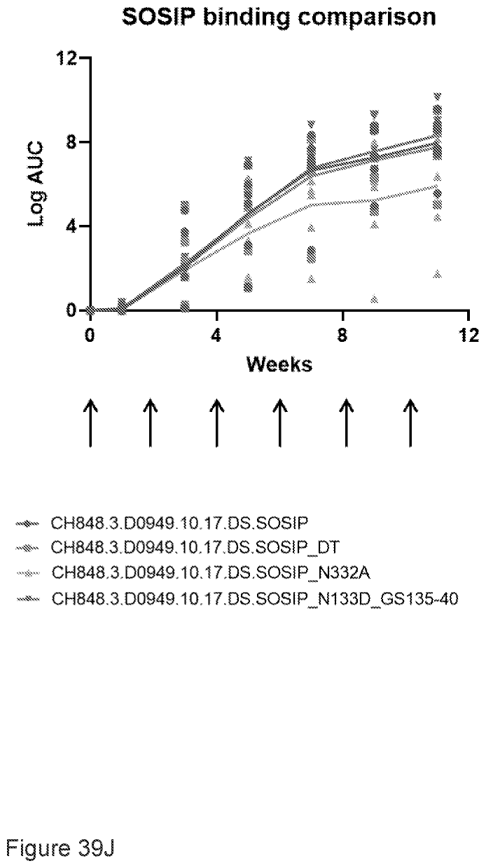

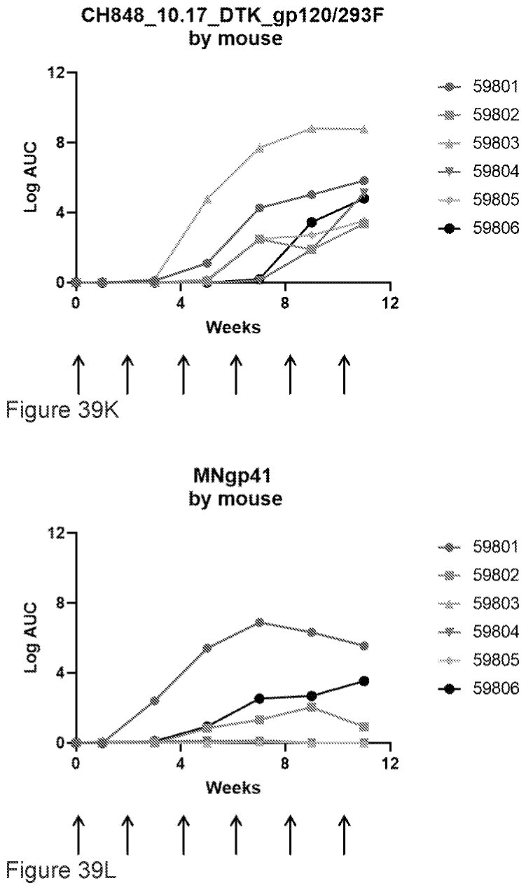

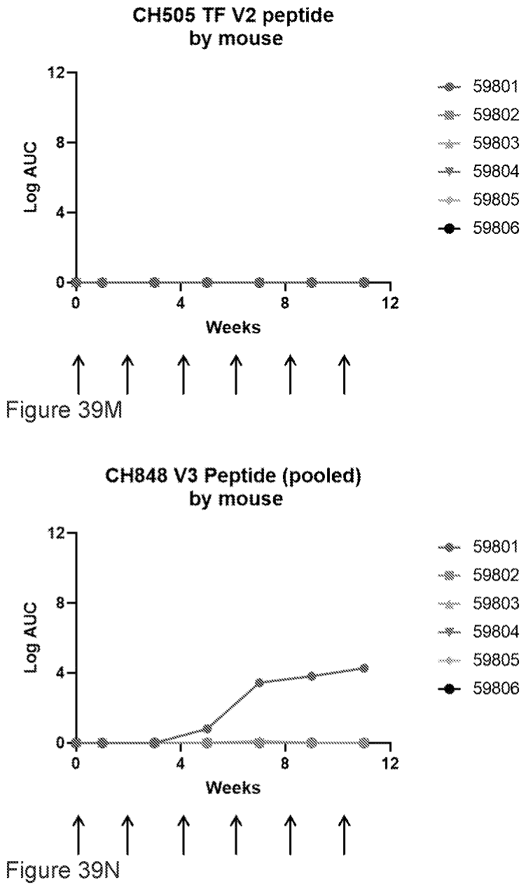

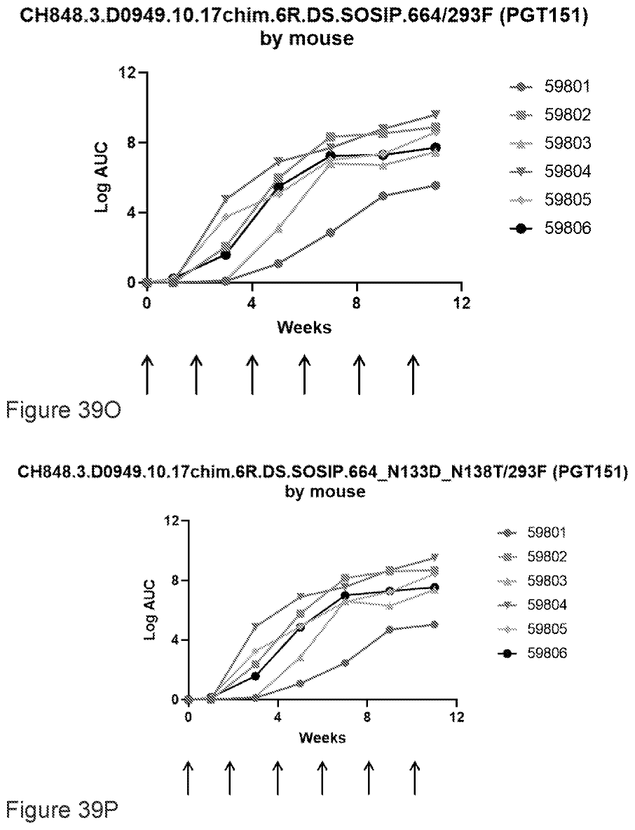

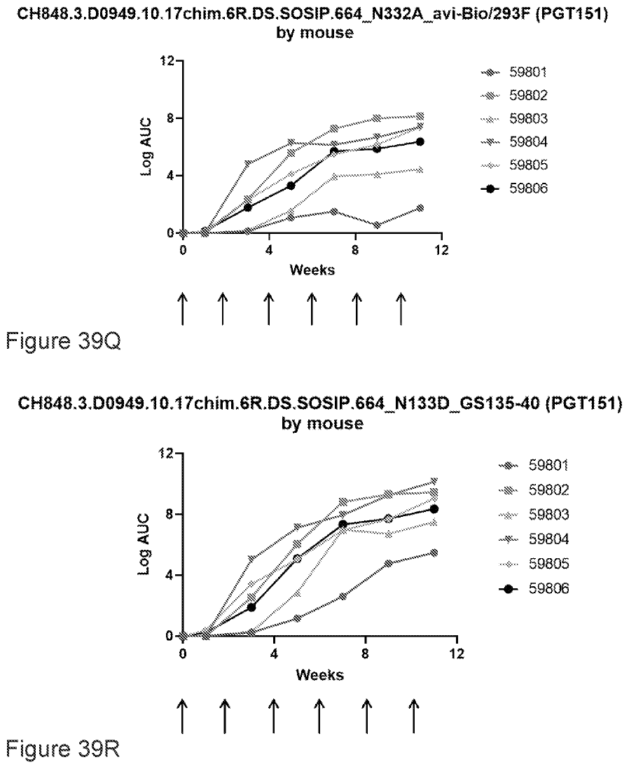

Table 2 shows a summary of modifications to envelopes described herein

| | | | V3 | UCA and |
|---|---|---|---|---|
| | | | glycosylation | other Ab |
| Envelope | FIG./SEQ ID No | V1 region | sites | binding |
| 10.17 | See | 17aa | N301 and N332 | |
| | US2020/0113997 | | | |
| | incorporated | | | |
| | herein by | | | |
| | reference in its | | | |
| | entirety including | | | |
| | FIGS. 39A-B and | | | |
| | SEQ ID NOs | | | |
| | disclosed therein | | | |
| 10.17 DT | See | 17aa N133D | N301 and N332 | DH270UCA |
| | US2020/0113997 | N138T effectively | | |
| | incorporated | lacks | | |
| | herein by | glycosylation sites | | |
| | reference in its | | | |
| | entirety including | | | |
| | FIG. 49A, 50B, | | | |
| | 51, 52A, 53D, 54A- | | | |
| | C, 77D-G, 77I and | | | |
| | SEQ ID NOs | | | |
| | disclosed therein | | | |
| 10.19 | FIG. 1 | 17aa V1 region | No | CH01 UCA |
| | | lacks N133 and | glycosylation | |
| | | N138 | sites at N295, | |
| | | glycosylation sites | N301, N332 | |
| 10.19 plus V3 loop | FIG. 1, FIG. 3 | 17aa V1 region | Add V3 regions | CH01 UCA |
| of 10.17 (19CV3) | | lacks N133 and | from 10.17 has | DH270UCA |
| | | N138 | five aa | VRC26 |
| | | glycosylation sites | difference from | UCA |
| | | | 10.19 | |
| 10.19 env based with fewer | FIG. 14 | | At least changes | |
| than five aa changes | FIG. 21A-B | | #2, 4, 5, and/or | |
| compared to 19CV3; | | | “GDIR” | |
| “GDIR/K” | | | sequence | |
| Ferritin Linker | FIG. 22A-B | | | |
| E169K | FIG. 21A-B | | | |
| Glycan hole filled | FIG. 21A-B, 22A-B, | | | |
| | 24A-B | | | |

DH270 light chain binds to N301 glycan. In some embodiments, a N301 gly site is used (e.g. change #2 in row 5 of Table 2, supra).

DH270 heavy chain binds to N332 glycan. In some embodiments, a N332 gly site is used (e.g. changes #4 and #5 in row 5 of Table 2, supra).

V3 glycan Abs bind GDIR. In some embodiments, a change #3 to “GDIR” is needed (e.g. “GDIR” sequence in row 5 of Table 2, supra).

GDIR/K motif: V3-glycan broadly neutralizing antibodies typically contact the c-terminal end of the third variable region on HIV-1 envelope. There are four amino acids, Gly324, Asp325, Ile326, and Arg327, bound by V3-glycan neutralizing antibodies. While Arg327 is highly conserved among HIV-1 isolates, Lys327 also occurs at this site. The CH848.3.D0949.10.17 isolate naturally encodes the less common Lys327. In contrast to CH848.3.D0949.10.17 with the Lys327, the precursor antibody of the DH270 V3-glycan broadly neutralizing antibody lineage barely binds to CH848.3.D0949.10.17 encoding Arg327. Thus, Arg327 is critical for the precursor to bind and the lineage of neutralizing antibodies to begin maturation. However, somatically mutating antibodies on the path to developing neutralization breadth bind better to Env encoding Arg327. See FIG. 14. Thus, Env must encode Lys327 to initiate DH270 lineage development. However, to best interact with affinity maturing DH270 lineage members the Env should encode Arg327. Thus, a plausible vaccine regimen to initiate and select for developing bnAbs would include a priming immunogen encoding, Lys327 and a boosting immunogen encoding Arg327. The Arg327 boosting immunogen would optimally target the affinity maturing DH270 lineage members, while not optimally binding the DH270 antibodies that lack affinity maturation. Non-limiting embodiments of vaccination regimens could include: priming with CH848.3.D0949.10.17 based envelope design also with Lys327, followed by administering of CH848.3.D0949.10.17 based envelope design with Arg327. Non-limiting embodiments of vaccination regimens could include: priming with 19CV3 based envelope design also with Lys327, followed by administering of CH848 3. D0949.10 17 based envelope design with Arg327.

E169K modification: One approach to designing a protective HIV-1 vaccine is to elicit broadly neutralizing antibodies (bnAbs). However, bnAbs against two or more epitopes will likely need to be elicited to prevent HIV-1 escape. Thus, optimal HIV-1 immunogens should be antigenic for multiple bnAbs in order to elicit bnAbs to more than one epitope. The CH848.D949.10.17 HIV-1 isolate was antigenic for V3-glycan antibodies but lacked binding to VIV2-glycan antibodies Not all viruses from the CH848 individual lacked binding to VIV2-glycan antibodies. For example, the CH848.D1305.10.19 isolate bound well to V1V2-glycan antibody PGT145. We compared the sequence of CH848.D949.10.17 and CH848.D1305.10.19 in the region that is contacted by V1V2-glycan antibodies in crystal structures (Mclellan JS, Pancera M, Carrico C, Gorman J, Julien JP, Khayat R, et al. Structure of HIV-1 gp120 V1/V2 domain with broadly neutralizing antibody PG9. Nature. 2011; 480 (7377): 336-43). Interestingly, the CH848.D949.10.17 and CH848.D1305.10.19 differed in sequence at a known contact site for V1V2-glycan antibodies-position 169 (Doria-Rose NA, Georgiev I, O'Dell S, Chuang GY, Staupe RP, Mclellan JS, et al. A short segment of the HIV-1 gp120 V1/V2 region is a major determinant of resistance to V1/V2 neutralizing antibodies. J Virol. 2012; 86 (15): 8319-23). It has been previously shown that mutation of lysine at position 169 eliminates binding to VIV2-glycan antibody PG9 (Doria-Rose NA, Georgiev I, O'Dell S, Chuang GY, Staupe RP, Mclellan JS, et al. A short segment of the HIV-1 gp120 V1/V2 region is a major determinant of resistance to V1/V2 neutralizing antibodies. J Virol. 2012; 86 (15): 8319-23). CH848.D1305.10.19 sequence encoded a lysine at position 169 whereas CH848.D949.10.17 sequence encoded a glutamate. Thus, we changed the glutamate (E) to lysine (K) at position 169 of CH848. D949.10.17. This single change in CH848.D949.10.17 enabled VIV2-glycan antibody binding to the envelope. Thus, the E169K adds the V1V2-glycan epitope to the other bnAb epitopes present on CH848.D949.10.17-based envelopes. Overall, the result of the E169K is a CH848.D949.10.17 envelope capable of eliciting more different types of bnAbs.

The invention contemplates any other design, e.g. stabilized trimer, of the sequences described here in. For non-limiting embodiments of additional stabilized trimers see US2015/0366961 (DU4061), US2020/0002383 (DU4716), US2021/0187091 (DU4918) and US2020/0113997 (DU4918), F14 and/or VT8 designs (US2021/0379177) all of which are incorporated by reference in their entirety, and

In certain embodiments the invention provides an envelope comprising 17aa V1 region without N133 and N138 glycosylation, and N301 and N332 glycosylation sites, and further comprising “GDIR” motif (see Example 1, FIG. 8B), wherein the envelope binds to UCAs of VIV2 Abs and V3 Abs.

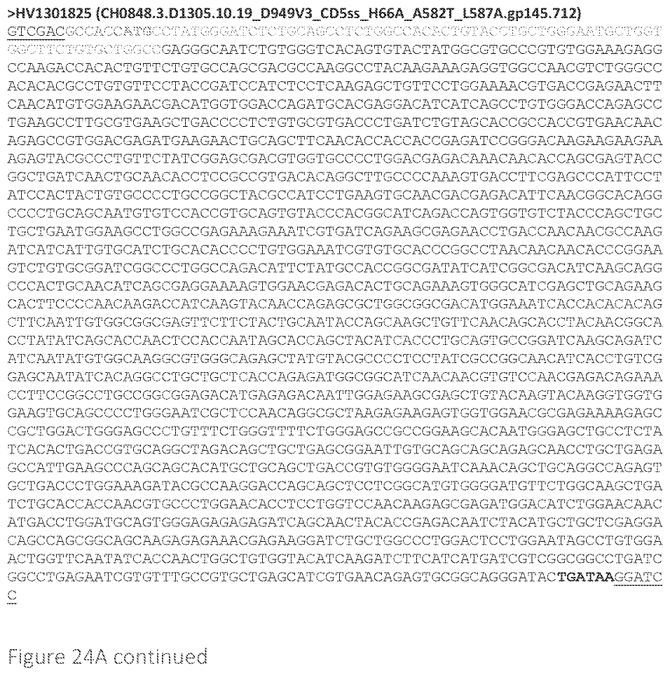

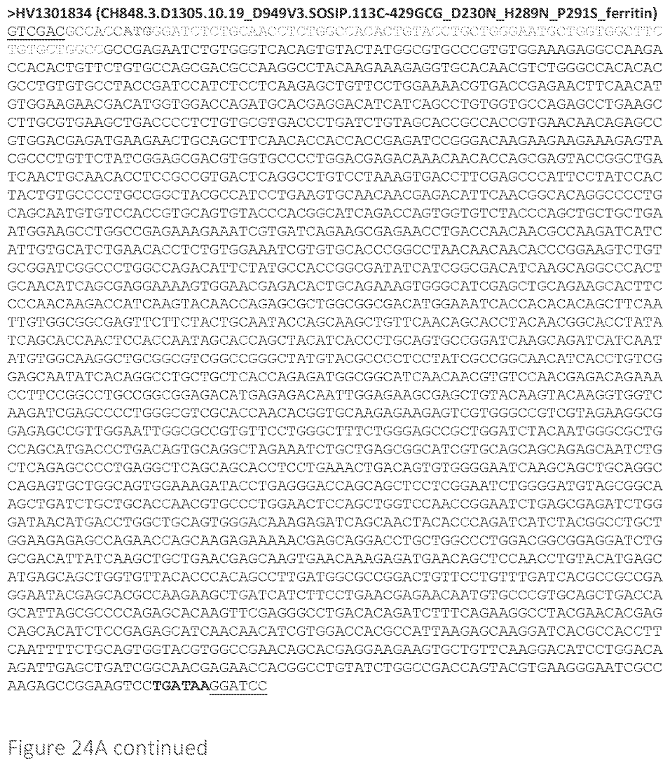

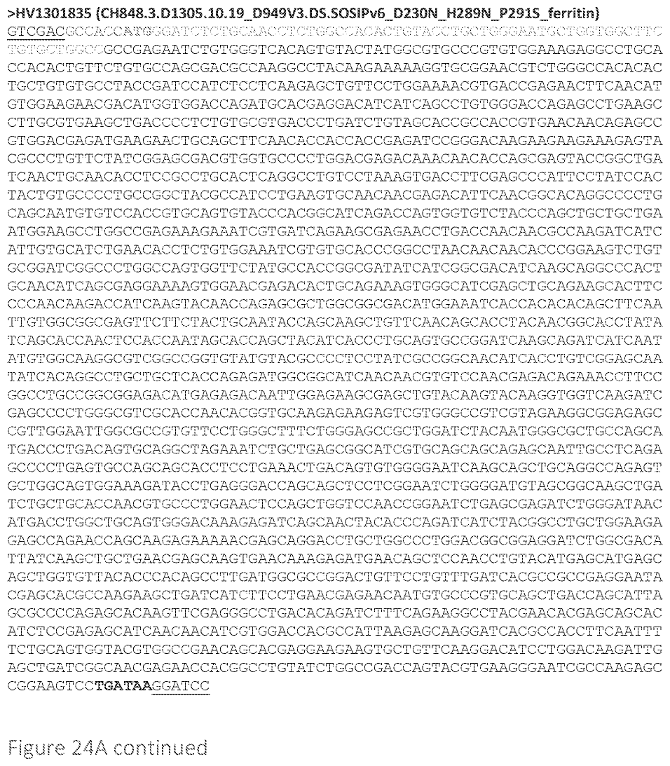

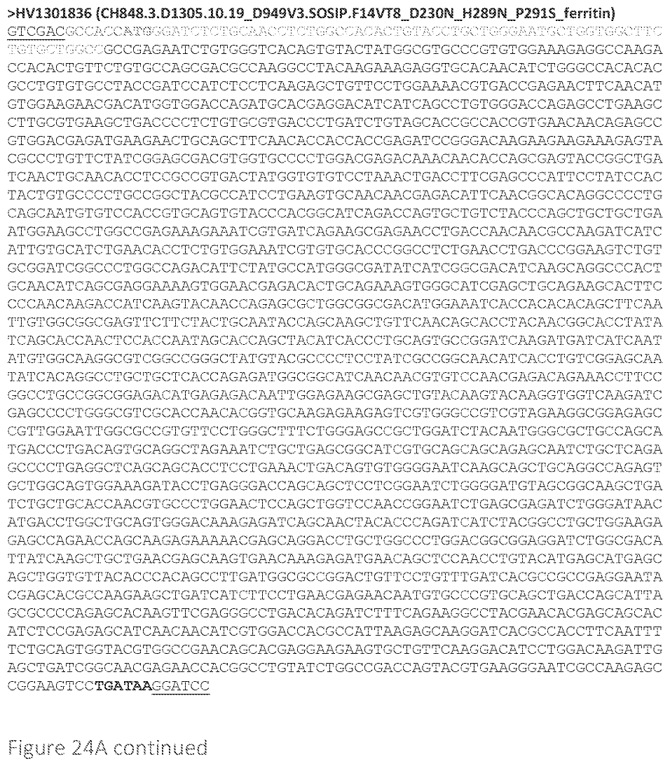

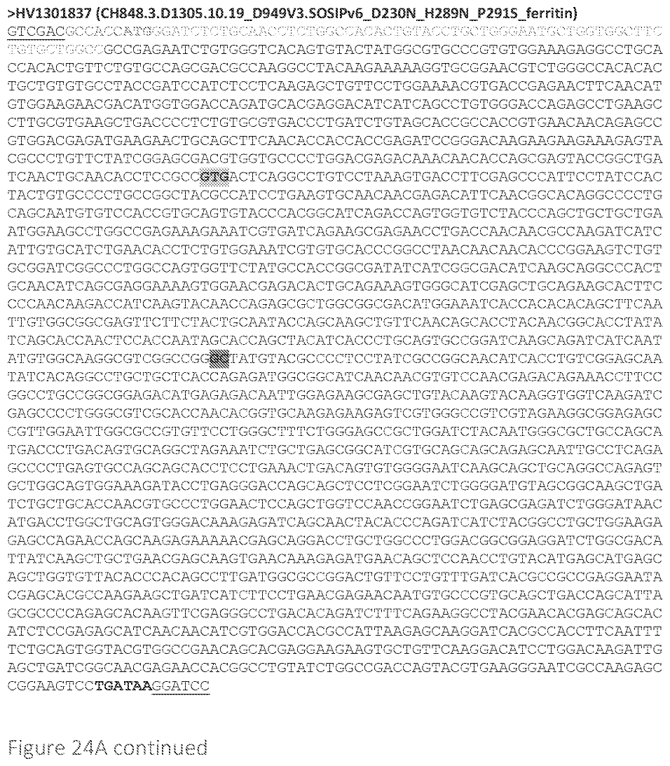

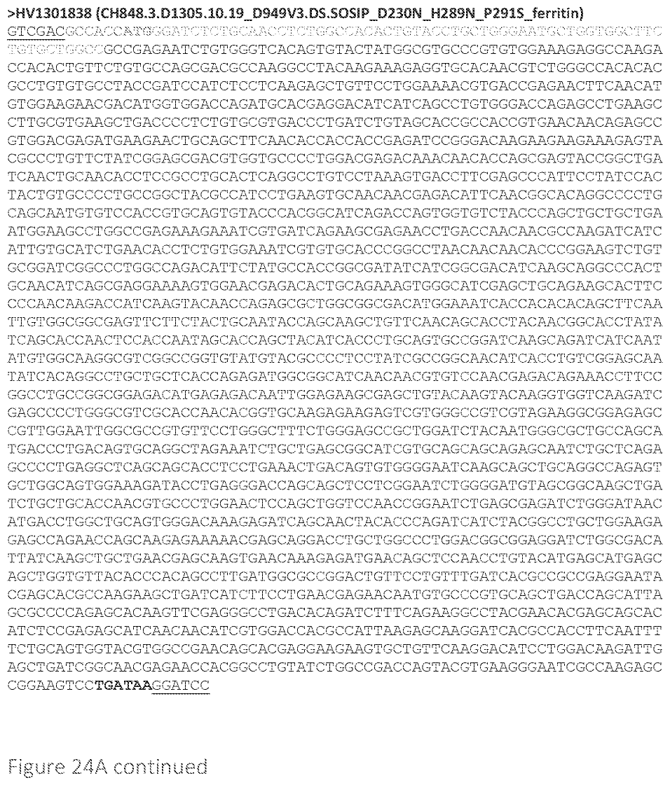

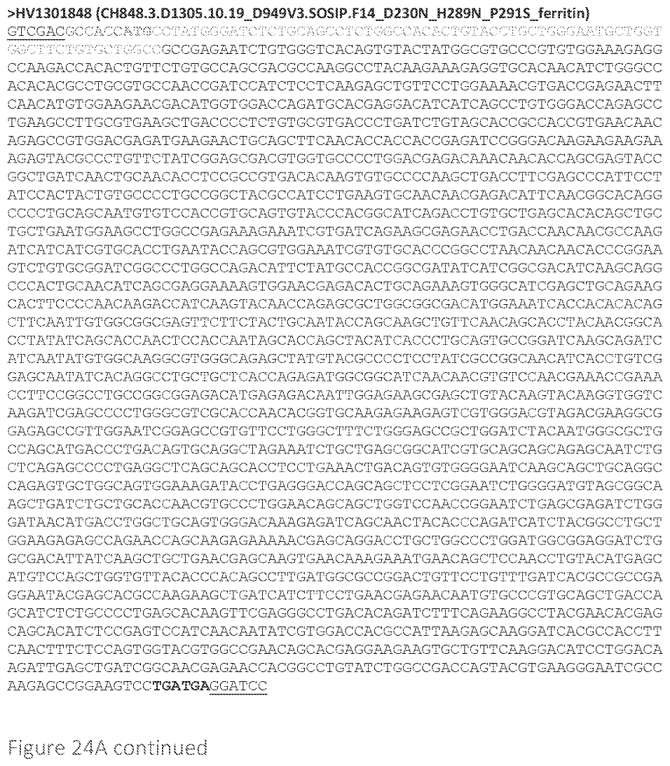

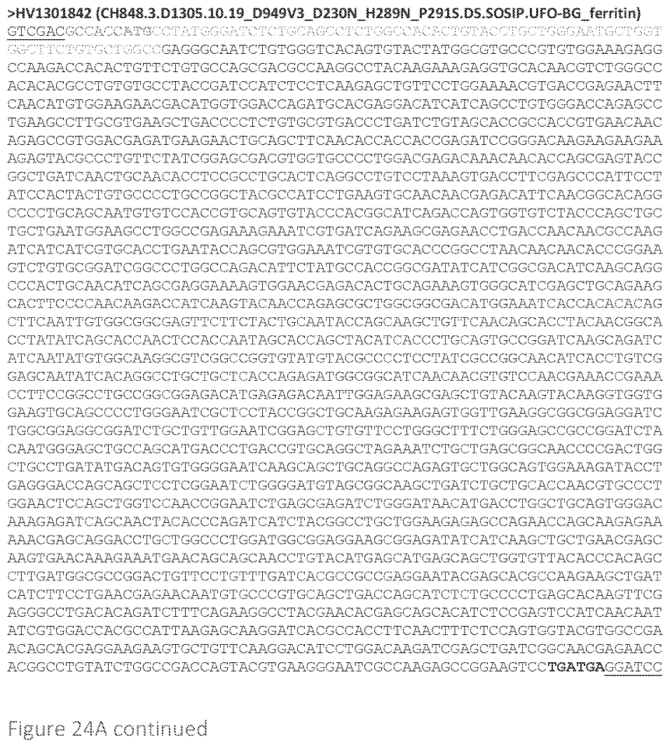

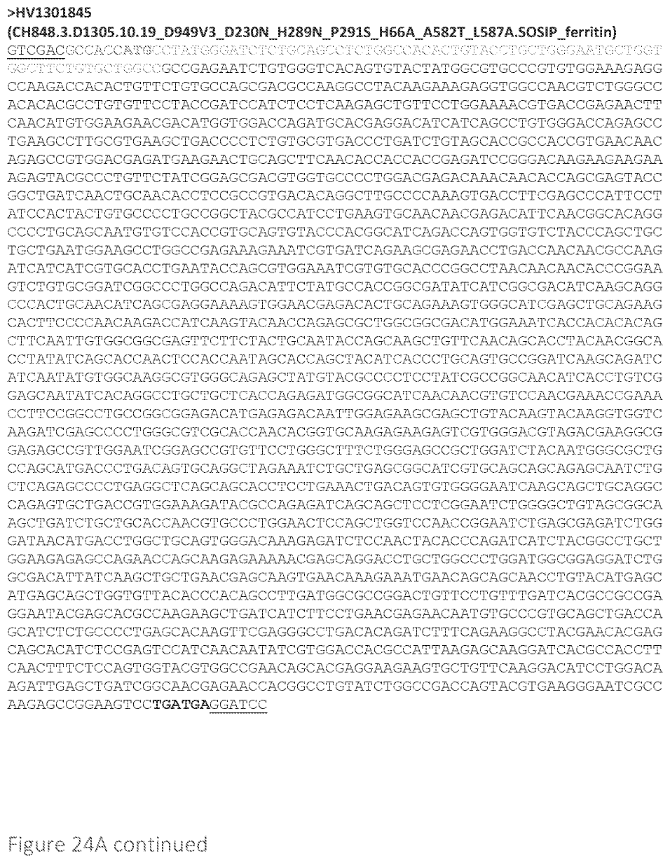

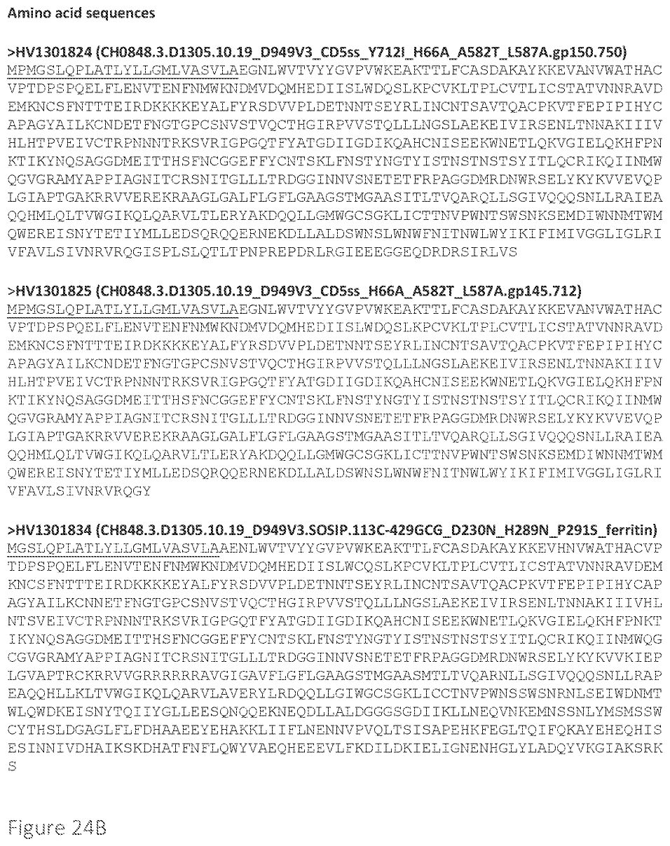

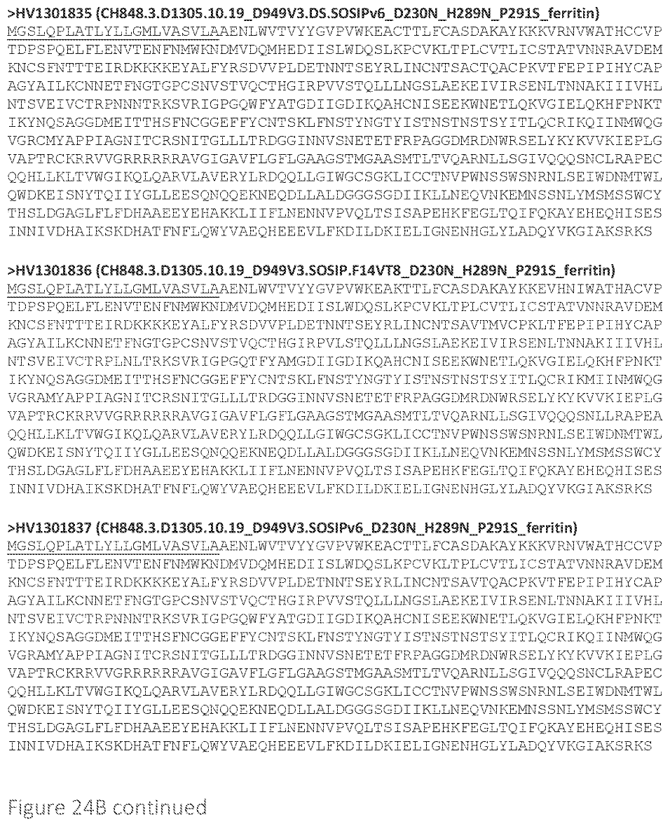

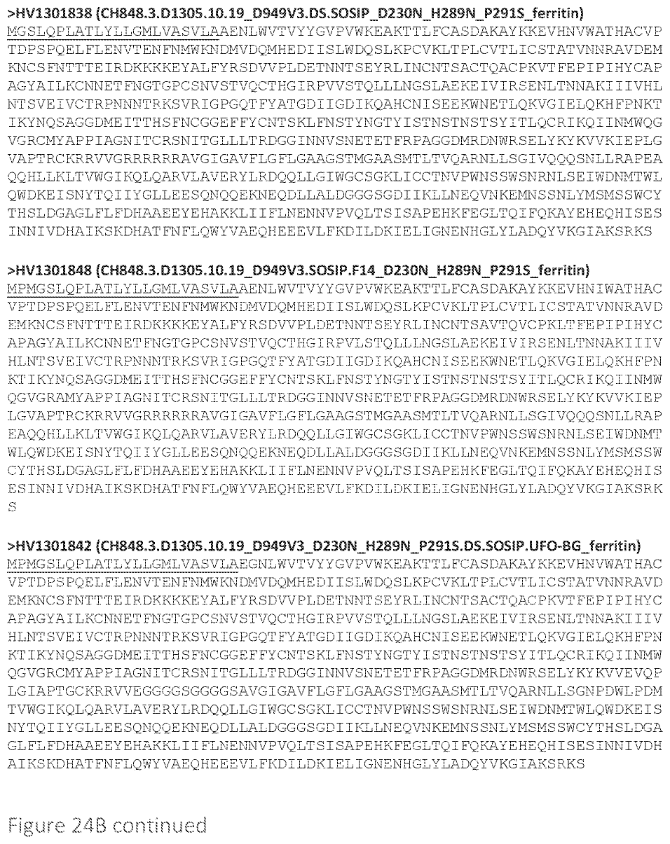

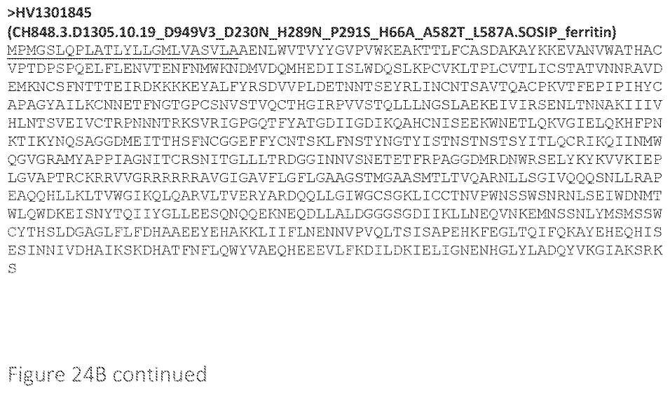

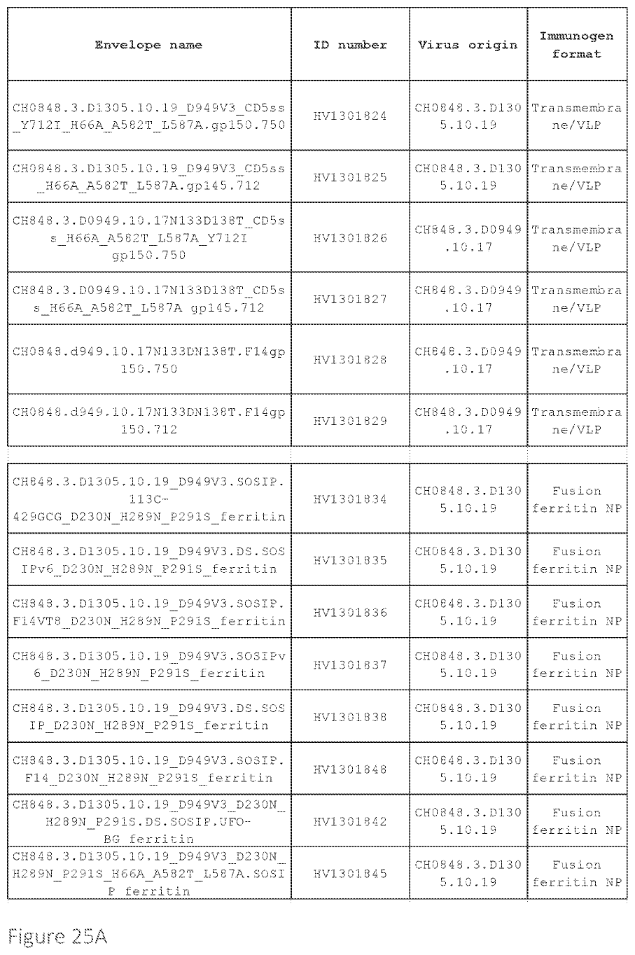

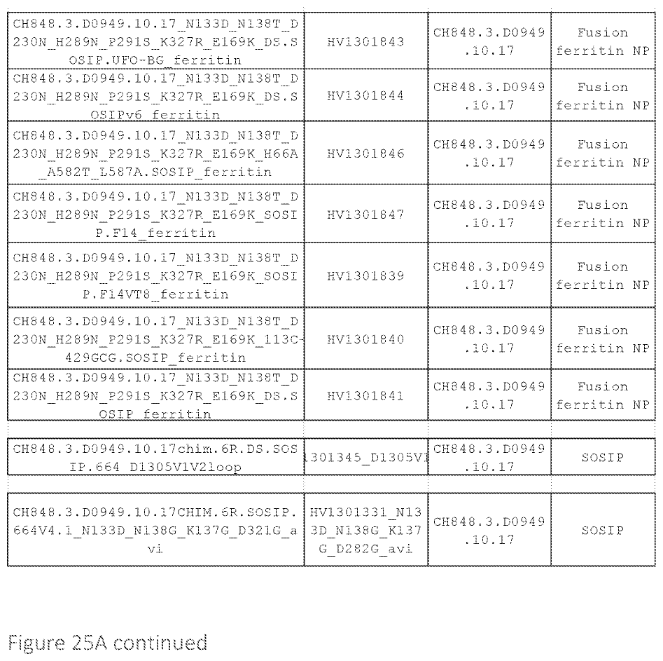

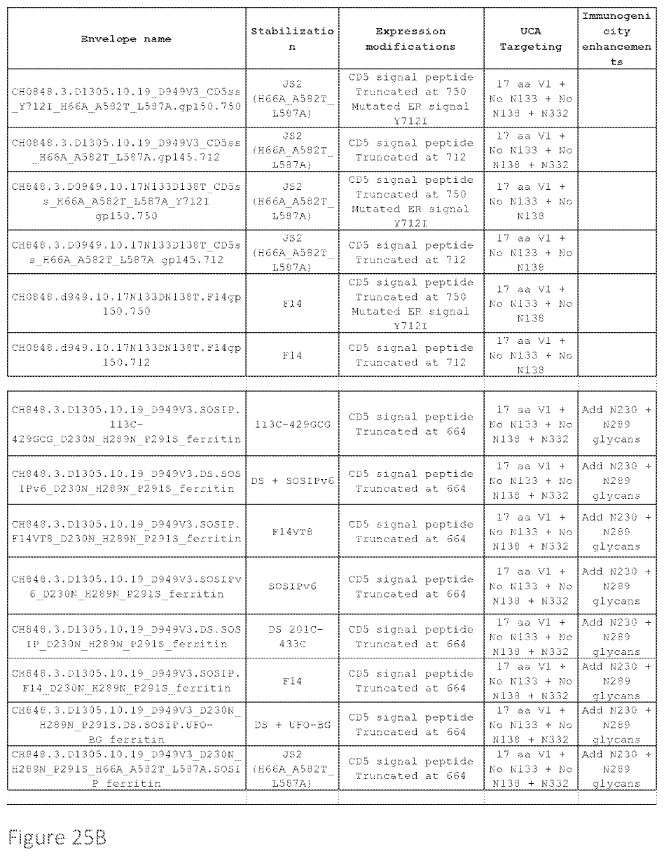

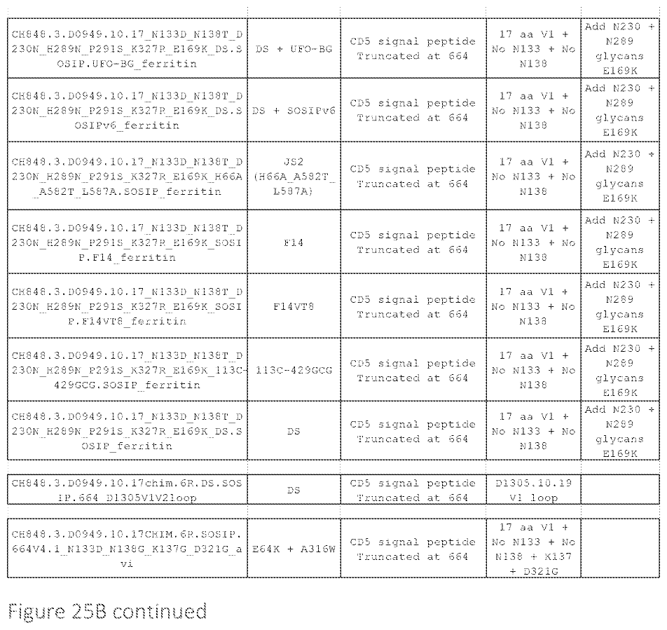

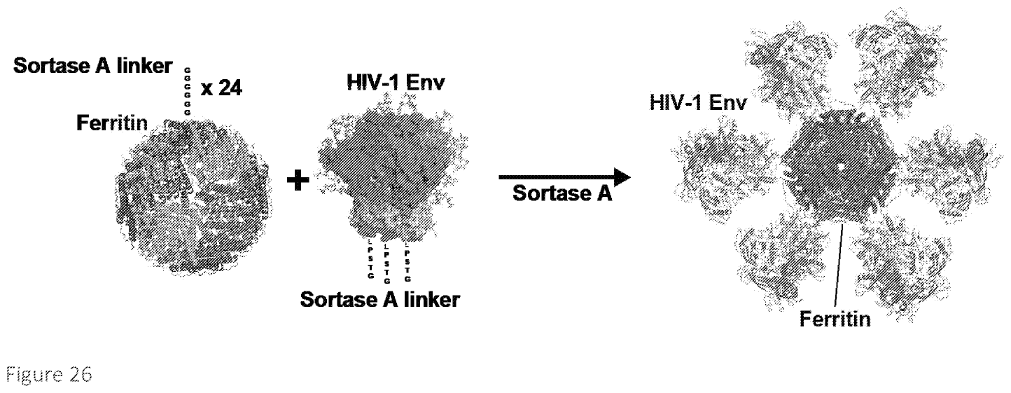

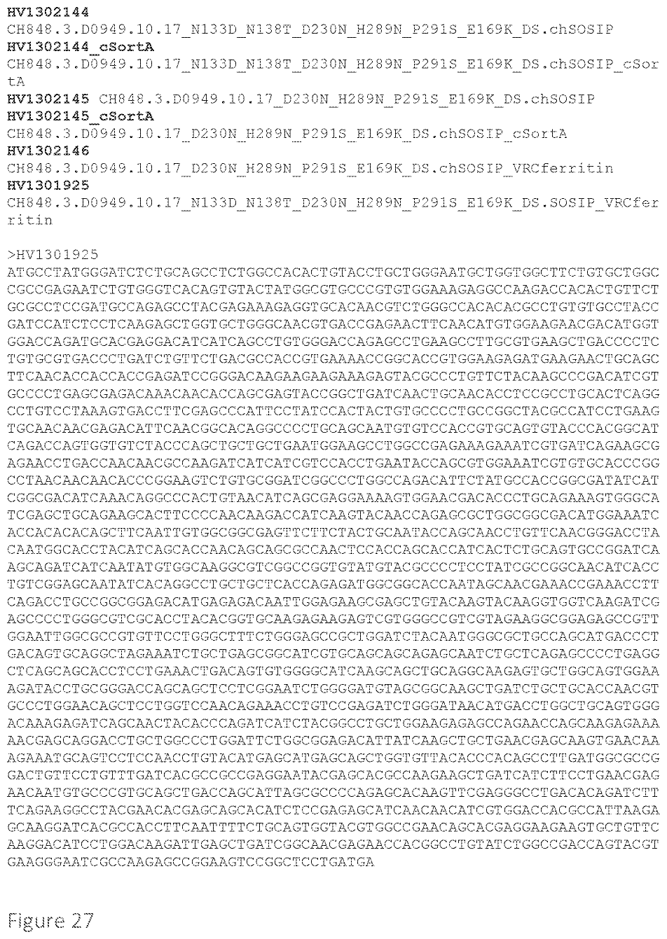

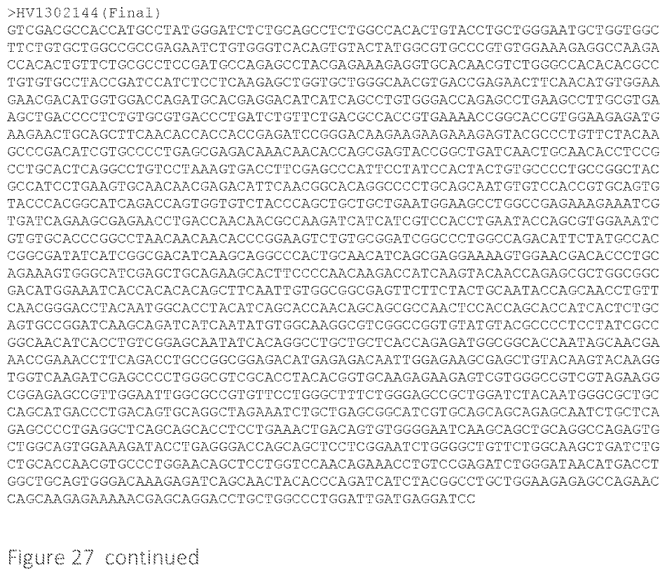

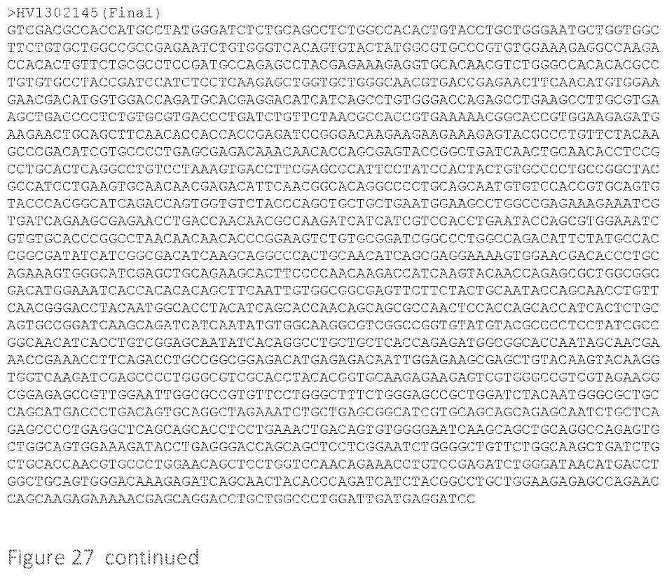

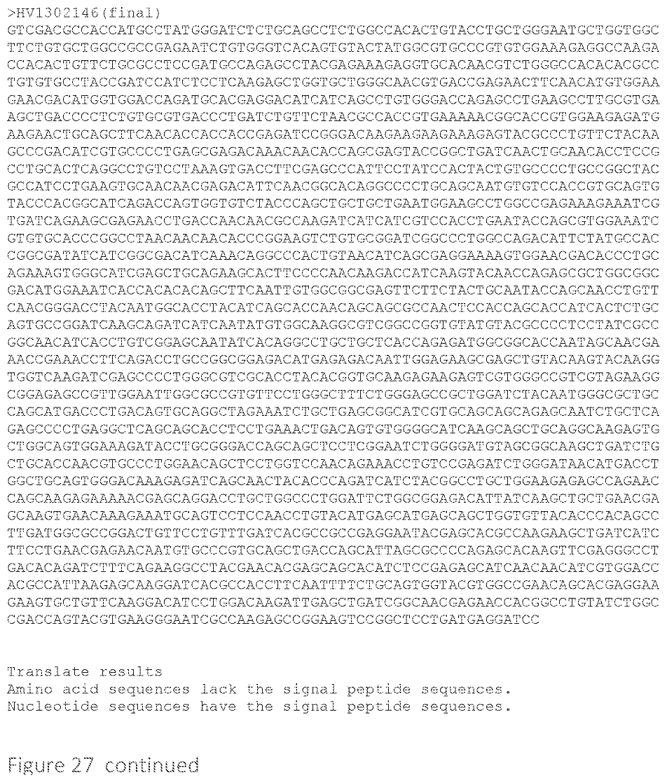

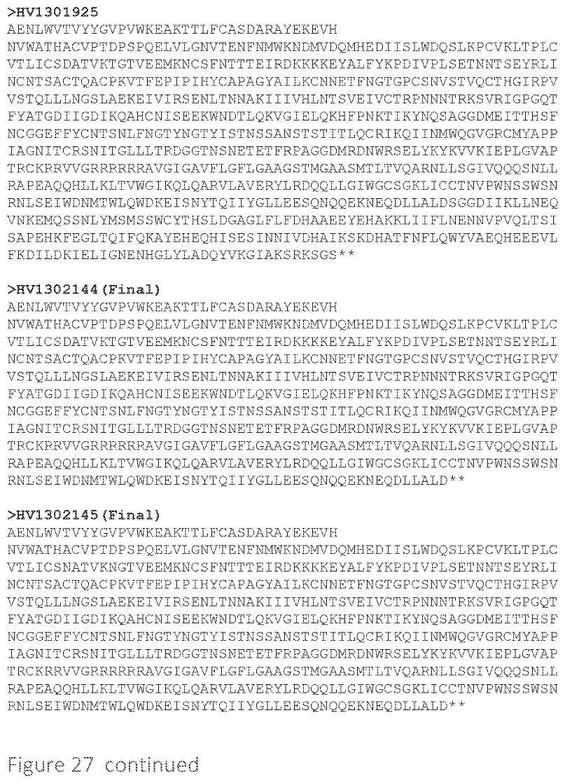

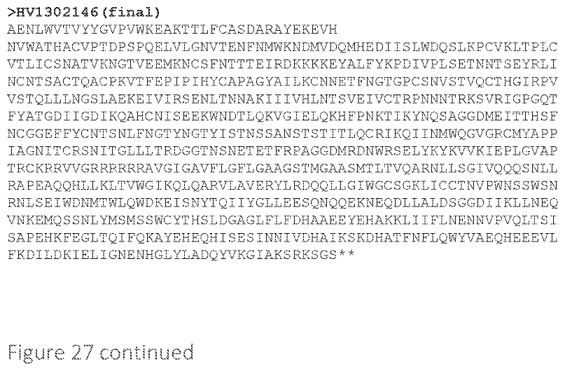

TABLE 3
Summary of envelope designs for use in prime and boost regimens

| | Envelope to be used as protein | Linker | Ferritin for | |
|---|---|---|---|---|
| HV name | and/or mRNA | sequence | multimerization | FIG. |
| HV1302144 | 10.17 DT protein trimer | no | No | FIG. 27 |
| | CH848.3.D0949.10.17_N133D_N138T_{—} | | | |
| | D230N_H289N_P291S_E169K_DS.chSOSIP | | | |
| HV1302144_{—} | 10.17 DT protein sortaNP | For Sortase | Any Ferritin is | |
| cSortA | CH848.3.D0949.10.17_N133D_N138T_{—} | conjugation | added via | |
| | D230N_H289N_P291S_E169K_DS.chSOSIP_{—} | | Sortase | |
| | cSortA | | conjugation at C- | |
| | | | terminus | |
| HV1301925 | 10.17 DT protein NP | SGG | could be any | FIG. 27 |
| | CH848.3.D0949.10.17_N133D_N138T_{—} | | suitable ferritin | |
| | D230N_H289N_P291S_E169K_DS.chSOSIP_{—} | | | |
| | VRCferritin | | | |
| HV1302145 | 10.17 protein trimer | no | | FIG. 27 |
| | CH848.3.D0949.10.17_D230N_H289N_{—} | | | |
| | P291S_E169K_DS.chSOSIP | | | |
| HV1302145_{—} | CH848.3.D0949.10.17_D230N_H289N_{—} | For Sortase | Any Ferritin is | |
| cSortA | P291S_E169K_DS.chSOSIP_cSortA | conjugation | added via | |
| | | | Sortase | |
| | | | conjugation at C- | |
| | | | terminus | |
| HV1302146 | CH848.3.D0949.10.17_D230N_H289N_{—} | SGG | could be any | FIG. 27 |
| | P291S_E169K_DS.chSOSIP_VRCferritin | | suitable ferritin | |

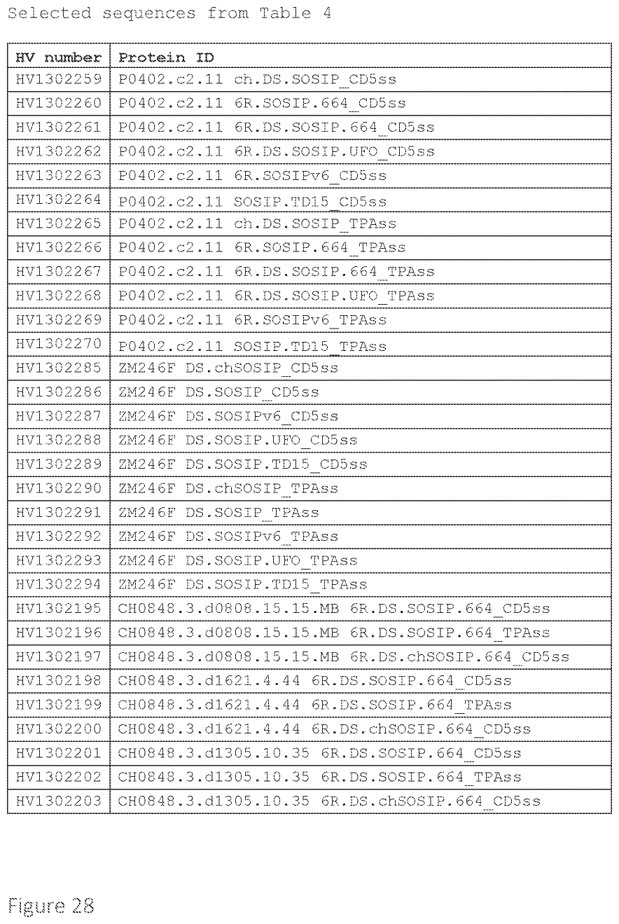

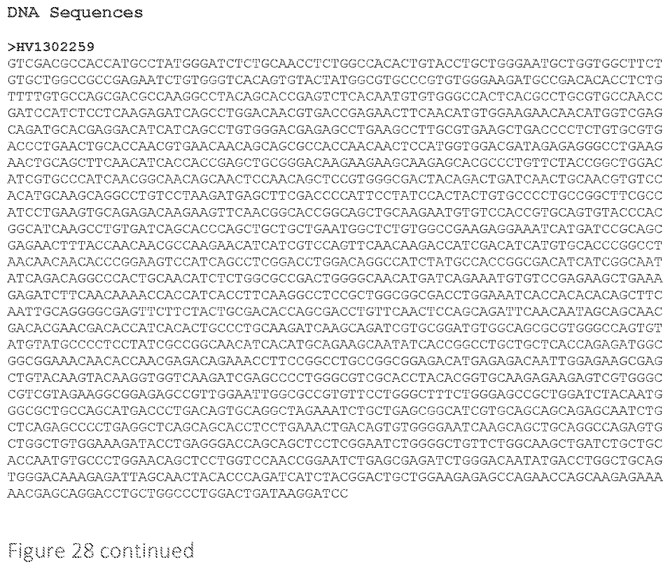

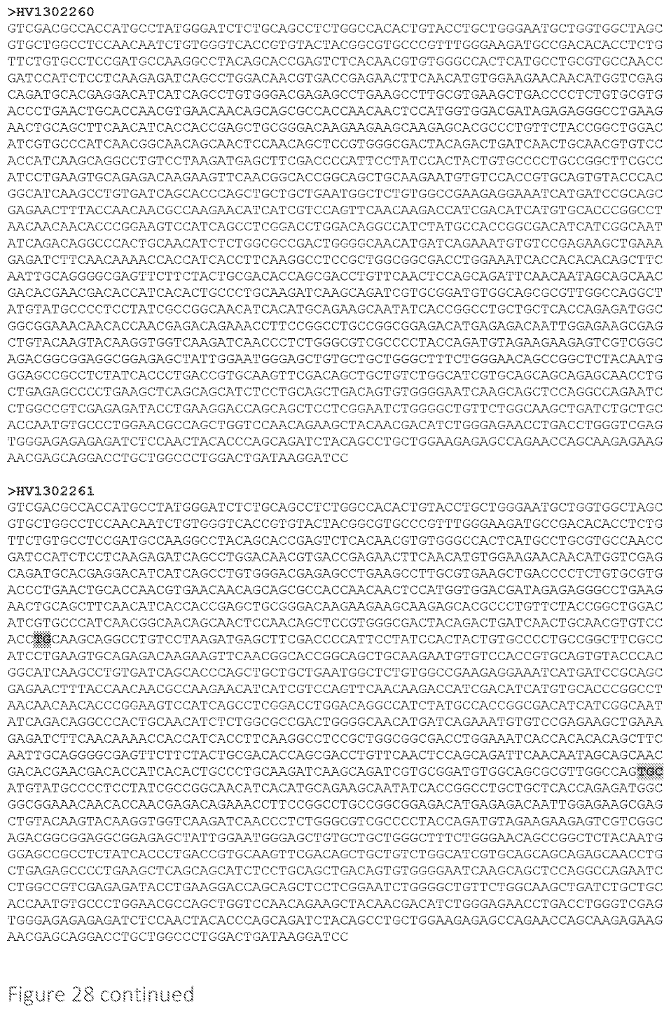

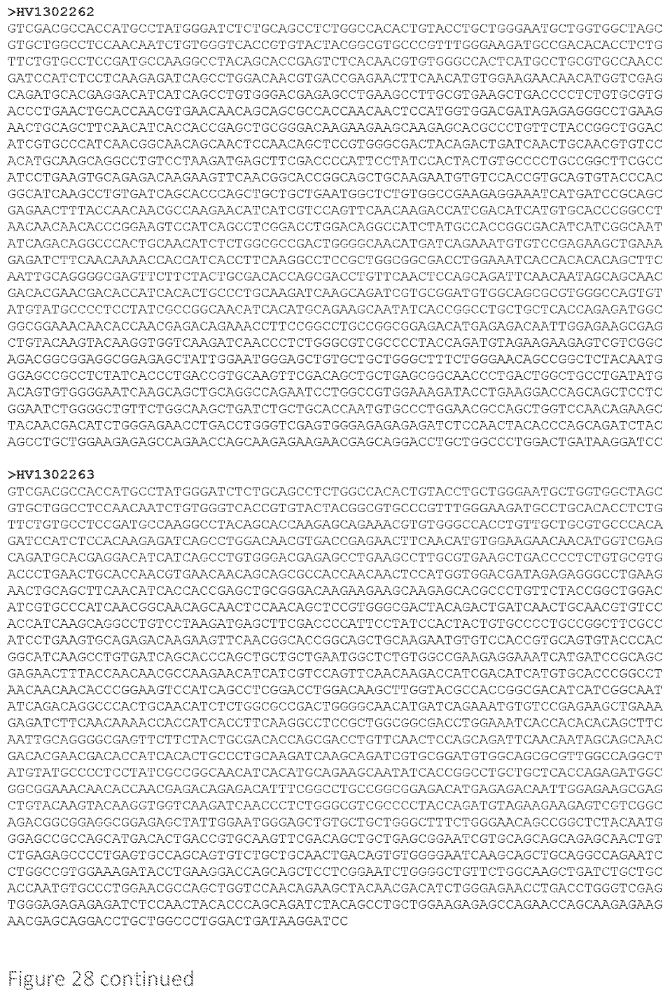

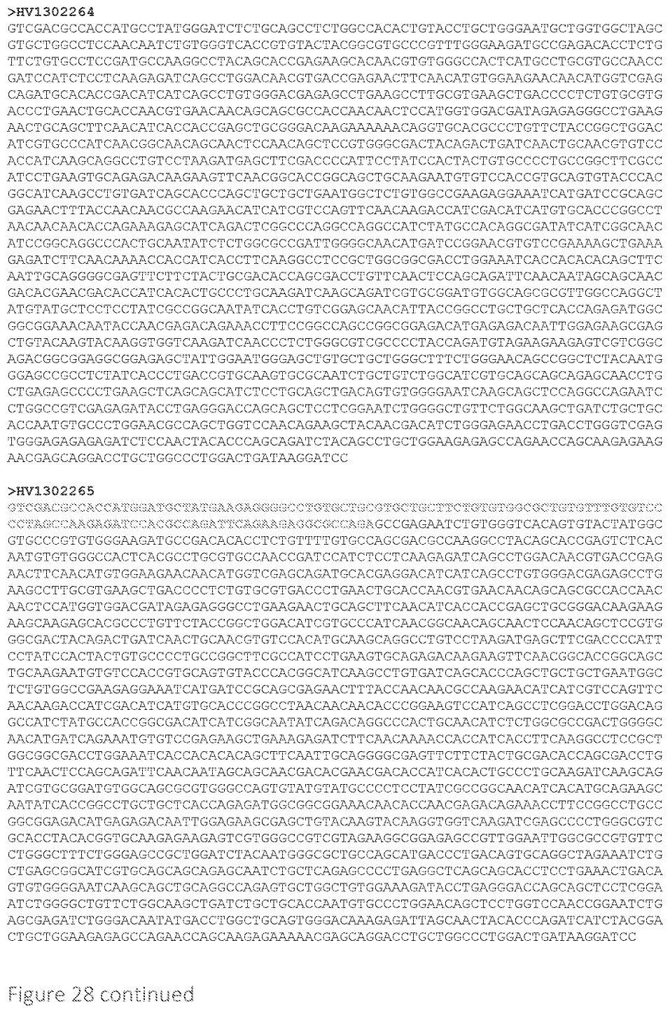

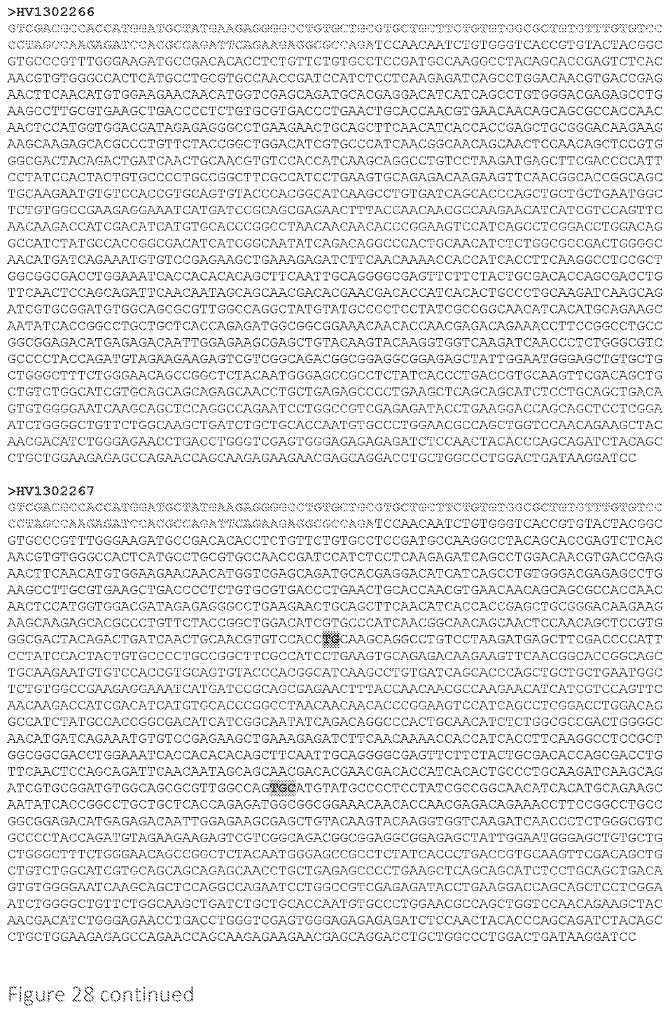

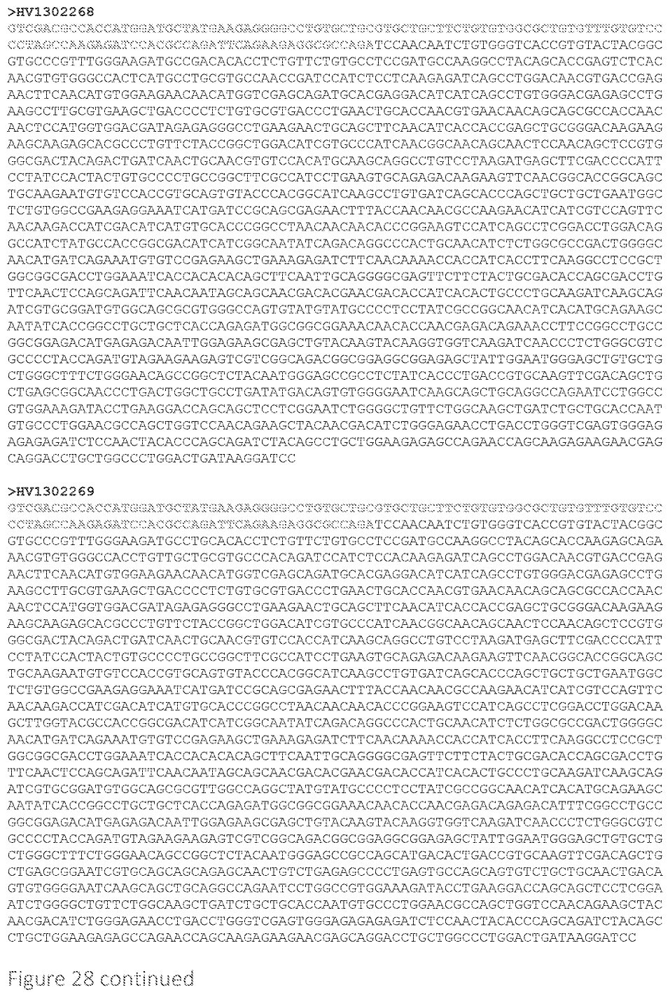

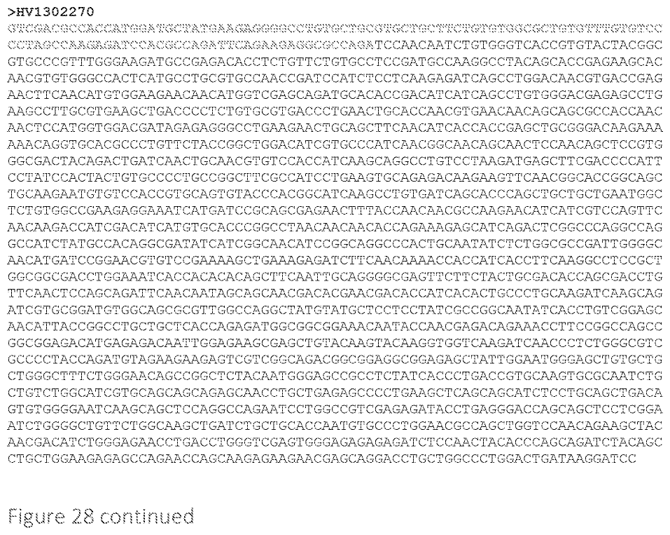

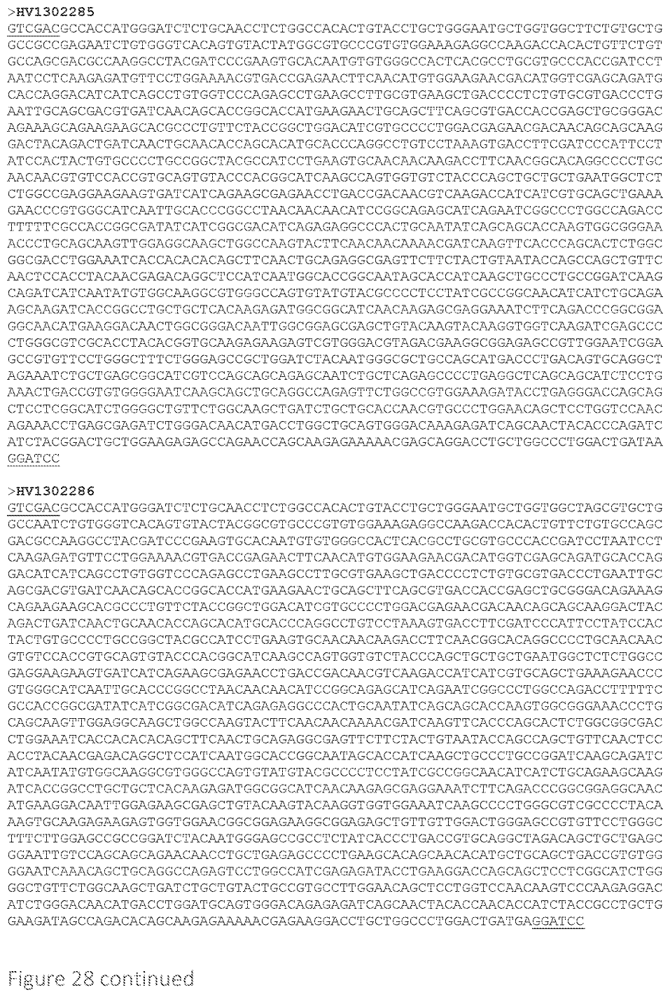

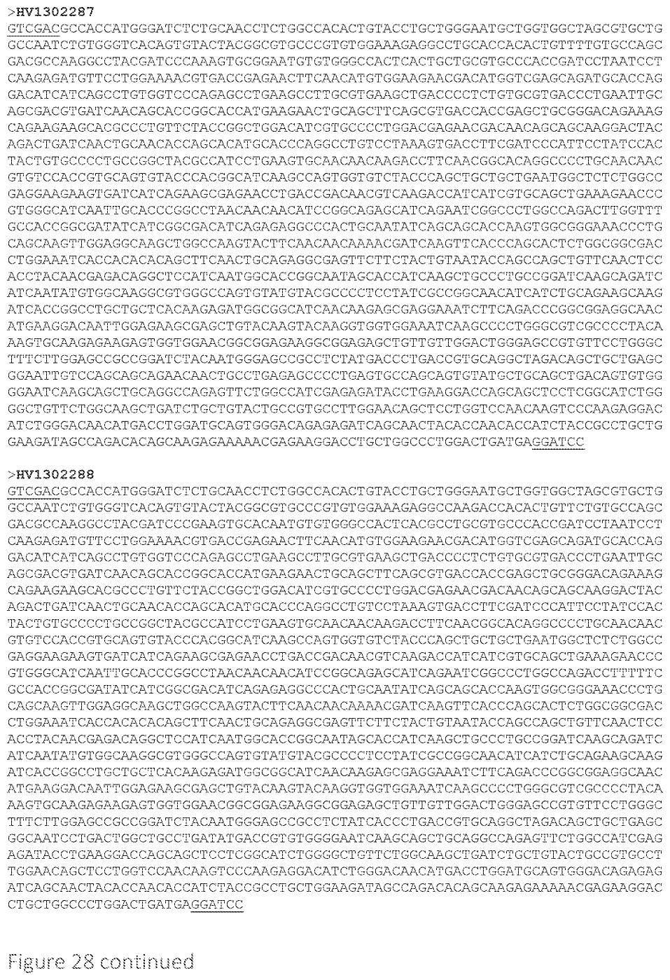

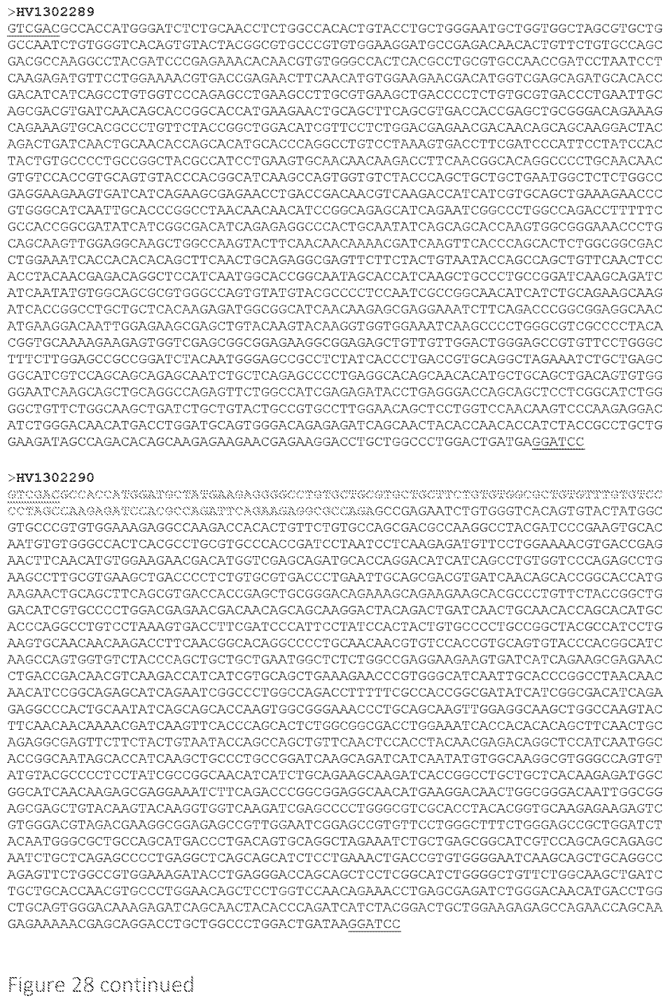

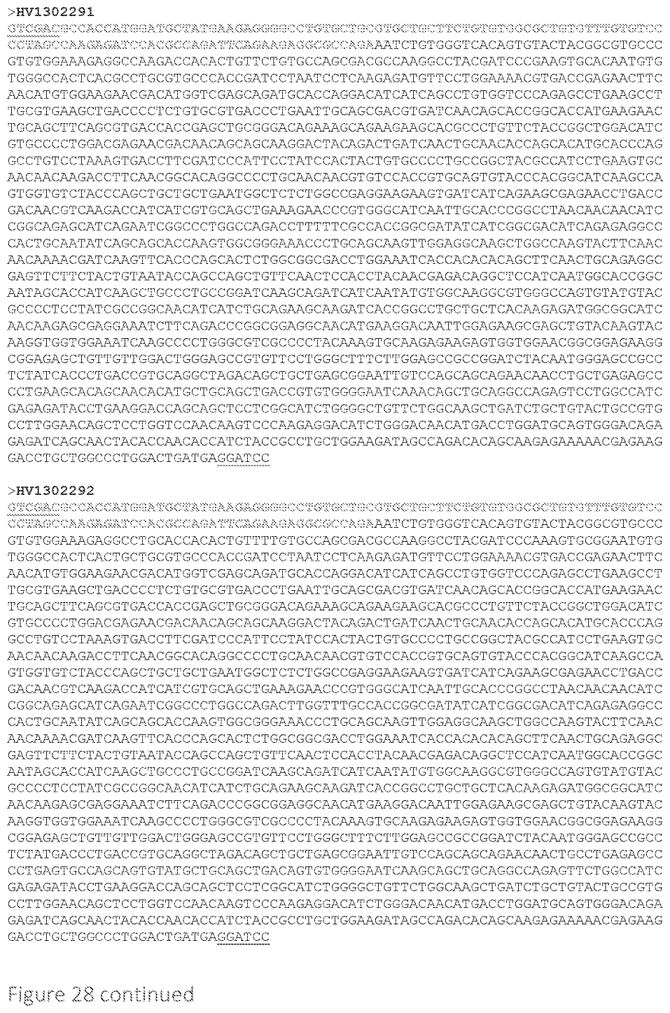

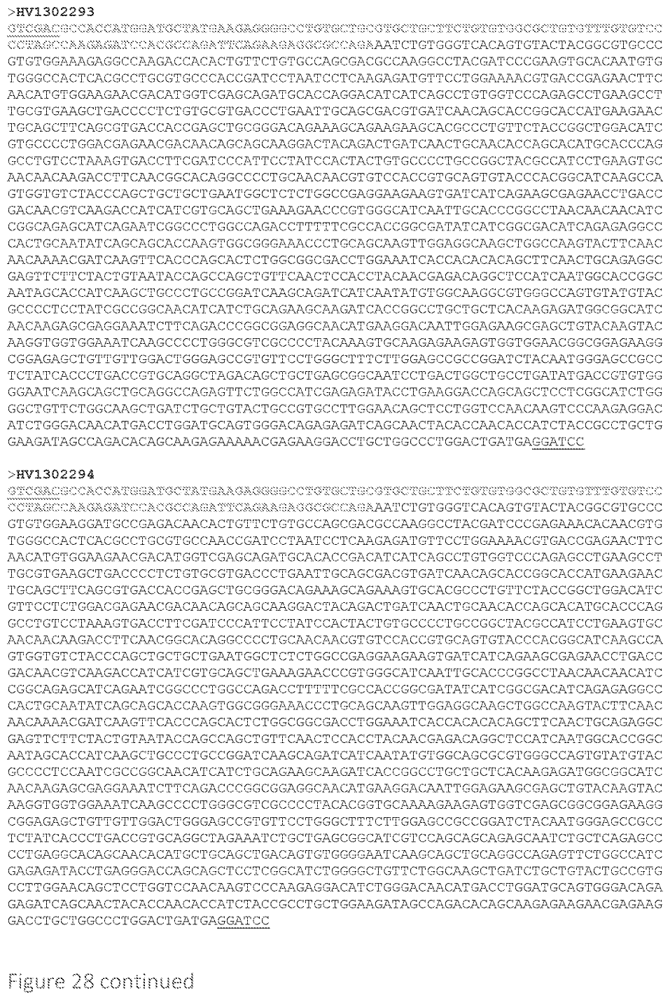

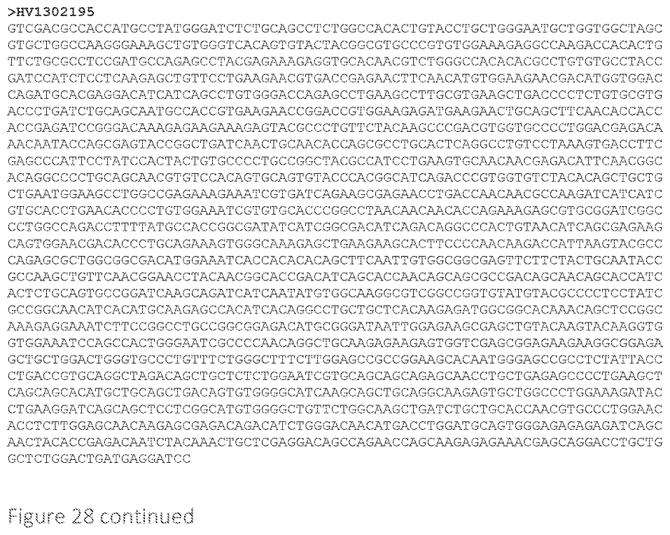

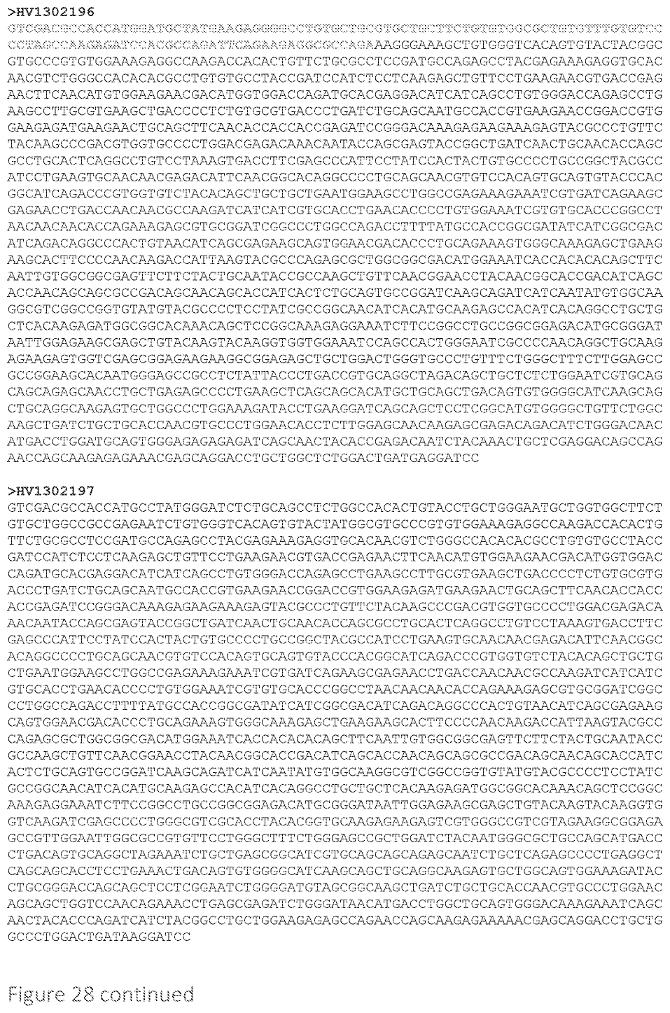

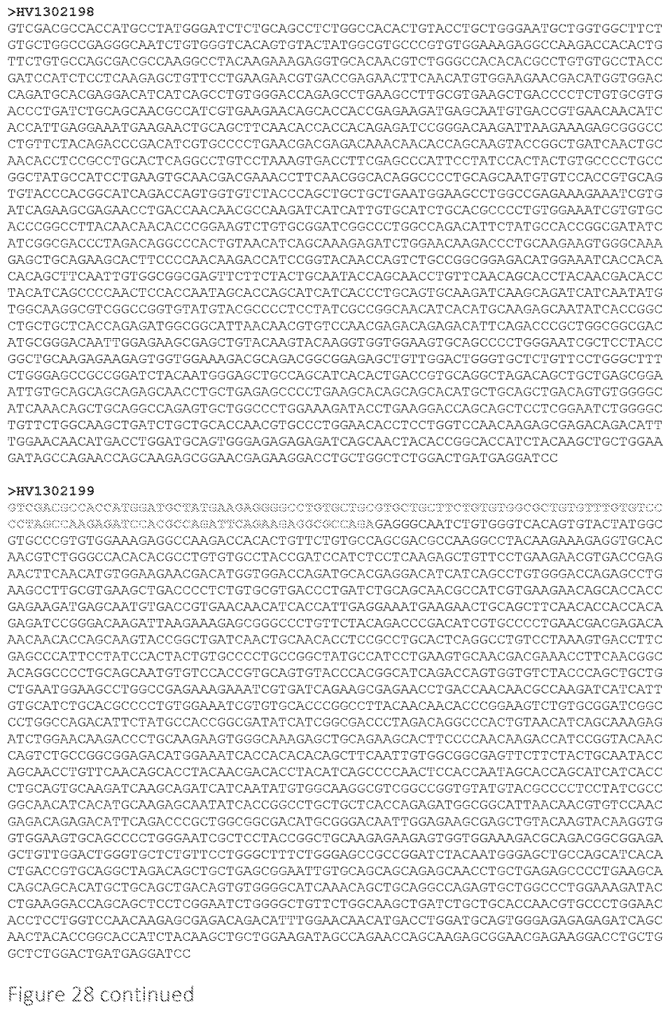

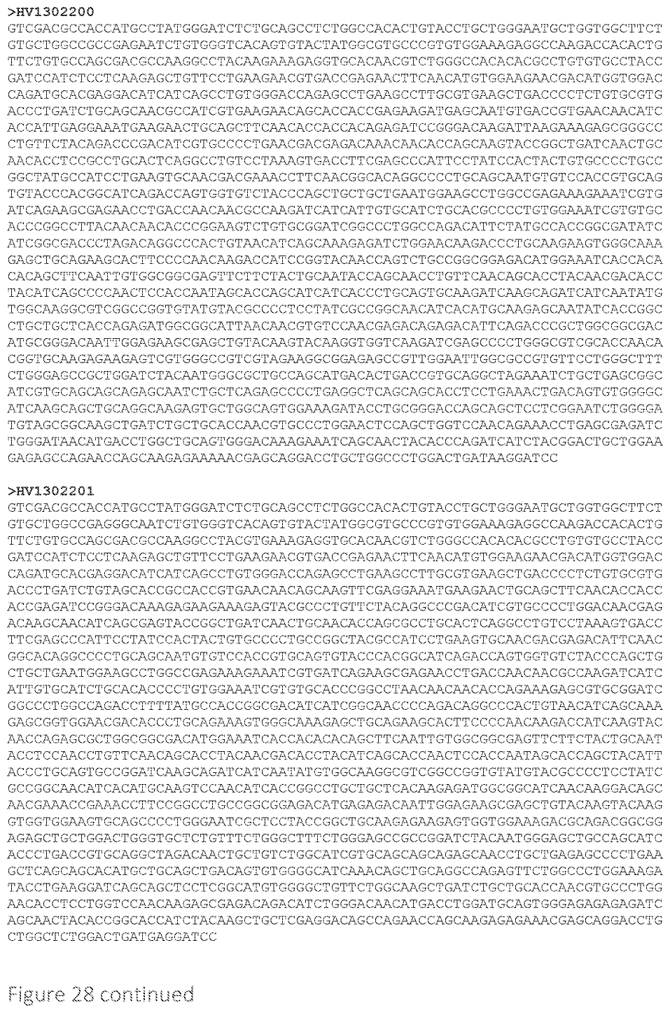

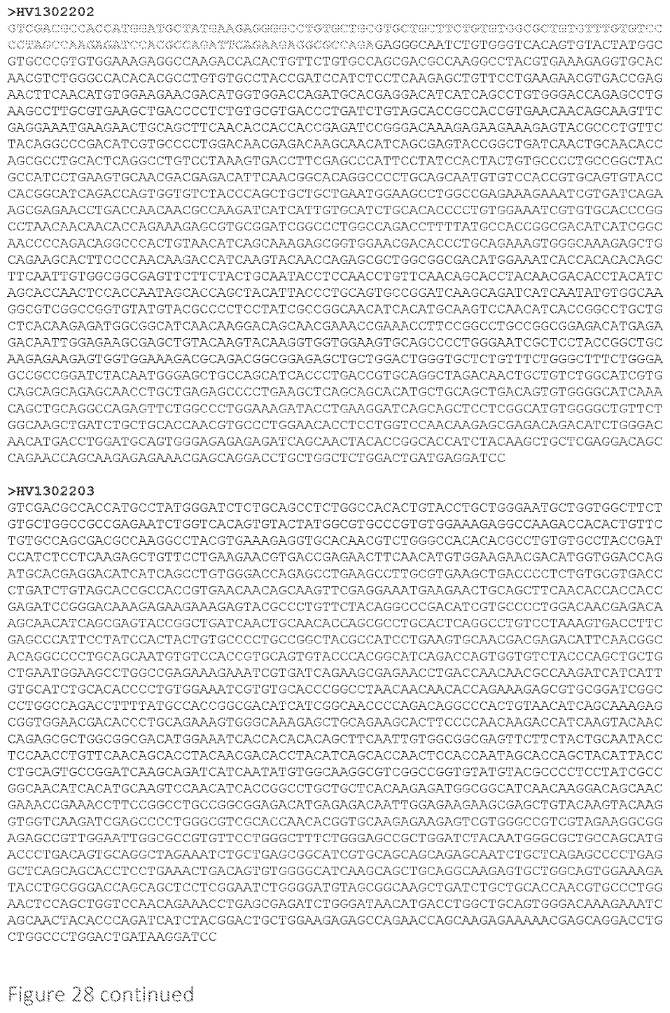

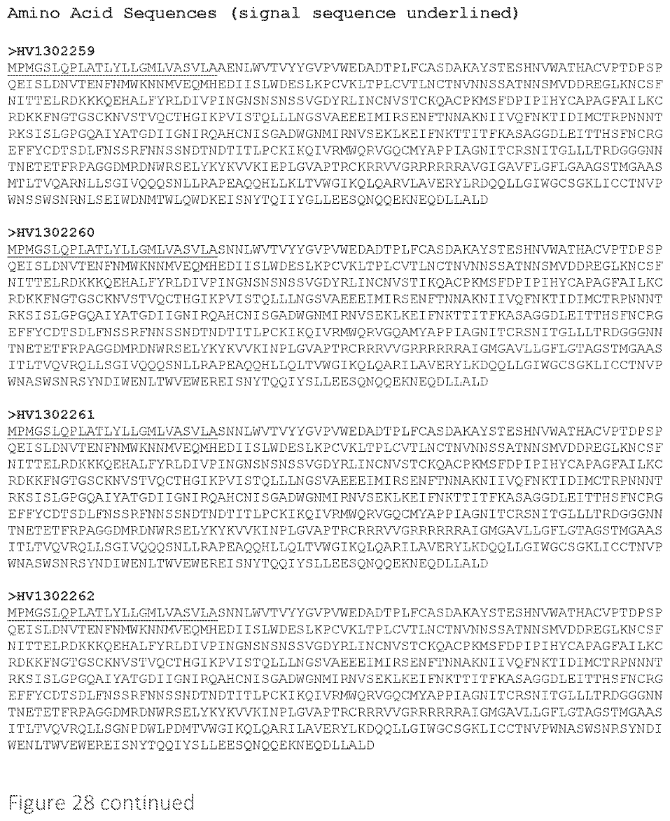

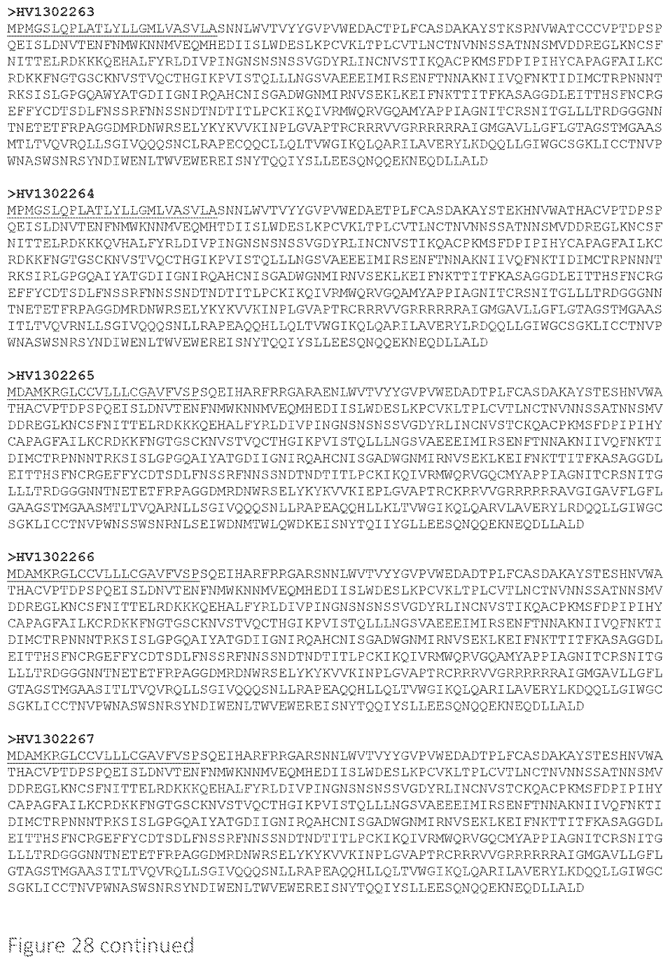

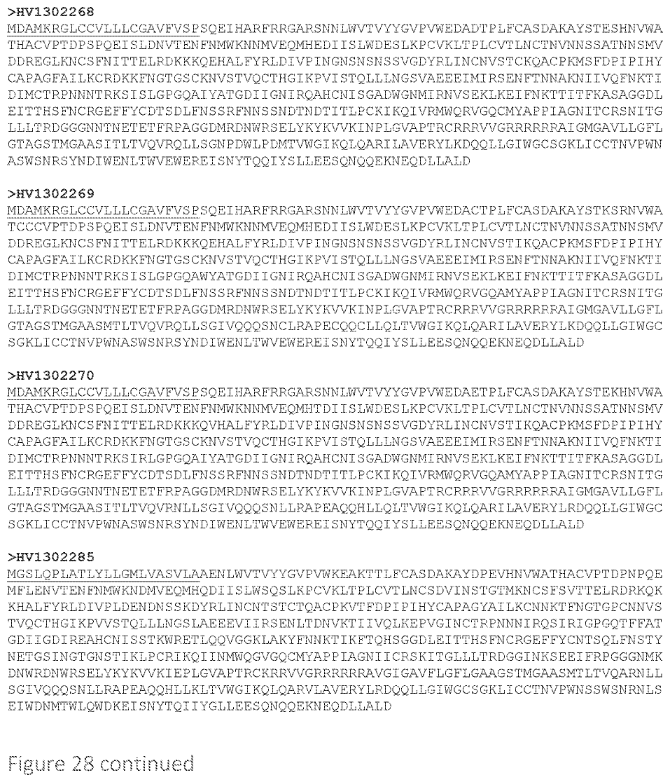

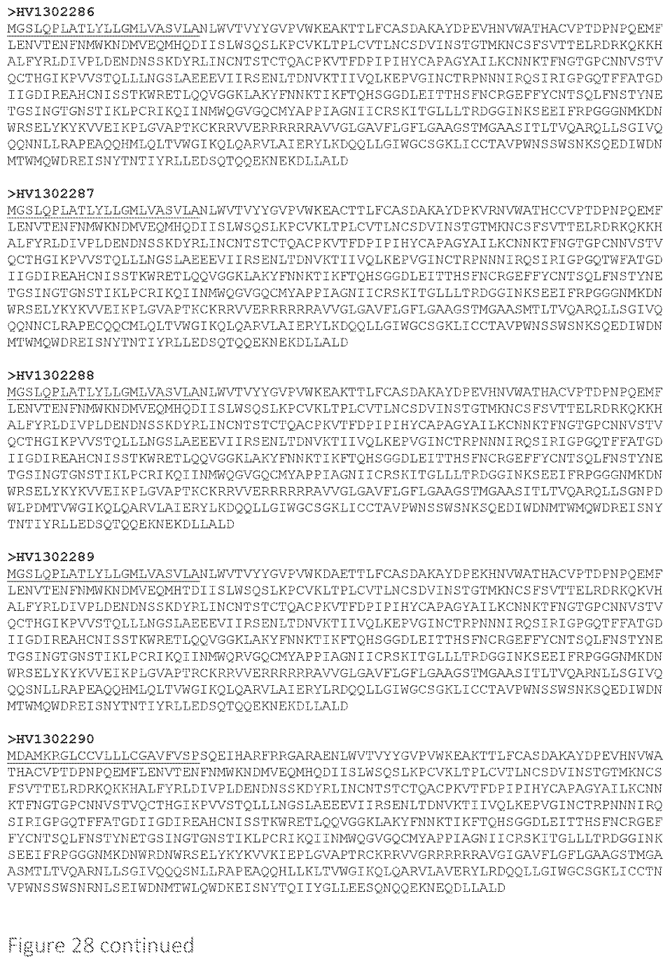

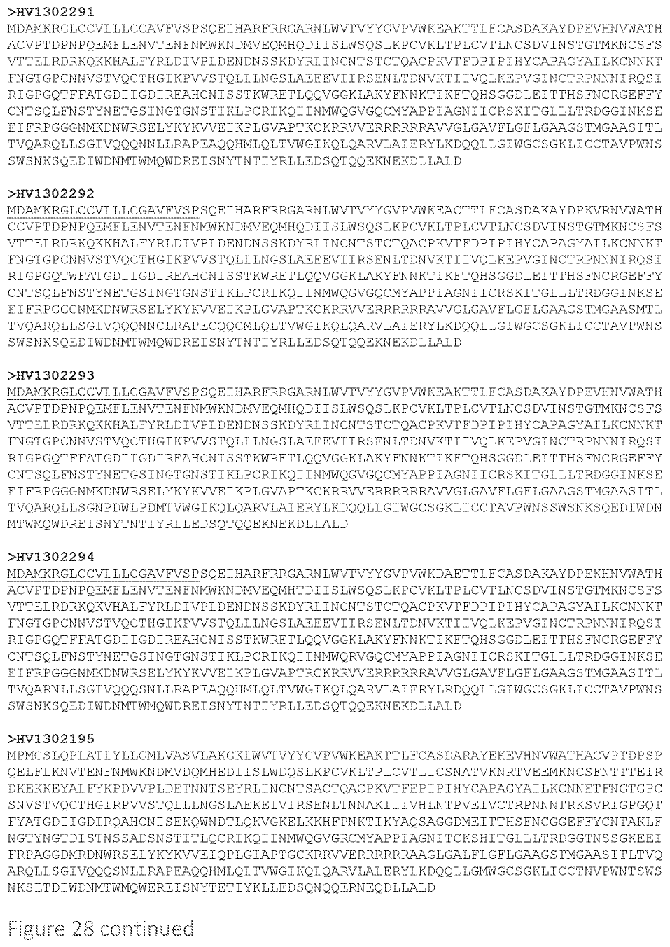

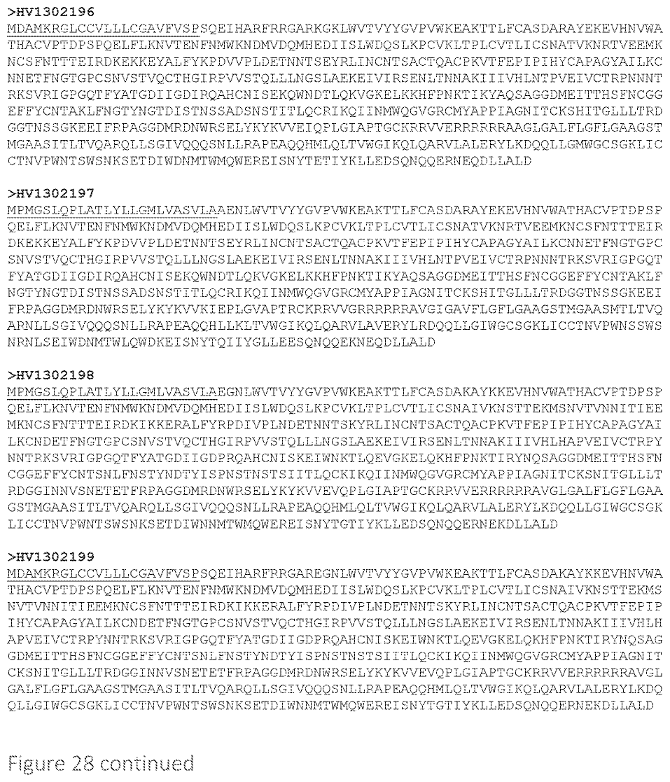

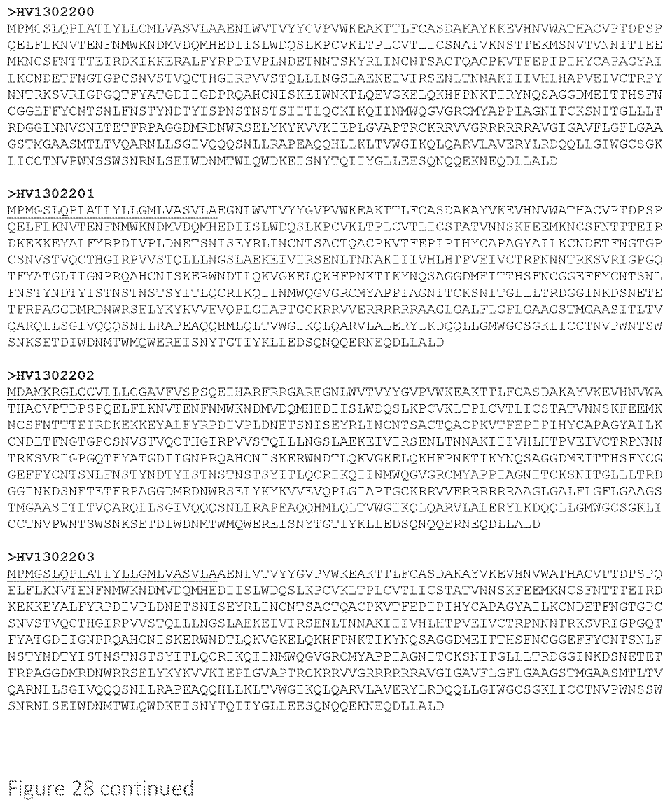

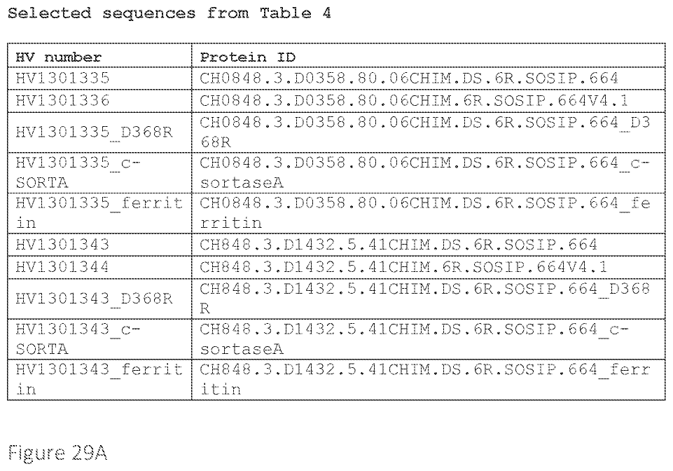

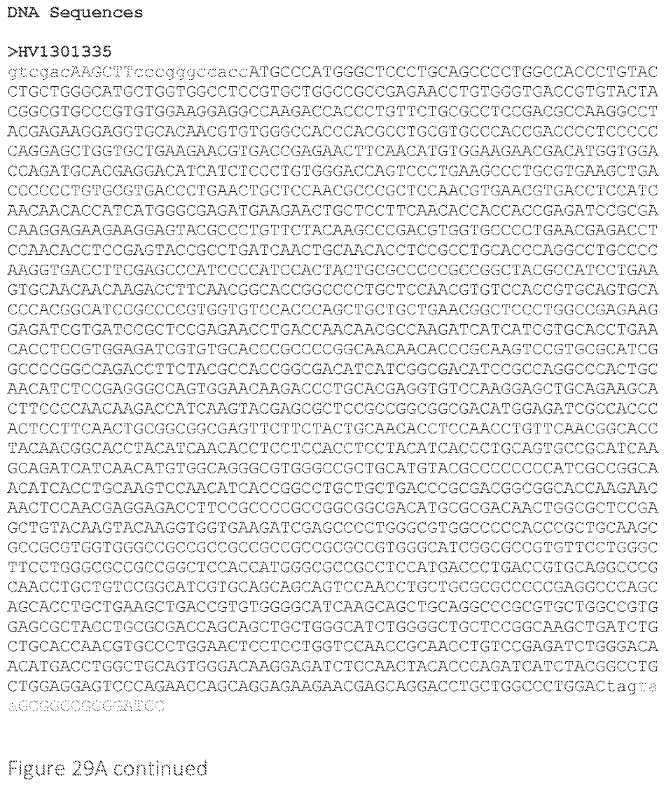

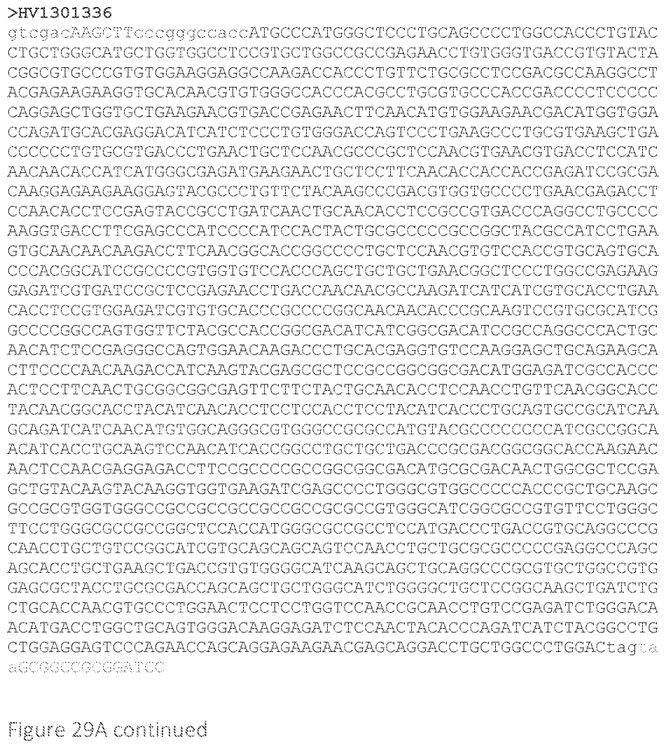

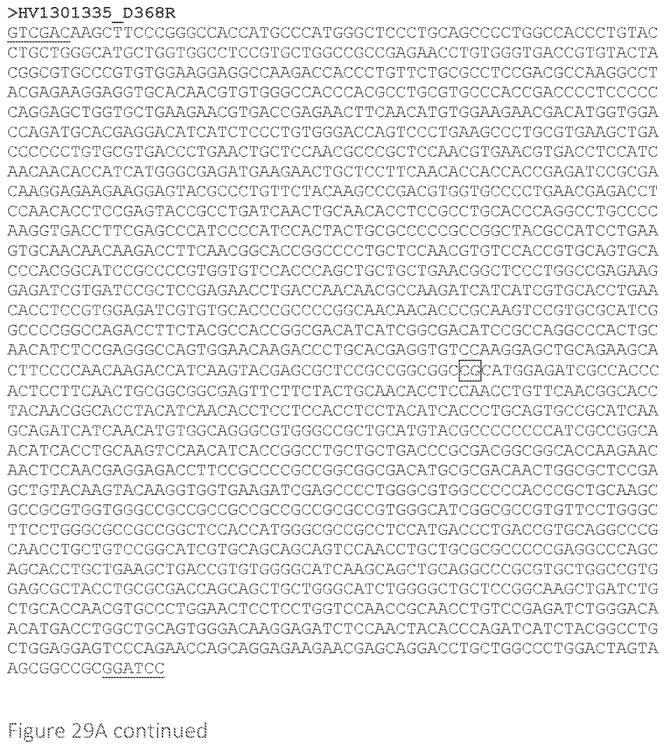

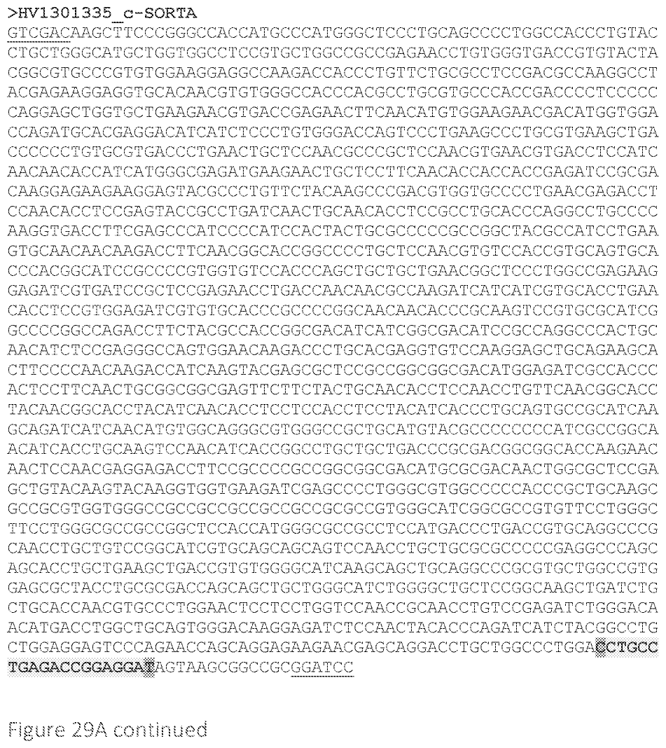

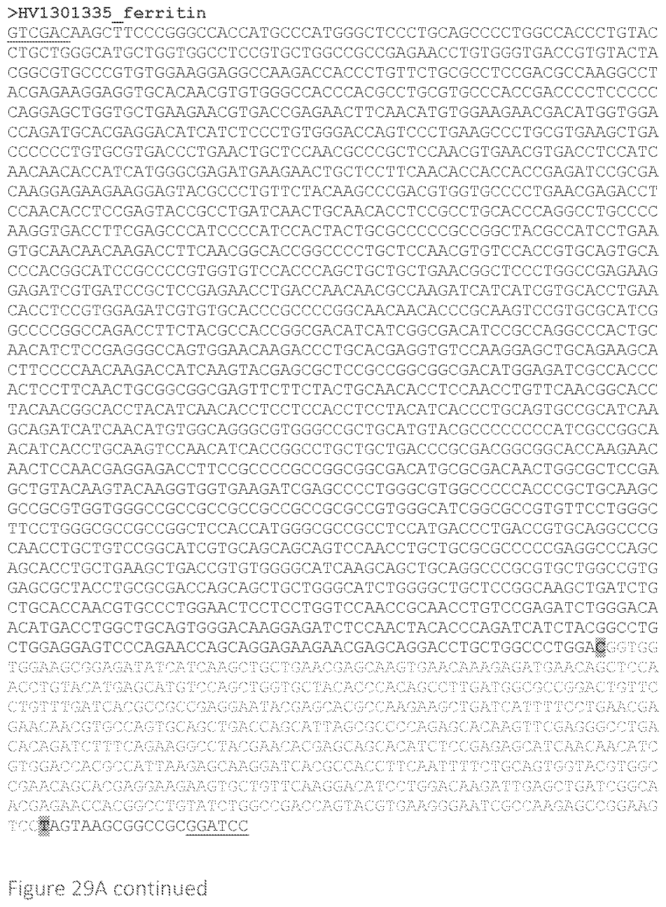

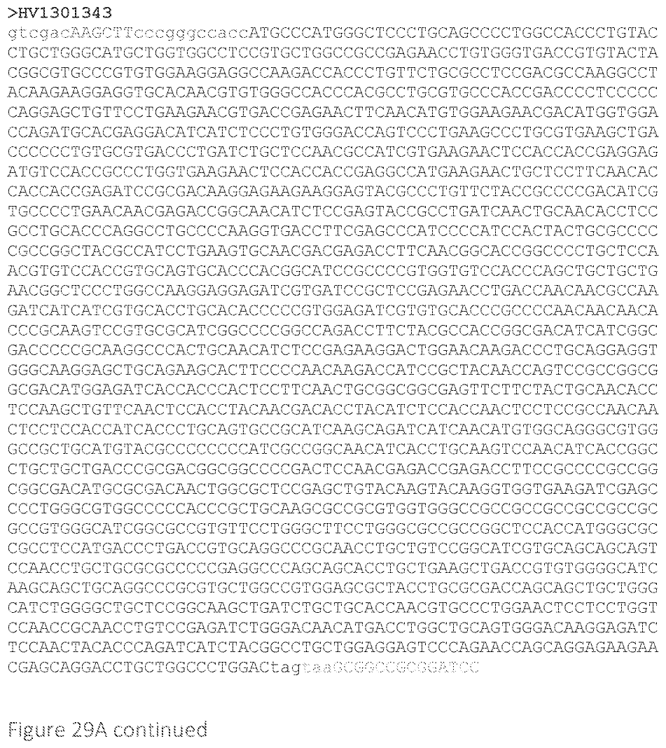

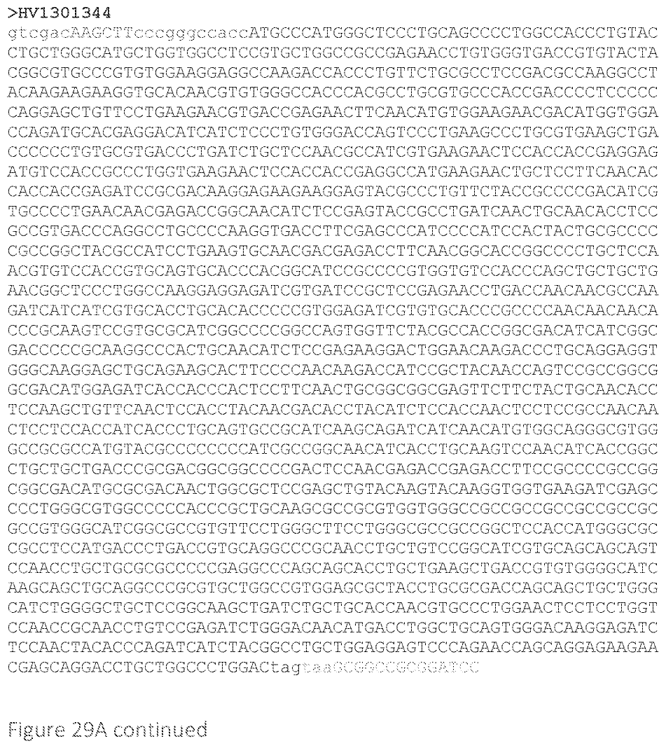

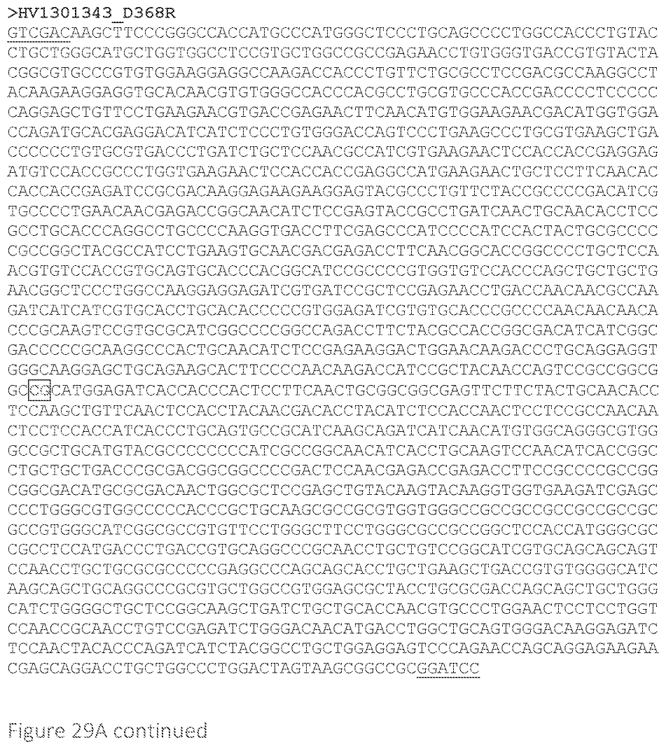

TABLE 4
Summary of selection of immunogens for induction of neutralizing antibodies.

| | | NEW selection | One embodiment | aa FIGS. | Envelope protomer |
|---|---|---|---|---|---|
| prime | | CH848.0949.10.17DT | See Table 3, FIG. 27 | | Various protomer |
| | | | | | designs, including |
| | | | | | without limitation |
| | | | | | various stabilized |
| | | | | | designs. |
| boost | x | CH848.0949.10.17 | See Table 3, FIG. 27 | | Various protomer |
| | | | | | designs, including |
| | | | | | without limitation |
| | | | | | various stabilized |
| | | | | | designs. |
| boost | | CH848.0808.15.15 | CH0848.3.d0808.15.15.MB6R.DS.SOSIP.664_{—} | FIG. 28 | CH0848.3.d0808.15.15.MB |
| | | | CD5ss | | 6R.DS.SOSIP.664 |
| | | | | | With various signal |
| | | | | | peptides |
| | | | | | Various protomer |
| | | | | | designs, including |
| | | | | | without limitation |
| | | | | | various stabilized |
| | | | | | designs. |
| boost | x | CH848.0358.80.06 | | FIGS. 29A, | |
| | | | | 29B | |
| boost | x | CH848.1432.5.41 | | FIGS. 29A, | |
| | | | | 29B | |
| boost | | CH848.1621.4.44 | CH0848.3.d1621.4.446R.DS.SOSIP.664_{—} | FIG. 28 | CH0848.3.d1621.4.446R.DS.SOSIP.664 |
| | | | CD5ss | | With various signal |
| | | | | | peptides |
| | | | | | Various protomer |
| | | | | | designs, including |
| | | | | | without limitation |
| | | | | | various stabilized |
| | | | | | designs |
| boost | | CH848.1305.10.35 | CH0848.3.d1305.10.356R.DS.SOSIP.664_{—} | FIG. 28 | CH0848.3.d1305.10.356R.DS.SOSIP.664 |
| | | | CD5ss | | With various signal |
| | | | | | peptides |
| | | | | | Various protomer |
| | | | | | designs, including |
| | | | | | without limitation |
| | | | | | various stabilized |
| | | | | | designs |
| boost | | P0402.c2.11 (G) | | FIG. 28 | Various protomer |
| | | | | | designs, including |
| | | | | | without limitation |
| | | | | | various stabilized |
| | | | | | designs |
| boost | | ZM246F (C) | | FIG. 28 | Various protomer |
| | | | | | designs, including |
| | | | | | without limitation |
| | | | | | various stabilized |
| | | | | | designs |

(x) indicates non-limiting embodiments of boost envelopes described in Table 3.

Throughout the specification, the name CH848.d0949.10.17 DT is interchangeably used as CH848.d0949.10.17.N133D.N138T. Throughout the specification, the name CH848.d0949.10.17 is interchangeably used as CH848.d0949.10.17WT. In certain embodiments, CH848.d0949.10.17DT envelope comprises additional modifications D230N.H289N.P291S.E169K and is referred to as CH848.d0949.10.17 DTe. In certain embodiments, CH848.d0949.10.17 envelope comprises additional modifications D230N.H289N.P291S.E169K and is referred to as CH848.d0949.10.17WTe.

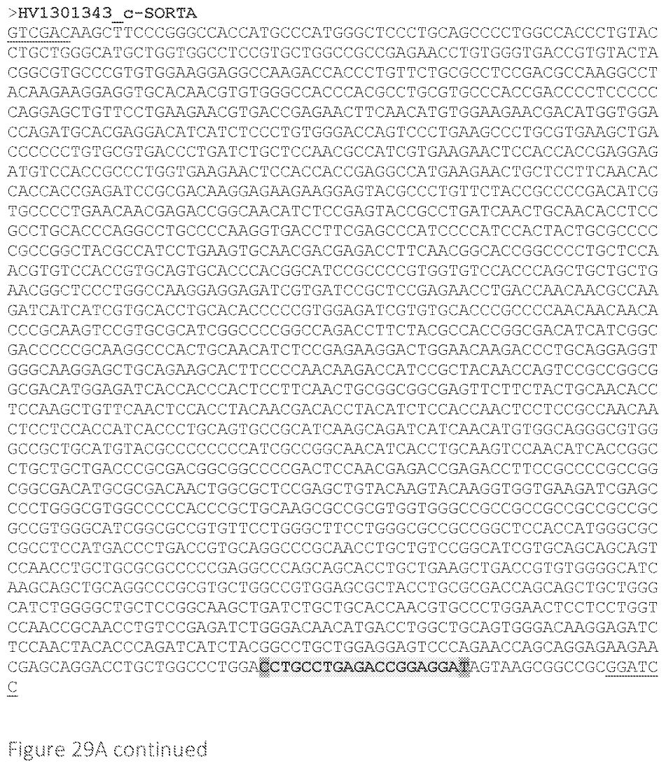

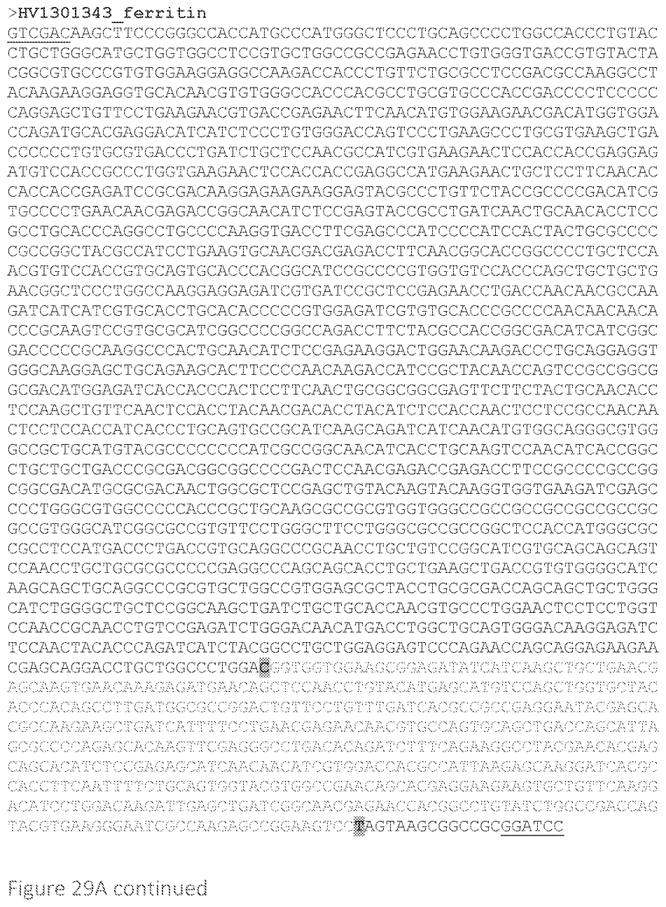

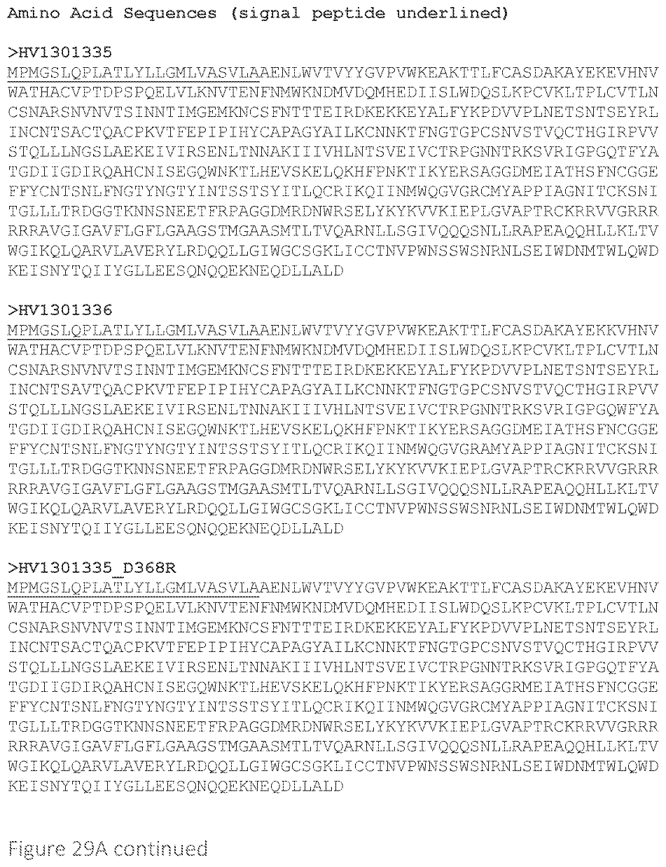

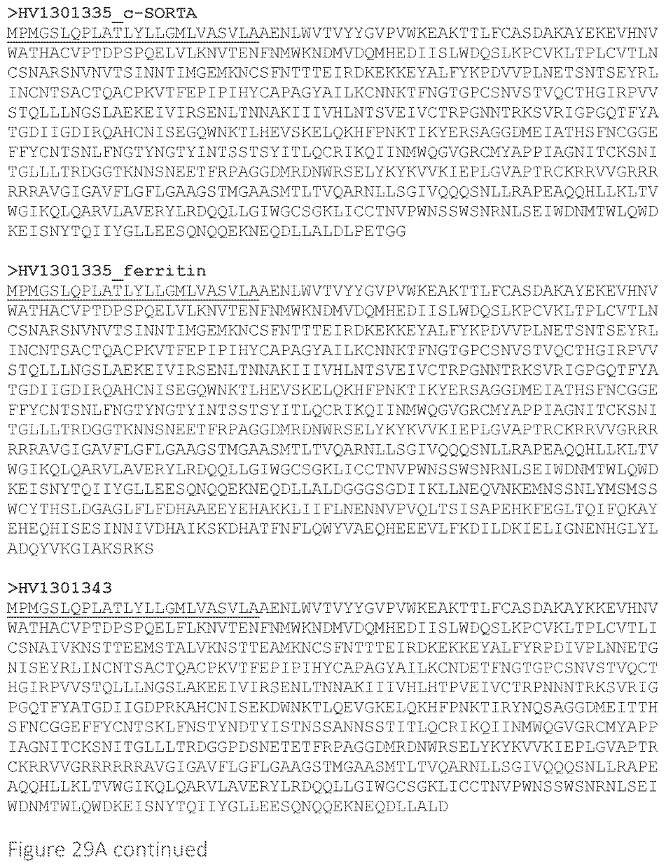

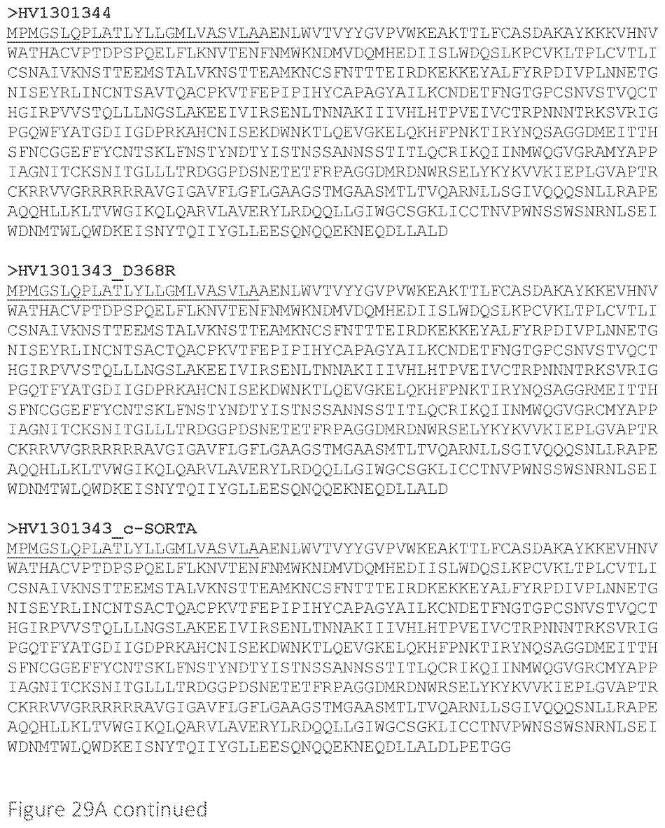

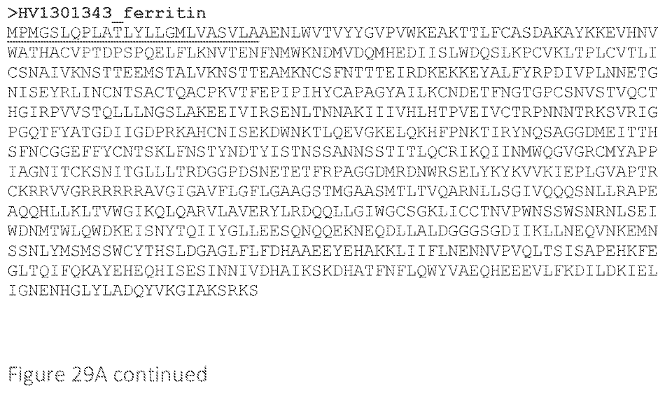

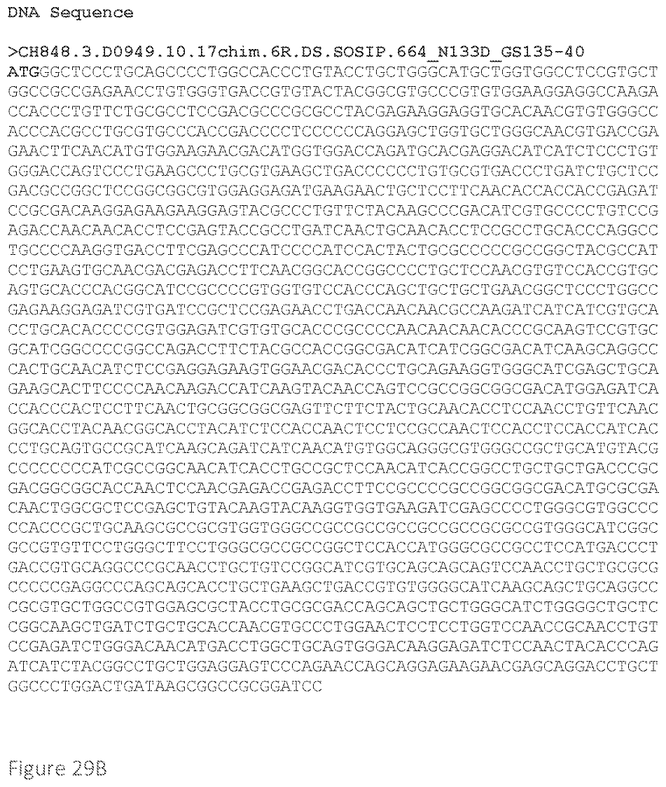

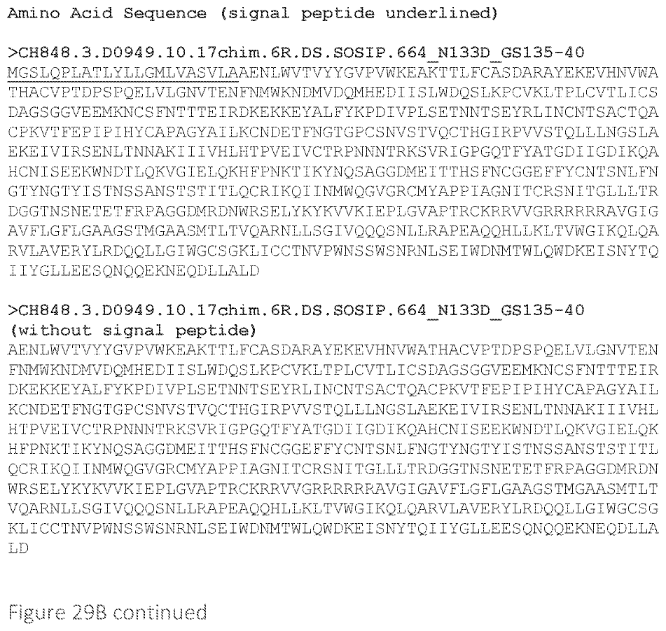

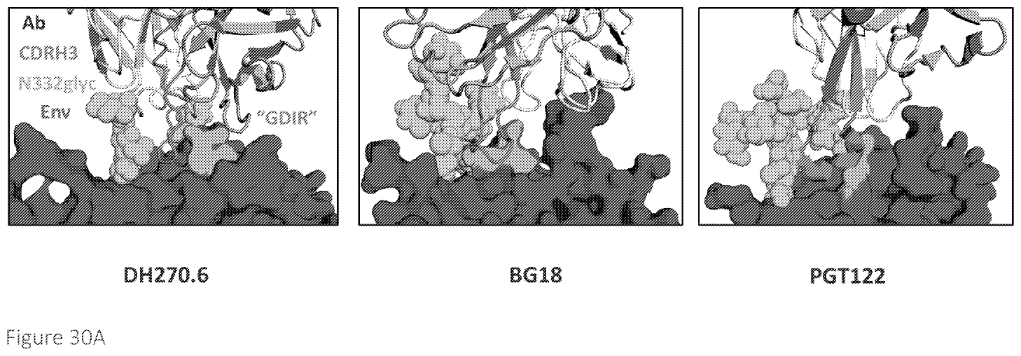

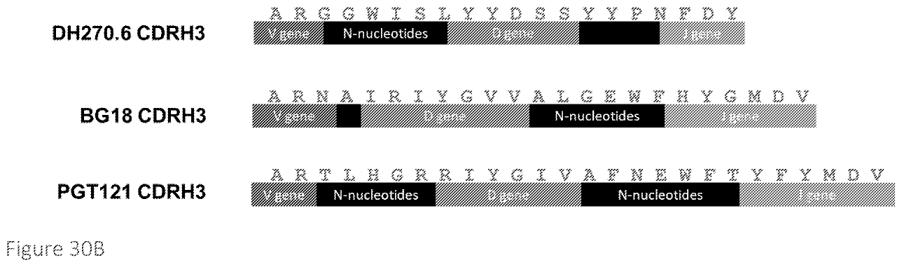

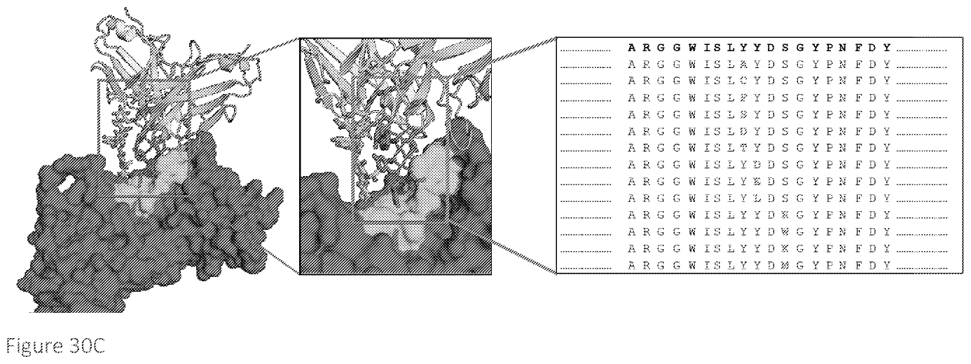

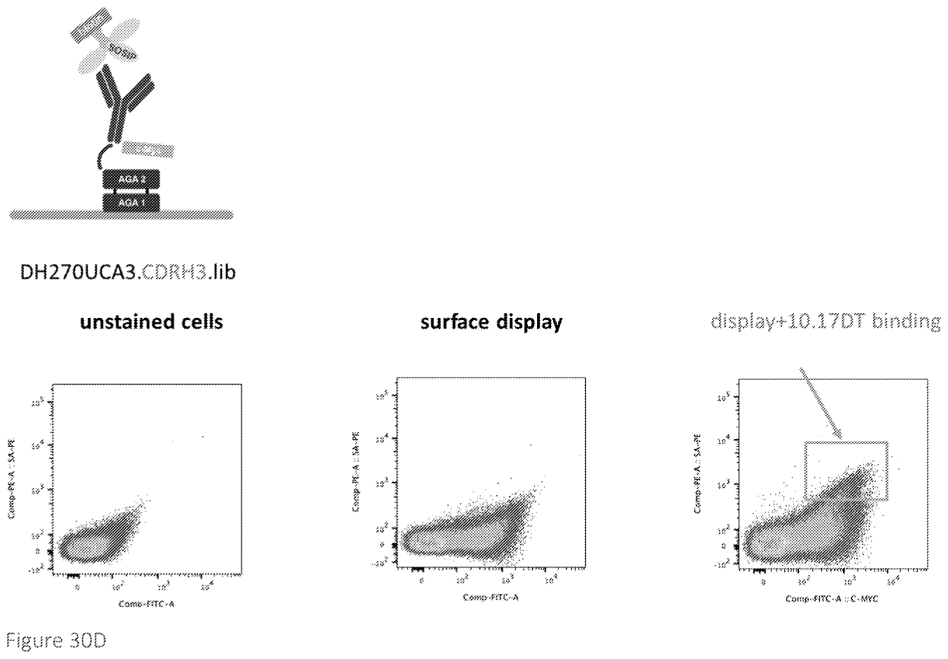

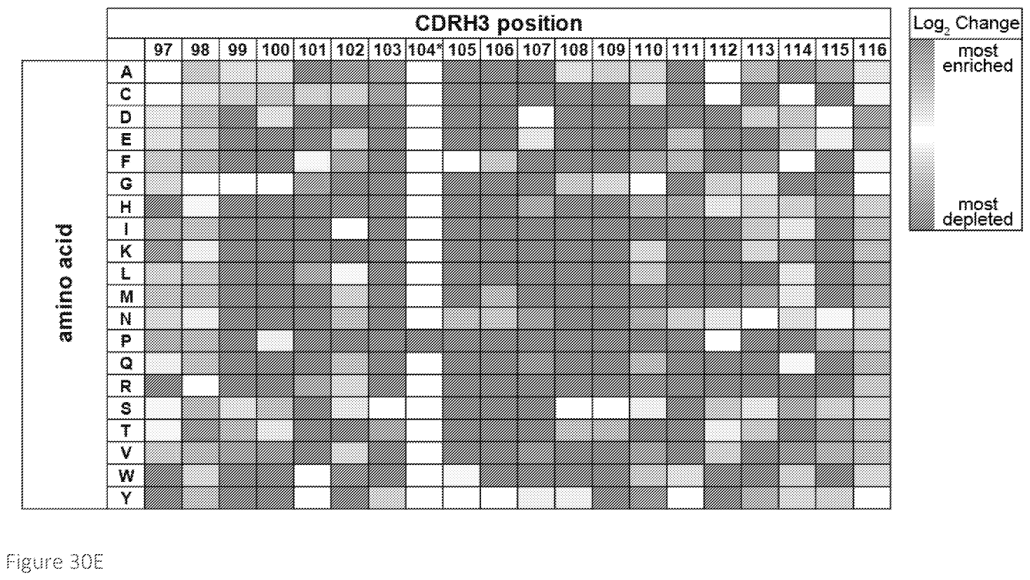

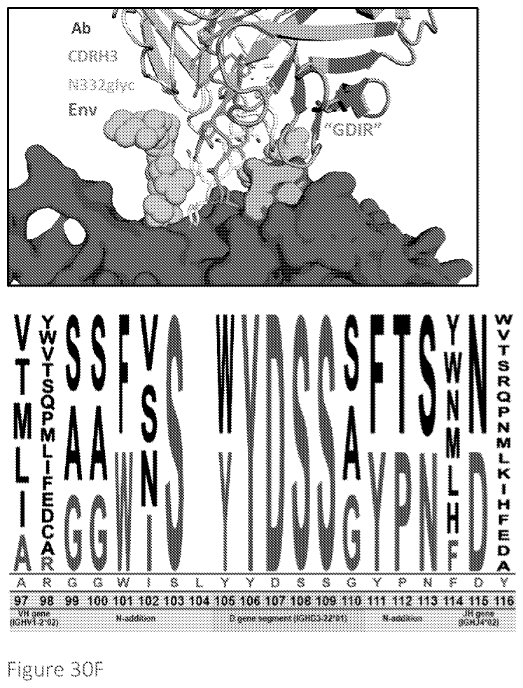

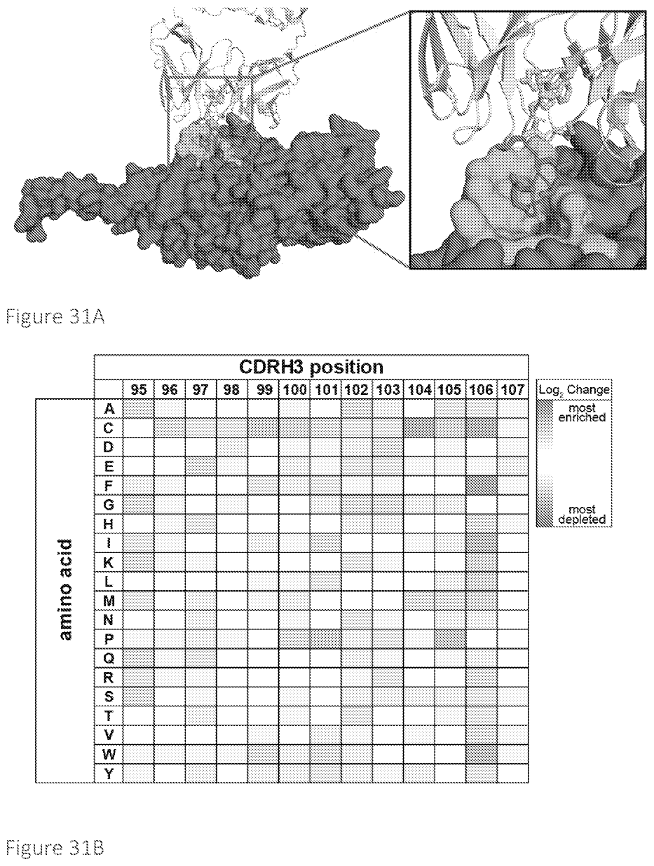

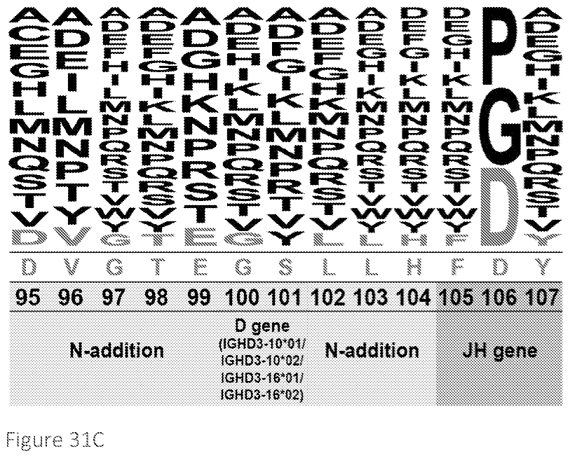

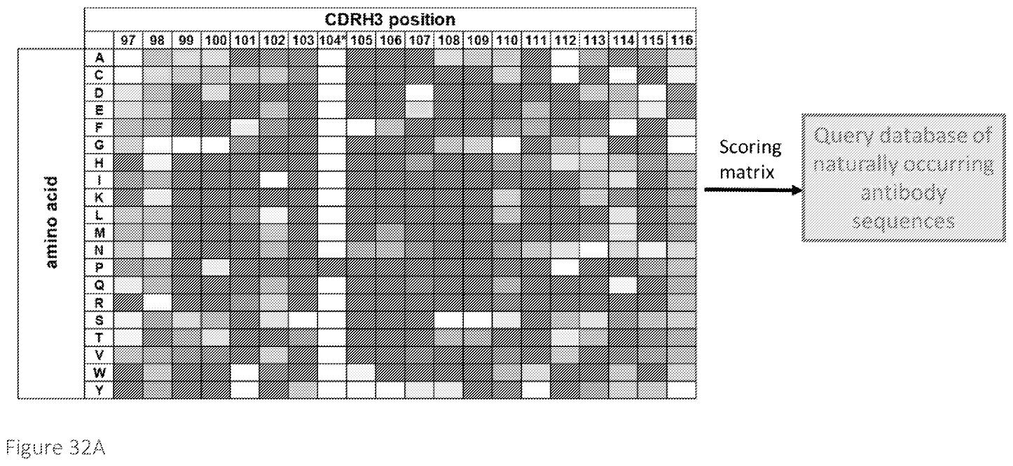

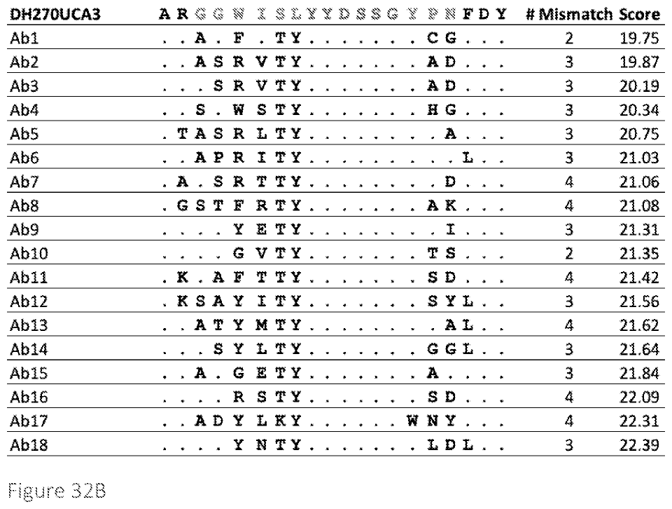

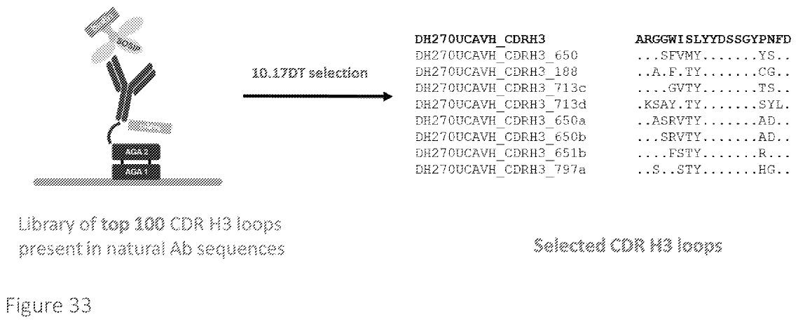

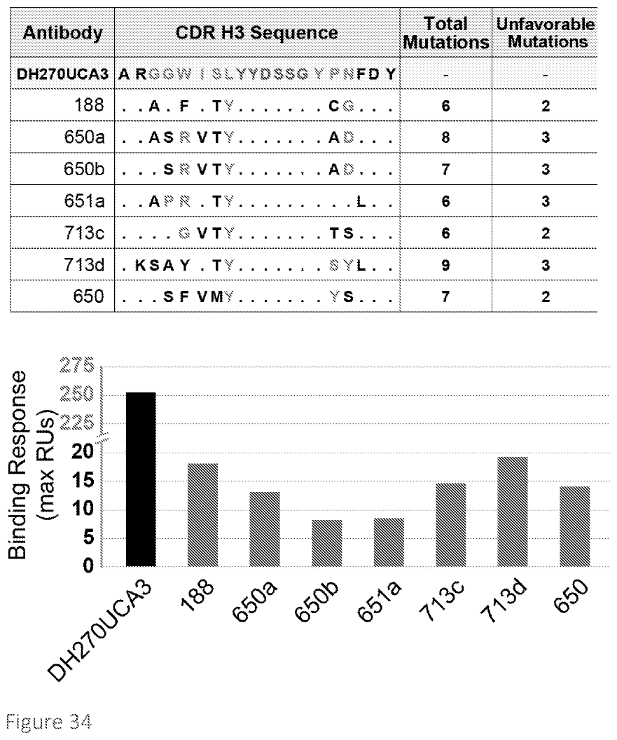

TABLE 5
Summary of 10.17 DT.GS protein envelope designs. See Example 5 and
FIGS. 30A-30F, 31A-31C, 32A-32B, 33, 34, 35A-35C, and 36A-36B.

| | | | | FIG. and |
|---|---|---|---|---|
| HV | Envelope to be used as protein | Linker | Ferritin for | paragraphs |
| name | and/or mRNA | sequence | multimerization | [0177]-[0202] |
| | 10.17 DT.GS protein trimer | no | No | FIG. 29B |
| | CH848.3.D0949.10.17chim.6R.DS.SOSIP.664_{—} | | | |
| | N133D_GS135-40 | | | |
| | 10.17 DT.GS protein sortase NP | For Sortase | Any Ferritin is | |
| | CH848.3.D0949.10.17chim.6R.DS.SOSIP.664_{—} | conjugation | added via | |
| | N133D_GS135-40_cSortA | | Sortase | |
| | | | conjugation at | |
| | | | C-terminus | |
| | 10.17 DT.GS protein NP | SGG | could be any | |
| | CH848.3.D0949.10.17chim.6R.DS.SOSIP.664_{—} | | suitable ferritin | |
| | N133D_GS135-40 | | | |
| | VRCferritin | | | |
| HV1301866 | CH848.3.D0949.10.17chim.6R.DS.SOSIP.664_{—} | | | |
| | N133D_GS135-40 | | | |
| HV1302670 | CH848.3.D0949.10.17_N133D_GS135- | | | |
| | 140_D230N_H289N_P291S_E169K_DS.chSOSIP | | | |
| HV1301925_{—} | CH848.3.D0949.10.17_N133D_GS135- | Any linker | Any suitable | |
| GS135-140 | 140_D230N_H289N_P291S_E169K_DS.SOSIP_{—} | | ferritin | |
| | VRCferritin | | | |
| HV1301925_{—} | CH848.3.D0949.10.17_N133D_135GNS_{—} | Any linker | Any suitable | |
| 135GNS | D230N_H289N_P291S_E169K_DS.SOSIP_{—} | | ferritin | |
| | VRCferritin | | | |
| HV1302671 | CH848.3.D0949.10.17_N133D_GS135- | Any linker | Any suitable | |
| | 140_N408A_N442A_D230N_H289N_P291S_{—} | | ferritin | |
| | E169K_DS.SOSIP_VRCferritin | | | |
| HV1302672 | CH848.3.D0949.10.17_N133D_135GNS_{—} | Any linker | Any suitable | |
| | N408A_N442A_D230N_H289N_P291S_{—} | | ferritin | |
| | E169K_DS.SOSIP_VRCferritin | | | |
| HV1302673 | CH848.3.D0949.10.17_N133D_GS135- | | | |
| | 140_D230N_H289N_P291S_E169K_{—} | | | |
| | DS.chSOSIP_10InQQ-avi | | | |
| HV1302674 | CH848.3.D0949.10.17_N133D_135GNS_{—} | | | |
| | D230N_H289N_P291S_E169K_DS.chSOSIP | | | |
| HV1302675 | CH848.3.D0949.10.17_N133D_135GNS_{—} | | | |
| | D230N_H289N_P291S_E169K_DS.chSOSIP_{—} | | | |
| | 10InQQ-avi | | | |
| HV1301866_{—} | CH848.3.D0949.10.17chim.6R.DS.SOSIP.664_{—} | | | |
| avi3_GN13 | N133D_GS135-40 avi3_GN135mut | | | |
| 5mut | | | | |
| HV1301866_{—} | CH848.3.D0949.10.17chim.6R.DS.SOSIP.664_{—} | | | |
| 3C8HtwST2_{—} | N133D_GS135- | | | |
| GN135mut | 40_3C8HtwST2_GN135mut | | | |
| HV1301866_{—} | CH848.3.D0949.10.17chim.6R.DS.SOSIP.664_{—} | | | |
| avi3 | N133D_GS135-40_avi3 | | | |
| HV1301866_{—} | CH848.3.D0949.10.17chim.6R.DS.SOSIP.664_{—} | | | |
| 3C8HtwST2 | N133D_GS135-40_3C8HtwST2 | | | |

Any suitable signal peptide could be used. In designs comprising ferritin for multimerization, any suitable linker could be used between the envelope sequence and a ferritin sequence.

Below paragraphs show non-limiting embodiments of DNA sequences of encoding proteins listed in Table 5 and non-limiting embodiments of amino acid sequences of proteins listed in Table 5. Table 5 encompasses sequences in paragraphs [0177]-[0202].

>HV1301866,CH848.3.D0949.10.17chim.6R.DS.SOSIP.664_N133D_GS135-40
ATGGGCTCCCTGCAGCCCCTGGCCACCCTGTACCTGCTGGGCATGCTGGTGGCCTCCGTGCTGGCCGCCGAGAAC
CTGTGGGTGACCGTGTACTACGGCGTGCCCGTGTGGAAGGAGGCCAAGACCACCCTGTTCTGCGCCTCCGACGCC
CGCGCCTACGAGAAGGAGGTGCACAACGTGTGGGCCACCCACGCCTGCGTGCCCACCGACCCCTCCCCCCAGGAG
CTGGTGCTGGGCAACGTGACCGAGAACTTCAACATGTGGAAGAACGACATGGTGGACCAGATGCACGAGGACATC
ATCTCCCTGTGGGACCAGTCCCTGAAGCCCTGCGTGAAGCTGACCCCCCTGTGCGTGACCCTGATCTGCTCCGAC
GCCGGCTCCGGCGGCGTGGAGGAGATGAAGAACTGCTCCTTCAACACCACCACCGAGATCCGCGACAAGGAGAAG
AAGGAGTACGCCCTGTTCTACAAGCCCGACATCGTGCCCCTGTCCGAGACCAACAACACCTCCGAGTACCGCCTG
ATCAACTGCAACACCTCCGCCTGCACCCAGGCCTGCCCCAAGGTGACCTTCGAGCCCATCCCCATCCACTACTGC
GCCCCCGCCGGCTACGCCATCCTGAAGTGCAACGACGAGACCTTCAACGGCACCGGCCCCTGCTCCAACGTGTCC
ACCGTGCAGTGCACCCACGGCATCCGCCCCGTGGTGTCCACCCAGCTGCTGCTGAACGGCTCCCTGGCCGAGAAG
GAGATCGTGATCCGCTCCGAGAACCTGACCAACAACGCCAAGATCATCATCGTGCACCTGCACACCCCCGTGGAG
ATCGTGTGCACCCGCCCCAACAACAACACCCGCAAGTCCGTGCGCATCGGCCCCGGCCAGACCTTCTACGCCACC
GGCGACATCATCGGCGACATCAAGCAGGCCCACTGCAACATCTCCGAGGAGAAGTGGAACGACACCCTGCAGAAG
GTGGGCATCGAGCTGCAGAAGCACTTCCCCAACAAGACCATCAAGTACAACCAGTCCGCCGGCGGCGACATGGAG
ATCACCACCCACTCCTTCAACTGCGGCGGCGAGTTCTTCTACTGCAACACCTCCAACCTGTTCAACGGCACCTAC
AACGGCACCTACATCTCCACCAACTCCTCCGCCAACTCCACCTCCACCATCACCCTGCAGTGCCGCATCAAGCAG
ATCATCAACATGTGGCAGGGCGTGGGCCGCTGCATGTACGCCCCCCCCATCGCCGGCAACATCACCTGCCGCTCC
AACATCACCGGCCTGCTGCTGACCCGCGACGGCGGCACCAACTCCAACGAGACCGAGACCTTCCGCCCCGCCGGC
GGCGACATGCGCGACAACTGGCGCTCCGAGCTGTACAAGTACAAGGTGGTGAAGATCGAGCCCCTGGGCGTGGCC
CCCACCCGCTGCAAGCGCCGCGTGGTGGGCCGCCGCCGCCGCCGCCGCGCCGTGGGCATCGGCGCCGTGTTCCTG
GGCTTCCTGGGCGCCGCCGGCTCCACCATGGGCGCCGCCTCCATGACCCTGACCGTGCAGGCCCGCAACCTGCTG
TCCGGCATCGTGCAGCAGCAGTCCAACCTGCTGCGCGCCCCCGAGGCCCAGCAGCACCTGCTGAAGCTGACCGTG
TGGGGCATCAAGCAGCTGCAGGCCCGCGTGCTGGCCGTGGAGCGCTACCTGCGCGACCAGCAGCTGCTGGGCATC
TGGGGCTGCTCCGGCAAGCTGATCTGCTGCACCAACGTGCCCTGGAACTCCTCCTGGTCCAACCGCAACCTGTCC
GAGATCTGGGACAACATGACCTGGCTGCAGTGGGACAAGGAGATCTCCAACTACACCCAGATCATCTACGGCCTG
CTGGAGGAGTCCCAGAACCAGCAGGAGAAGAACGAGCAGGACCTGCTGGCCCTGGACTGATAAGCGGCCGC
>HV1302670,CH848.3.D0949.10.17_N133D_GS135-
140_D230N_H289N_P291S_E169K_DS.chSOSIP
ATGCCTATGGGATCTCTGCAGCCTCTGGCCACACTGTACCTGCTGGGAATGCTGGTGGCTTCTGTGCTGGCCGCC
GAGAATCTGTGGGTCACAGTGTACTATGGCGTGCCCGTGTGGAAAGAGGCCAAGACCACACTGTTCTGCGCCTCC
GATGCCAGAGCCTACGAGAAAGAGGTGCACAACGTCTGGGCCACACACGCCTGTGTGCCTACCGATCCATCTCCT
CAAGAGCTGGTGCTGGGCAACGTGACCGAGAACTTCAACATGTGGAAGAACGACATGGTGGACCAGATGCACGAG
GACATCATCAGCCTGTGGGACCAGAGCCTGAAGCCTTGCGTGAAGCTGACCCCTCTGTGCGTGACCCTGATCTGT

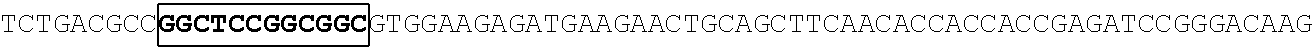

AAGAAGAAAGAGTACGCCCTGTTCTACAAGCCCGACATCGTGCCCCTGAGCGAGACAAACAACACCAGCGAGTAC
CGGCTGATCAACTGCAACACCTCCGCCTGCACTCAGGCCTGTCCTAAAGTGACCTTCGAGCCCATTCCTATCCAC
TACTGTGCCCCTGCCGGCTACGCCATCCTGAAGTGCAACAACGAGACATTCAACGGCACAGGCCCCTGCAGCAAT
GTGTCCACCGTGCAGTGTACCCACGGCATCAGACCAGTGGTGTCTACCCAGCTGCTGCTGAATGGAAGCCTGGCC
GAGAAAGAAATCGTGATCAGAAGCGAGAACCTGACCAACAACGCCAAGATCATCATCGTCCACCTGAATACCAGC
GTGGAAATCGTGTGCACCCGGCCTAACAACAACACCCGGAAGTCTGTGCGGATCGGCCCTGGCCAGACATTCTAT
GCCACCGGCGATATCATCGGCGACATCAAGCAGGCCCACTGCAACATCAGCGAGGAAAAGTGGAACGACACCCTG
CAGAAAGTGGGCATCGAGCTGCAGAAGCACTTCCCCAACAAGACCATCAAGTACAACCAGAGCGCTGGCGGCGAC
ATGGAAATCACCACACACAGCTTCAATTGTGGCGGCGAGTTCTTCTACTGCAATACCAGCAACCTGTTCAACGGG
ACCTACAATGGCACCIACATCAGCACCAACAGCAGCGCCAACTCCACCAGCACCATCACTCTGCAGTGCCGGATC
AAGCAGATCATCAATATGTGGCAAGGCGTCGGCCGGTGTATGTACGCCCCTCCTATCGCCGGCAACATCACCTGT
CGGAGCAATATCACAGGCCTGCTGCTCACCAGAGATGGCGGCACCAATAGCAACGAAACCGAAACCTTCAGACCT
GCCGGCGGAGACATGAGAGACAATTGGAGAAGCGAGCTGTACAAGTACAAGGTGGTCAAGATCGAGCCCCTGGGC
GTCGCACCTACACGGTGCAAGAGAAGAGTCGTGGGCCGTCGTAGAAGGCGGAGAGCCGTTGGAATTGGCGCCGTG
TTCCTGGGCTTTCTGGGAGCCGCTGGATCTACAATGGGCGCTGCCAGCATGACCCTGACAGTGCAGGCTAGAAAT
CTGCTGAGCGGCATCGTGCAGCAGCAGAGCAATCTGCTCAGAGCCCCTGAGGCTCAGCAGCACCTCCTGAAACTG
ACAGTGTGGGGAATCAAGCAGCTGCAGGCCAGAGTGCTGGCAGTGGAAAGATACCTGAGGGACCAGCAGCTCCTC
GGAATCTGGGGCTGTTCTGGCAAGCTGATCTGCTGCACCAACGTGCCCTGGAACAGCTCCTGGTCCAACAGAAAC
CTGTCCGAGATCTGGGATAACATGACCTGGCTGCAGTGGGACAAAGAGATCAGCAACTACACCCAGATCATCTAC
GGCCTGCTGGAAGAGAGCCAGAACCAGCAAGAGAAAAACGAGCAGGACCTGCTGGCCCTGGATTGATGA
>HV1301925_GS135-140,CH848.3.D0949.10.17_N133D_GS135-
140_D230N_H289N_P291S_E169K_DS.SOSIP_VRCferritin
ATGCCTATGGGATCTCTGCAGCCTCTGGCCACACTGTACCTGCTGGGAATGCTGGTGGCTTCTGTGCTGGCCGCC
GAGAATCTGTGGGTCACAGTGTACTATGGCGTGCCCGTGTGGAAAGAGGCCAAGACCACACTGTTCTGCGCCTCC
GATGCCAGAGCCTACGAGAAAGAGGTGCACAACGTCTGGGCCACACACGCCTGTGTGCCTACCGATCCATCTCCT
CAAGAGCTGGTGCTGGGCAACGTGACCGAGAACTTCAACATGTGGAAGAACGACATGGTGGACCAGATGCACGAG
GACATCATCAGCCTGTGGGACCAGAGCCTGAAGCCTTGCGTGAAGCTGACCCCTCTGTGCGTGACCCTGATCTGT

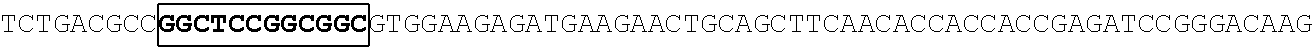

AAGAAGAAAGAGTACGCCCTGTTCTACAAGCCCGACATCGTGCCCCTGAGCGAGACAAACAACACCAGCGAGTAC
CGGCTGATCAACTGCAACACCTCCGCCTGCACTCAGGCCTGTCCTAAAGTGACCTTCGAGCCCATTCCTATCCAC
TACTGTGCCCCTGCCGGCTACGCCATCCTGAAGTGCAACAACGAGACATTCAACGGCACAGGCCCCTGCAGCAAT
GTGTCCACCGTGCAGTGTACCCACGGCATCAGACCAGTGGTGTCTACCCAGCTGCTGCTGAATGGAAGCCTGGCC
GAGAAAGAAATCGTGATCAGAAGCGAGAACCTGACCAACAACGCCAAGATCATCATCGTCCACCTGAATACCAGC
GTGGAAATCGTGTGCACCCGGCCTAACAACAACACCCGGAAGTCTGTGCGGATCGGCCCTGGCCAGACATTCTAT
GCCACCGGCGATATCATCGGCGACATCAAACAGGCCCACTGTAACATCAGCGAGGAAAAGTGGAACGACACCCTG
CAGAAAGTGGGCATCGAGCTGCAGAAGCACTTCCCCAACAAGACCATCAAGTACAACCAGAGCGCTGGCGGCGAC
ATGGAAATCACCACACACAGCTTCAATTGTGGCGGCGAGTTCTTCTACTGCAATACCAGCAACCTGTTCAACGGG
ACCTACAATGGCACCIACATCAGCACCAACAGCAGCGCCAACTCCACCAGCACCATCACTCTGCAGTGCCGGATC
AAGCAGATCATCAATATGTGGCAAGGCGTCGGCCGGTGTATGTACGCCCCTCCTATCGCCGGCAACATCACCTGT
CGGAGCAATATCACAGGCCTGCTGCTCACCAGAGATGGCGGCACCAATAGCAACGAAACCGAAACCTTCAGACCT
GCCGGCGGAGACATGAGAGACAATTGGAGAAGCGAGCTGTACAAGTACAAGGTGGTCAAGATCGAGCCCCTGGGC
GTCGCACCTACACGGTGCAAGAGAAGAGTCGTGGGCCGTCGTAGAAGGCGGAGAGCCGTTGGAATTGGCGCCGTG
TTCCTGGGCTTTCTGGGAGCCGCTGGATCTACAATGGGCGCTGCCAGCATGACCCTGACAGTGCAGGCTAGAAAT
CTGCTGAGCGGCATCGTGCAGCAGCAGAGCAATCTGCTCAGAGCCCCTGAGGCTCAGCAGCACCTCCTGAAACTG
ACAGTGTGGGGCATCAAGCAGCTGCAGGCAAGAGTGCTGGCAGTGGAAAGATACCTGCGGGACCAGCAGCTCCTC
GGAATCTGGGGATGTAGCGGCAAGCTGATCTGCTGCACCAACGTGCCCTGGAACAGCTCCTGGTCCAACAGAAAC
CTGTCCGAGATCTGGGATAACATGACCTGGCTGCAGTGGGACAAAGAGATCAGCAACTACACCCAGATCATCTAC
GGCCTGCTGGAAGAGAGCCAGAACCAGCAAGAGAAAAACGAGCAGGACCTGCTGGCCCTGGATTCTGGCGGAGAC
ATTATCAAGCTGCTGAACGAGCAAGTGAACAAAGAAATGCAGTCCTCCAACCTGTACATGAGCATGAGCAGCTGG
TGTTACACCCACAGCCTTGATGGCGCCGGACTGTTCCTGTTTGATCACGCCGCCGAGGAATACGAGCACGCCAAG
AAGCTGATCATCTTCCTGAACGAGAACAATGTGCCCGTGCAGCTGACCAGCATTAGCGCCCCAGAGCACAAGTTC
GAGGGCCTGACACAGATCTTTCAGAAGGCCTACGAACACGAGCAGCACATCTCCGAGAGCATCAACAACATCGTG
GACCACGCCATTAAGAGCAAGGATCACGCCACCTTCAATTTTCTGCAGTGGTACGTGGCCGAACAGCACGAGGAA
GAAGTGCTGTTCAAGGACATCCTGGACAAGATTGAGCTGATCGGCAACGAGAACCACGGCCTGTATCTGGCCGAC
CAGTACGTGAAGGGAATCGCCAAGAGCCGGAAGTCCGGCTCCTGATGA
>HV1301925_135GNS,CH848.3.D0949.10.17_N133D_135GNS_D230N_H289N_P
291S_E169K_DS.SOSIP_VRCferritin
ATGCCTATGGGATCTCTGCAGCCTCTGGCCACACTGTACCTGCTGGGAATGCTGGTGGCTTCTGTGCTGGCCGCC
GAGAATCTGTGGGTCACAGTGTACTATGGCGTGCCCGTGTGGAAAGAGGCCAAGACCACACTGTTCTGCGCCTCC
GATGCCAGAGCCTACGAGAAAGAGGTGCACAACGTCTGGGCCACACACGCCTGTGTGCCTACCGATCCATCTCCT
CAAGAGCTGGTGCTGGGCAACGTGACCGAGAACTTCAACATGTGGAAGAACGACATGGTGGACCAGATGCACGAG
GACATCATCAGCCTGTGGGACCAGAGCCTGAAGCCTTGCGTGAAGCTGACCCCTCTGTGCGTGACCCTGATCTGT

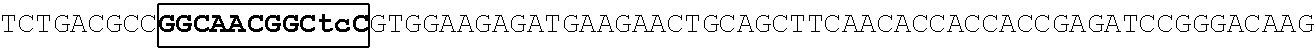

AAGAAGAAAGAGTACGCCCTGTTCTACAAGCCCGACATCGTGCCCCTGAGCGAGACAAACAACACCAGCGAGTAC
CGGCTGATCAACTGCAACACCTCCGCCTGCACTCAGGCCTGTCCTAAAGTGACCTTCGAGCCCATTCCTATCCAC
TACTGTGCCCCTGCCGGCTACGCCATCCTGAAGTGCAACAACGAGACATTCAACGGCACAGGCCCCTGCAGCAAT
GTGTCCACCGTGCAGTGTACCCACGGCATCAGACCAGTGGTGTCTACCCAGCTGCTGCTGAATGGAAGCCTGGCC
GAGAAAGAAATCGTGATCAGAAGCGAGAACCTGACCAACAACGCCAAGATCATCATCGTCCACCTGAATACCAGC
GTGGAAATCGTGTGCACCCGGCCTAACAACAACACCCGGAAGTCTGTGCGGATCGGCCCTGGCCAGACATTCTAT
GCCACCGGCGATATCATCGGCGACATCAAACAGGCCCACTGTAACATCAGCGAGGAAAAGTGGAACGACACCCTG
CAGAAAGTGGGCATCGAGCTGCAGAAGCACTTCCCCAACAAGACCATCAAGTACAACCAGAGCGCTGGCGGCGAC
ATGGAAATCACCACACACAGCTTCAATTGTGGCGGCGAGTTCTTCTACTGCAATACCAGCAACCTGTTCAACGGG
ACCTACAATGGCACCIACATCAGCACCAACAGCAGCGCCAACTCCACCAGCACCATCACTCTGCAGTGCCGGATC
AAGCAGATCATCAATATGTGGCAAGGCGTCGGCCGGTGTATGTACGCCCCTCCTATCGCCGGCAACATCACCTGT
CGGAGCAATATCACAGGCCTGCTGCTCACCAGAGATGGCGGCACCAATAGCAACGAAACCGAAACCTTCAGACCT
GCCGGCGGAGACATGAGAGACAATTGGAGAAGCGAGCTGTACAAGTACAAGGTGGTCAAGATCGAGCCCCTGGGC
GTCGCACCTACACGGTGCAAGAGAAGAGTCGTGGGCCGTCGTAGAAGGCGGAGAGCCGTTGGAATTGGCGCCGTG
TTCCTGGGCTTTCTGGGAGCCGCTGGATCTACAATGGGCGCTGCCAGCATGACCCTGACAGTGCAGGCTAGAAAT
CTGCTGAGCGGCATCGTGCAGCAGCAGAGCAATCTGCTCAGAGCCCCTGAGGCTCAGCAGCACCTCCTGAAACTG
ACAGTGTGGGGCATCAAGCAGCTGCAGGCAAGAGTGCTGGCAGTGGAAAGATACCTGCGGGACCAGCAGCTCCTC
GGAATCTGGGGATGTAGCGGCAAGCTGATCTGCTGCACCAACGTGCCCTGGAACAGCTCCTGGTCCAACAGAAAC
CTGTCCGAGATCTGGGATAACATGACCTGGCTGCAGTGGGACAAAGAGATCAGCAACTACACCCAGATCATCTAC
GGCCTGCTGGAAGAGAGCCAGAACCAGCAAGAGAAAAACGAGCAGGACCTGCTGGCCCTGGATTCTGGCGGAGAC
ATTATCAAGCTGCTGAACGAGCAAGTGAACAAAGAAATGCAGTCCTCCAACCTGTACATGAGCATGAGCAGCTGG
TGTTACACCCACAGCCTTGATGGCGCCGGACTGTTCCTGTTTGATCACGCCGCCGAGGAATACGAGCACGCCAAG
AAGCTGATCATCTTCCTGAACGAGAACAATGTGCCCGTGCAGCTGACCAGCATTAGCGCCCCAGAGCACAAGTTC
GAGGGCCTGACACAGATCTTTCAGAAGGCCTACGAACACGAGCAGCACATCTCCGAGAGCATCAACAACATCGTG
GACCACGCCATTAAGAGCAAGGATCACGCCACCTTCAATTTTCTGCAGTGGTACGTGGCCGAACAGCACGAGGAA
GAAGTGCTGTTCAAGGACATCCTGGACAAGATTGAGCTGATCGGCAACGAGAACCACGGCCTGTATCTGGCCGAC
CAGTACGTGAAGGGAATCGCCAAGAGCCGGAAGTCCGGCTCCTGATGA
>HV1302671, CH848.3.D0949.10.17_N133D_GS135-
140_N408A_N442A_D230N_H289N_P291S_E169K_DS.SOSIP_VRCferritin
ATGCCTATGGGATCTCTGCAGCCTCTGGCCACACTGTACCTGCTGGGAATGCTGGTGGCTTCTGTGCTGGCCGCC
GAGAATCTGTGGGTCACAGTGTACTATGGCGTGCCCGTGTGGAAAGAGGCCAAGACCACACTGTTCTGCGCCTCC
GATGCCAGAGCCTACGAGAAAGAGGTGCACAACGTCTGGGCCACACACGCCTGTGTGCCTACCGATCCATCTCCT
CAAGAGCTGGTGCTGGGCAACGTGACCGAGAACTTCAACATGTGGAAGAACGACATGGTGGACCAGATGCACGAG
GACATCATCAGCCTGTGGGACCAGAGCCTGAAGCCTTGCGTGAAGCTGACCCCTCTGTGCGTGACCCTGATCTGT

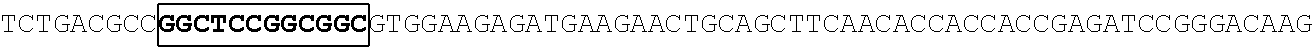

AAGAAGAAAGAGTACGCCCTGTTCTACAAGCCCGACATCGTGCCCCTGAGCGAGACAAACAACACCAGCGAGTAC
CGGCTGATCAACTGCAACACCTCCGCCTGCACTCAGGCCTGTCCTAAAGTGACCTTCGAGCCCATTCCTATCCAC
TACTGTGCCCCTGCCGGCTACGCCATCCTGAAGTGCAACAACGAGACATTCAACGGCACAGGCCCCTGCAGCAAT
GTGTCCACCGTGCAGTGTACCCACGGCATCAGACCAGTGGTGTCTACCCAGCTGCTGCTGAATGGAAGCCTGGCC
GAGAAAGAAATCGTGATCAGAAGCGAGAACCTGACCAACAACGCCAAGATCATCATCGTCCACCTGAATACCAGC
GTGGAAATCGTGTGCACCCGGCCTAACAACAACACCCGGAAGTCTGTGCGGATCGGCCCTGGCCAGACATTCTAT
GCCACCGGCGATATCATCGGCGACATCAAACAGGCCCACTGTAACATCAGCGAGGAAAAGTGGAACGACACCCTG
CAGAAAGTGGGCATCGAGCTGCAGAAGCACTTCCCCAACAAGACCATCAAGTACAACCAGAGCGCTGGCGGCGAC
ATGGAAATCACCACACACAGCTTCAATTGTGGCGGCGAGTTCTTCTACTGCAATACCAGCAACCTGTTCAACGGG
ACCTACAATGGCACCIACATCAGCACCAACAGCAGCGCCGCCTCCACCAGCACCATCACTCTGCAGTGCCGGATC
AAGCAGATCATCAATATGTGGCAAGGCGTCGGCCGGTGTATGTACGCCCCTCCTATCGCCGGCGCCATCACCTGT
CGGAGCAATATCACAGGCCTGCTGCTCACCAGAGATGGCGGCACCAATAGCAACGAAACCGAAACCTTCAGACCT
GCCGGCGGAGACATGAGAGACAATTGGAGAAGCGAGCTGTACAAGTACAAGGTGGTCAAGATCGAGCCCCTGGGC
GTCGCACCTACACGGTGCAAGAGAAGAGTCGTGGGCCGTCGTAGAAGGCGGAGAGCCGTTGGAATTGGCGCCGTG
TTCCTGGGCTTTCTGGGAGCCGCTGGATCTACAATGGGCGCTGCCAGCATGACCCTGACAGTGCAGGCTAGAAAT
CTGCTGAGCGGCATCGTGCAGCAGCAGAGCAATCTGCTCAGAGCCCCTGAGGCTCAGCAGCACCTCCTGAAACTG
ACAGTGTGGGGCATCAAGCAGCTGCAGGCAAGAGTGCTGGCAGTGGAAAGATACCTGCGGGACCAGCAGCTCCTC
GGAATCTGGGGATGTAGCGGCAAGCTGATCTGCTGCACCAACGTGCCCTGGAACAGCTCCTGGTCCAACAGAAAC
CTGTCCGAGATCTGGGATAACATGACCTGGCTGCAGTGGGACAAAGAGATCAGCAACTACACCCAGATCATCTAC
GGCCTGCTGGAAGAGAGCCAGAACCAGCAAGAGAAAAACGAGCAGGACCTGCTGGCCCTGGATTCTGGCGGAGAC
ATTATCAAGCTGCTGAACGAGCAAGTGAACAAAGAAATGCAGTCCTCCAACCTGTACATGAGCATGAGCAGCTGG
TGTTACACCCACAGCCTTGATGGCGCCGGACTGTTCCTGTTTGATCACGCCGCCGAGGAATACGAGCACGCCAAG
AAGCTGATCATCTTCCTGAACGAGAACAATGTGCCCGTGCAGCTGACCAGCATTAGCGCCCCAGAGCACAAGTTC
GAGGGCCTGACACAGATCTTTCAGAAGGCCTACGAACACGAGCAGCACATCTCCGAGAGCATCAACAACATCGTG
GACCACGCCATTAAGAGCAAGGATCACGCCACCTTCAATTTTCTGCAGTGGTACGTGGCCGAACAGCACGAGGAA
GAAGTGCTGTTCAAGGACATCCTGGACAAGATTGAGCTGATCGGCAACGAGAACCACGGCCTGTATCTGGCCGAC
CAGTACGTGAAGGGAATCGCCAAGAGCCGGAAGTCCGGCTCCTGATGA
>HV1302672,CH848.3.D0949.10.17 N133D_135GNS_N408A_N442A_D230N_H2
89N_P291S_E169K_DS.SOSIP_VRCferritin
ATGCCTATGGGATCTCTGCAGCCTCTGGCCACACTGTACCTGCTGGGAATGCTGGTGGCTTCTGTGCTGGCCGCC
GAGAATCTGTGGGTCACAGTGTACTATGGCGTGCCCGTGTGGAAAGAGGCCAAGACCACACTGTTCTGCGCCTCC
GATGCCAGAGCCTACGAGAAAGAGGTGCACAACGTCTGGGCCACACACGCCTGTGTGCCTACCGATCCATCTCCT
CAAGAGCTGGTGCTGGGCAACGTGACCGAGAACTTCAACATGTGGAAGAACGACATGGTGGACCAGATGCACGAG
GACATCATCAGCCTGTGGGACCAGAGCCTGAAGCCTTGCGTGAAGCTGACCCCTCTGTGCGTGACCCTGATCTGT

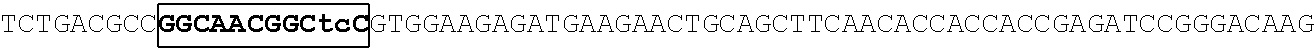

AAGAAGAAAGAGTACGCCCTGTTCTACAAGCCCGACATCGTGCCCCTGAGCGAGACAAACAACACCAGCGAGTAC
CGGCTGATCAACTGCAACACCTCCGCCTGCACTCAGGCCTGTCCTAAAGTGACCTTCGAGCCCATTCCTATCCAC
TACTGTGCCCCTGCCGGCTACGCCATCCTGAAGTGCAACAACGAGACATTCAACGGCACAGGCCCCTGCAGCAAT
GTGTCCACCGTGCAGTGTACCCACGGCATCAGACCAGTGGTGTCTACCCAGCTGCTGCTGAATGGAAGCCTGGCC
GAGAAAGAAATCGTGATCAGAAGCGAGAACCTGACCAACAACGCCAAGATCATCATCGTCCACCTGAATACCAGC
GTGGAAATCGTGTGCACCCGGCCTAACAACAACACCCGGAAGTCTGTGCGGATCGGCCCTGGCCAGACATTCTAT
GCCACCGGCGATATCATCGGCGACATCAAACAGGCCCACTGTAACATCAGCGAGGAAAAGTGGAACGACACCCTG
CAGAAAGTGGGCATCGAGCTGCAGAAGCACTTCCCCAACAAGACCATCAAGTACAACCAGAGCGCTGGCGGCGAC
ATGGAAATCACCACACACAGCTTCAATTGTGGCGGCGAGTTCTTCTACTGCAATACCAGCAACCTGTTCAACGGG
ACCTACAATGGCACCTACATCAGCACCAACAGCAGCGCCGCCTCCACCAGCACCATCACTCTGCAGTGCCGGATC
AAGCAGATCATCAATATGTGGCAAGGCGTCGGCCGGTGTATGTACGCCCCTCCTATCGCCGGCGCCATCACCTGT
CGGAGCAATATCACAGGCCTGCTGCTCACCAGAGATGGCGGCACCAATAGCAACGAAACCGAAACCTTCAGACCT
GCCGGCGGAGACATGAGAGACAATTGGAGAAGCGAGCTGTACAAGTACAAGGTGGTCAAGATCGAGCCCCTGGGC
GTCGCACCTACACGGTGCAAGAGAAGAGTCGTGGGCCGTCGTAGAAGGCGGAGAGCCGTTGGAATTGGCGCCGTG
TTCCTGGGCTTTCTGGGAGCCGCTGGATCTACAATGGGCGCTGCCAGCATGACCCTGACAGTGCAGGCTAGAAAT
CTGCTGAGCGGCATCGTGCAGCAGCAGAGCAATCTGCTCAGAGCCCCTGAGGCTCAGCAGCACCTCCTGAAACTG
ACAGTGTGGGGCATCAAGCAGCTGCAGGCAAGAGTGCTGGCAGTGGAAAGATACCTGCGGGACCAGCAGCTCCTC
GGAATCTGGGGATGTAGCGGCAAGCTGATCTGCTGCACCAACGTGCCCTGGAACAGCTCCTGGTCCAACAGAAAC
CTGTCCGAGATCTGGGATAACATGACCTGGCTGCAGTGGGACAAAGAGATCAGCAACTACACCCAGATCATCTAC
GGCCTGCTGGAAGAGAGCCAGAACCAGCAAGAGAAAAACGAGCAGGACCTGCTGGCCCTGGATTCTGGCGGAGAC
ATTATCAAGCTGCTGAACGAGCAAGTGAACAAAGAAATGCAGTCCTCCAACCTGTACATGAGCATGAGCAGCTGG
TGTTACACCCACAGCCTTGATGGCGCCGGACTGTTCCTGTTTGATCACGCCGCCGAGGAATACGAGCACGCCAAG
AAGCTGATCATCTTCCTGAACGAGAACAATGTGCCCGTGCAGCTGACCAGCATTAGCGCCCCAGAGCACAAGTTC
GAGGGCCTGACACAGATCTTTCAGAAGGCCTACGAACACGAGCAGCACATCTCCGAGAGCATCAACAACATCGTG
GACCACGCCATTAAGAGCAAGGATCACGCCACCTTCAATTTTCTGCAGTGGTACGTGGCCGAACAGCACGAGGAA
GAAGTGCTGTTCAAGGACATCCTGGACAAGATTGAGCTGATCGGCAACGAGAACCACGGCCTGTATCTGGCCGAC
CAGTACGTGAAGGGAATCGCCAAGAGCCGGAAGTCCGGCTCCTGATGA
>HV1302673,CH848.3.D0949.10.17_N133D_GS135-
140_D230N_H289N_P291S_E169K_DS.chSOSIP_10InQQ-avi
ATGCCTATGGGATCTCTGCAGCCTCTGGCCACACTGTACCTGCTGGGAATGCTGGTGGCTTCTGTGCTGGCCGCC
GAGAATCTGTGGGTCACAGTGTACTATGGCGTGCCCGTGTGGAAAGAGGCCAAGACCACACTGTTCTGCGCCTCC
GATGCCAGAGCCTACGAGAAAGAGGTGCACAACGTCTGGGCCACACACGCCTGTGTGCCTACCGATCCATCTCCT
CAAGAGCTGGTGCTGGGCAACGTGACCGAGAACTTCAACATGTGGAAGAACGACATGGTGGACCAGATGCACGAG
GACATCATCAGCCTGTGGGACCAGAGCCTGAAGCCTTGCGTGAAGCTGACCCCTCTGTGCGTGACCCTGATCTGT

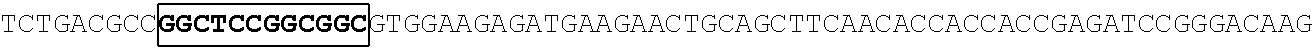

AAGAAGAAAGAGTACGCCCTGTTCTACAAGCCCGACATCGTGCCCCTGAGCGAGACAAACAACACCAGCGAGTAC
CGGCTGATCAACTGCAACACCTCCGCCTGCACTCAGGCCTGTCCTAAAGTGACCTTCGAGCCCATTCCTATCCAC
TACTGTGCCCCTGCCGGCTACGCCATCCTGAAGTGCAACAACGAGACATTCAACGGCACAGGCCCCTGCAGCAAT
GTGTCCACCGTGCAGTGTACCCACGGCATCAGACCAGTGGTGTCTACCCAGCTGCTGCTGAATGGAAGCCTGGCC
GAGAAAGAAATCGTGATCAGAAGCGAGAACCTGACCAACAACGCCAAGATCATCATCGTCCACCTGAATACCAGC
GTGGAAATCGTGTGCACCCGGCCTAACAACAACACCCGGAAGTCTGTGCGGATCGGCCCTGGCCAGACATTCTAT
GCCACCGGCGATATCATCGGCGACATCAAGCAGGCCCACTGCAACATCAGCGAGGAAAAGTGGAACGACACCCTG
CAGAAAGTGGGCATCGAGCTGCAGAAGCACTTCCCCAACAAGACCATCAAGTACAACCAGAGCGCTGGCGGCGAC
ATGGAAATCACCACACACAGCTTCAATTGTGGCGGCGAGTTCTTCTACTGCAATACCAGCAACCTGTTCAACGGG
ACCTACAATGGCACCTACATCAGCACCAACAGCAGCGCCAACTCCACCAGCACCATCACTCTGCAGTGCCGGATC
AAGCAGATCATCAATATGTGGCAAGGCGTCGGCCGGTGTATGTACGCCCCTCCTATCGCCGGCAACATCACCTGT
CGGAGCAATATCACAGGCCTGCTGCTCACCAGAGATGGCGGCACCAATAGCAACGAAACCGAAACCTTCAGACCT
GCCGGCGGAGACATGAGAGACAATTGGAGAAGCGAGCTGTACAAGTACAAGGTGGTCAAGATCGAGCCCCTGGGC
GTCGCACCTACACGGTGCAAGAGAAGAGTCGTGGGCCGTCGTAGAAGGCGGAGAGCCGTTGGAATTGGCGCCGTG
TTCCTGGGCTTTCTGGGAGCCGCTGGATCTACAATGGGCGCTGCCAGCATGACCCTGACAGTGCAGGCTAGAAAT
CTGCTGAGCGGCATCGTGCAGCAGCAGAGCAATCTGCTCAGAGCCCCTGAGGCTCAGCAGCACCTCCTGAAACTG
ACAGTGTGGGGAATCAAGCAGCTGCAGGCCAGAGTGCTGGCAGTGGAAAGATACCTGAGGGACCAGCAGCTCCTC
GGAATCTGGGGCTGTTCTGGCAAGCTGATCTGCTGCACCAACGTGCCCTGGAACAGCTCCTGGTCCAACAGAAAC
CTGTCCGAGATCTGGGATAACATGACCTGGCTGCAGTGGGACAAAGAGATCAGCAACTACACCCAGATCATCTAC

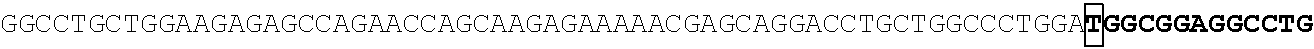

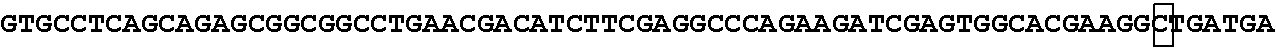

>HV1302674,CH848.3.D0949.10.17_N133D_135GNS_D230N_H289N_P291S_E16
9K_DS.chSOSIP
ATGCCTATGGGATCTCTGCAGCCTCTGGCCACACTGTACCTGCTGGGAATGCTGGTGGCTTCTGTGCTGGCCGCC
GAGAATCTGTGGGTCACAGTGTACTATGGCGTGCCCGTGTGGAAAGAGGCCAAGACCACACTGTTCTGCGCCTCC
GATGCCAGAGCCTACGAGAAAGAGGTGCACAACGTCTGGGCCACACACGCCTGTGTGCCTACCGATCCATCTCCT
CAAGAGCTGGTGCTGGGCAACGTGACCGAGAACTTCAACATGTGGAAGAACGACATGGTGGACCAGATGCACGAG
GACATCATCAGCCTGTGGGACCAGAGCCTGAAGCCTTGCGTGAAGCTGACCCCTCTGTGCGTGACCCTGATCTGT

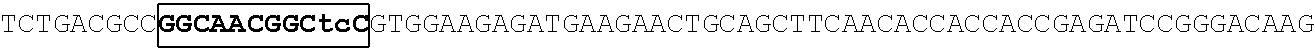

AAGAAGAAAGAGTACGCCCTGTTCTACAAGCCCGACATCGTGCCCCTGAGCGAGACAAACAACACCAGCGAGTAC
CGGCTGATCAACTGCAACACCTCCGCCTGCACTCAGGCCTGTCCTAAAGTGACCTTCGAGCCCATTCCTATCCAC
TACTGTGCCCCTGCCGGCTACGCCATCCTGAAGTGCAACAACGAGACATTCAACGGCACAGGCCCCTGCAGCAAT
GTGTCCACCGTGCAGTGTACCCACGGCATCAGACCAGTGGTGTCTACCCAGCTGCTGCTGAATGGAAGCCTGGCC
GAGAAAGAAATCGTGATCAGAAGCGAGAACCTGACCAACAACGCCAAGATCATCATCGTCCACCTGAATACCAGC
GTGGAAATCGTGTGCACCCGGCCTAACAACAACACCCGGAAGTCTGTGCGGATCGGCCCTGGCCAGACATTCTAT
GCCACCGGCGATATCATCGGCGACATCAAGCAGGCCCACTGCAACATCAGCGAGGAAAAGTGGAACGACACCCTG
CAGAAAGTGGGCATCGAGCTGCAGAAGCACTTCCCCAACAAGACCATCAAGTACAACCAGAGCGCTGGCGGCGAC
ATGGAAATCACCACACACAGCTTCAATTGTGGCGGCGAGTTCTTCTACTGCAATACCAGCAACCTGTTCAACGGG
ACCTACAATGGCACCTACATCAGCACCAACAGCAGCGCCAACTCCACCAGCACCATCACTCTGCAGTGCCGGATC
AAGCAGATCATCAATATGTGGCAAGGCGTCGGCCGGTGTATGTACGCCCCTCCTATCGCCGGCAACATCACCTGT
CGGAGCAATATCACAGGCCTGCTGCTCACCAGAGATGGCGGCACCAATAGCAACGAAACCGAAACCTTCAGACCT
GCCGGCGGAGACATGAGAGACAATTGGAGAAGCGAGCTGTACAAGTACAAGGTGGTCAAGATCGAGCCCCTGGGC
GTCGCACCTACACGGTGCAAGAGAAGAGTCGTGGGCCGTCGTAGAAGGCGGAGAGCCGTTGGAATTGGCGCCGTG
TTCCTGGGCTTTCTGGGAGCCGCTGGATCTACAATGGGCGCTGCCAGCATGACCCTGACAGTGCAGGCTAGAAAT
CTGCTGAGCGGCATCGTGCAGCAGCAGAGCAATCTGCTCAGAGCCCCTGAGGCTCAGCAGCACCTCCTGAAACTG
ACAGTGTGGGGAATCAAGCAGCTGCAGGCCAGAGTGCTGGCAGTGGAAAGATACCTGAGGGACCAGCAGCTCCTC
GGAATCTGGGGCTGTTCTGGCAAGCTGATCTGCTGCACCAACGTGCCCTGGAACAGCTCCTGGTCCAACAGAAAC
CTGTCCGAGATCTGGGATAACATGACCTGGCTGCAGTGGGACAAAGAGATCAGCAACTACACCCAGATCATCTAC
GGCCTGCTGGAAGAGAGCCAGAACCAGCAAGAGAAAAACGAGCAGGACCTGCTGGCCCTGGATTGATGA
>HIV1302675,CH848.3.D0949.10.17_N133D_135GNS_D230N_H289N_P291S_E16
9K_DS.chSOSIP_10InQQ-avi
ATGCCTATGGGATCTCTGCAGCCTCTGGCCACACTGTACCTGCTGGGAATGCTGGTGGCTTCTGTGCTGGCCGCC
GAGAATCTGTGGGTCACAGTGTACTATGGCGTGCCCGTGTGGAAAGAGGCCAAGACCACACTGTTCTGCGCCTCC
GATGCCAGAGCCTACGAGAAAGAGGTGCACAACGTCTGGGCCACACACGCCTGTGTGCCTACCGATCCATCTCCT
CAAGAGCTGGTGCTGGGCAACGTGACCGAGAACTTCAACATGTGGAAGAACGACATGGTGGACCAGATGCACGAG
GACATCATCAGCCTGTGGGACCAGAGCCTGAAGCCTTGCGTGAAGCTGACCCCTCTGTGCGTGACCCTGATCTGT

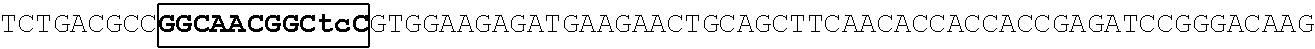

AAGAAGAAAGAGTACGCCCTGTTCTACAAGCCCGACATCGTGCCCCTGAGCGAGACAAACAACACCAGCGAGTAC
CGGCTGATCAACTGCAACACCTCCGCCTGCACTCAGGCCTGTCCTAAAGTGACCTTCGAGCCCATTCCTATCCAC
TACTGTGCCCCTGCCGGCTACGCCATCCTGAAGTGCAACAACGAGACATTCAACGGCACAGGCCCCTGCAGCAAT
GTGTCCACCGTGCAGTGTACCCACGGCATCAGACCAGTGGTGTCTACCCAGCTGCTGCTGAATGGAAGCCTGGCC
GAGAAAGAAATCGTGATCAGAAGCGAGAACCTGACCAACAACGCCAAGATCATCATCGTCCACCTGAATACCAGC
GTGGAAATCGTGTGCACCCGGCCTAACAACAACACCCGGAAGTCTGTGCGGATCGGCCCTGGCCAGACATTCTAT
GCCACCGGCGATATCATCGGCGACATCAAGCAGGCCCACTGCAACATCAGCGAGGAAAAGTGGAACGACACCCTG
CAGAAAGTGGGCATCGAGCTGCAGAAGCACTTCCCCAACAAGACCATCAAGTACAACCAGAGCGCTGGCGGCGAC
ATGGAAATCACCACACACAGCTTCAATTGTGGCGGCGAGTTCTICTACTGCAATACCAGCAACCTGTTCAACGGG
ACCTACAATGGCACCTACATCAGCACCAACAGCAGCGCCAACTCCACCAGCACCATCACTCTGCAGTGCCGGATC
AAGCAGATCATCAATATGTGGCAAGGCGTCGGCCGGTGTATGTACGCCCCTCCTATCGCCGGCAACATCACCTGT
CGGAGCAATATCACAGGCCTGCTGCTCACCAGAGATGGCGGCACCAATAGCAACGAAACCGAAACCTTCAGACCT
GCCGGCGGAGACATGAGAGACAATTGGAGAAGCGAGCTGTACAAGTACAAGGTGGTCAAGATCGAGCCCCTGGGC
GTCGCACCTACACGGTGCAAGAGAAGAGTCGTGGGCCGTCGTAGAAGGCGGAGAGCCGTTGGAATTGGCGCCGTG
TTCCTGGGCTTTCTGGGAGCCGCTGGATCTACAATGGGCGCTGCCAGCATGACCCTGACAGTGCAGGCTAGAAAT
CTGCTGAGCGGCATCGTGCAGCAGCAGAGCAATCTGCTCAGAGCCCCTGAGGCTCAGCAGCACCTCCTGAAACTG
ACAGTGTGGGGAATCAAGCAGCTGCAGGCCAGAGTGCTGGCAGTGGAAAGATACCTGAGGGACCAGCAGCTCCTC
GGAATCTGGGGCTGTTCTGGCAAGCTGATCTGCTGCACCAACGTGCCCTGGAACAGCTCCTGGTCCAACAGAAAC
CTGTCCGAGATCTGGGATAACATGACCTGGCTGCAGTGGGACAAAGAGATCAGCAACTACACCCAGATCATCTAC

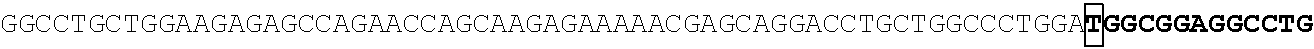

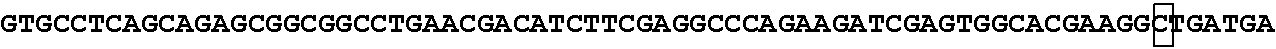

>HV1301866_3C8HtwST2,CH848.3.D0949.10.17chim.6R.DS.SOSIP.664_N133D_
GS135-40_3C8HtwST2
ATGGGCTCCCTGCAGCCCCTGGCCACCCTGTACCTGCTGGGCATGCTGGTGGCCTCCGTGCTGGCCGCCGAGAAC
CTGTGGGTGACCGTGTACTACGGCGTGCCCGTGTGGAAGGAGGCCAAGACCACCCTGTTCTGCGCCTCCGACGCC
CGCGCCTACGAGAAGGAGGTGCACAACGTGTGGGCCACCCACGCCTGCGTGCCCACCGACCCCTCCCCCCAGGAG
CTGGTGCTGGGCAACGTGACCGAGAACTTCAACATGTGGAAGAACGACATGGTGGACCAGATGCACGAGGACATC
ATCTCCCTGTGGGACCAGTCCCTGAAGCCCTGCGTGAAGCTGACCCCCCTGTGCGTGACCCTGATCTGCTCCGAC
GCCGGCTCCGGCGGCGTGGAGGAGATGAAGAACTGCTCCTTCAACACCACCACCGAGATCCGCGACAAGGAGAAG
AAGGAGTACGCCCTGTTCTACAAGCCCGACATCGTGCCCCTGTCCGAGACCAACAACACCTCCGAGTACCGCCTG
ATCAACTGCAACACCTCCGCCTGCACCCAGGCCTGCCCCAAGGTGACCTTCGAGCCCATCCCCATCCACTACTGC
GCCCCCGCCGGCTACGCCATCCTGAAGTGCAACGACGAGACCTTCAACGGCACCGGCCCCTGCTCCAACGTGTCC
ACCGTGCAGTGCACCCACGGCATCCGCCCCGTGGTGTCCACCCAGCTGCTGCTGAACGGCTCCCTGGCCGAGAAG
GAGATCGTGATCCGCTCCGAGAACCTGACCAACAACGCCAAGATCATCATCGTGCACCTGCACACCCCCGTGGAG
ATCGTGTGCACCCGCCCCAACAACAACACCCGCAAGTCCGTGCGCATCGGCCCCGGCCAGACCTTCTACGCCACC
GGCGACATCATCGGCGACATCAAGCAGGCCCACTGCAACATCTCCGAGGAGAAGTGGAACGACACCCTGCAGAAG
GTGGGCATCGAGCTGCAGAAGCACTTCCCCAACAAGACCATCAAGTACAACCAGTCCGCCGGCGGCGACATGGAG
ATCACCACCCACTCCTTCAACTGCGGCGGCGAGTTCTTCTACTGCAACACCTCCAACCTGTTCAACGGCACCTAC
AACGGCACCTACATCTCCACCAACTCCTCCGCCAACTCCACCTCCACCATCACCCTGCAGTGCCGCATCAAGCAG
ATCATCAACATGTGGCAGGGCGTGGGCCGCTGCATGTACGCCCCCCCCATCGCCGGCAACATCACCTGCCGCTCC
AACATCACCGGCCTGCTGCTGACCCGCGACGGCGGCACCAACTCCAACGAGACCGAGACCTTCCGCCCCGCCGGC
GGCGACATGCGCGACAACTGGCGCTCCGAGCTGTACAAGTACAAGGTGGTGAAGATCGAGCCCCTGGGCGTGGCC
CCCACCCGCTGCAAGCGCCGCGTGGTGGGCCGCCGCCGCCGCCGCCGCGCCGTGGGCATCGGCGCCGTGTTCCTG
GGCTTCCTGGGCGCCGCCGGCTCCACCATGGGCGCCGCCTCCATGACCCTGACCGTGCAGGCCCGCAACCTGCTG
TCCGGCATCGTGCAGCAGCAGTCCAACCTGCTGCGCGCCCCCGAGGCCCAGCAGCACCTGCTGAAGCTGACCGTG
TGGGGCATCAAGCAGCTGCAGGCCCGCGTGCTGGCCGTGGAGCGCTACCTGCGCGACCAGCAGCTGCTGGGCATC
TGGGGCTGCTCCGGCAAGCTGATCTGCTGCACCAACGTGCCCTGGAACTCCTCCTGGTCCAACCGCAACCTGTCC
GAGATCTGGGACAACATGACCTGGCTGCAGTGGGACAAGGAGATCTCCAACTACACCCAGATCATCTACGGCCTG
CTGGAGGAGTCCCAGAACCAGCAGGAGAAGAACGAGCAGGACCTGCTGGCCCTGGACCTGGAGGTGCTGTTCCAG
GGCCCAGGCCATCACCACCATCACCACCATCATAGCGCCTGGTCCCACCCCCAGTTCGAGAAGGGCGGCGGTAGT
GGAGGGGGCGGATCTGGCGGCTCAGCTTGGAGCCACCCCCAGTTCGAAAAGTGATAA
>HV1301866_avi3,CH848.3.D0949.10.17chim.6R.DS.SOSIP.664_N133D_GS135-
40_avi3
ATGGGCTCCCTGCAGCCCCTGGCCACCCTGTACCTGCTGGGCATGCTGGTGGCCTCCGTGCTGGCCGCCGAGAAC
CTGTGGGTGACCGTGTACTACGGCGTGCCCGTGTGGAAGGAGGCCAAGACCACCCTGTTCTGCGCCTCCGACGCC
CGCGCCTACGAGAAGGAGGTGCACAACGTGTGGGCCACCCACGCCTGCGTGCCCACCGACCCCTCCCCCCAGGAG
CTGGTGCTGGGCAACGTGACCGAGAACTTCAACATGTGGAAGAACGACATGGTGGACCAGATGCACGAGGACATC
ATCTCCCTGTGGGACCAGTCCCTGAAGCCCTGCGTGAAGCTGACCCCCCTGTGCGTGACCCTGATCTGCTCCGAC
GCCGGCTCCGGCGGCGTGGAGGAGATGAAGAACTGCTCCTTCAACACCACCACCGAGATCCGCGACAAGGAGAAG
AAGGAGTACGCCCTGTTCTACAAGCCCGACATCGTGCCCCTGTCCGAGACCAACAACACCTCCGAGTACCGCCTG
ATCAACTGCAACACCTCCGCCTGCACCCAGGCCTGCCCCAAGGTGACCTTCGAGCCCATCCCCATCCACTACTGC
GCCCCCGCCGGCTACGCCATCCTGAAGTGCAACGACGAGACCTTCAACGGCACCGGCCCCTGCTCCAACGTGTCC
ACCGTGCAGTGCACCCACGGCATCCGCCCCGTGGTGTCCACCCAGCTGCTGCTGAACGGCTCCCTGGCCGAGAAG
GAGATCGTGATCCGCTCCGAGAACCTGACCAACAACGCCAAGATCATCATCGTGCACCTGCACACCCCCGTGGAG
ATCGTGTGCACCCGCCCCAACAACAACACCCGCAAGTCCGTGCGCATCGGCCCCGGCCAGACCTTCTACGCCACC
GGCGACATCATCGGCGACATCAAGCAGGCCCACTGCAACATCTCCGAGGAGAAGTGGAACGACACCCTGCAGAAG
GTGGGCATCGAGCTGCAGAAGCACTTCCCCAACAAGACCATCAAGTACAACCAGTCCGCCGGCGGCGACATGGAG
ATCACCACCCACTCCTTCAACTGCGGCGGCGAGTTCTTCTACTGCAACACCTCCAACCTGTTCAACGGCACCTAC
AACGGCACCTACATCTCCACCAACTCCTCCGCCAACTCCACCTCCACCATCACCCTGCAGTGCCGCATCAAGCAG
ATCATCAACATGTGGCAGGGCGTGGGCCGCTGCATGTACGCCCCCCCCATCGCCGGCAACATCACCTGCCGCTCC
AACATCACCGGCCTGCTGCTGACCCGCGACGGCGGCACCAACTCCAACGAGACCGAGACCTTCCGCCCCGCCGGC
GGCGACATGCGCGACAACTGGCGCTCCGAGCTGTACAAGTACAAGGTGGTGAAGATCGAGCCCCTGGGCGTGGCC
CCCACCCGCTGCAAGCGCCGCGTGGTGGGCCGCCGCCGCCGCCGCCGCGCCGTGGGCATCGGCGCCGTGTTCCTG
GGCTTCCTGGGCGCCGCCGGCTCCACCATGGGCGCCGCCTCCATGACCCTGACCGTGCAGGCCCGCAACCTGCTG
TCCGGCATCGTGCAGCAGCAGTCCAACCTGCTGCGCGCCCCCGAGGCCCAGCAGCACCTGCTGAAGCTGACCGTG
TGGGGCATCAAGCAGCTGCAGGCCCGCGTGCTGGCCGTGGAGCGCTACCTGCGCGACCAGCAGCTGCTGGGCATC
TGGGGCTGCTCCGGCAAGCTGATCTGCTGCACCAACGTGCCCTGGAACTCCTCCTGGTCCAACCGCAACCTGTCC
GAGATCTGGGACAACATGACCTGGCTGCAGTGGGACAAGGAGATCTCCAACTACACCCAGATCATCTACGGCCTG
CTGGAGGAGTCCCAGAACCAGCAGGAGAAGAACGAGCAGGACCTGCTGGCCCTGGACGGAGGAGGCCTGGTGCCA
CAGCAGAGCGGAGGCCTGAACGACATCTTCGAGGCCCAGAAGATCGAGTGGCACGAGGGCTGATAA
>HV1301866_avi3_GN135mut,CH848.3.D0949.10.17chim.6R.DS.SOSIP.664_N13
3D_GS135-40_avi3_GN135mut
ATGGGCTCCCTGCAGCCCCTGGCCACCCTGTACCTGCTGGGCATGCTGGTGGCCTCCGTGCTGGCCGCCGAGAAC
CTGTGGGTGACCGTGTACTACGGCGTGCCCGTGTGGAAGGAGGCCAAGACCACCCTGTTCTGCGCCTCCGACGCC
CGCGCCTACGAGAAGGAGGTGCACAACGTGTGGGCCACCCACGCCTGCGTGCCCACCGACCCCTCCCCCCAGGAG
CTGGTGCTGGGCAACGTGACCGAGAACTTCAACATGTGGAAGAACGACATGGTGGACCAGATGCACGAGGACATC
ATCTCCCTGTGGGACCAGTCCCTGAAGCCCTGCGTGAAGCTGACCCCCCTGTGCGTGACCCTGATCTGCTCCGAC
GCCGGCAACGGCAGCGTGGAGGAGATGAAGAACTGCTCCTTCAACACCACCACCGAGATCCGCGACAAGGAGAAG
AAGGAGTACGCCCTGTICTACAAGCCCGACATCGTGCCCCTGTCCGAGACCAACAACACCTCCGAGTACCGCCTG
ATCAACTGCAACACCTCCGCCTGCACCCAGGCCTGCCCCAAGGTGACCTTCGAGCCCATCCCCATCCACTACTGC
GCCCCCGCCGGCTACGCCATCCTGAAGTGCAACGACGAGACCTTCAACGGCACCGGCCCCTGCTCCAACGTGTCC
ACCGTGCAGTGCACCCACGGCATCCGCCCCGTGGTGTCCACCCAGCTGCTGCTGAACGGCTCCCTGGCCGAGAAG
GAGATCGTGATCCGCTCCGAGAACCTGACCAACAACGCCAAGATCATCATCGTGCACCTGCACACCCCCGTGGAG
ATCGTGTGCACCCGCCCCAACAACAACACCCGCAAGTCCGTGCGCATCGGCCCCGGCCAGACCTICTACGCCACC
GGCGACATCATCGGCGACATCAAGCAGGCCCACTGCAACATCTCCGAGGAGAAGTGGAACGACACCCTGCAGAAG
GTGGGCATCGAGCTGCAGAAGCACTTCCCCAACAAGACCATCAAGTACAACCAGTCCGCCGGCGGCGACATGGAG
ATCACCACCCACTCCTTCAACTGCGGCGGCGAGTTCTTCTACTGCAACACCTCCAACCTGTTCAACGGCACCTAC
AACGGCACCTACATCTCCACCAACTCCTCCGCCAACTCCACCTCCACCATCACCCTGCAGTGCCGCATCAAGCAG
ATCATCAACATGTGGCAGGGCGTGGGCCGCTGCATGTACGCCCCCCCCATCGCCGGCAACATCACCTGCCGCTCC
AACATCACCGGCCTGCTGCTGACCCGCGACGGCGGCACCAACTCCAACGAGACCGAGACCTTCCGCCCCGCCGGC
GGCGACATGCGCGACAACTGGCGCTCCGAGCTGTACAAGTACAAGGTGGTGAAGATCGAGCCCCTGGGCGTGGCC
CCCACCCGCTGCAAGCGCCGCGTGGTGGGCCGCCGCCGCCGCCGCCGCGCCGTGGGCATCGGCGCCGTGTTCCTG
GGCTTCCTGGGCGCCGCCGGCTCCACCATGGGCGCCGCCTCCATGACCCTGACCGTGCAGGCCCGCAACCTGCTG
TCCGGCATCGTGCAGCAGCAGTCCAACCTGCTGCGCGCCCCCGAGGCCCAGCAGCACCTGCTGAAGCTGACCGTG
TGGGGCATCAAGCAGCTGCAGGCCCGCGTGCTGGCCGTGGAGCGCTACCTGCGCGACCAGCAGCTGCTGGGCATC
TGGGGCTGCTCCGGCAAGCTGATCTGCTGCACCAACGTGCCCTGGAACTCCTCCTGGTCCAACCGCAACCTGTCC
GAGATCTGGGACAACATGACCTGGCTGCAGTGGGACAAGGAGATCTCCAACTACACCCAGATCATCTACGGCCTG
CTGGAGGAGTCCCAGAACCAGCAGGAGAAGAACGAGCAGGACCTGCTGGCCCTGGACGGAGGAGGCCTGGTGCCA
CAGCAGAGCGGAGGCCTGAACGACATCTTCGAGGCCCAGAAGATCGAGTGGCACGAGGGCTGATAA
>HV1301866_3C8HtwST2_GN135mut,CH848.3.D0949.10.17chim.6R.
DS.SOSIP.664_N133D_GS135-40_3C8HtwST2_GN135mut
ATGGGCTCCCTGCAGCCCCTGGCCACCCTGTACCTGCTGGGCATGCTGGTGGCCTCCGTGCTGGCCGCCGAGAAC
CTGTGGGTGACCGTGTACTACGGCGTGCCCGTGTGGAAGGAGGCCAAGACCACCCTGTTCTGCGCCTCCGACGCC
CGCGCCTACGAGAAGGAGGTGCACAACGTGTGGGCCACCCACGCCTGCGTGCCCACCGACCCCTCCCCCCAGGAG
CTGGTGCTGGGCAACGTGACCGAGAACTTCAACATGTGGAAGAACGACATGGTGGACCAGATGCACGAGGACATC
ATCTCCCTGTGGGACCAGTCCCTGAAGCCCTGCGTGAAGCTGACCCCCCTGTGCGTGACCCTGATCTGCTCCGAC
GCCGGCAACGGCAGCGTGGAGGAGATGAAGAACTGCTCCTTCAACACCACCACCGAGATCCGCGACAAGGAGAAG
AAGGAGTACGCCCTGTTCTACAAGCCCGACATCGTGCCCCTGTCCGAGACCAACAACACCTCCGAGTACCGCCTG
ATCAACTGCAACACCTCCGCCTGCACCCAGGCCTGCCCCAAGGTGACCTTCGAGCCCATCCCCATCCACTACTGC
GCCCCCGCCGGCTACGCCATCCTGAAGTGCAACGACGAGACCTTCAACGGCACCGGCCCCTGCTCCAACGTGTCC
ACCGTGCAGTGCACCCACGGCATCCGCCCCGTGGTGTCCACCCAGCTGCTGCTGAACGGCTCCCTGGCCGAGAAG
GAGATCGTGATCCGCTCCGAGAACCTGACCAACAACGCCAAGATCATCATCGTGCACCTGCACACCCCCGTGGAG
ATCGTGTGCACCCGCCCCAACAACAACACCCGCAAGTCCGTGCGCATCGGCCCCGGCCAGACCTICTACGCCACC
GGCGACATCATCGGCGACATCAAGCAGGCCCACTGCAACATCTCCGAGGAGAAGTGGAACGACACCCTGCAGAAG
GTGGGCATCGAGCTGCAGAAGCACTTCCCCAACAAGACCATCAAGTACAACCAGTCCGCCGGCGGCGACATGGAG
ATCACCACCCACTCCTTCAACTGCGGCGGCGAGTTCTTCTACTGCAACACCTCCAACCTGTTCAACGGCACCTAC
AACGGCACCTACATCTCCACCAACTCCTCCGCCAACTCCACCTCCACCATCACCCTGCAGTGCCGCATCAAGCAG
ATCATCAACATGTGGCAGGGCGTGGGCCGCTGCATGTACGCCCCCCCCATCGCCGGCAACATCACCTGCCGCTCC
AACATCACCGGCCTGCTGCTGACCCGCGACGGCGGCACCAACTCCAACGAGACCGAGACCTTCCGCCCCGCCGGC
GGCGACATGCGCGACAACTGGCGCTCCGAGCTGTACAAGTACAAGGIGGTGAAGATCGAGCCCCTGGGCGTGGCC
CCCACCCGCTGCAAGCGCCGCGTGGTGGGCCGCCGCCGCCGCCGCCGCGCCGTGGGCATCGGCGCCGTGTTCCTG
GGCTTCCTGGGCGCCGCCGGCTCCACCATGGGCGCCGCCTCCATGACCCTGACCGTGCAGGCCCGCAACCTGCTG
TCCGGCATCGTGCAGCAGCAGTCCAACCTGCTGCGCGCCCCCGAGGCCCAGCAGCACCTGCTGAAGCTGACCGTG
TGGGGCATCAAGCAGCTGCAGGCCCGCGTGCTGGCCGTGGAGCGCTACCTGCGCGACCAGCAGCTGCTGGGCATC
TGGGGCTGCTCCGGCAAGCTGATCTGCTGCACCAACGTGCCCTGGAACTCCTCCTGGTCCAACCGCAACCTGTCC
GAGATCTGGGACAACATGACCTGGCTGCAGTGGGACAAGGAGATCTCCAACTACACCCAGATCATCTACGGCCTG
CTGGAGGAGTCCCAGAACCAGCAGGAGAAGAACGAGCAGGACCTGCTGGCCCTGGACCTGGAGGTGCTGTTCCAG
GGCCCAGGCCATCACCACCATCACCACCATCATAGCGCCTGGTCCCACCCCCAGTTCGAGAAGGGCGGCGGTAGT
GGAGGGGGCGGATCTGGCGGCTCAGCTTGGAGCCACCCCCAGTTCGAAAAGTGATAA
Amino Acid Sequence (signal peptide underlined)
Translate results
>HV1301866,CH848.3.D0949.10.17chim.6R.DS.SOSIP.664_N133D_GS135-40
MGSLQPLATLYLLGMLVASVLAAENLWVTVYYGVPVWKEAKTTLFCASDARAYEKEVHNVWATHACVPTDPSPQE
LVLGNVTENFNMWKNDMVDQMHEDIISLWDQSLKPCVKLTPLCVTLICSDAGSGGVEEMKNCSENTTTEIRDKEK
KEYALFYKPDIVPLSETNNTSEYRLINCNTSACTOACPKVTFEPIPIHYCAPAGYAILKCNDETENGTGPCSNVS
TVQCTHGIRPVVSTOLLINGSLAEKEIVIRSENLINNAKIIIVHLHTPVEIVCTRPNNNTRKSVRIGPGQTFYAT
GDIIGDIKQAHCNISEEKWNDTLQKVGIELQKHFPNKTIKYNQSAGGDMEITTHSFNCGGEFFYCNTSNLENGTY
NGTYISTNSSANSTSTITLQCRIKQIINMWQGVGRCMYAPPIAGNITCRSNITGLLLTRDGGINSNETETFRPAG
GDMRDNWRSELYKYKVVKIEPLGVAPTRCKRRVVGRRRRRRAVGIGAVELGFLGAAGSTMGAASMTLTVQARNLL
SGIVQQQSNLLRAPEAQQHLLKLTVWGIKQLQARVLAVERYLRDQQLLGIWGCSGKLICCTNVPWNSSWSNRNLS
EIWDNMTWLQWDKEISNYTQIIYGLLEESQNQQEKNEQDLLALD**AA
>HV1302670,CH848.3.D0949.10.17_N133D_GS135-
140_D230N_H289N_P291S_E169K_DS.chSOSIP
MPMGSLQPLATLYLLGMLVASVLAAENLWVTVYYGVPVWKEAKTTLFCASDARAYEKEVHNVWATHACVPTDPSP
QELVLGNVTENFNMWKNDMVDQMHEDIISLWDQSLKPCVKLTPLCVTLICSDAGSGGVEEMKNCSENTTTEIRDK
KKKEYALFYKPDIVPLSETNNTSEYRLINCNTSACTQACPKVTFEPIPIHYCAPAGYAILKCNNETENGTGPCSN
VSTVQCTHGIRPVVSTQLLINGSLAEKEIVIRSENLINNAKIIIVHLNTSVEIVCTRPNNNTRKSVRIGPGQTFY
ATGDIIGDIKQAHCNISEEKWNDTLQKVGIELQKHFPNKTIKYNQSAGGDMEITTHSENCGGEFFYCNTSNLFNG
TYNGTYISTNSSANSISTITLQCRIKQIINMWQGVGRCMYAPPIAGNITCRSNITGLLLTRDGGINSNETETERP
AGGDMRDNWRSELYKYKVVKIEPLGVAPTRCKRRVVGRRRRRRAVGIGAVELGFLGAAGSTMGAASMTLTVQARN
LLSGIVQQQSNLLRAPEAQQHLLKLTVWGIKQLQARVLAVERYLRDQQLLGIWGCSGKLICCTNVPWNSSWSNRN
LSEIWDNMTWLQWDKEISNYTQII YGLLEESQNQQEKNEQDLLALD**
>HV1301925_GS135-140,CH848.3.D0949.10.17_N133D_GS135-
140_D230N_H289N_P291S_E169K_DS.SOSIP_VRCferritin
MPMGSLQPLATLYLLGMLVASVLAAENLWVTVYYGVPVWKEAKTTLFCASDARAYEKEVHNVWATHACVPTDPSP
QELVIGNVTENFNMWKNDMVDQMHEDIISLWDQSLKPCVKLTPLCVTLICSDAGSGGVEEMKNCSFNITTEIRDK
KKKEYALFYKPDIVPLSETNNTSEYRLINCNTSACTQACPKVTFEPIPIHYCAPAGYAILKCNNETENGTGPCSN
VSTVQCTHGIRPVVSTQLLINGSLAEKEIVIRSENLINNAKIIIVHINTSVEIVCTRPNNNTRKSVRIGPGQTFY
ATGDIIGDIKQAHCNISEEKWNDTLQKVGIELQKHEPNKTIKYNQSAGGDMEITTHSFNCGGEFFYCNTSNLENG
TYNGTYISTNSSANSTSTITLQCRIKQIINMWQGVGRCMYAPPIAGNITCRSNITGLLLTRDGGINSNETETERP
AGGDMRDNWRSELYKYKVVKIEPLGVAPTRCKRRVVGRRRRRRAVGIGAVFLGFLGAAGSTMGAASMTLTVQARN
LLSGIVQQQSNLLRAPEAQQHLLKLTVWGIKQLQARVLAVERYLRDQQLLGIWGCSGKLICCTNVPWNSSWSNRN
LSEIWDNMTWLQWDKEISNYTQIIYGLLEESQNQQEKNEQDLLALDSGGDIIKLLNEQVNKEMQSSNLYMSMSSW
CYTHSLDGAGLFLFDHAAEEYEHAKKLIIFLNENNVPVQLTSISAPEHKFEGLTQIFQKAYEHEQHISESINNIV
DHAIKSKDHATENFLQWYVAEQHEEEVLEKDILDKIELIGNENHGLYLADQYVKGIAKSRKSGS**
>HV1301925_135GNS,CH848.3.D0949.10.17_N133D_135GNS_D230N_H289N_
P291S_E169K_DS.SOSIP_VRCferritin
MPMGSLQPLATLYLLGMLVASVLAAENLWVTVYYGVPVWKEAKTTLFCASDARAYEKEVHNVWATHACVPTDPSP
QELVLGNVTENFNMWKNDMVDQMHEDIISLWDQSLKPCVKLTPLCVTLICSDAGNGSVEEMKNCSENTTTEIRDK
KKKEYALFYKPDIVPLSETNNTSEYRLINCNTSACTQACPKVTFEPIPIHYCAPAGYAILKCNNETENGTGPCSN
VSTVQCTHGIRPVVSTQLLLNGSLAEKEIVIRSENLINNAKIIIVHINTSVEIVCTRPNNNTRKSVRIGPGQTFY
ATGDIIGDIKQAHCNISEEKWNDTLQKVGIELQKHFPNKTIKYNQSAGGDMEITTHSFNCGGEFFYCNTSNLFNG
TYNGTYISTNSSANSISTITLQCRIKQIINMWQGVGRCMYAPPIAGNITCRSNITGLLLTRDGGINSNETETERP
AGGDMRDNWRSELYKYKVVKIEPLGVAPTRCKRRVVGRRRRRRAVGIGAVELGFLGAAGSTMGAASMTLTVQARN
LLSGIVQQQSNLLRAPEAQQHLLKLTVWGIKQLQARVLAVERYLRDQQLLGIWGCSGKLICCTNVPWNSSWSNRN
LSEIWDNMTWLQWDKEISNYTQIIYGLLEESQNQQEKNEQDLLALDSGGDIIKLLNEQVNKEMQSSNLYMSMSSW
CYTHSLDGAGLFLFDHAAEEYEHAKKLIIFLNENNVPVQLTSISAPEHKFEGLTQIFQKAYEHEQHISESINNIV
DHAIKSKDHATENFLQWYVAEQHEEEVLFKDILDKIELIGNENHGLYLADQYVKGIAKSRKSGS**XXXXXXXXX
XMPMGSLQPLATLYLLGMLVASVLAAENLWVTVYYGVPVWKEAKTTLFCASDARAYEKEVHNVWATHACVPTDPS
PQELVLGNVTENFNMWKNDMVDQMHEDIISLWDQSLKPCVKLTPLCVTLICSDAGSGGVEEMKNCSENTTTEIRD
KKKKEYALFYKPDIVPLSETNNTSEYRLINCNTSACTQACPKVTFEPIPIHYCAPAGYAILKCNNETENGTGPCS
NVSTVQCTHGIRPVVSTQLLINGSLAEKEIVIRSENLINNAKIIIVHLNTSVEIVCTRPNNNTRKSVRIGPGQTF
YATGDIIGDIKQAHCNISEEKWNDTLQKVGIELQKHFPNKTIKYNQSAGGDMEITTHSENCGGEFFYCNTSNLEN
GTYNGTYISTNSSAASTSTITLQCRIKQIINMWQGVGRCMYAPPIAGAITCRSNITGLLLTRDGGINSNETETFR
PAGGDMRDNWRSELYKYKVVKIEPLGVAPTRCKRRVVGRRRRRRAVGIGAVELGFLGAAGSTMGAASMTLTVQAR
NLLSGIVQQQSNLLRAPEAQQHLLKLTVWGIKQLQARVLAVERYLRDQQLLGIWGCSGKLICCTNVPWNSSWSNR
NLSEIWDNMTWLQWDKEISNYTQIIYGLLEESQNQQEKNEQDLLALDSGGDIIKLLNEQVNKEMQSSNLYMSMSS
WCYTHSLDGAGLFLFDHAAEEYEHAKKLIIFLNENNVPVQLTSISAPEHKFEGLTQIFQKAYEHEQHISESINNI
VDHAIKSKDHATFNFLQWYVAEQHEEEVLFKDILDKIELIGNENHGLYLADQYVKGIAKSRKSGS**
>HV1302672,CH848.3.D0949.10.17_N133D_135GNS_N408A_N442A_D230N_
H289N_P291S_E169K_DS.SOSIP_VRCferritin
MPMGSLQPLATLYLLGMLVASVLAAENLWVTVYYGVPVWKEAKTTLFCASDARAYEKEVHNVWATHACVPTDPSP
QELVLGNVTENFNMWKNDMVDQMHEDIISLWDQSLKPCVKLTPLCVTLICSDAGNGSVEEMKNCSENTTTEIRDK
KKKEYALFYKPDIVPLSETNNTSEYRLINCNTSACTQACPKVTFEPIPIHYCAPAGYAILKCNNETENGTGPCSN
VSTVQCTHGIRPVVSTQLLINGSLAEKEIVIRSENLINNAKIIIVHLNTSVEIVCTRPNNNTRKSVRIGPGQTFY
ATGDIIGDIKQAHCNISEEKWNDTLQKVGIELQKHFPNKTIKYNQSAGGDMEITTHSENCGGEFFYCNTSNLENG
TYNGTYISTNSSAASTSTITLQCRIKQIINMWQGVGRCMYAPPIAGAITCRSNITGLLLTRDGGTNSNETETFRP
AGGDMRDNWRSELYKYKVVKIEPLGVAPTRCKRRVVGRRRRRRAVGIGAVFLGFLGAAGSTMGAASMTLTVQARN
LLSGIVQQQSNLLRAPEAQQHLLKLTVWGIKQLQARVLAVERYLRDQQLLGIWGCSGKLICCTNVPWNSSWSNRN
LSEIWDNMTWLQWDKEISNYTQIIYGLLEESQNQQEKNEQDLLALDSGGDIIKLLNEQVNKEMQSSNLYMSMSSW
CYTHSLDGAGLFLEDHAAEEYEHAKKLIIFLNENNVPVQLTSISAPEHKFEGLTQIFQKAYEHEQHISESINNIV
DHAIKSKDHATFNFLQWYVAEQHEEEVLFKDILDKIELIGNENHGLYLADQYVKGIAKSRKSGS**
>HV1302673,CH848.3.D0949.10.17_N133D_GS135-
140_D230N_H289N_P291S_E169K_DS.chSOSIP_10InQQ-avi
MPMGSLQPLATLYLLGMLVASVLAAENLWVTVYYGVPVWKEAKTTLFCASDARAYEKEVHNVWATHACVPTDPSP
QELVLGNVTENFNMWKNDMVDQMHEDIISLWDQSLKPCVKLTPLCVTLICSDAGSGGVEEMKNCSFNTTTEIRDK
KKKEYALFYKPDIVPLSETNNTSEYRLINCNTSACTQACPKVTFEPIPIHYCAPAGYAILKCNNETENGTGPCSN
VSTVQCTHGIRPVVSTQLLINGSLAEKEIVIRSENLINNAKIIIVHLNTSVEIVCTRPNNNTRKSVRIGPGQTFY
ATGDIIGDIKQAHCNISEEKWNDTLQKVGIELQKHFPNKTIKYNQSAGGDMEITTHSENCGGEFFYCNTSNLENG
TYNGTYISTNSSANSTSTITLQCRIKQIINMWQGVGRCMYAPPIAGNITCRSNITGLLLTRDGGINSNETETFRP
AGGDMRDNWRSELYKYKVVKIEPLGVAPTRCKRRVVGRRRRRRAVGIGAVELGFLGAAGSTMGAASMTLTVQARN
LLSGIVQQQSNLLRAPEAQQHLLKLTVWGIKQLQARVLAVERYLRDQQLLGIWGCSGKLICCTNVPWNSSWSNRN
LSEIWDNMTWLQWDKEISNYTQIIYGLLEESQNQQEKNEQDLLALDGGGLVPQQSGGLNDIFEAQKIEWHEG**
>HV1302674,CH848.3.D0949.10.17_N133D_135GNS_D230N_H289N_P291S_E
169K_DS.chSOSIP
MPMGSLQPLATLYLLGMLVASVLAAENLWVTVYYGVPVWKEAKTTLFCASDARAYEKEVHNVWATHACVPTDPSP
QELVLGNVTENFNMWKNDMVDQMHEDIISLWDQSLKPCVKLTPLCVTLICSDAGNGSVEEMKNCSENTTTEIRDK
KKKEYALFYKPDIVPLSETNNTSEYRLINCNTSACTQACPKVTFEPIPIHYCAPAGYAILKCNNETENGTGPCSN
VSTVQCTHGIRPVVSTQLLINGSLAEKEIVIRSENLINNAKIIIVHLNTSVEIVCTRPNNNTRKSVRIGPGQTFY
ATGDIIGDIKQAHCNISEEKWNDTLQKVGIELQKHFPNKTIKYNQSAGGDMEITTHSENCGGEFFYCNTSNLENG
TYNGTYISTNSSANSTSTITLQCRIKQIINMWQGVGRCMYAPPIAGNITCRSNITGLLLTRDGGINSNETETERP
AGGDMRDNWRSELYKYKVVKIEPLGVAPTRCKRRVVGRRRRRRAVGIGAVFLGFLGAAGSTMGAASMTLTVQARN
LLSGIVQQQSNLLRAPEAQQHLLKLTVWGIKQLQARVLAVERYLRDQQLLGIWGCSGKLICCTNVPWNSSWSNRN
LSEIWDNMTWLQWDKEISNYTQIIYGLLEESQNQQEKNEQDLLALD**
>HV1302675,CH848.3.D0949.10.17_N133D_135GNS_D230N_H289N_P291S_E
169K_DS.chSOSIP_10InQQ-avi
MPMGSLQPLATLYLLGMLVASVLAAENLWVTVYYGVPVWKEAKTTLFCASDARAYEKEVHNVWATHACVPTDPSP
QELVLGNVTENFNMWKNDMVDQMHEDIISLWDQSLKPCVKLTPLCVTLICSDAGNGSVEEMKNCSENTTTEIRDK
KKKEYALFYKPDIVPLSETNNTSEYRLINCNTSACTQACPKVTFEPIPIHYCAPAGYAILKCNNETFNGTGPCSN
VSTVQCTHGIRPVVSTQLLINGSLAEKEIVIRSENLINNAKIIIVHINTSVEIVCTRPNNNTRKSVRIGPGQTFY
ATGDIIGDIKQAHCNISEEKWNDTLQKVGIELQKHFPNKTIKYNQSAGGDMEITTHSFNCGGEFFYCNTSNLFNG
TYNGTYISTNSSANSISTITLQCRIKQIINMWQGVGRCMYAPPIAGNITCRSNITGLLLTRDGGINSNETETFRP
AGGDMRDNWRSELYKYKVVKIEPLGVAPTRCKRRVVGRRRRRRAVGIGAVELGFLGAAGSTMGAASMTLTVQARN
LLSGIVQQQSNLLRAPEAQQHLLKLTVWGIKQLQARVLAVERYLRDQQLLGIWGCSGKLICCTNVPWNSSWSNRN
LSEIWDNMTWLQWDKEISNYTQIIYGLLEESQNQQEKNEQDLLALDGGGLVPQQSGGLNDIFEAQKIEWHEG**
>HV1301866_3C8HtwST2,CH848.3.D0949.10.17chim.6R.DS.SOSIP.664_N133D_
GS135-40_3C8HtwST2
MGSLQPLATLYLLGMLVASVLAAENLWVTVYYGVPVWKEAKTTLFCASDARAYEKEVHNVWATHACVPTDPSPQE
LVLGNVTENFNMWKNDMVDQMHEDIISLWDQSLKPCVKLTPLCVTLICSDAGSGGVEEMKNCSENTTTEIRDKEK
KEYALFYKPDIVPLSEINNTSEYRLINCNTSACTQACPKVTFEPIPIHYCAPAGYAILKCNDETENGTGPCSNVS
TVQCTHGIRPVVSTQLLINGSLAEKEIVIRSENLINNAKIIIVHLHTPVEIVCTRPNNNTRKSVRIGPGQTFYAT
GDIIGDIKQAHCNISEEKWNDTLQKVGIELQKHFPNKTIKYNQSAGGDMEITTHSFNCGGEFFYCNTSNLENGTY
NGTYISTNSSANSTSTITLQCRIKQIINMWQGVGRCMYAPPIAGNITCRSNITGLLLTRDGGINSNETETFRPAG
GDMRDNWRSELYKYKVVKIEPLGVAPTRCKRRVVGRRRRRRAVGIGAVELGFLGAAGSTMGAASMTLTVQARNLL
SGIVQQQSNLLRAPEAQQHLLKLTVWGIKQLQARVLAVERYLRDQQLLGIWGCSGKLICCTNVPWNSSWSNRNLS
EIWDNMTWLQWDKEISNYTQIIYGLLEESQNQQEKNEQDLLALDLEVLFQGPGHHHHHHHHSAWSHPQFEKGGGS
GGGGSGGSAWSHPQFEK**
>HV1301866_avi3,CH848.3.D0949.10.17chim.6R.DS.SOSIP.664 N133D GS135-
40_avi3
MGSLQPLATLYLLGMLVASVLAAENLWVTVYYGVPVWKEAKTTLFCASDARAYEKEVHNVWATHACVPTDPSPQE
LVLGNVTENFNMWKNDMVDQMHEDIISLWDQSLKPCVKLTPLCVTLICSDAGSGGVEEMKNCSENTTTEIRDKEK
KEYALFYKPDIVPLSETNNTSEYRLINCNTSACTQACPKVTFEPIPIHYCAPAGYAILKCNDETENGTGPCSNVS
TVQCTHGIRPVVSTQLLINGSLAEKEIVIRSENLINNAKIIIVHLHTPVEIVCTRPNNNTRKSVRIGPGQTFYAT
GDIIGDIKQAHCNISEEKWNDTLQKVGIELQKHFPNKTIKYNQSAGGDMEITTHSENCGGEFFYCNTSNLENGTY
NGTYISTNSSANSTSTITLQCRIKQIINMWQGVGRCMYAPPIAGNITCRSNITGLLLTRDGGINSNETETFRPAG
GDMRDNWRSELYKYKVVKIEPLGVAPTRCKRRVVGRRRRRRAVGIGAVFLGFLGAAGSTMGAASMTLTVQARNLL
SGIVQQQSNLLRAPEAQQHLLKLTVWGIKQLQARVLAVERYLRDQQLLGIWGCSGKLICCTNVPWNSSWSNRNLS
EIWDNMTWLQWDKEISNYTQIIYGLLEESQNQQEKNEQDLLALDGGGLVPQQSGGLNDIFEAQKIEWHEG**
>HV1301866_avi3 GN135mut,CH848.3.D0949.10.17chim.6R.DS.SOSIP.664_N1
33D_GS135-40_avi3_GN135mut
MGSLQPLATLYLLGMLVASVLAAENLWVTVYYGVPVWKEAKTTLFCASDARAYEKEVHNVWATHACVPTDPSPQE
LVLGNVTENFNMWKNDMVDQMHEDIISLWDQSLKPCVKLTPLCVTLICSDAGNGSVEEMKNCSENTTTEIRDKEK
KEYALFYKPDIVPLSETNNTSEYRLINCNTSACTQACPKVTFEPIPIHYCAPAGYAILKCNDETENGTGPCSNVS
TVQCTHGIRPVVSTQLLINGSLAEKEIVIRSENLINNAKIIIVHLHTPVEIVCTRPNNNTRKSVRIGPGQTFYAT
GDIIGDIKQAHCNISEEKWNDTLQKVGIELQKHFPNKTIKYNQSAGGDMEITTHSFNCGGEFFYCNTSNLENGTY
NGTYISTNSSANSTSTITLQCRIKQIINMWQGVGRCMYAPPIAGNITCRSNITGLLLTRDGGINSNETETERPAG
GDMRDNWRSELYKYKVVKIEPLGVAPTRCKRRVVGRRRRRRAVGIGAVFLGFLGAAGSTMGAASMTLTVQARNLL
SGIVQQQSNLLRAPEAQQHLLKLTVWGIKQLQARVLAVERYLRDQQLLGIWGCSGKLICCTNVPWNSSWSNRNLS
ETWDNMTWLQWDKEISNYTQIIYGLLEESQNQQEKNEQDLLALDGGGLVPQQSGGLNDIFEAQKIEWHEG**
>HV1301866_3C8HtwST2_GN135mut,CH848.3.D0949.10.17chim.6R.DS.SOSIP.
664_N133D_GS135-40_3C8HtwST2_GN135mut
MGSLQPLATLYLLGMLVASVLAAENLWVTVYYGVPVWKEAKTTLFCASDARAYEKEVHNVWATHACVPTDPSPQE
LVLGNVTENENMWKNDMVDQMHEDIISLWDQSLKPCVKLTPLCVTLICSDAGNGSVEEMKNCSENTTTEIRDKEK
KEYALFYKPDIVPLSETNNTSEYRLINCNTSACTQACPKVTFEPIPIHYCAPAGYAILKCNDETENGTGPCSNVS
TVQCTHGIRPVVSTQLLINGSLAEKEIVIRSENLINNAKIIIVHLHTPVEIVCTRPNNNTRKSVRIGPGQTFYAT
GDIIGDIKQAHCNISEEKWNDTLQKVGIELQKHFPNKTIKYNQSAGGDMEITTHSENCGGEFFYCNTSNLENGTY
NGTYISTNSSANSTSTITLQCRIKQIINMWQGVGRCMYAPPIAGNITCRSNITGLLLTRDGGINSNETETFRPAG
GDMRDNWRSELYKYKVVKIEPLGVAPTRCKRRVVGRRRRRRAVGIGAVELGFLGAAGSTMGAASMTLTVQARNLL
SGIVQQQSNLLRAPEAQQHLLKLTVWGIKQLQARVLAVERYLRDQQLLGIWGCSGKLICCTNVPWNSSWSNRNLS
EIWDNMTWLQWDKEISNYTQIIYGLLEESQNQQEKNEQDLLALDLEVLFQGPGHHHHHHHHSAWSHPQFEKGGGS
GGGGSGGSAWSHPQFEK**

### EXAMPLES

### Example 1: Pan-bnAb-Engaging Immunogens

This example describes design of HIV-1 envelopes antigenic for cross-epitope bnAb UCAs.

The discovery of broadly neutralizing antibodies (bnAbs) in HIV-1 infected individuals has provided evidence that the human immune system can target highly conserved epitopes on HIV-1 envelope. However, bnAbs have not been reproducibly induced with a vaccine in primates. One approach to improve the induction of bnAbs is to specifically design immunogens that bind to the precursor B cell that gives rise to the bnAb. While highly affinity matured HIV-1 bnAbs react with many Envelope proteins, their precursors bind only to select Envs. Currently, immunogens exist that can bind to a single bnAb precursor. These Envs have the disadvantage of relying on a single bnAb precursor to be present in most individuals. If the bnAb precursor antibody is not present in that individual, then the vaccine will not have the intended effect of inducing a specific type of antibody response. To improve the chances that an individual has the bnAb precursor that can engage the vaccine immunogen, we created a vaccine immunogen that can bind to multiple bnAb precursors. We designed the immunogen to interact with bnAbs precursors that interact with the first and second variable loop and glycans proximal to this loop—an epitope called VIV2-glycan. Secondly, the immunogen was also designed to interact with a bnAb precursor that bound to the third variable region and surrounding glycans on HIV-1 envelope—the V3-glycan site.

The immunogen was designed by creating a chimera of two HIV-1 envelope sequences that were derived from the HIV-1 infected individual CH0848 (See WO/2017152146 and WO/2018161049). The first Env CH0848.3.D0949.10.17 is antigenic for V3-glycan antibodies and was selected because it had a short first variable region in Env and bound to a V3-glycan antibody that possessed only 5 mutations (Bonsignori et al STM 2017). We modified this Env by removing glycosylation sites at 133 and 138 and found V3-glycan antibodies bound better to the Env when the glycosylation site was removed. These two glycosylation sites were identified as inhibitory in a neutralization screen where glycosylation sites on Env were removed to determine which glycans were required for neutralization by V3-glycan antibodies. For the CH0848.3 D0949.10.17 envelope we removed the glycosylation by substituting asparagine for amino acids that normally occur at positions 133 and 138 in other viruses. This glycan-modified Env bound with low nanomolar affinity to the V3-glycan bnAb precursor DH270 UCA3. To determine if a similar Env may have been present in the infected individual and could have potentially initiated the V3-glycan lineage in vivo, we screened all of the autologous virus sequences isolated from the infected individual CH0848 for viruses with a 17 amino acid variable region 1 and no glycans within the variable region except at position 156. We identified two sequences, with these characteristics. The first sequence CH0848.3.D1305.10.19 was produced as a recombinant protein. In biolayer interferometry assays it did not bind to V3-glycan antibodies. We created a pseudovirus expressing this Env and also found that V3 glycan antibodies did not neutralize it. However, we found that V1V2-glycan antibodies could bind to the recombinant protein. This was in contrast to CH0848.3.D0949.10.17 which lacked binding to VIV2-glycan bnAbs and precursors but was antigenic for V3-glycan antibodies. We inspected the sequences of the VIV2 and V3 regions and found that CH0848.3.D1305.10.19 lacked three glycans at positions 295, 301, and 332 usually bound by V3-glycan antibodies. To restore these V3 proximal glycosylation sites in CH0848.3.D1305.10.19 we used the V3 sequence of CH0848.3.D0949.10.17—the new envelope referenced as 19CV3. The modification of the CH0848.3.D1305.10.19 sequence to 19CV3 resulted in the addition of glycosylation sites at positions 301 and 332. We again made a recombinant protein of the chimeric envelope and found it bound to VIV2-glycan bnAbs as well as V3-glycan bnAbs—a combination of the phenotypes of the two parental envelopes. We next tested the binding of the bnAb precursors for V1V2 and V3-glycan sites. We found that 19CV3 bout to the bn Ab precursor for two VIV2 glycan bnAb, CH01 and VRC26, and V3 glycan Ab DH270.

With reference to CH0848 10.17DT SOSIP sequence see WO2018/161049, incorporated by reference in its entirety.

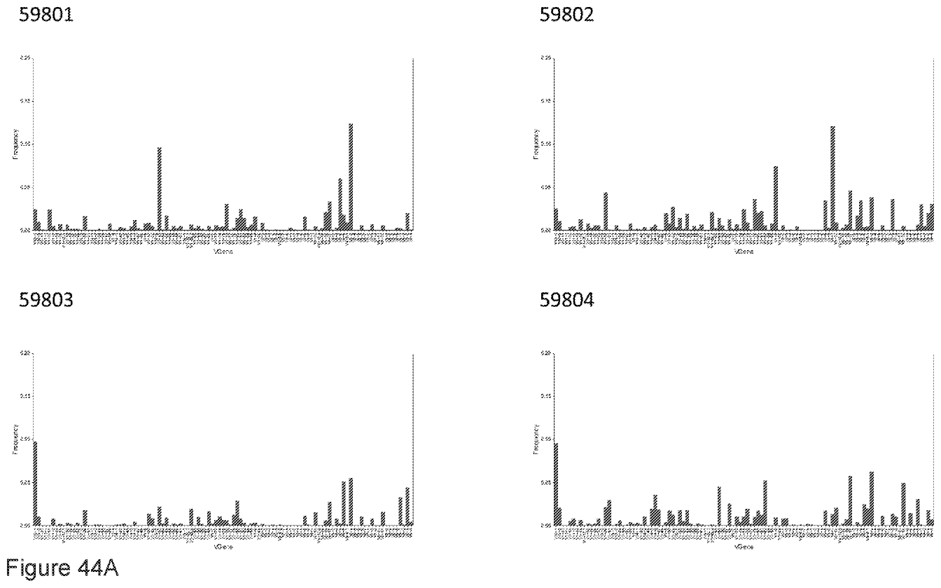

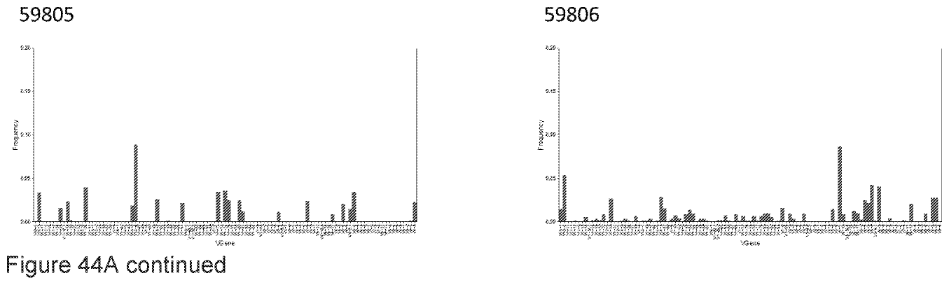

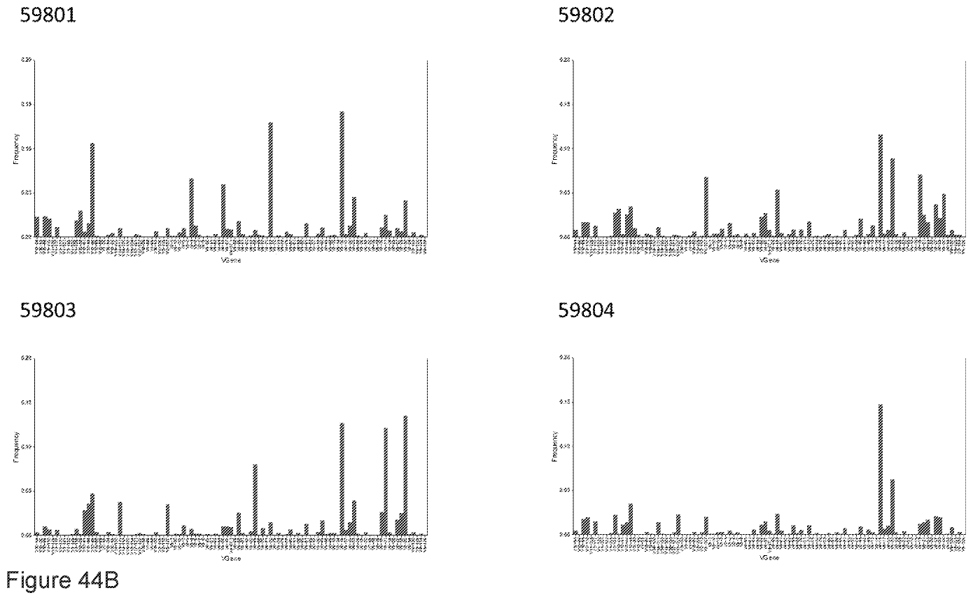

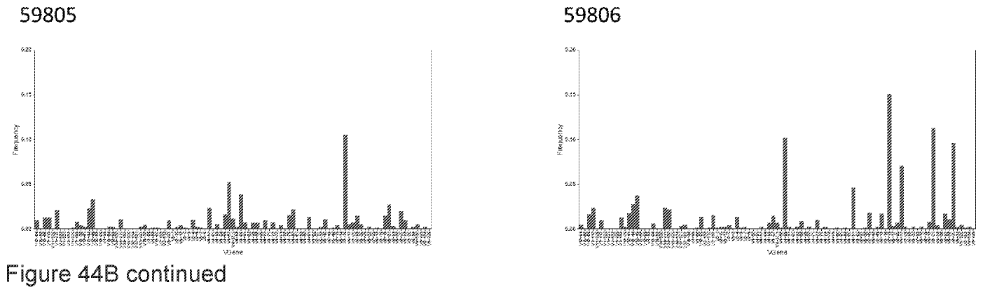

For non-limiting examples of hole-filled CH848 703010848.3.d0949.10.17envelopes, see WO/2017152146 and WO2018/161049, inter alia without limitation, FIGS. 44A-D and paragraph [0091], incorporated by reference in its entirety.

The immunogens of the invention can be delivered by any suitable mechanism.

In non-limiting embodiments, theses could be Adeno-associated virus (AAV) vectors. Characteristics of AAVs may include:
Being non-replicating viral vectors;
Providing sustained expression of the immunogen;
The ability to transduce dendritic cells, which present transgene (immunogen) in complex with MHCII to naïve T cells;
Constant antigen production which could lead to improved clonal persistence, enhanced germinal center reactions, and higher somatic mutation; and
Can be used a multivalent mixture to mimic chronic HIV-1 infection.

In certain embodiments, the immunogens could be multimerized.

Any of the inventive envelope designs could be tested functionally in any suitable assay. Non-limiting assays including analysis of antigenicity or immunogenicity.

### Example 2 Animal Study

19CV3 SOSIP trimer was used to immunize non-human primates.

Design of NHP study using 19CV3

| | | | | | Neutral- |
|---|---|---|---|---|---|
| Study | Animal | | | Binding | izing |
| # | Model | Synopsis | Adjuvant | antibody | antibody |
| NHP158 | Rhesus | 4X 19CV3 | GLA-SE | TBD | TBD |
| | | every 4 weeks | | | |

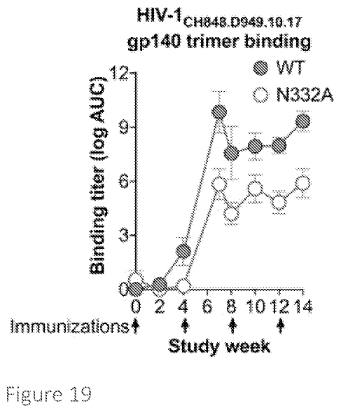

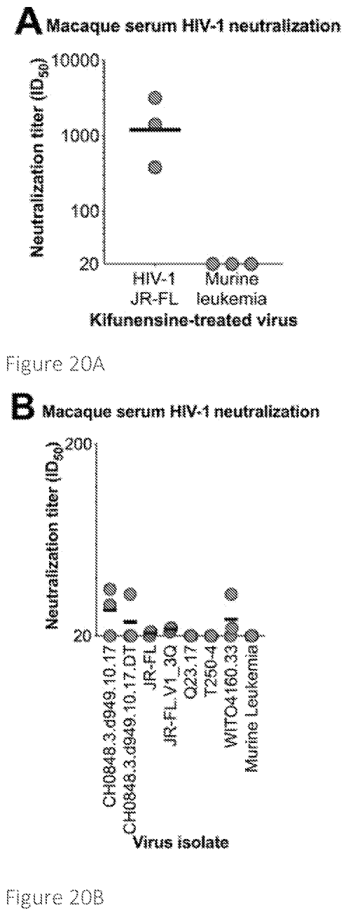

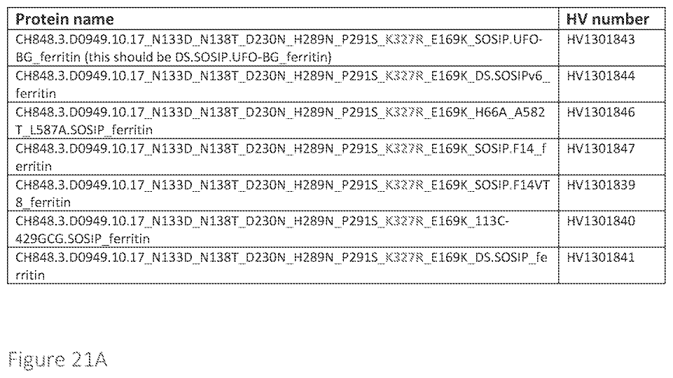

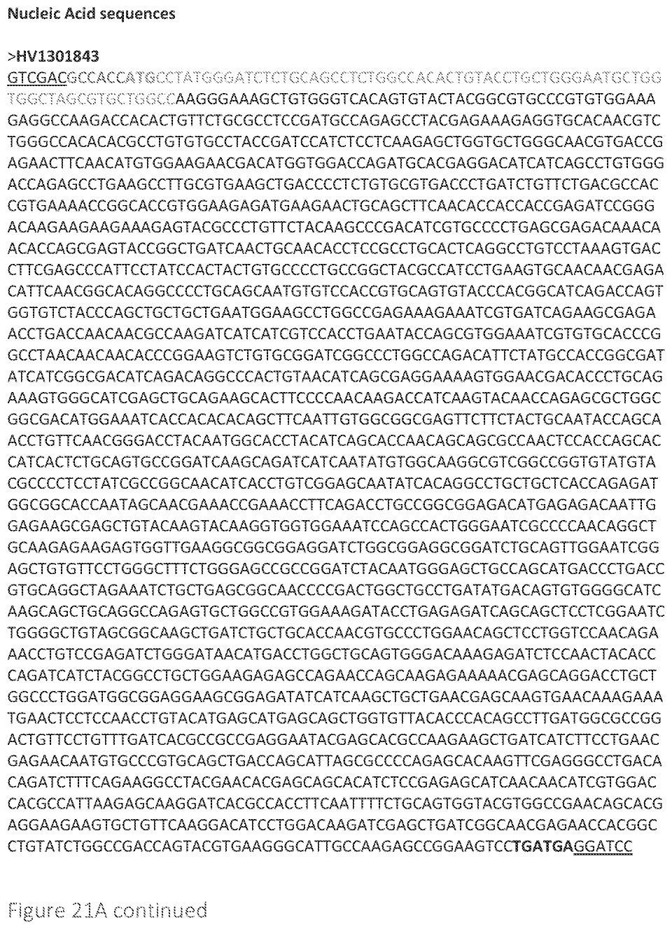

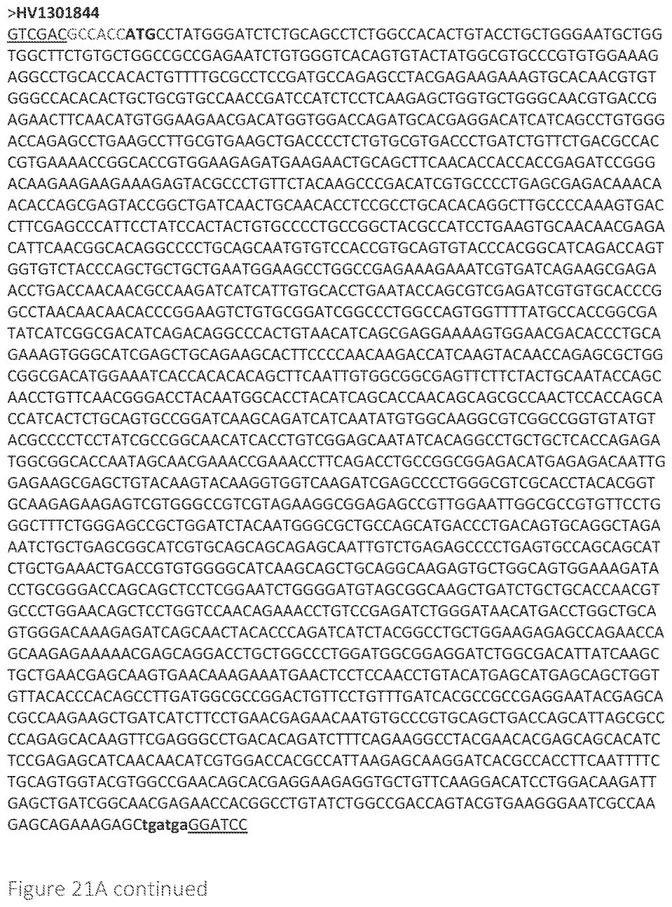

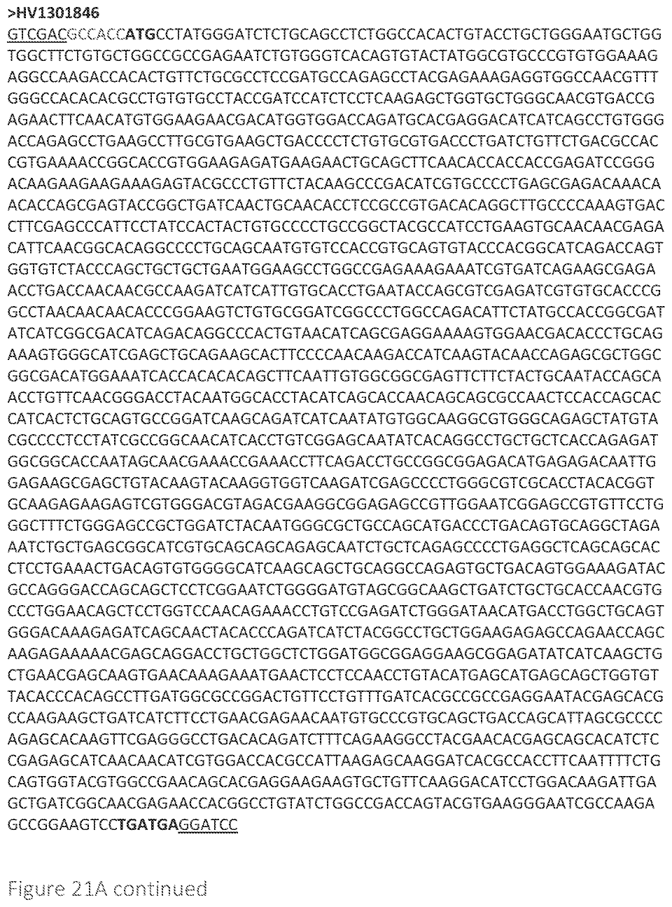

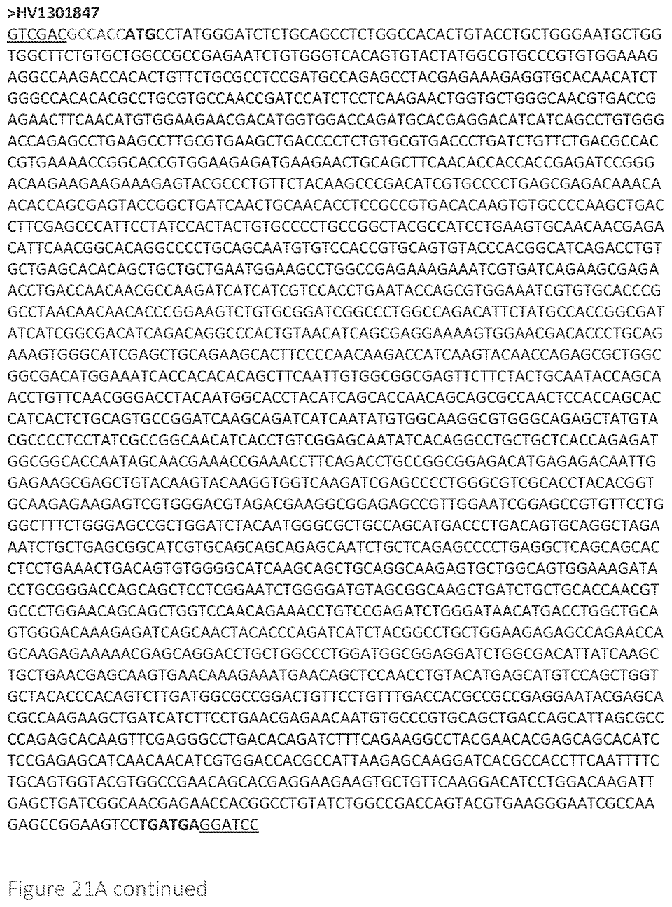

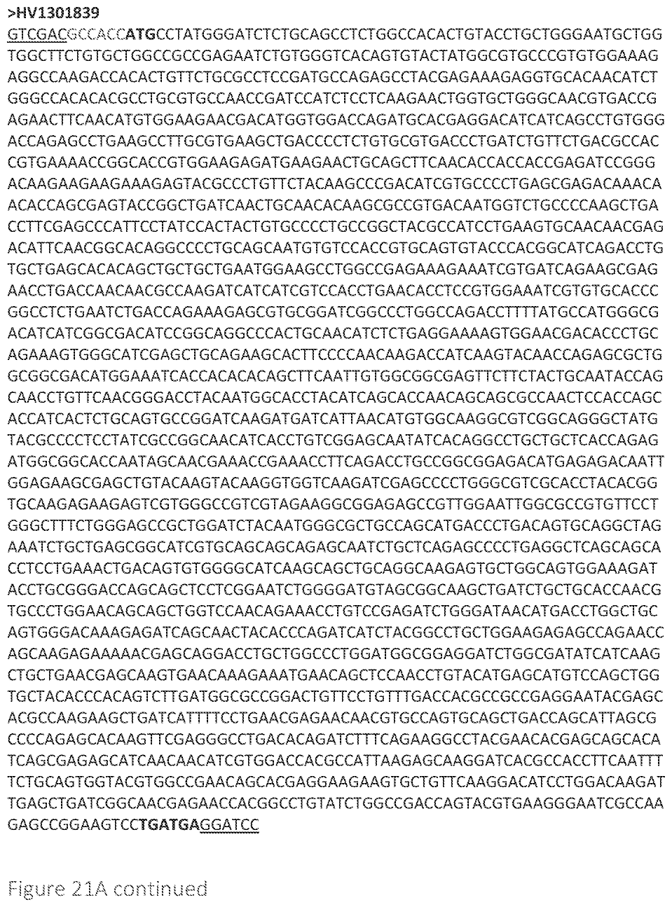

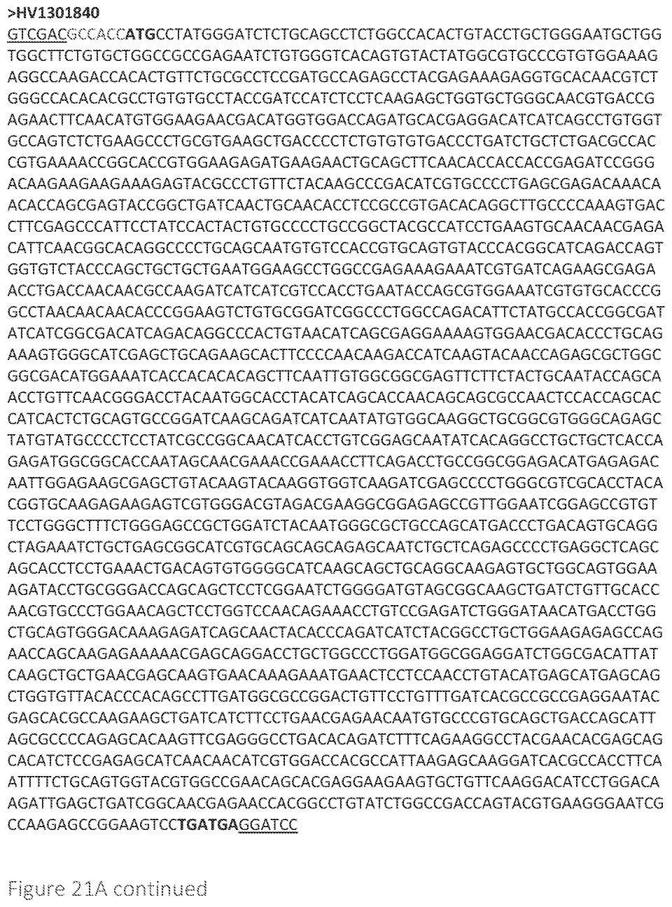

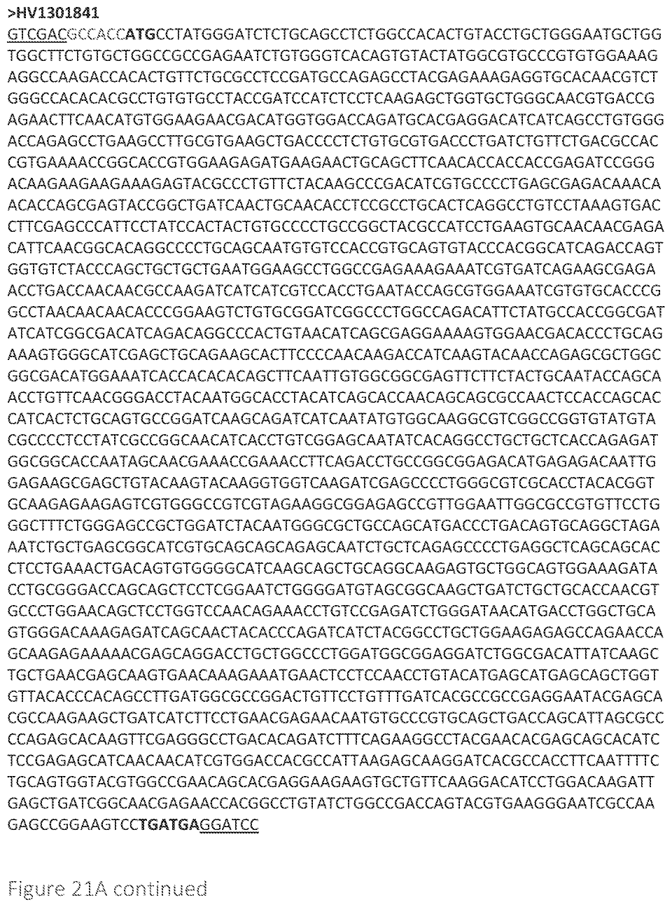

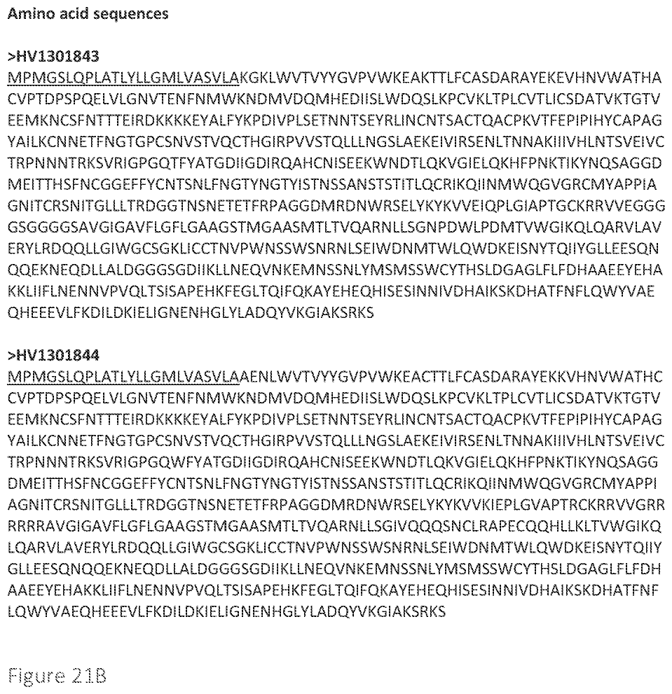

FIGS. 19-20B show data from NHP study #158.

### Example 3

This example describes animal studies with HIV-1 envelopes designed to prime and boost V3 glycan antibodies lineages.

The envelopes described in Table 3, expressed as recombinant proteins or modified mRNA formulated in LNP, are analyzed in animal studies including mouse and NHP animal models. The mouse animal model could be any model, including an animal model comprising a DH270UCA transgene.

Any suitable adjuvant will be used.

The envelopes in Table 3 will be produced under cGMP conditions as a recombinant protein and/or mRNA formulated in LNP for use in Phase I clinical trial.

### Example 4

This example provides analyses and selection of a new set of immunogens for induction of HIV-1 neutralizing antibodies.

Vaccines that can induce anti-HIV-1 broadly neutralizing antibodies (bNAbs) remain highly sought after as they will induce broad protective responses that will prevent infection by the globally diverse HIV-1 strains. We and others have shown that such bNAbs arise in HIV-1 infected individuals through multiple rounds of virus escape followed by antibody hypermutation to learn recognition of these escaped viruses (e.g. Bonsignori et al. PMID: 28298420). In this application we outline the selection of a set of sequential immunogens that are designed to mimic this process through vaccination.

In Bonsignori et al., PMID: 28298420 we reported the development of DH270.6, a bNAb targeting V3 glycan epitope, in the HIV-1 infected individual CH848. This antibody lineage was traced to identify intermediates along the evolutionary trajectory, and several viruses from CH848 were tested for neutralization against these intermediate and mature bNAbs.

In this work, we first identified signatures, defined as amino acids, glycan sites and hypervariable loop characteristics that are statistically associated with sensitivity or resistance to DH270 lineage Abs (Bricault et al PMID: 30629920). These signatures were calculated for both CH848 viruses as well as global HIV-1 viruses. We found that 6 positions (HXB2: 230, 241, 300, 301, 325 & 328) and hypervariable V1 loop lengths were statistically significant signatures that were overlapping between the two analyses. We hypothesize that these common signature sites of viral sensitivity/escape against DH270 antibodies in the CH848 patient viruses as well as global HIV-1 viruses are key positions at which CH848 viral evolution “teaches” the DH270 lineage to recognize heterologous HIV-1 diversity.

We used this hypothesis to guide the selection of seven CH848 Envs that not only show appropriate neutralization profiles against DH270 Abs but also expose critical amino acids at the above 6 positions and appropriate hypervariable V1 loops in a sequential manner that upon vaccination are designed to initiate and mature antibody responses similar to DH270. These Envs are: 1) CH848.d0949.10.17 DT, 2) CH848.d0949.10.17, 3) CH848.d0808.15.15, 4) CH848.d0358.80.06, 5) CH848.d1432.5.41, 6) CH848.d1621.4.44 and 7) CH848.d1305.10.35.

We have also calculated signatures that are associated with restricting the breadth of the broadest DH270 Ab (DH270.6), and have chosen two natural Envs (P0402.c2.11 and ZM246F) that expose such resistant signatures at sites 325 and 301, respectively, with the rationale that boosting with these two Env immunogens could induce DH270-like Abs that can show higher breadth than DH270.6.

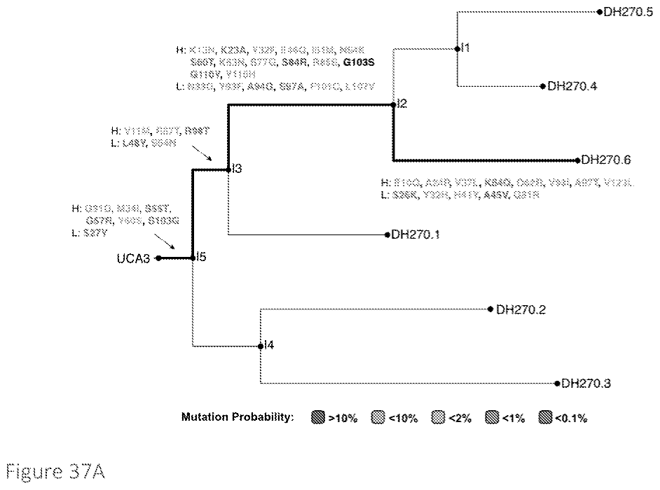

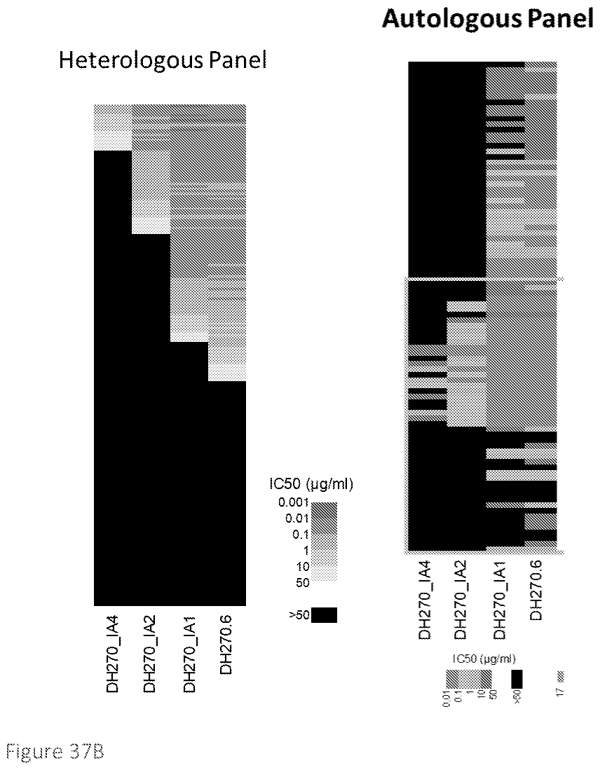

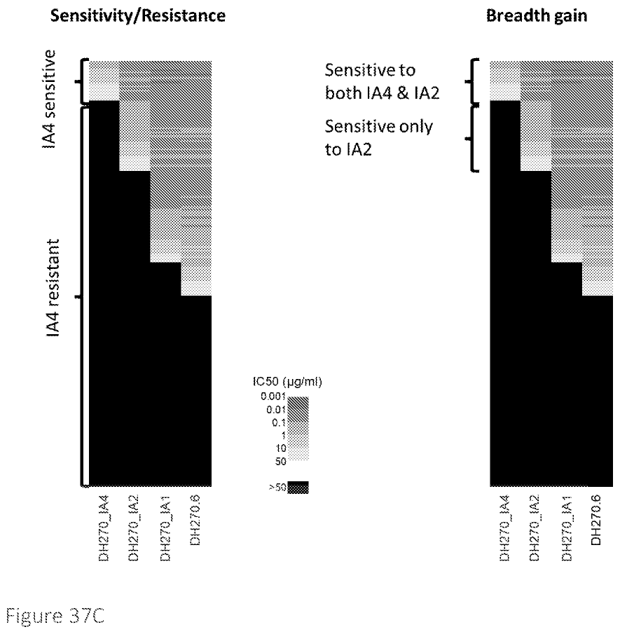

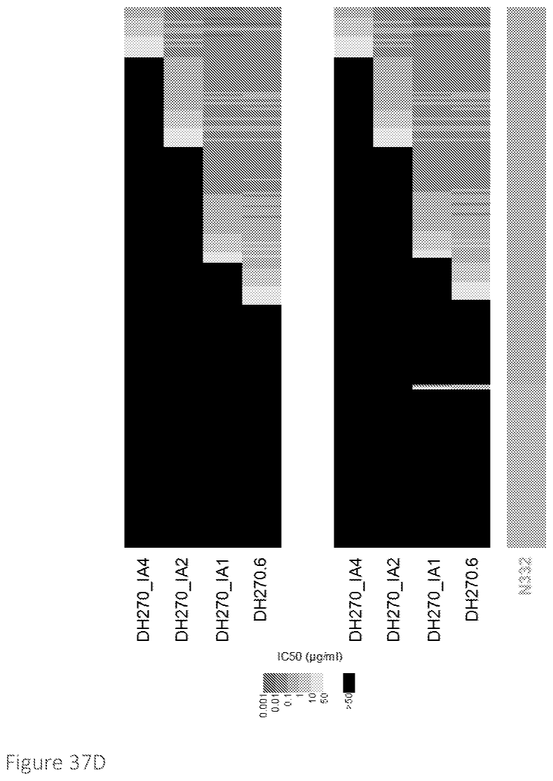

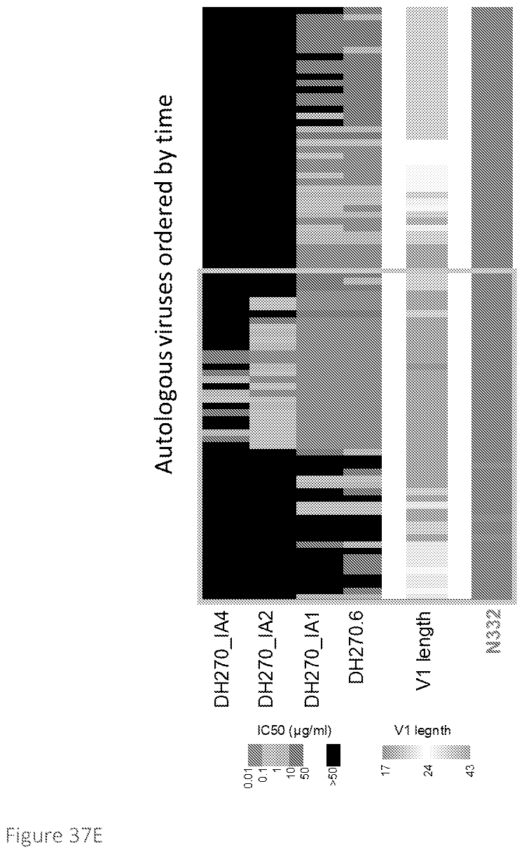

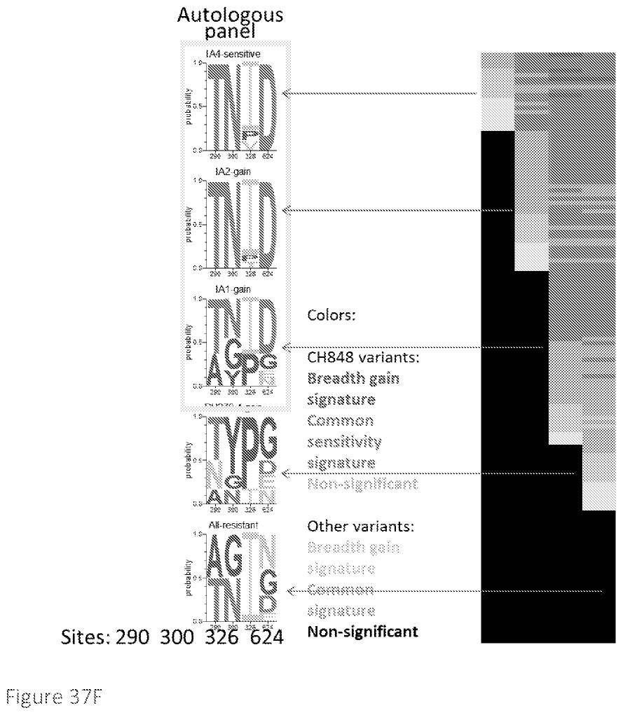

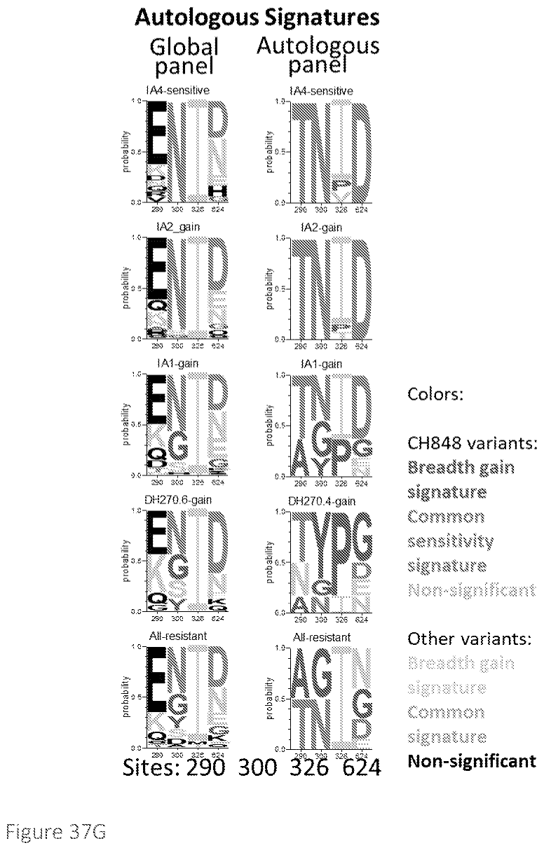

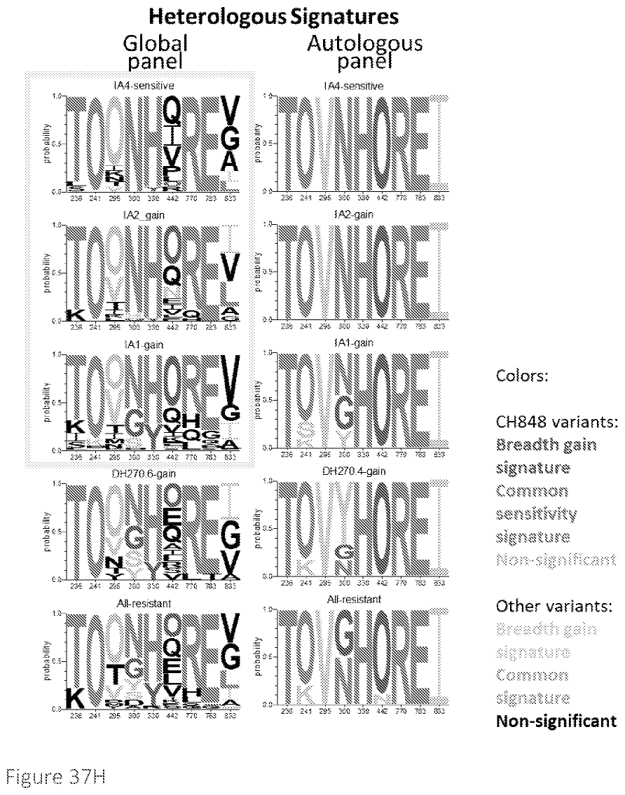

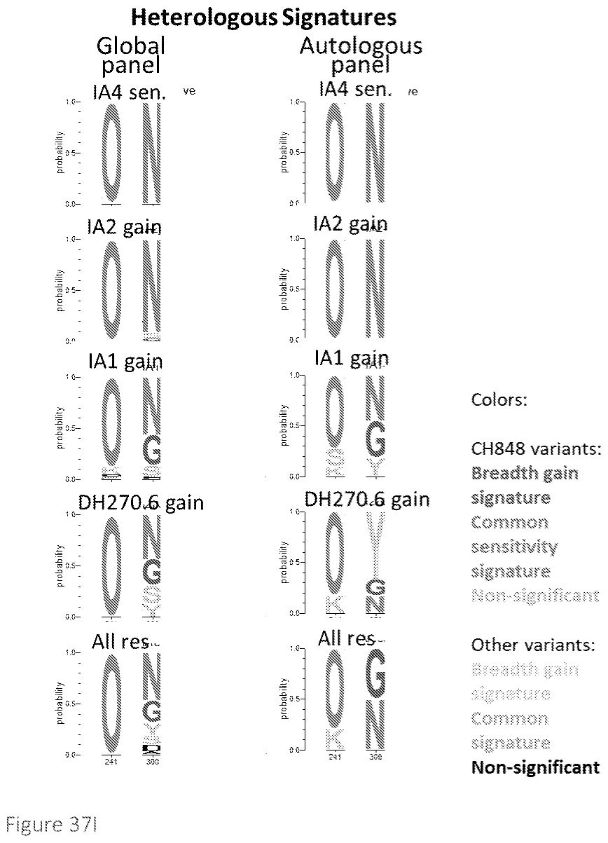

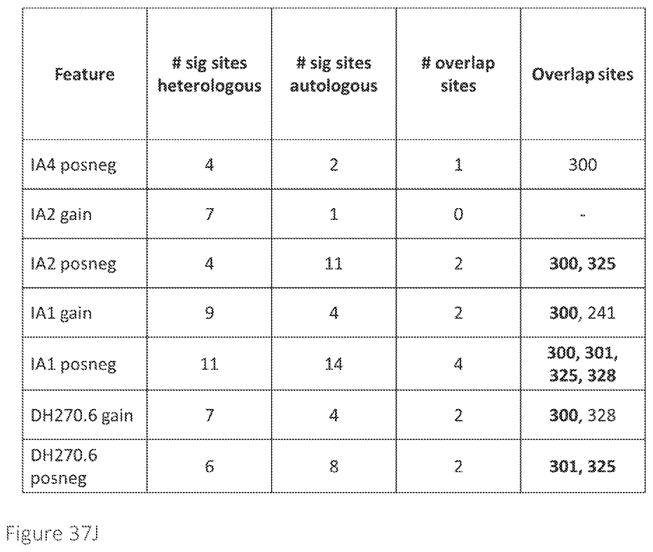

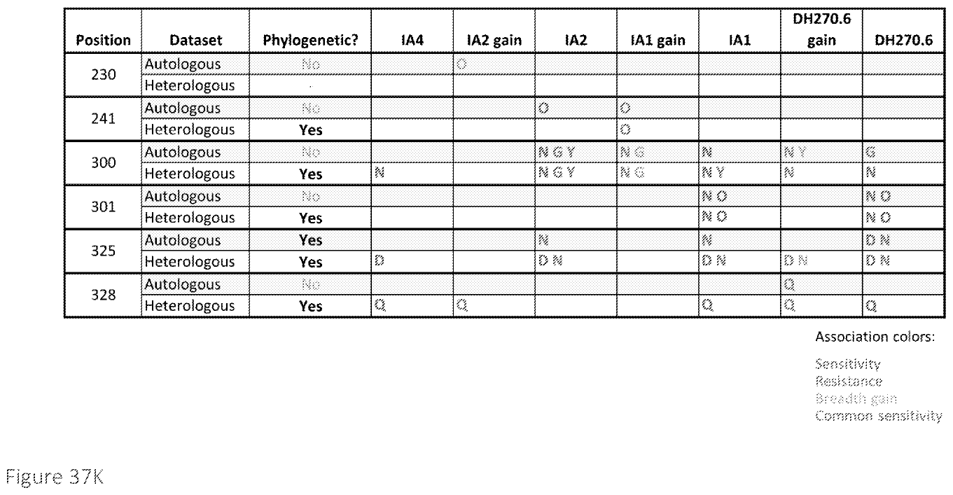

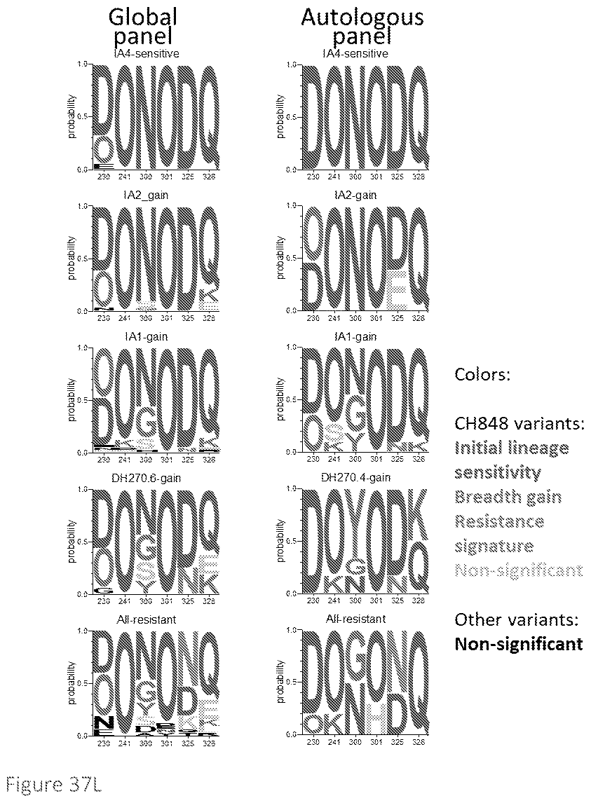

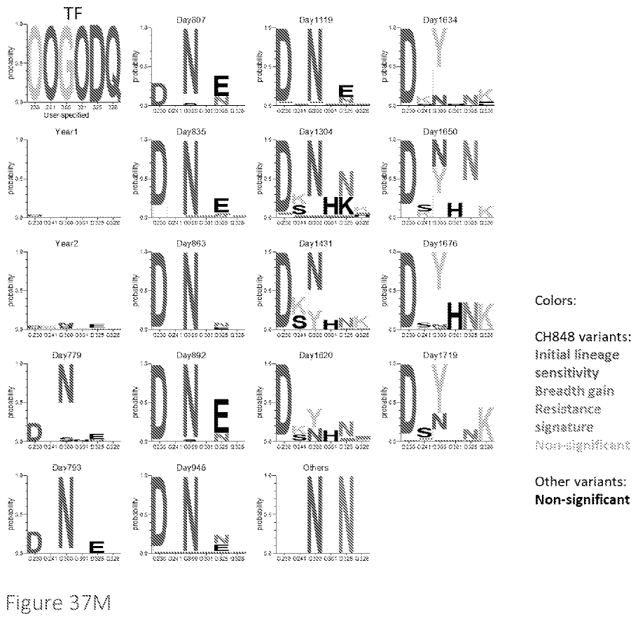

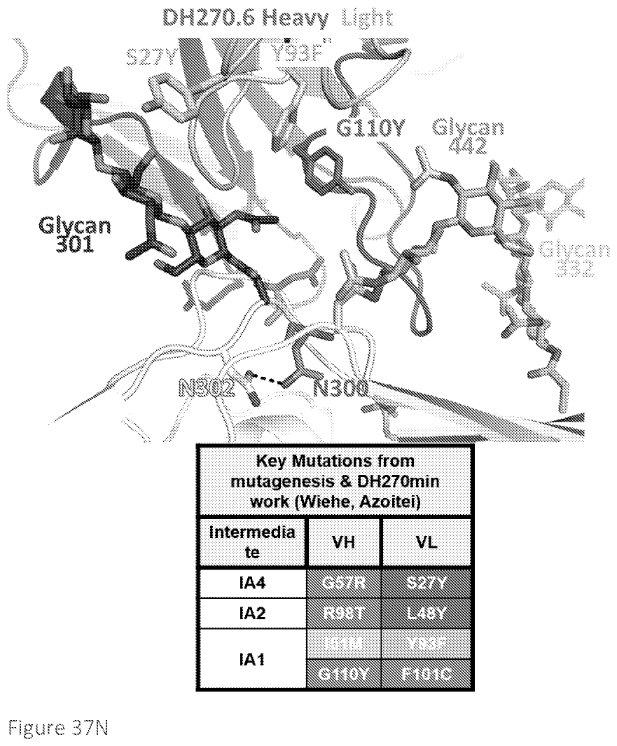

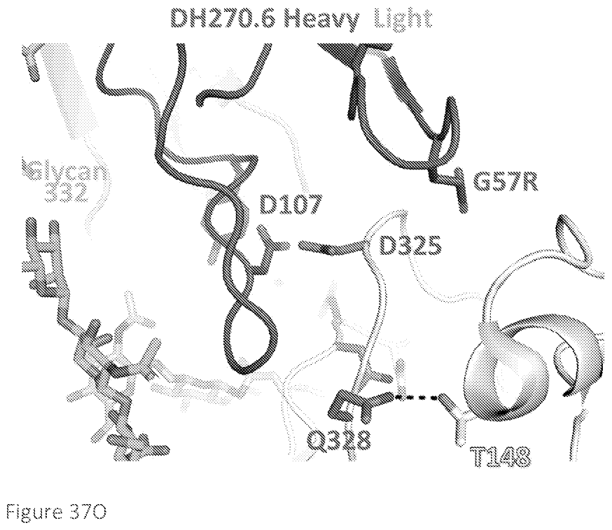

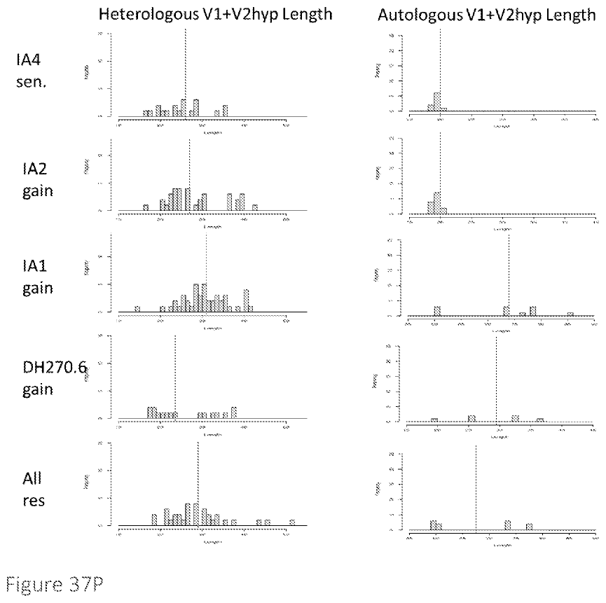

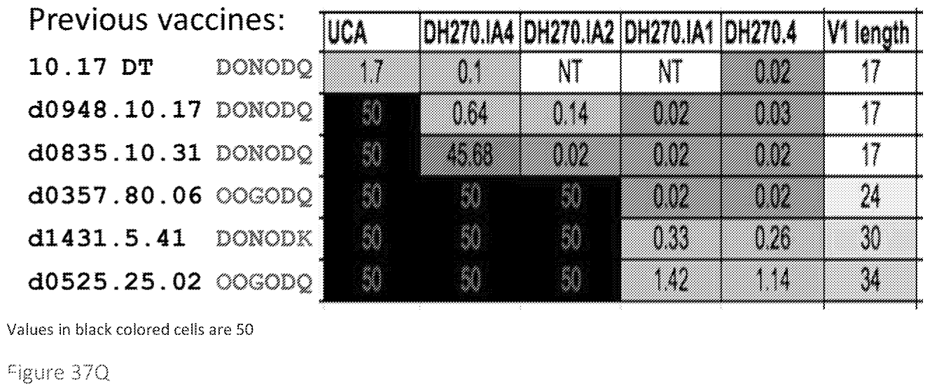

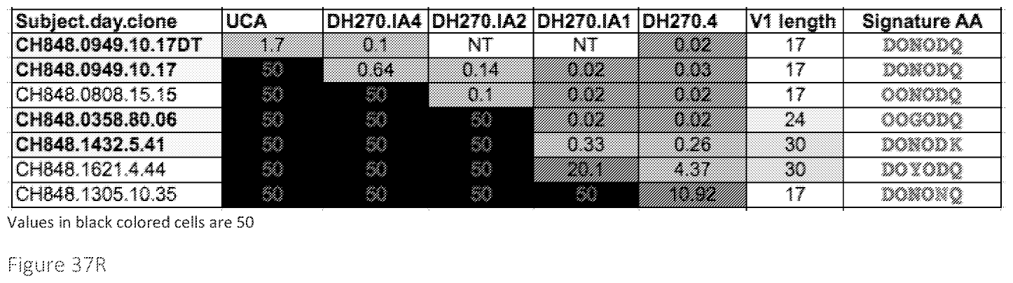

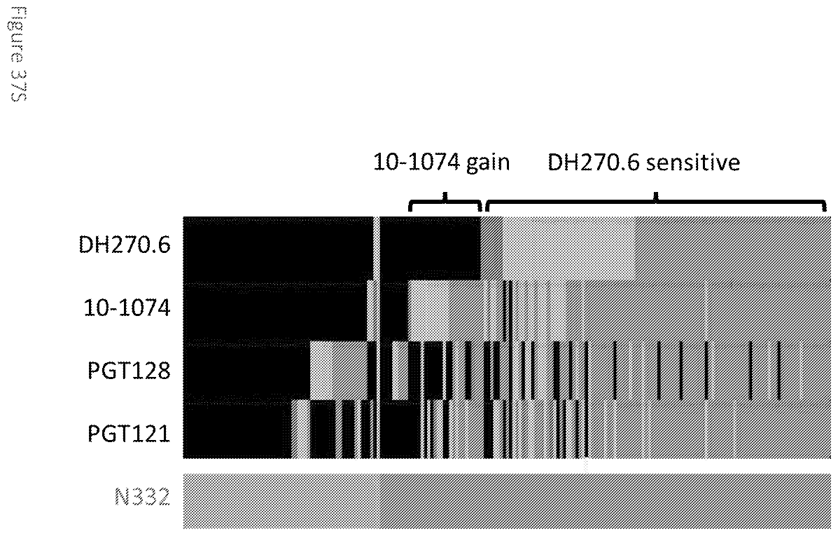

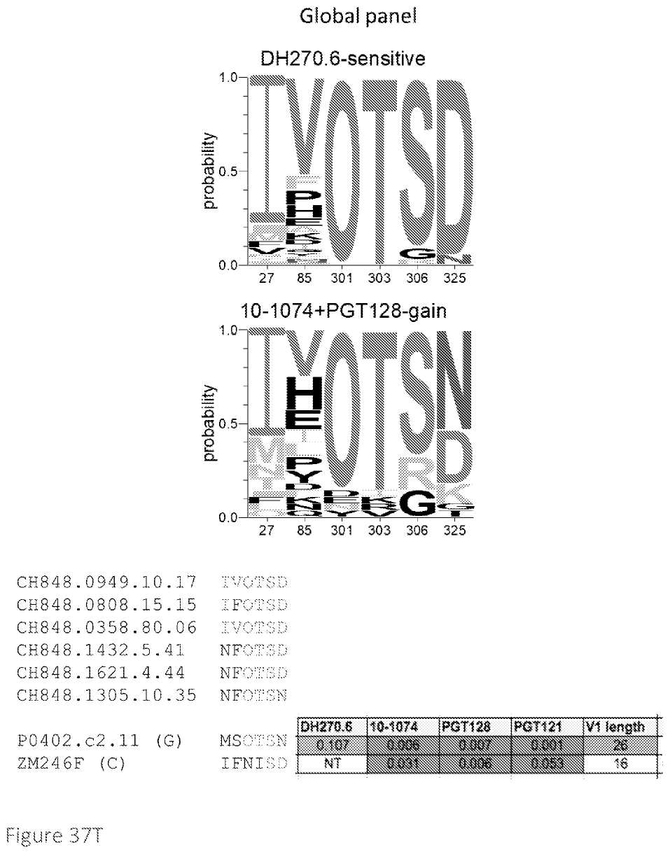

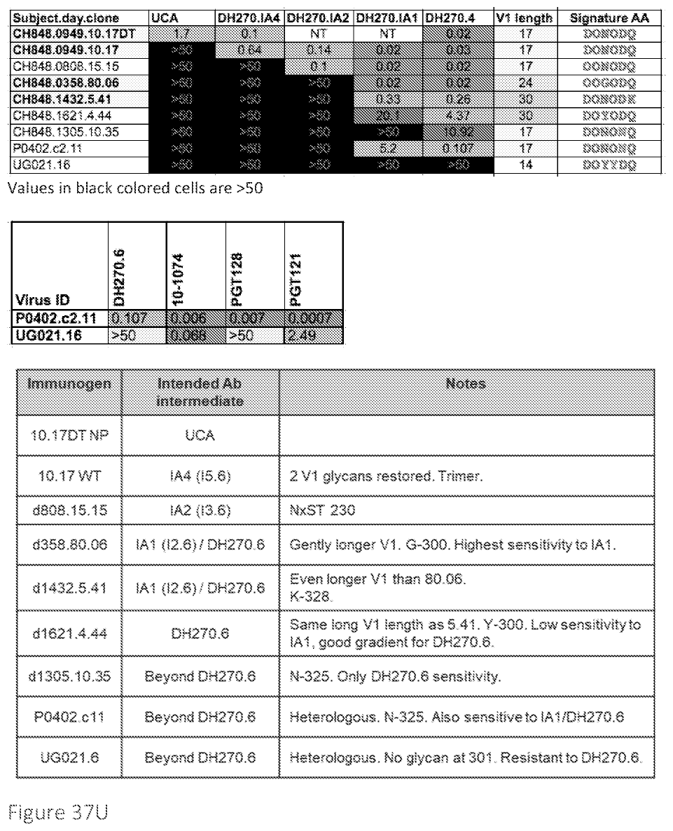

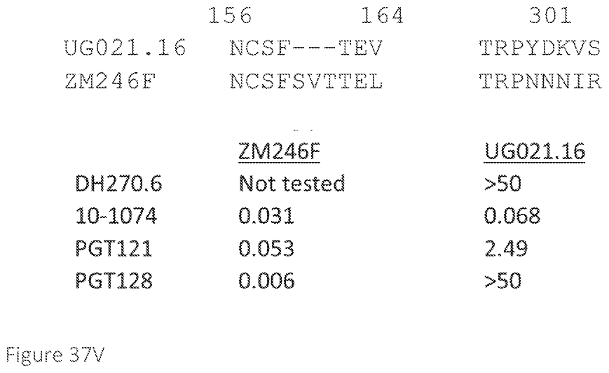

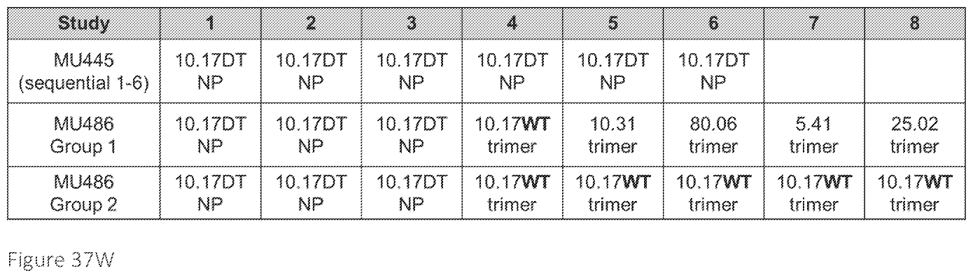

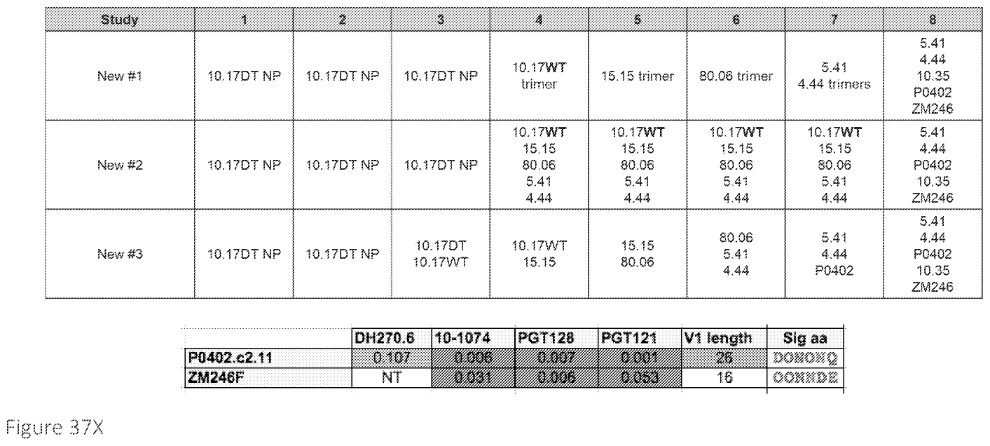

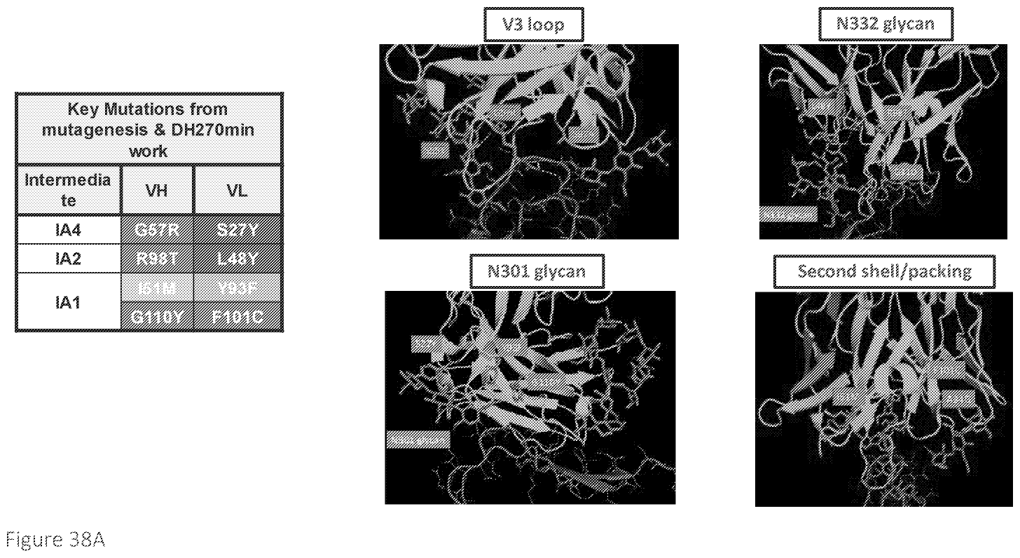

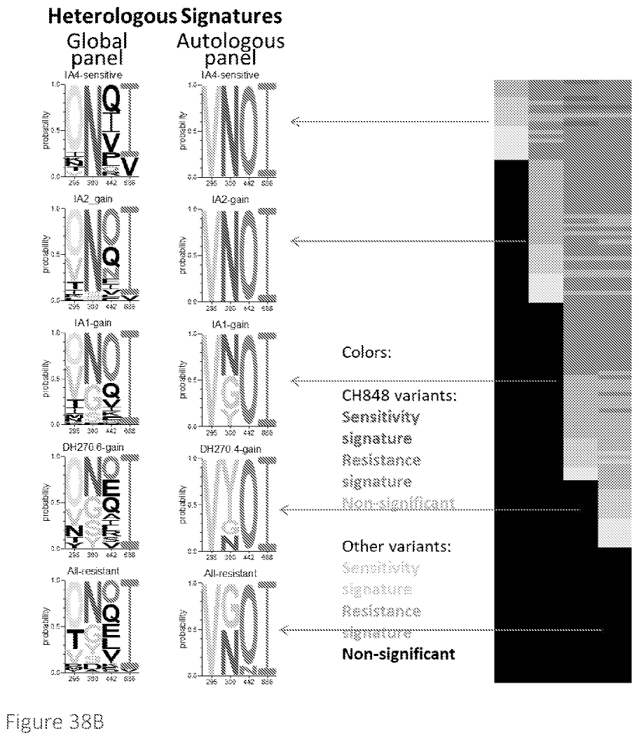

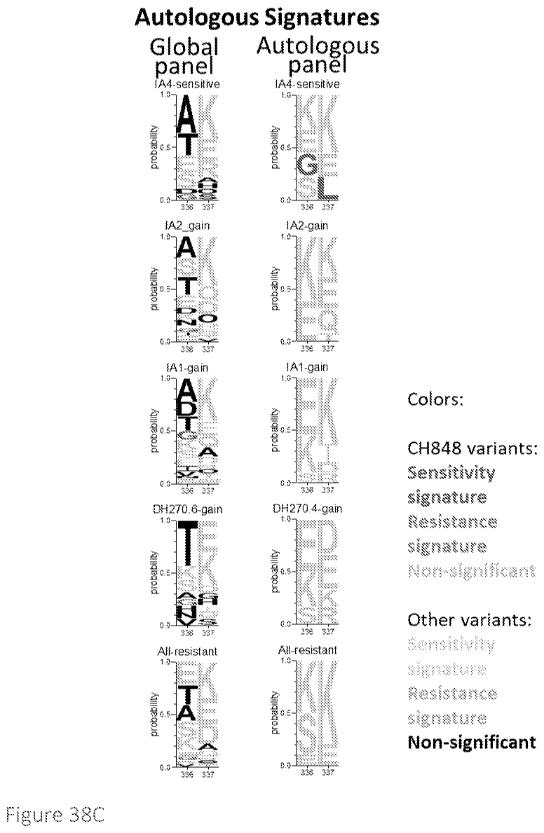

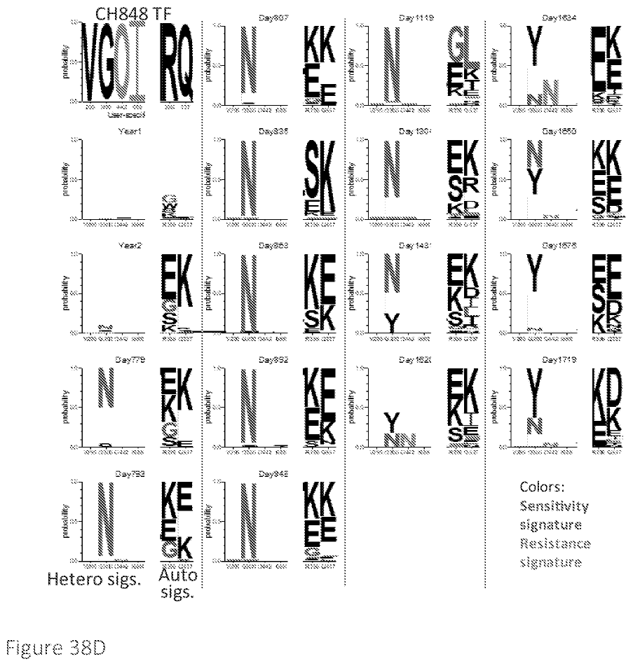

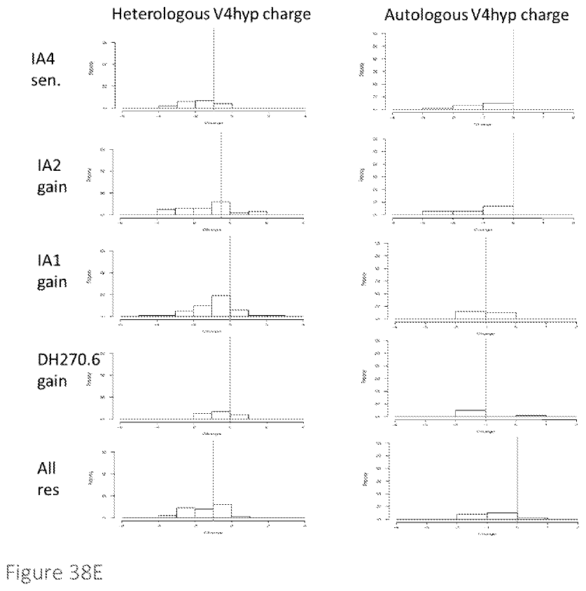

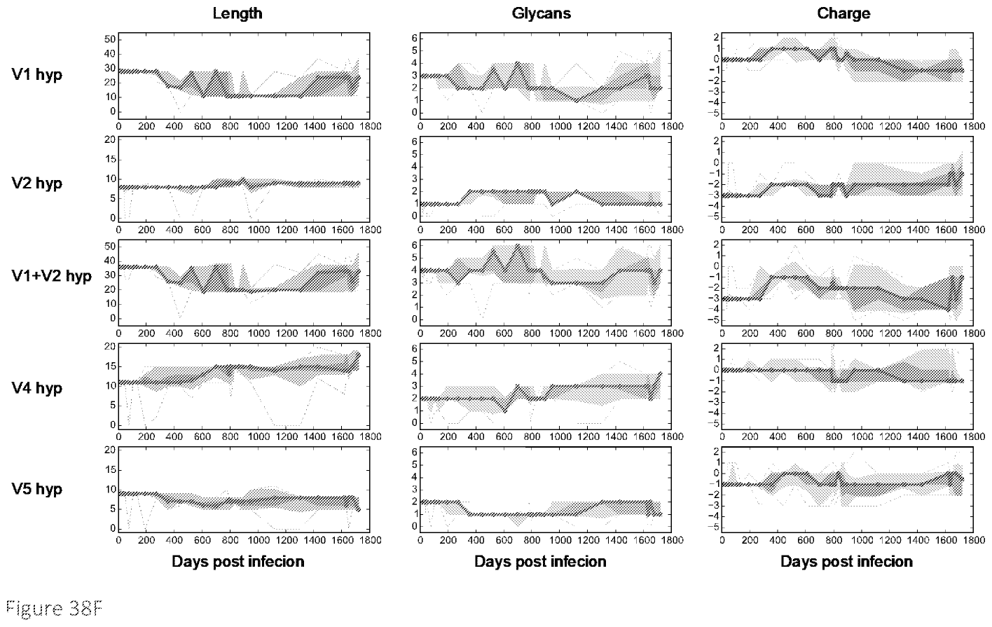

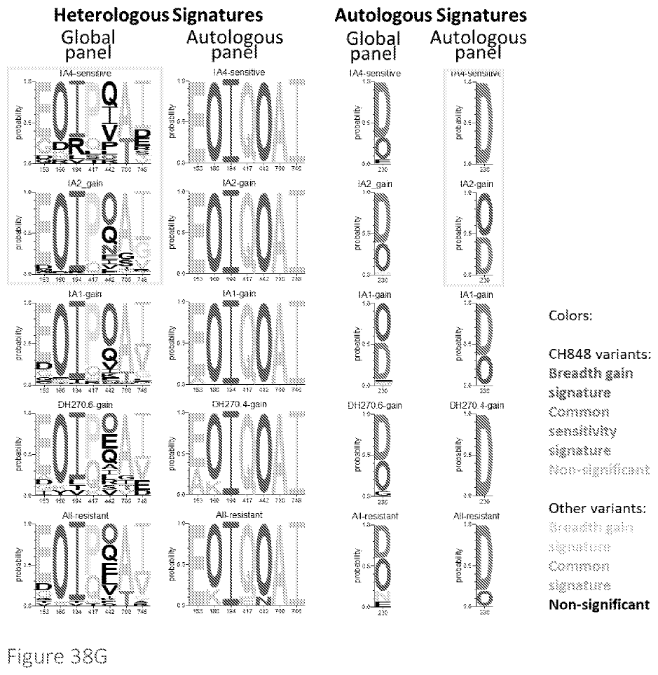

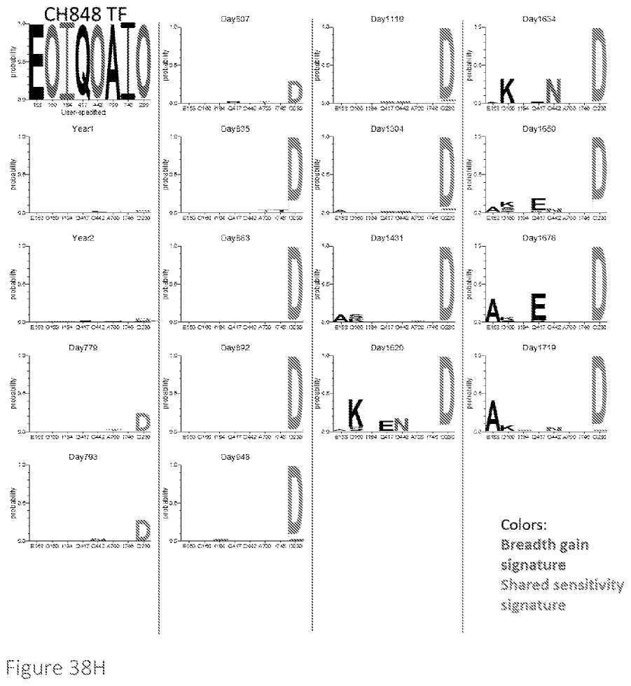

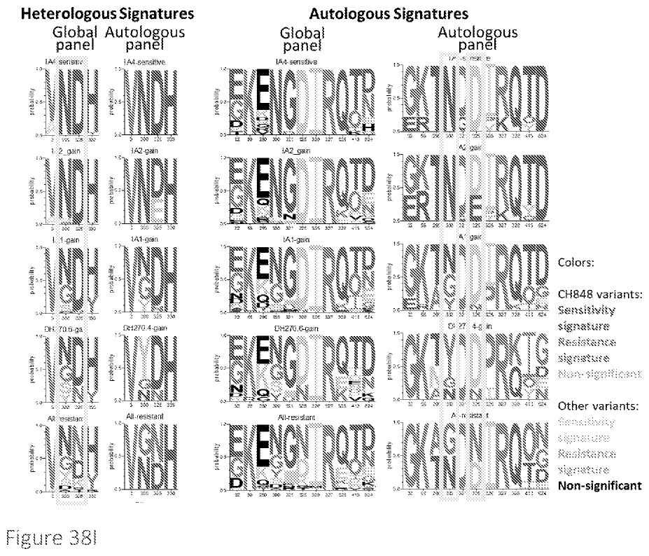

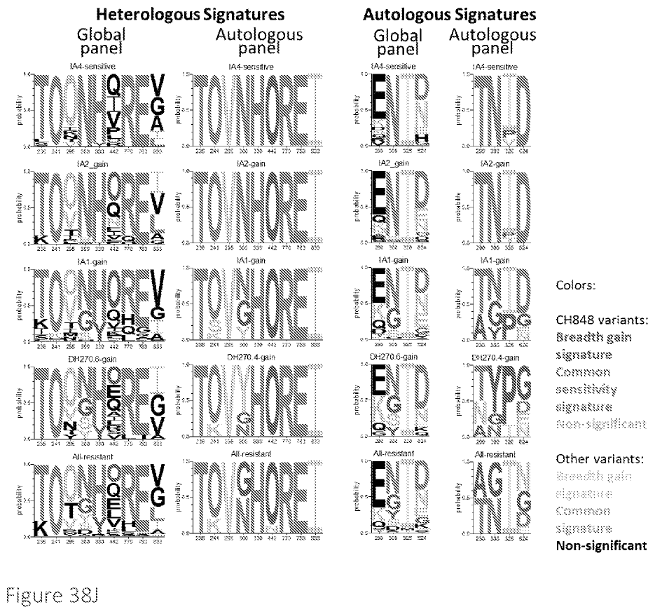

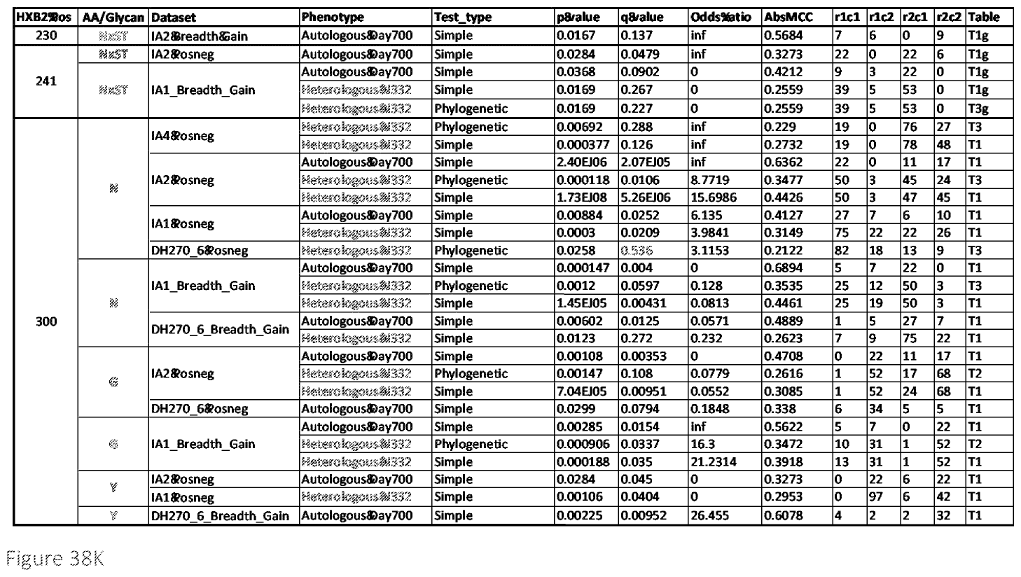

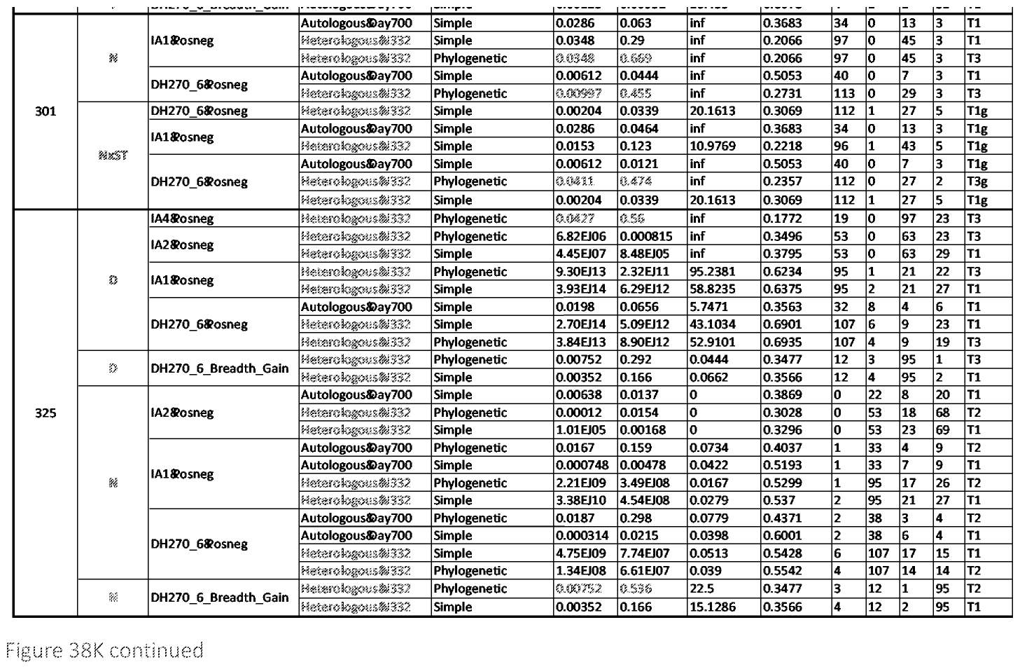

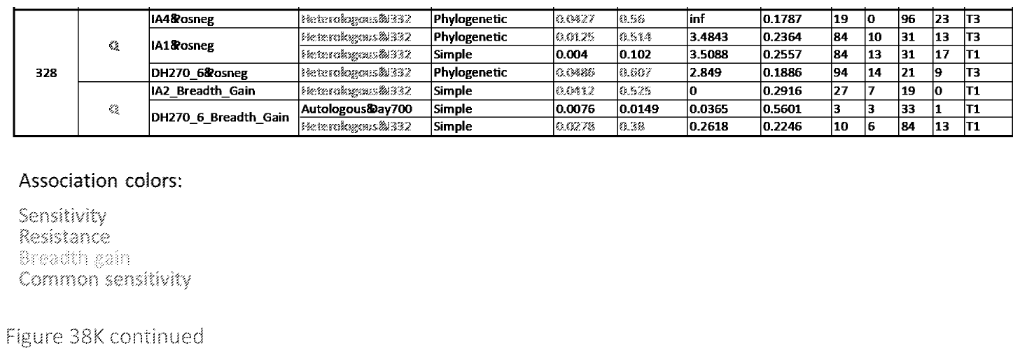

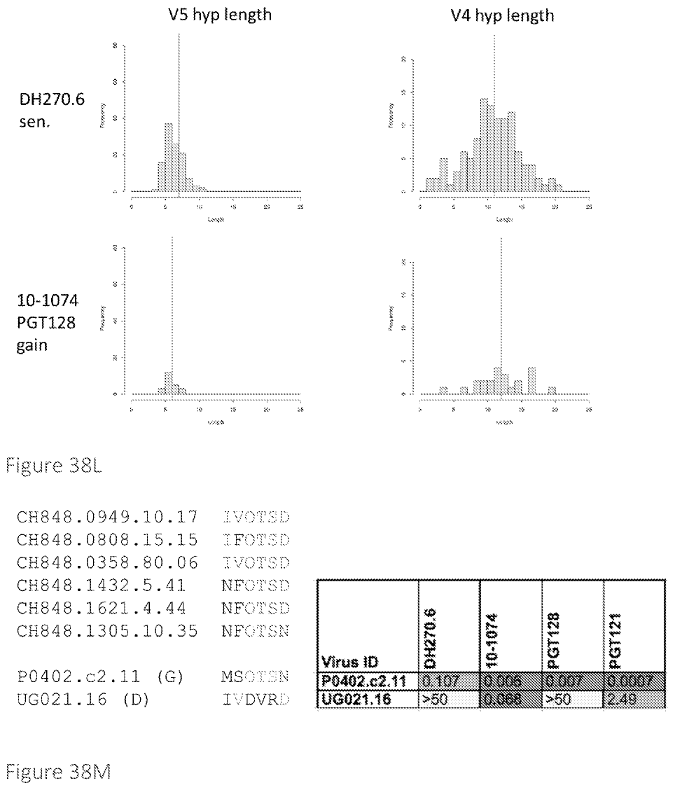

In FIGS. 37A-37X and 38A-38Q in this example, the term “global” panel is the same as “heterologous” panel. The heterologous viruses refers to a standard panel of 208 global circulating Envs made as pseudotyped viruses that is used for testing neutralization breadth and potency of antibodies. This was the same panel that was used in Bonsignori et al STM (PMID: 28298420). The “autologous” panel is the 90 pseudovirus panel made using strategically chosen longitudinal CH848 Envs from Bonsignori et al.

In FIGS. 37A-37X and 38A-38Q in this example, the phylogenetic correction refers to a particular strategy that accounts for potential biases arising from clade effects in signature calculations, as described in previous publications (Bhattacharya et al PMID: 17363674; Gnanakaran et al PMID: 20949103; Bricault et al. PMID: 30629920).

The symbol “O” is a short-hand of indicating an Asparagine in a potential N-linked glycosylation site motif (Asn-x-Ser/Thr, where x is any amino acid other than Pro). “N” refers to Asn not in such motifs.

The envelope selection is based on comparison of heterologous and autologous signatures to find overlap. This analysis identified 6 sites that have similar patterns across DH270 Abs between heterologous and autologous datasets-bNAb education. Based on these analyses, we designed a set of immunogens.

FIGS. 37A-37X provides a summary of the analyses and selection. These analyses and selection concern the autologous escape and heterologous breadth development in the DH270 lineage. Autologous escape leads to DH270 lineage evolution which leads to heterologous breadth development. FIG. 37A. The goal of this example was to determine which Env patterns are associated with autologous escape and how autologous escape confers heterologous breadth gain.

Datasets were used to determine neutralization data for DH270 intermediates (15.6˜ IA4, 13.6˜ IA2, and 12.6˜ IA1) and mature bNAbs against heterologous (n=208) and autologous (n=90) virus panels. FIG. 37B. Signature analyses were performed to identify Env amino acids/glycans that are associated with sensitivity/resistance of each DH270 Ab, and gain of breadth between consecutive intermediates. Hypervariable loop length, charge and number of glycans associations were also examined. Autologous and heterologous signatures were compared. Signatures are amino acids/glycans that are statistically enriched in one group of viruses vs other. Identification includes multiple test correction and phylogenetic correction. Selection criteria was either phylogenetic and/or contact. Two viral phenotypes were used: sensitive or resistant for a given Ab; breadth gain were sensitive to both intermediates versus sensitive to only next intermediate. FIG. 37C.

DH270 bNAbs were very strongly dependent on NxST332. Out of 62 global panel viruses without NxST332, only 2 viruses are neutralized by IA1 & DH270.6. Heterologous signatures analyses were reduced for the 143 viruses with NxST 332+2 sensitive non-NxST332 viruses for a total of 145 viruses. FIG. 37D. DH270 lineage bNAbs are strongly dependent on V1 length in the autologous dataset. Around day 700 (cyan box), V1 loop length starts decreasing. DH270 lineage start not known, but close to day 949. Because early viruses resistant to DH270 IA4-1A2, signatures were dominated by early forms. It is unclear as to how the viruses escape DH270. Signatures for day 700 and beyond viruses (n=50 out of 90 total) were selected for further examination FIG. 37E.

Four signature sites were associated with IA1 breadth gain in the autologous dataset: positions 290, 300, 326, 624. A-290, G/Y-300, P-326 and G-624 are associated with IA1 breadth gain. FIG. 37F. Red amino acids are associated with common sensitivity to IA2 and IA1. IA4 and IA2 are strictly require T-290, N-300 and D-624. Looking at the DH270.6 sensitive group, these breadth gain signatures are even more enriched. Continued bNAb maturation to incorporate these breadth gain signatures.

How these autologous signature patterns behave in the heterologous dataset was investigated. The global panel depicts heterologous viruses split by DH270 Abs sensitivity/resistance at the 4 autologous signature sites FIG. 37G. Color-coding is according to autologous signatures. The only common pattern found at site 300 (also a heterologous signature). Signature amino acids at 290 and 326 are very rare in heterologous viruses. For the heterologous dataset 9 signature sites were associated with IA1 gain: 236, 241, 295, 300, 330, 442, 770, 783, 833. FIG. 37H. For most sites, no single amino acid was associated with breadth gain but with more variability tolerated. Exceptions were for positions 300, 442, and 883. Most of signature sites show no variation in the autologous panel. CH848 quasispecies did not sample these escape pathways. Two exceptions were positions 300 (signature in autologous) and 241 (borderline signature in autologous panel).

Common signature sites between heterologous and autologous datasets involved in “bNAb education”. FIG. 371. At such sites, autologous viral escape from an intermediate Ab generates diversity that mimics heterologous diversity. This “educates” the maturing bNAb lineage to recognize heterologous diversity. Such sites important for immunogen design. Overlapping signature sites were systemically probed based on: overlapping signature sites; borderline sites where the signature defined in one dataset, is also a borderline signature in the other (Phylogenetic or not; p<0.05; same association with the same feature); for IA2 breadth gain, only one borderline signature found in autologous dataset (NxST230). This gave 6 sites: 230, 241, 300, 301, 325, 328. FIG. 37J. Positions 241, 300 and 325 are phylogenetic.

Positional characterization of bNAb education signature sites was performed. FIGS. 37K, 37L. CH848 d0949.10 17 matches all IA4-sensitive variants. Only autologous signatures are G-336 and L-337 sensitive. 10.17 does not match these (E-336 K-337). 230 NxST associated with IA2 breadth gain (only autologous). E-325 is quite rare in heterologous viruses and associated with IA2 breadth gain. IA1 Breadth gain variants include K-241, G/Y-300 and K-328. N-325 recognized at very low frequency with IA1 breadth gain. DH270.6/4 gain is associated with Y-300 and K-328 which are better recognized. N-325 is still rarely recognized. The main route of escape is loss of NxST301 & N325 (heterologous only NxST-332 viruses considered) leading to resistance to all DH270 lineage. Design principles included: (1) in addition to appropriate neutralizing/binding profiles, select Envs that match these bnab education patterns; (2) factor in the only common loop signature V1+V2 length; (3) add 1-2 boosts to improve breadth beyond the natural DH270.6.

Longitudinal evolution of the variants was investigated. FIG. 37M. TF variants are associated with resistance to early Abs at sites 230 and 300. These evolve to sensitive variants 779-1119 days post infection. Timeline for IA4˜ 779-892 days post infection. Relapse to NxST-230 is never at high frequency, but at low frequency at day 948. IA2 likely arises. At day 1304-1634 onwards, escape at 241, 300, 301, 325, 328 towards breadth gain variants is observed. IA1 likely arises. At day 1650 onwards Y-300 becomes dominant, and full resistance associated H-301 and N-325 become more prevalent. DH270.6 likely arises.

The structural relevance of select mutations was investigated. FIG. 37N. IA4 and IA2 both require Asn-300 but later lineage members can tolerate the mutation of N300 to G/Y. N-300 forms a polar contact with N-302. N300G or N300Y could disrupt this, and potentially change orientation of 301 glycan. 301 glycan is important-critical and improbable mutations might interact S27Y, Y93F (light) and G110Y (heavy). NxST 442 is quite rare in M-group. It is unclear how the 301 glycan changes when NxST 442 is absent. IA4 and IA2 prefer Q-328. IA1 begins to see K-328 and DH270.6 can tolerate Q and K equally. Q-328 forms a polar contact with T-148. Q-328 might be involved in sequestering V1 loop away from V3. IA1 onwards tolerate longer V1 loops. D-325 is strictly required by IA4 and IA2. N-325 is rarely tolerated by IA1 and DH270.6, and enriched in viruses resistant to all DH270 Abs. The mechanism is unclear. D325 inserts between CDRH2 and CDRH3, could have made contacts, but do not. The closest Ab amino acid is D107. R-57 is not as close (˜9 Å). FIG. 37O.

Hypervariable V1 and V2 lengths are significantly associated in both heterologous and autologous datasets (p=0.0002-0.0033). Strikingly, IA4 and IA2 in autologous dataset recognize very small loops, but IA1 onwards can tolerate longer loops (V1 and V2). For CH848 viruses, dramatic length change for V1 are observed, but very little for V2. Only two significant heterologous associations identified: V4 hyp charge (p=0.008, q=0 11); V1V2hyp length (p=0.02, q=0.14). 2 borderline autologous associations observed: V1 hyp length (p=0.025, q=0.13); VIV2hyp length (p=0.059, q=0.22). Both these are super significant if seqs before d700 also considered. FIG. 37P.

In addition to appropriate neutralization/binding profiles, immunogens that match these heterologous patterns were desired. The previous vaccines did not include Y-300, N-325 and K-241. FIG. 37Q. Newer immunogens identified were based on neutralization profiles and coverage of key breadth-gain and resistance signatures. 3 previous immunogens retained: d949.10.17, d358.80.06 and d1432.5.41. 3 new immunogens: D808.15.15 introduces NxST-230; d1621.4.44 introduces Y-300; d1305.10.35 introduces N-325. FIG. 37R No suitable Envs with K-241 found (either too short or too long V1) were identified. It is rare in M-group, so ignored.

DH270.6 has lower breadth than other V3g bNAbs. Signatures for gain of breadth of 10-1074 or 10-1074+PGT128 over DH270.6 (only in NxST332 viruses) were sought. FIG. 37S. Immunogens for breadth gain signatures of 10-1074 and PGT128 over DH270.6 were also observed. FIG. 37T. Some N-325 viruses are sensitive to DH270.6. Chose this for a gentler heterologous boost. The most sensitive virus P0402.c2.11 (subtype G, tier 2) was the only virus that also provided coverage at other sites (27 and 85). No virus lacking NxST 301 was neutralized by DH270.6. From CATNAP we found ZM246F that is sensitive all other V3g bNAbs, but not tested on DH270.6. NxST301 (and by definition T-303) have strict requirement for DH270.6. Strong requirement of D-325. Other signatures are subtle. New proposed immunogens cover 27 and 85. So key targets for breadth increase are removing NxST 301 and introducing N-325.

New sequential immunogens were identified. FIG. 37U. The sites include 230, 241, 300, 301, 325, 328. Only autologous signatures are G-336 and L-337 sensitive. 10.17 does not match these (E-336 K-337).

UG021.16 has potential issues. UG021.16 possesses a rare 3-amino acid deletion at positions 160-162 which could disrupt the structure at the apex and potentially V3. FIG. 37V. It was found that ZM246F (clade C; TF; Tier 2) does not have the 160-162 deletion (no 160 glycan). It also does not have the NxST 301. ZM246F is more sensitive to V3 glycan bNAbs than UG021.16.

Previously, DH270 UCA knock-in mice were used in immunization studies. FIG. 37W. Individual critical and improbable mutation frequency plateaus after 3 or 4 immunizations with 10.17 DT nanoparticle (NP). Combination of mutations low overall, but keeps on increasing with immunizations. MU492 (not shown) has few immunizations and longer waiting times worse than repeated immunization. MU486 group 1 (sequential Envs) comparable or slightly lower mutation frequency and neutralization than group 2 (repeated 10.17WT boosts). Mutation frequency not much better than just repeated 10.17DT immunization (MU445). Each of the 4 critical light chain mutations induced at very low frequency (0.06-1.1%). Also 2 out of 4 heavy chain critical mutations at only 0-0.003% (other 2 at 10-18% individually).

New knock-in mice may provide further immunization information. FIG. 37X. Individual critical & improbable mutation frequency plateaus after 3 or 4 immunizations with 10.17 DT nanoparticle (NP). Combination of mutations low overall, but keeps on increasing with immunizations. MU492 (not shown) has few immunizations and longer waiting times worse than repeated immunization. MU486 group 1 (sequential Envs) comparable or slightly lower mutation frequency and neutralization than group 2 (repeated 10.17WT boosts). Mutation frequency not much better than just repeated 10.17DT immunization (MU445). Each of the 4 critical light chain mutations induced at very low frequency (0.06-1.1%). Also 2 out of 4 heavy chain critical mutations at only 0-0.003% (other 2 at 10-18% individually). New #1 (top row): Sequential boosting. 5.41 and 4.44 grouped together as they show similar neutralization profiles (only sensitive to IA1 & DH270.6). 10.35 (N-325) and the two heterologous viruses come at the last step. Can compare Ab responses before and after this to study the impact of these immunogens designed to go beyond DH270.6. 5.41 and 4.44 also included so that the more resistant viruses (10.35+2 het) could drive off-target responses. Alternative: 8th could be a repeat of 7th and 9th could be 10.35 and 2 heterologous. New #2 (middle row): Mixed boosting. Immunogens from 10 17 WT (for IA4 targeting) to 4.44 (IA 1 and DH270.6 targeting) co-delivered 4 times to test if boosting with mixture can lead to better Ab responses (compared with New #1). Final step is again for going beyond DH270.6. New #3 (bottom row): Staggered mixture boosting, High diversity mixtures in New #2 may require too big a jump in Ab recognition. Each boost is designed to target 2 Ab intermediates in each step. (e.g. 3rd boost for UCA+1A4, 4th boost for IA4 & IA2, etc.) 7th boost-P0402 is included before 10.35 (which is in 8th immunization) because P0402 sensitive to both IA1 and DH270.6, while 10.35 only for DH270.6 (UG021 completely resistant).

FIGS. 37A-37E, 37J, 37L, 37P-37T (discussed above) show signature analyses. FIGS. 38A-38Q show signature analyses for a new set of immunogens for induction of HIV-1 neutralizing antibodies. FIG. 38A depicts key mutations for DH270 lineage Abs. FIG. 38B depicts IA4 heterologous signatures. 3 contact sites are 295, 300, and 442; one out of contact. Heterologous signature sites rarely change in the autologous panel: Borderline autologous signature at 300 (N sensitive, T1, p=0.02, q=0.33). Except 300, and maybe 337, poor correspondence between heterologous and autologous datasets. FIG. 38C depicts IA4 autologous signatures. No overlap in signature between heterologous and autologous were observed. Weak correspondence at position 337 between heterologous and autologous signatures.

IA4 signatures were studied longitudinally. FIG. 38D. Of the heterologous signatures only 2 sites show evolution-300 and 442. 300 escape from N form starts at day 1304. 442 escape is late (day 1620) and only to low frequency. By definition, autologous signature sites have to see some evolution. For both sites, almost complete replacement over time, starting with year 2. By day 1119 sensitive are dominant, replaced by non-significant next time point onwards.

The IA4 loop signature was also examined. For heterologous more positive charge associated with IA4 resistance (p=0.008) and breadth gain up to IA1. FIG. 38E. For autologous, negative trend (not significant). Other V4 associations in autologous with number of glycans for IA2 resistance and DH270.4 breadth gain. Longitudinal loop evolution was examined. FIG. 38F.

IA2 breadth gain signatures were identified FIG. 38G. No autologous signatures found using original criteria. Only one site with p<0.05:230 NxST is with breadth gain, D with common (p=0.017, q=0.14-0.31). No overlap in signatures between heterologous and autologous. Not even borderline (p<0.05). These IA2 breadth gain signatures were examined longitudinally. Except for the autologous 230, very few sites show late and low frequency mutations. After D-230 is established, low frequency of NxST-230 was found at day 948. FIG. 38H. 2 signature sites were shared between the heterologous and autologous signature panels: 300 and 325. FIG. 381. At these sites, even the non-significant amino acids are sampled in the autologous viruses. Some autologous signature sites show concordant patterns in the heterologous panel: 624 and 328. 321 and 413 show strikingly different patterns.

The only common breadth gain signature for IA1 is at 300. FIG. 38J. Some concordance at 241 for heterologous and 624 for autologous. The strength of the signatures was examined. FIG. 38K. Test type can be phylogenetic or simple.

The strongest hypervariable loop association was with V5 hypervariable length (p=0.036) in the breadth gain signatures of 10-1074 and PCT128 over DH270 6. FIG. 38L. Shorter V5 loop for breadth gain. Borderline association with V4 hypervariable length (p=0.055). Longer V4 loop for breadth gain. The only virus without NxST 301 and neutralized by 2 other V3g bNAbs is UG021.16 (subtype D, tier unknown) for immunogens for breadth gain beyond DH270.6. FIGS. 37T, 38M. Two heterologous viruses lacking NxST332 that are sensitive to DH270.6 not having NxST334 were identified. FIG. 38N. No autologous viruses lacking NxST332 are sensitive to DH270 Abs.

Alternate choices include four NxST-230 options with the same variants, V1 length, similar IA2 sensitivity (0.1-0.3 μg/ml). FIG. 38O. The most sensitive was selected. G-300 was based on previous choice. K-328 was only two options available. The more sensitive form was selected. Y-300 chose d1432.5.18 out of 4 because it contributes K-241 & has the longest V1 loop. N-325 chose 1305.10 35 for bigger affinity gradient between IA1 & DH270.4. The only shared IA1 signature is 300. FIG. 38P. Some concordance for 301, 325, 328, and 509. For breadth gain, beyond this, sites of interest are 300, 325, and 413. FIG. 38Q depicts IA1 heterologous signatures.

These immunogens will be tested in mouse studies with new immunogens.

Animal studies analyzing the immunogens from this example will be conducted to evaluate the immune responses induced by this selection of immunogen.

Any suitable adjuvant will be used. The number and time interval between boost can be determined experimentally.

### Example 5: DH270UCA Targeting Immunogen

This example describes a rationale and development of V3 glycan/DH270.6 germline targeting immunogens that recognize precursors with diverse CDR H3 loops.

Multiple antibody lineages that are the current focus of vaccine development efforts against HIV, influenza or coronavirus, contain rare features, such as long CDR H3 loops. These unusual characteristics may limit the number of available B cells in the natural repertoire that can evolve to secrete such antibodies by vaccination. In order to measure the ability of a given immunogen to engage naturally occurring B cell receptors of interest, here we describe a mixed experimental and bioinformatic approach focused on determining the frequency and sequence of CDR H3 loops in the immune repertoire that can be recognized by a vaccine candidate. By combining deep mutational scanning and computational analysis, CDR H3 loops that can be engaged by two existing HIV immunogens were identified and characterized, thus illustrating how the methods described here can be used to evaluate candidate immunogens based on their ability to bind diverse B cell receptors.

For an effective vaccine, it is important to ensure that B cells exist in the human repertoire that can be engaged and activated by a candidate immunogen. As previously shown, B cell activation depends on the overall frequency of the target B cell population and the affinity of the immunogen for the respective BCRs. Many antibodies against diverse viruses employ unusually long CDR H3 loops for neutralization, which may limit the number of precursor B cells in the immune repertoire that can be engaged by vaccination to elicit related humoral responses. For example, HIV bnAbs that target the V2 apex and glycan-V3 epitopes on Env typically contain CDR H3 loops of over 20 amino acids. Similarly, broadly cross-reactive antibodies that bind to the influenza neuraminidase or the sialic acid receptor binding site utilize long CDR H3 loops for the majority of their viral contacts. Recently, some isolated antibodies that neutralize both existing and emergent coronaviruses were founds to also rely on long CDR H3 loops for recognition. Because of their potency and broad recognition of diverse viral isolates, the elicitation of these types of antibodies is currently of interest for HIV, universal influenza or pancoronavirus vaccine development efforts.

The heavy chain complementarity determining region 3 (CDR H3) loop is the major antibody site involved in antigen recognition. Compared to the other five antibody CDR segments, CDR H3 loops exhibit significantly higher sequence and structural diversity, which allow them to recognize various antigens¹. The median length of human CDRH3 loops is around 15 amino acids⁶⁻⁸, although longer loops of over 20 amino acids are present with low frequencies). CDR H3 loops are formed through VDJ recombination, a process that involves the introduction of double-strand breaks in DNA to join the V, D, and J genes, and a break repair mechanism that adds random nucleotides, called N-nucleotides, at the junction sites⁹⁻¹¹. Therefore, CDR H3 loops contain genetically encoded segments as well as non-templated, stochastically generated regions. While the sequence of CDR H3 loops typically evolves during antibody affinity maturation in response to antigen stimulation, length altering insertions and deletions are rare^{12, 13}. In order to elicit antibody lineages that rely primarily on CDR H3 contacts for virus neutralization, it is therefore critical to ensure that candidate immunogens can bind with high affinity to natural BCRs that contain CDR H3 loops related to those of target antibodies. This is particularly important when such antibodies contain unusually long CDR H3 loops that are typically rare among natural BCRs, like the ones described above against HIV, influenza, and coronaviruses.

Currently, natural BCRs engaged by a given immunogen are identified by labeling human PBMCs with the target molecule, followed by Florescence Activated Cell Sorting (FACS) to select B cells with the desired phenotype. The sequence of the isolated BCRs is subsequently determined and corresponding IgGs are typically produced recombinantly and characterized for binding to the target immunogen. This approach requires the ability to access and manipulate large number of human B cells, involves laborious and challenging experimental methods, can be biased by the selection strategy, and is expensive. To address these limitations, here we describe an experimental and bioinformatic approach to identify BCRs in the natural human repertoire that contain CDR H3 loops predicted to be bound by a candidate immunogen. For a given antibody, our approach utilizes deep scanning mutagenesis to rapidly identify possible changes in the sequence of its CDR H3 loop that are tolerated by a candidate immunogen. Using the resulting amino acid substitution profile, a public database containing the sequences of >100 million human BCRs is subsequently queried to identify CDR H3 sequences predicted to be bound by the candidate immunogen. CDR H3 loops of interest can be subsequently selected for experimental characterization.

We applied this platform to analyze two immunogens, CH505.MS.G458Y and 10.17DT, which have been shown previously to activate precursors of HIV broadly neutralizing antibodies (bnAbs) CH235.12 and DH270.6 in animal models 14. 15 DH270.6 and CH235.12 neutralize an estimated 51% and 89% respectively of the circulating HIV viruses, and their elicitation is the focus of HIV vaccine development efforts. The initial step in the induction of HIV bnAbs is to activate B cells whose BCRs can be subsequently matured to breadth. To this end, the 10.17DT immunogen was engineered to bind the DH270UCA3 mAb, which is the inferred unmutated common ancestor (UCA) of the HIV bnAb DH270.6. DH270UCA3 contains a 20 amino acid CDR H3 loop that is the major site of interaction with the glycan-V3 epitope on HIV Env. Similarly, CH505.M5.G458Y is a germline targeting immunogens against the UCA of CH235.12, a HIV bnAb that targets the CD4 receptor binding site. CH235UCA contains a 13 amino acids CDR H3 loop, which is an important, but not major, site of antigen interactions. Using our platform, we found that the natural B cell repertoire contains a high number of BCRs with CDR H3 loop sequences that should permit engagement by CH505.M5.G458Y. In contrast, B cell activation by the 10.17DT immunogen will be more limited given the restrictive sequence requirements of CDR H3 loops recognized by this molecule. Natural CDR H3 loops that are bound by the DH270.6 germline targeting immunogen 10.17DT were identified and validated, thus illustrating how our approach can be employed to evaluate the ability of vaccine candidates to engage BCRs present in the human B cell repertoire.

Identification of CDR H3 loop variants recognized by germline-targeting immunogens that bind CH235 and DH270.6 bnAb precursors.

The goal of this project were to determine the ability of DH270.6 GL-targeting immunogen 10.17DT to recognize precursors with diverse CDR H3 loops. The loops investigated were DH270UCA3 variants with CDR H3 mutations. FIGS. 30C-30F, 31A-31C.

Structural analysis revealed that the 13 amino acid CDR H3 loop of CH235UCA contributes ˜30% (276 Å² out of 956 Å²) of the total antibody buried surface area at the interface with priming immunogen CH505.M5.G458Y. In contrast, CDR H3 loop mediated interactions between the DH270UCA antibody and the 10.17DT immunogen are more substantial. The 20 amino acid DH270UCA CDR H3 loop contributes-50% of the antibody buried surface in the 10.17DT binding complex (244 Å² out of 466 Å²), by making significant interactions with both the V3 loop as well as the glycan present at position N332. Based on this analysis, we hypothesized that 10.17DT binding to DH270UCA3 will be sensitive to the CDR H3 loop composition, while CH505.M5.G458Y will maintain high affinity interaction with diverse CDR H3 loop variants of CH235UCA.

To determine the ability of 10.17DT and CH505.M5.G458Y to engage bn Ab precursors with diverse CDR H3 loops, we first developed site saturation mutagenesis libraries that sampled all the single amino acid variants in the CDR H3 loops of DH270UCA3 and CH235UCA mAbs. Libraries of scFv versions of these mutated antibodies were displayed on the surface of yeast and clones that maintained binding to the target immunogen were isolated by FACS. The DNA of selected clones was subsequently extracted and analyzed by next generation sequencing. The ability of an immunogen to bind a particular CDR H3 loop mutation was measured by determining the frequency of the respective amino acid substitution in the clones selected with the immunogen, relative to the frequency of the same substitution in the clones of the naïve, unsorted library. An increase in the presence of a mutation among the sorted clones indicated that the respective amino acid favors immunogen binding, while a decrease denoted that the respective mutation was detrimental to binding. Using this approach, we evaluated the effect of every single amino acid substitution in the CDR H3 loops of DH270UCA3 and CH235UCA towards binding by 10.17DT and CH505.M5.G458Y respectively.

As anticipated based on structural analysis, CH505.M5.G458Y maintained significant binding to a large number of CDR H3 substitutions in the CH235UCA. The 13 residue CDR H3 loop of CH235UCA is encoded by the VH1-46, D3-10*01 and JH4*02 genes, with 8 residues inserted by N-nucleotide additions. At the 10 residues in the N-addition and D gene regions, the immunogen recognized an average of 15.3 amino acids.

In contrast, a significantly smaller number of DH270UCA3 CDR H3 loop mutations resulted in antibodies that maintained 10.17DT binding. This CDR H3 loop is encoded by VH1-2*02, D3-22*01, and JH4*02 genes, with 9 amino acids inserted through non-templated N-nucleotide additions. Because limited diversity was observed in the naïve DH270UCA CDR H3 loop library at position 104 upon sequencing, the effect of substitution at this site on 10.17DT binding was determined experimentally. IgG antibody variants containing all the possible single 19 amino acid substitutions were expressed recombinantly and their binding to 10.17DT was compared to that of the native DH270UCA 3. On average, only ˜5 different amino acids were tolerated by 10.17DT at each CDR H3 loop site. The largest number of functional variants was found in the V_{H} and J_{H} templated regions, where only one site tolerated fewer than six different amino acid. In contrast, the composition of the D-gene and N-addition encoded residues was more restricted, with an average of ˜2 alternative amino acids that maintained immunogen binding identified at each site. In FIG. 30E, Log2 change gradient indicates amino acid substitutions at the particular sites of the CDR H3 loop that favor interactions with the immunogen. E.g. “most enriched” indicates CDR H3 loop mutations that maintain high affinity interactions with 10.17DT—e.g. position 98 and position 106. In FIG. 30E, “most depleted” indicates amino acid substitutions that significantly decrease interactions with 10.17DT.

These results indicate that CH505.M5.G458Y engagement of BCRs upon vaccination will not be restricted by their CDR H3 loop composition, since this immunogen can bind CH235UCA variants with highly divergent sequences in this region. In contrast, our data indicates that 10.17DT will only bind natural BCRs that have highly homologous CDR H3 loops to that of DH270UCA3.

To validate the results of scFV libraries screening, a subset of DH270UCA3 mutants were expressed recombinantly as IgGs and tested for binding to 10.17DT by SPR. DH270UCA3 antibodies containing single mutations in the CDR H3 loops were captured on a Protein A coated sensor chip and recombinant 10.17DT was injected as analyte. Across three positions tested, all amino acids substitutions predicted to maintain high affinity binding by screening showed at least 80% of the binding signal measured for WT DH270UCA3 binding to 10.17DT. Five of the seven point mutants that were predicted to strongly disfavor binding to 10.17DT resulted in almost complete abrogation of antibody binding. Two substitutions at position 111, tyrosine to tryptophan and histidine, only moderately affected binding of recombinant IgGs while their effect was found to be more substantial by library screening. Such discrepancies could be due to the two different platforms used to assess binding that utilize distinct antibody formats (FACS of scFvs displayed on the surface of yeast versus SPR of recombinantly produced IgG) Nevertheless, these results illustrate that high throughput screening of single site saturation libraries efficiently capture the affinity trends of IgGs containing single amino acid mutations.

Identification of CDR H3 Loops in the Native BCR Repertoire that can be Bound by 10.17DT and CH505.M5.G458Y.

The goals of this project were to determine the ability of DH270.6 GL-targeting immunogen 10.17DT to recognize precursors with diverse CDR H3 loops. The loops investigated were natural CDR H3 loops related to that of DH270UCA3. FIGS. 32A-32B, 33, 34.

Next, we sought to identify CDR H3 loops in the BCR repertoire of healthy individuals that can be engaged by 10.17DT and CH505.M5.G458Y as a way to estimate the number of B cells that these molecules may engage upon vaccination. The CDR H3 substitution profile recognized by either 10.17DT or CH505.M5.G458Y was used as a scoring matrix to search a public BCR database for CDR H3 loop sequences that are expected to be bound by these germline-targeting immunogens. CDR H3 loops were selected to have equal lengths and identical D genes located in the same relative position as in the CDR H3 loops of the target antibodies. For each CDR H3 loop in the database, a score was computed that sums up the enrichment values of each amino acid substitution relative to the DH270UCA3 CDR H3 loop. No CDR H3 sequences were found that were identical to those of DH270UCA3 and CH235UCA. Natural loops were predicted to interact with CH505.M5.G458Y with high affinity, because they contained sequence variants that were found to maintain binding in the library screen above. Based on this analysis it was predicted that 1 in 10,000 native BCRs contain a CDR H3 Joop that is compatible with CH505.M5.G458Y binding. The calculated frequency raises to 1 in 10 B cells if CDR H3 sequences are considered that contain no more than one amino acid substitution predicted to minimally affect binding (0.25<log 2 (enrichment)<0).

In contrast, no CDR H3 loops were found in the natural BCR database that were expected to bind 10.17DT with high affinity; all the sequences contained at least two amino acids that were determined by single site mutagenesis screening to significantly lower 10.17DT binding (enrichment log 2 values <−2). The queried BCR database contains only a small fraction of the BCRs found in an individual. Since none of the CDR H3 sequences analyzed were expected to bind 10.17DT with high affinity, we next wanted to determine if such loops may exist at sequence diversity depths normally present in the natural BCR repertoire. To address the limitation of exiting repertoire depth, we used IgOR to model CDR H3 sequences with the same immunogetics as the loops of DH270UCA3 and CH235UCA respectively.

To characterize the ability of 10.17DT to bind naturally occurring CDR H3 loops experimentally, we developed chimeric DH270UCA3 antibodies where the native loop was replaced with one of the 100 natural CDR H3 loops selected based on how well their sequence matches the amino acid substitution matrix. These 100 antibodies were expressed together as scFVs and displayed on the surface of yeast. Upon sorting for two rounds with 10.17DT, no high affinity binding was observed by FACS, although some scFV sequences were enriched in the selected clones, indicative of low affinity for the antigen. Seven such chimeric antibodies were expressed and purified as recombinant IgG and their binding to 10.17DT was measured by SPR. 10.17DT bound weakly to the DH270UCA3 chimeric antibodies containing natural CDR H3 loops, with binding levels less than 10% of that measured for the unmutated DH270UCA3 mAb. These results are in agreement with the substitution matrix analysis of naturally occurring CDR H3 loops, since all the loops tested experimentally contain at least two amino acids predicted to greatly reduce 10.17DT binding. Taken together, these results revealed that the natural B cell repertoire contains a high number of BCRs with CDR H3 loop sequence that permit engagement by CH505.M5.G458Y, while B cell activation by the 10.17DT immunogen will be significantly limited by the lack of BCRs containing CDR H3 loops expected to be bound by this immunogen. By analyzing a large collection of BCRs either isolated from the natural repertoire or generated synthetically, these methods can rapidly estimate the frequency of characteristic B cells that can be engaged by an immunogen upon vaccination.

### Immunogen Optimization to Recognize Diverse CDR H3 Loops of Naturally Occurring BCRs.

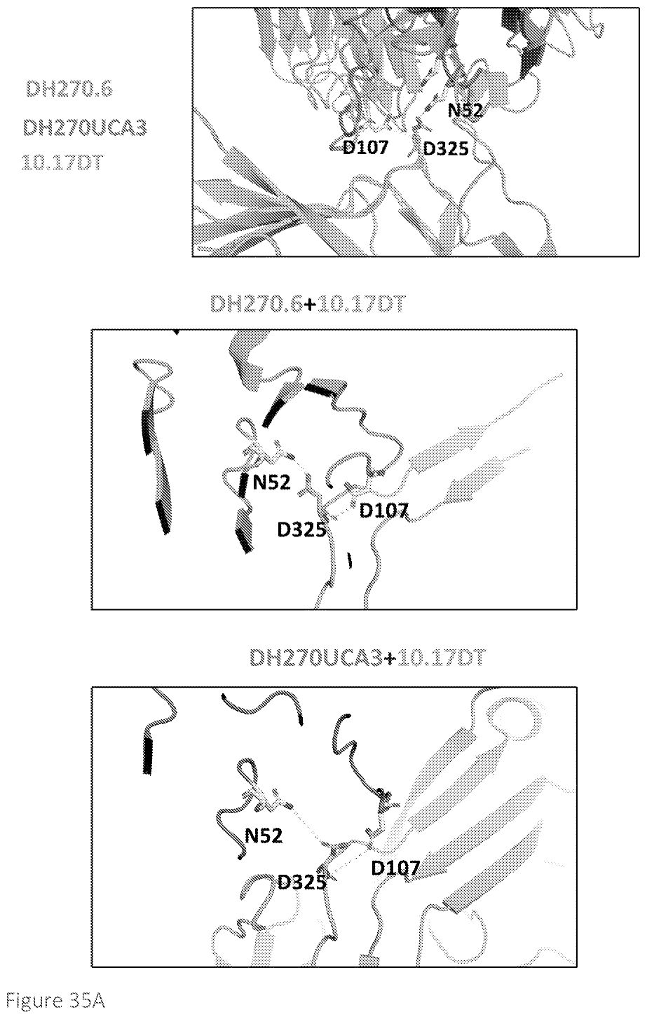

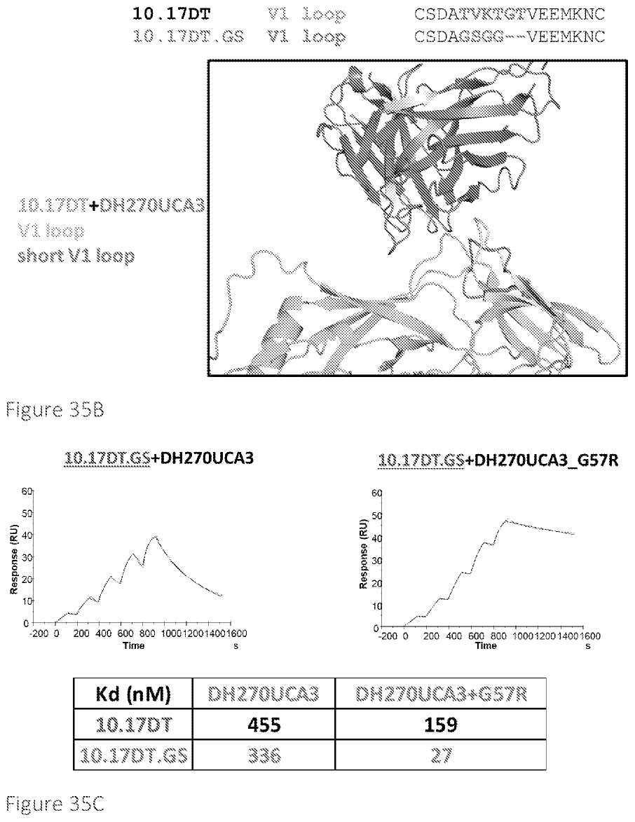

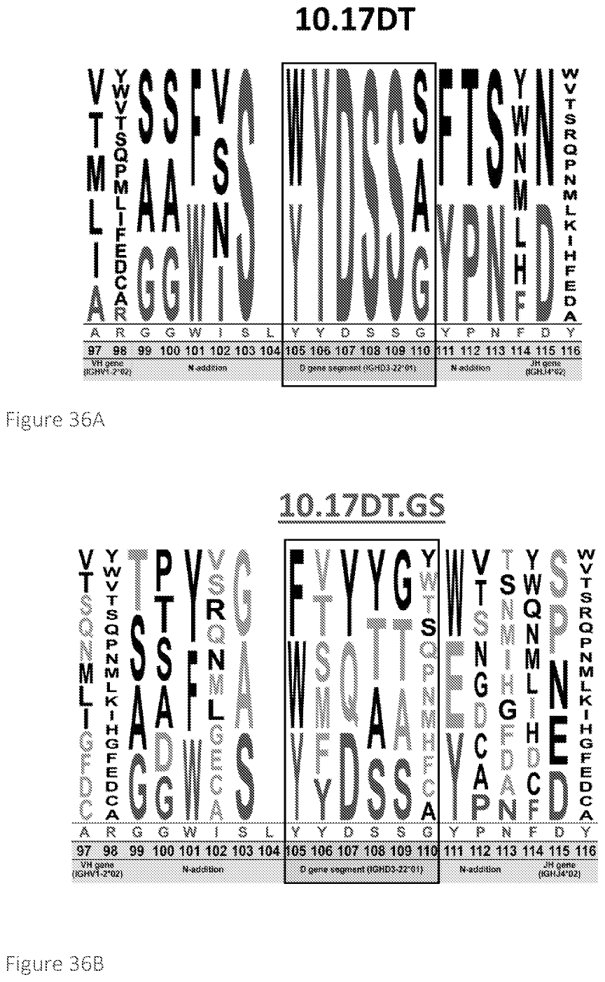

The goals of this project were to develop optimized 10.17DT immunogens with broader recognition of diverse CDR H3 loops FIGS. 35A-35C, 36A-36B show rationale and development of optimized 10.17DT immunogens with broader recognition of diverse CDR H3 loops—10.17DT.GS. In 10.17DT.GS the V1 loop is modified as shown in FIG. 35B. In FIGS. 36A and 36B position 104 was not interrogated.

Non-limiting embodiments of sequences comprising this modified V1 loop are shown in FIG. 29B. This modified V1 loop could be inserted in any other suitable envelope.

Given that 10.17DT was found to engage with low affinity only a limited number of CDR H3 loops present in naturally occurring BCRs, we next engineered this immunogen to increase its recognition of more diverse such loops. Our strategy was focused on improving the overall affinity of 10.17DT for DH270UCA3 and on developing molecules that may have different types of glycans present at position 332, a key interaction site for the CDR H3 loop.

It was previously shown that the V1 loop of 10.17DT interacts with the DH270UCA3. However, this loop rearranges away from the binding interface in DH270 lineage members beyond the UCA and does not contribute to binding by DH270.6, the lineage antibody with the broadest and most potent activity. We hypothesized that deletion of the V1 loop in 10.17DT may increase the accessibility of the epitope resulting in higher antibody affinity. Computational modeling with Rosetta was used to inform a two-residue deletion, such that the V1 was no longer expected to interact with the DH270UCA3. The resulting construct, called 10.17DT.GS had a “GSGG” linker connecting V1 residues 104 and 109. 10.17DT GS was expressed recombinantly and purified as SOSIP Env trimers. 10.17DT.GS showed higher binding to DH270UCA3 and to related antibodies that contain DH270.6 acquired mutations essential for binding and neutralization. See FIG. 35C.

This modified V1 loop immunogens bound with high affinity to an inferred lineage precursor, DH270UCA4, that contains a glycine substitution at position 103 in the CDR H3 loop of DH270UCA3. 10.17DT showed no binding to DH270UCA4 and tolerated no amino acids substitution at this site, suggesting that the novel immunogens may recognize more diverse CDR H3 loops.

The DH270UCA3 CDR H3 single site saturation mutagenesis library developed above was subsequently screened to identify clones that bind 10.17DT.GS. Upon sequence analysis, it was found that 10.17DT.GS bound a higher number of DH270UCA3 variants containing substitution in the CDR H3 loop compared to 10.17DT. At the CDR H3 sites encoded by the D gene and the non-templated nucleotide additions, 10.17DT.GS recognized an average of ˜6 alternate amino acids, a 3-fold increase over the number of variants bound by 10.17DT See FIG. 36A and FIG. 36B.

These studies demonstrate the development of a combined experimental-computational approach to identify natural CDR H3 loops that can be engaged by a candidate immunogen, the characterization of the ability of 10.17DT to engage DH270.6 precursors with diverse CDR H3 loops, and the development of a 10.17DT variant, 10.17DT.GS, that in vitro has broader recognition of CDR H3 loops related to that of DH270UCA3. Additional work to characterize 10.17DT.GS in vivo, immunize in DH270UCA3 and/or DH270UCA4 mice, and use sorts of (isolated) human B cell to validate increased recognition of precursors are envisioned.

Animal studies in any suitable model will be conducted to evaluate the immunogenicity of the 10.17DT.GS. Animal studies include testing the immunogen as recombinant trimer, nanoparticle and/or mRNA-LNP. Non-limiting embodiments of adjuvants include GLA-SE, alum, 3m052-SE or alum formulation, and/or LNPs. Animal studies include testing the immunogen as a prime, including multiple priming, and/or boost. Animal studies include testing the immunogen as a prime, could include boosting any suitable immunogen. Animal studies in mice could be conducted in DH270UCA3 and/or knock in mouse.

### Example 6—Animal Studies

Immunogens were tested in mouse study MU598. Immunogen CH848.3.D0949.10.17chim.6R.DS.SOSIP.664_N133D_GS135-40 was tested in DH270 UCA 4 VH+/−, VL+/− knock-in mice. 0.5 mcg 3M052-Alum was used as the adjuvant. FIG. 39A. Serum binding antibody responses following immunization with various envelopes, including CH848.3.D0949.10.17chim.6R.DS.SOSIP.664_N133D_GS135-40 were performed. FIGS. 39B-39R. The envelope with the “GS” modification is immunogenic in DH270 UCA 4 VH+/−, VL+/− knock-in mice. The serum has lower N332A levels of antibody versus N332 version (FIG. 39J), thus some of the antibodies in the serum are N332 dependent.

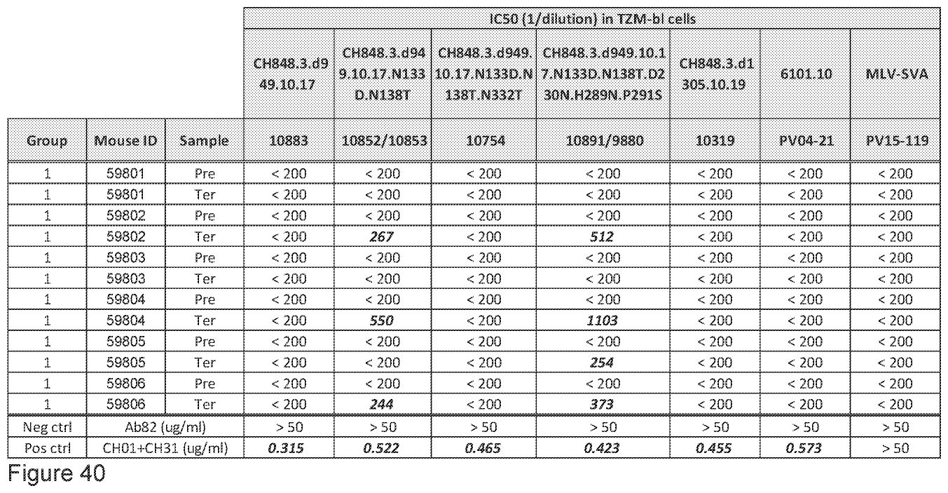

Neutralization studies were performed using TMZ-bl cells. Env-pseudotyped viruses produced by transfection in 293T cells were used. Samples were obtained at both pre-sera (“pre”) and terminal (“ter”) sera time points. Samples were heat-inactivated at 56 degrees Celsius for 15 minutes, diluted 1:10 in medium, and 11 microliters per well were used for 3-fold serial dilutions. Neutralization results elicited by the immunization regimen are depicted in FIG. 40. The results show MAb neutralization titer IC50 as mcg per mL required to inhibit 50% of virus replication. MLV-SVA is a negative control unrelated virus.

Off target antibodies (gp41, V2, V3) are sporadic and low.

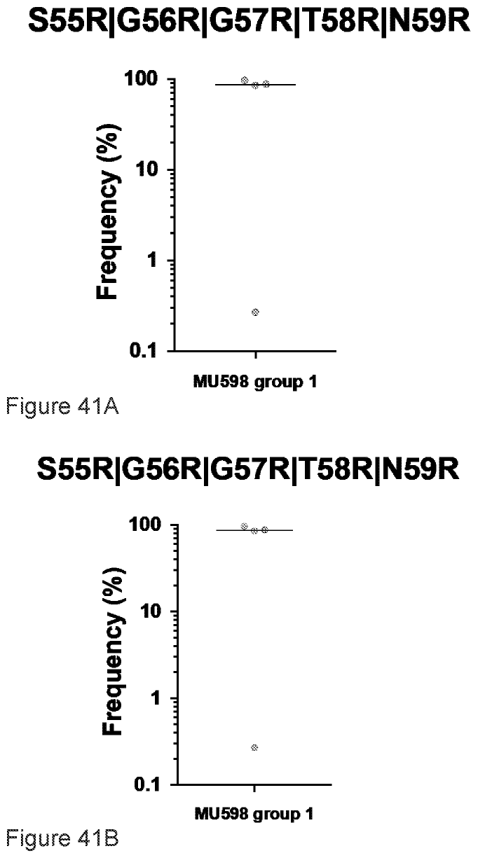

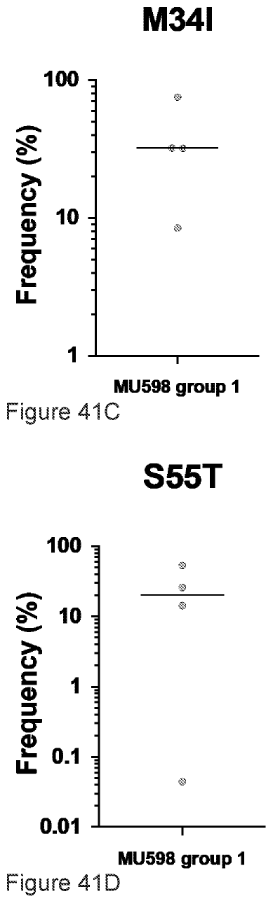

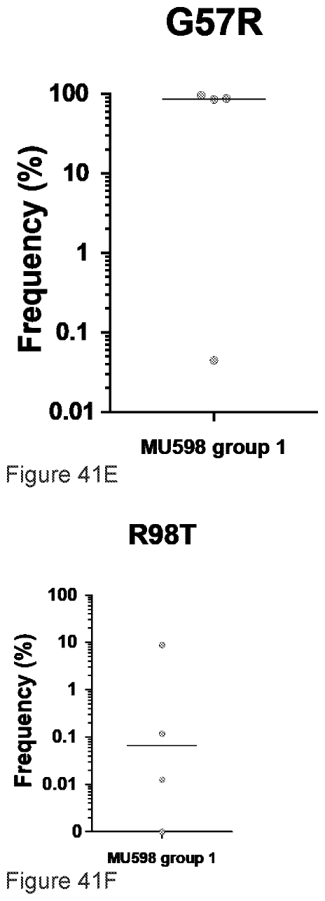

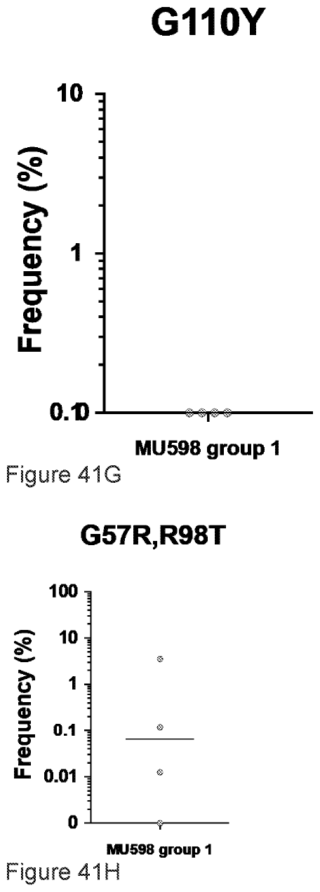

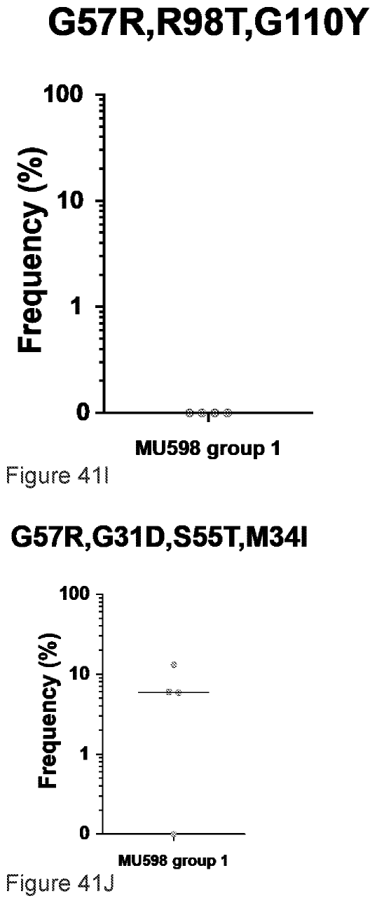

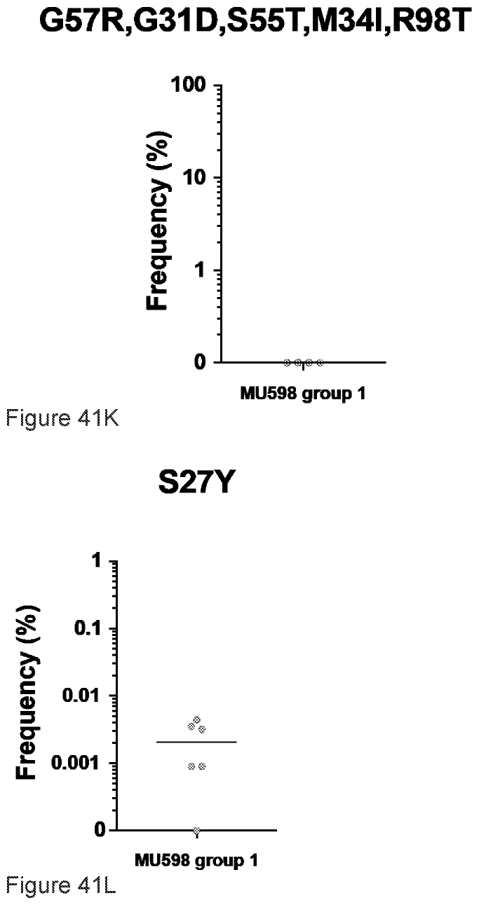

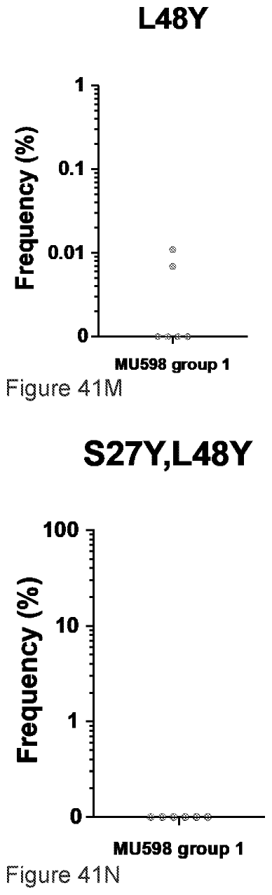

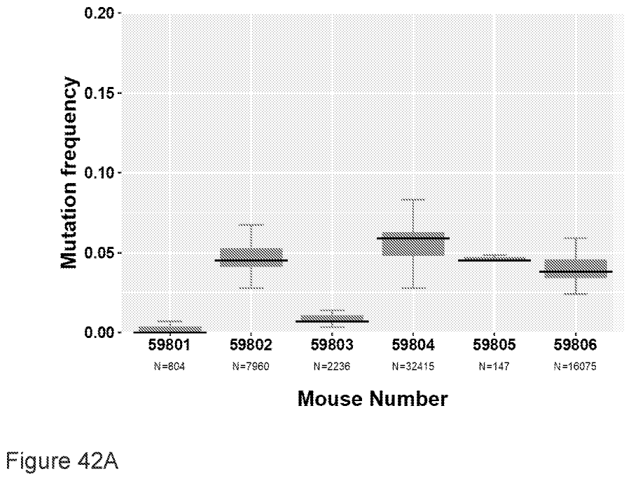

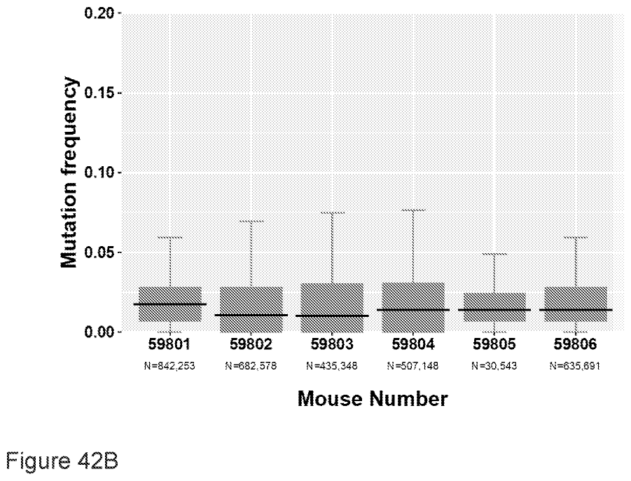

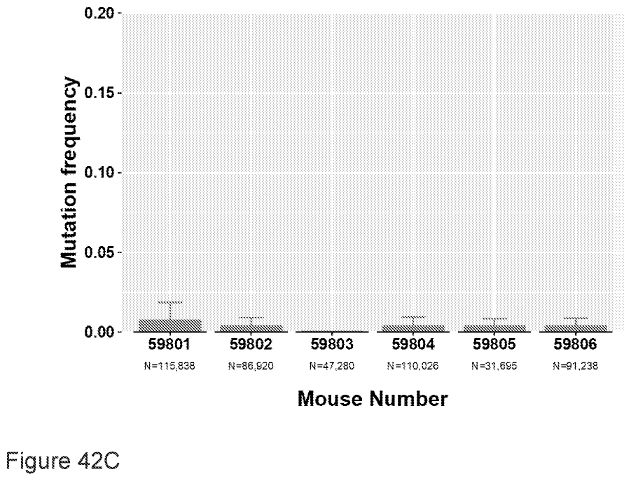

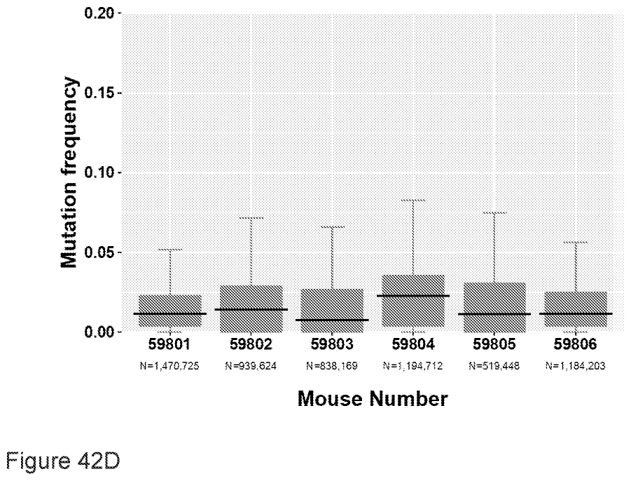

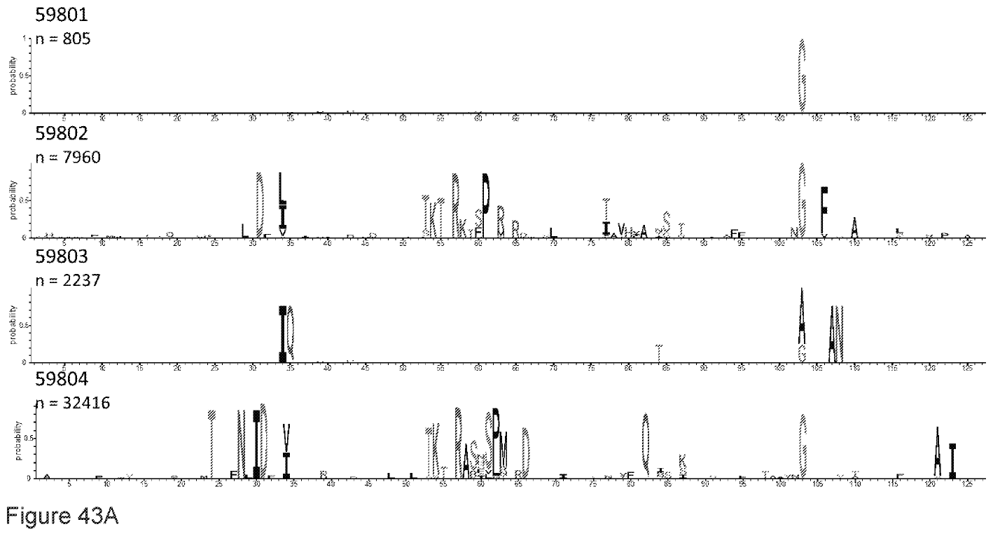

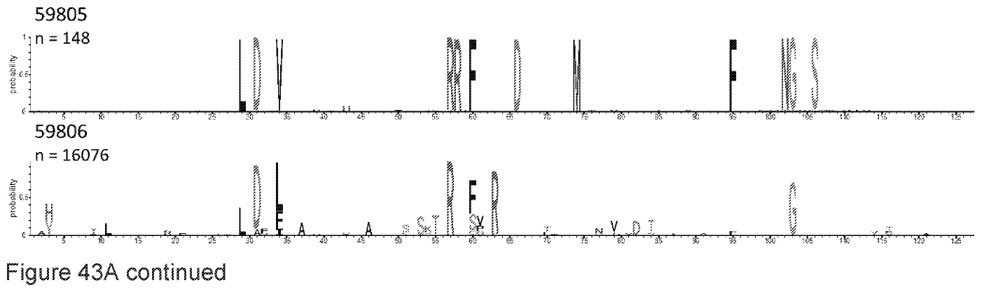

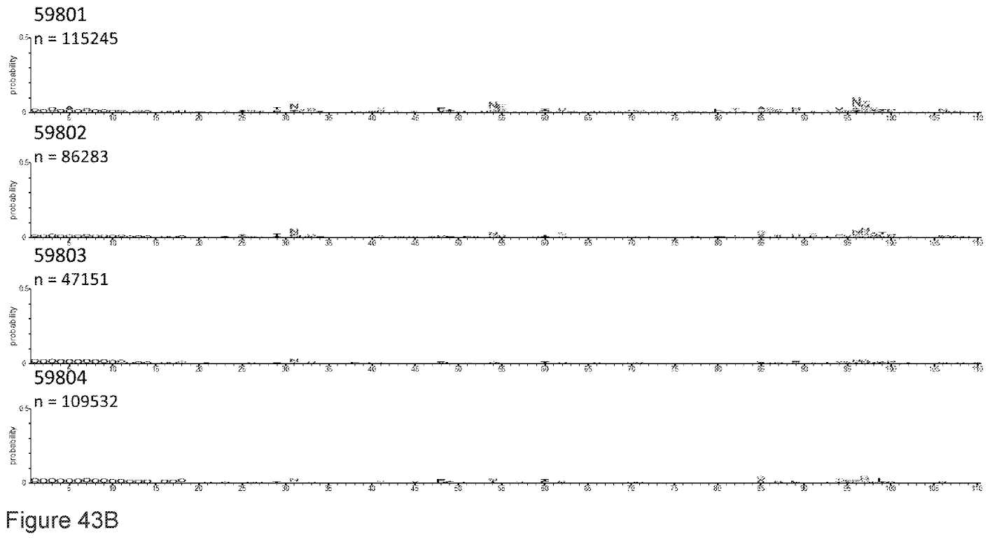

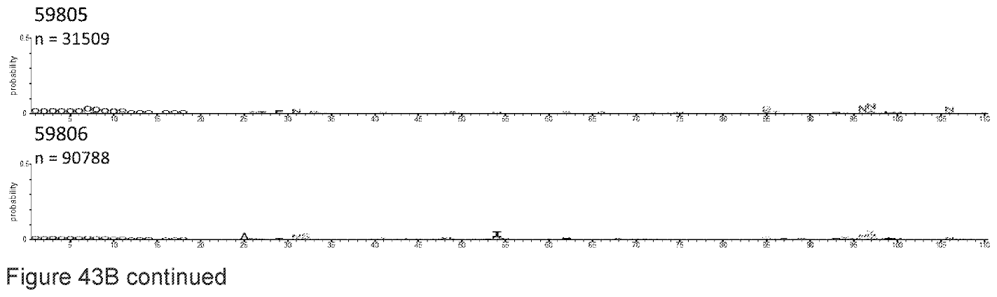

High throughput sequencing of heavy chain and light chain variable regions was performed on the mice to detect mutational frequency. FIGS. 41A-41N depict the frequency of specific amino acid sequences at the recited positions as inferred from nucleic acid sequencing of antibodies from the mice immunized per Group 1 of mouse study MU598 (see FIG. 39A). FIG. 42A-42D depicts mutation frequency in individual mice. Logo plots of the antibodies (both heavy and kappa chains) are depicted in FIGS. 43A-43B. FIGS. 44A and 44B depicts mutation frequencies of the V gene in the mice. This data shows that vaccination with an envelope comprising the GS mutation is inducing somatic mutation of DH270 antibodies and can select for improbable mutations G57R and R98T. Thus, vaccination is eliciting antibodies with the critical somatic mutations needed for broad neutralization.

1. A recombinant HIV-1 envelope selected from the envelope listed in Table 5, FIG. 29B.

2. A composition comprising the envelope of claim 1 and a carrier, wherein the envelope is a protomer comprised in a trimer.

3. The composition of claim 2, wherein the envelope is comprised in a stable trimer.

4. A composition comprising a nanoparticle and a carrier, wherein the nanoparticle comprises any one of the envelopes of claim 1.

5. The composition of claim 4, wherein the nanoparticle is ferritin self-assembling nanoparticle.

6. A composition comprising a nanoparticle and a carrier, wherein the nanoparticle comprises the trimer of claim 2.

7. The composition of claim 6 wherein the nanoparticle is ferritin self-assembling nanoparticle.

8. The composition of claim 7 wherein the nanoparticle comprises multimers of trimers.

9. The composition of claim 7 wherein the nanoparticle comprises 1-8 trimers.

10. A method of inducing an immune response in a subject comprising administering an immunogenic composition comprising any one of the recombinant envelope of claim 1, in an amount sufficient to induce an immune response.

11. The method of claim 10 wherein the composition is administered as a prime.

12. The method of claim 10 wherein the composition is administered as a boost.

13. A nucleic acid encoding the recombinant envelope of claim 1.

14. A composition comprising the nucleic acid of claim 13 and a carrier.

15. A method of inducing an immune response in a subject comprising administering an immunogenic composition comprising the nucleic acid of claim 13.

16. A method of inducing an immune response comprising administering an immunogenic composition comprising a prime immunogen from Table 5 followed by at least one boost immunogen from Table 3 or Table 4, wherein the boost immunogens are administered in the order appearing in Table 4, in an amount sufficient to induce an immune response.

17-25. (canceled)

26. The method of claim 16, wherein the prime or boost immunogen are administered as a nanoparticle.

27. The method of claim 16, wherein the nanoparticle is a ferritin nanoparticle.

28. The method of claim 16, wherein the prime or boost immunogen are administered as mRNA-LNP formulation.
